SEQUENCE LISTING (716 sequences)

SEQ ID NO: 1 - protein, 8 aa, Artificial Sequence
  Description of Artificial Sequence Synthetic peptide
STNTKLHA

SEQ ID NO: 2 - protein, 8 aa, Artificial Sequence
  Description of Artificial Sequence Synthetic peptide
SSDRTLRR

SEQ ID NO: 3 - protein, 8 aa, Artificial Sequence
  Description of Artificial Sequence Synthetic peptide
STKERLKT

SEQ ID NO: 4 - protein, 8 aa, Artificial Sequence
  Description of Artificial Sequence Synthetic peptide
SQRANLRA

SEQ ID NO: 5 - protein, 8 aa, Artificial Sequence
  Description of Artificial Sequence Synthetic peptide
SSPADLTR

SEQ ID NO: 6 - protein, 8 aa, Artificial Sequence
  Description of Artificial Sequence Synthetic peptide
SSHSDLVR

SEQ ID NO: 7 - protein, 8 aa, Artificial Sequence
  Description of Artificial Sequence Synthetic peptide
SNGGELIR

SEQ ID NO: 8 - protein, 8 aa, Artificial Sequence
  Description of Artificial Sequence Synthetic peptide
SNQLILLK

SEQ ID NO: 9 - protein, 8 aa, Artificial Sequence
  Description of Artificial Sequence Synthetic peptide
SSRMDLKR

SEQ ID NO: 10 - protein, 8 aa, Artificial Sequence
  Description of Artificial Sequence Synthetic peptide
SRSDHLTN

SEQ ID NO: 11 - protein, 8 aa, Artificial Sequence
  Description of Artificial Sequence Synthetic peptide
SQLAHLRA

SEQ ID NO: 12 - protein, 8 aa, Artificial Sequence
  Description of Artificial Sequence Synthetic peptide
SQASSLKA

SEQ ID NO: 13 - protein, 8 aa, Artificial Sequence
  Description of Artificial Sequence Synthetic peptide
SQKSSLIA

SEQ ID NO: 14 - protein, 8 aa, Artificial Sequence
  Description of Artificial Sequence Synthetic peptide
SRKDNLKN

SEQ ID NO: 15 - protein, 8 aa, Artificial Sequence
  Description of Artificial Sequence Synthetic peptide
SDSGNLRV

SEQ ID NO: 16 - protein, 8 aa, Artificial Sequence
  Description of Artificial Sequence Synthetic peptide
SDRRNLRR

SEQ ID NO: 17 - protein, 8 aa, Artificial Sequence
  Description of Artificial Sequence Synthetic peptide
SDKKDLSR

SEQ ID NO: 18 - protein, 8 aa, Artificial Sequence
  Description of Artificial Sequence Synthetic peptide
SDASHLHT

SEQ ID NO: 19 - protein, 8 aa, Artificial Sequence
  Description of Artificial Sequence Synthetic peptide
STNSGLKN

SEQ ID NO: 20 - protein, 8 aa, Artificial Sequence
  Description of Artificial Sequence Synthetic peptide
STRMSLST

SEQ ID NO: 21 - protein, 8 aa, Artificial Sequence
  Description of Artificial Sequence Synthetic peptide
SNHDALRA

SEQ ID NO: 22 - protein, 8 aa, Artificial Sequence
  Description of Artificial Sequence Synthetic peptide
SRRSACRR

SEQ ID NO: 23 - protein, 8 aa, Artificial Sequence
  Description of Artificial Sequence Synthetic peptide
SRRSSCRK

SEQ ID NO: 24 - protein, 8 aa, Artificial Sequence
  Description of Artificial Sequence Synthetic peptide
SRSDTLSN

SEQ ID NO: 25 - protein, 8 aa, Artificial Sequence
  Description of Artificial Sequence Synthetic peptide
SRMGNLIR

SEQ ID NO: 26 - protein, 8 aa, Artificial Sequence
  Description of Artificial Sequence Synthetic peptide
SRSDTLRD

SEQ ID NO: 27 - protein, 8 aa, Artificial Sequence
  Description of Artificial Sequence Synthetic peptide
SRAHDLVR

SEQ ID NO: 28 - protein, 8 aa, Artificial Sequence
  Description of Artificial Sequence Synthetic peptide
SRSDHLAE

SEQ ID NO: 29 - protein, 8 aa, Artificial Sequence
  Description of Artificial Sequence Synthetic peptide
SRRDALNV

SEQ ID NO: 30 - protein, 8 aa, Artificial Sequence
  Description of Artificial Sequence Synthetic peptide
STTGNLTV

SEQ ID NO: 31 - protein, 8 aa, Artificial Sequence
  Description of Artificial Sequence Synthetic peptide
STSGNLLV

SEQ ID NO: 32 - protein, 8 aa, Artificial Sequence
  Description of Artificial Sequence Synthetic peptide
STLTILKN

SEQ ID NO: 33 - protein, 8 aa, Artificial Sequence
  Description of Artificial Sequence Synthetic peptide
SRMSTLRH

SEQ ID NO: 34 - protein, 8 aa, Artificial Sequence
  Description of Artificial Sequence Synthetic peptide
STRSDLLR

SEQ ID NO: 35 - protein, 8 aa, Artificial Sequence
  Description of Artificial Sequence Synthetic peptide
STKTDLKR

SEQ ID NO: 36 - protein, 8 aa, Artificial Sequence
  Description of Artificial Sequence Synthetic peptide
STHIDLIR

SEQ ID NO: 37 - protein, 8 aa, Artificial Sequence
  Description of Artificial Sequence Synthetic peptide
SHRSTLLN

SEQ ID NO: 38 - protein, 8 aa, Artificial Sequence
  Description of Artificial Sequence Synthetic peptide
STSHGLTT

SEQ ID NO: 39 - protein, 8 aa, Artificial Sequence
  Description of Artificial Sequence Synthetic peptide
SHKNALQN

SEQ ID NO: 40 - protein, 7 aa, Artificial Sequence
  Description of Artificial Sequence Synthetic peptide
QRANLRA

SEQ ID NO: 41 - protein, 7 aa, Artificial Sequence
  Description of Artificial Sequence Synthetic peptide
DSGNLRV

SEQ ID NO: 42 - protein, 7 aa, Artificial Sequence
  Description of Artificial Sequence Synthetic peptide
RSDTLSN

SEQ ID NO: 43 - protein, 7 aa, Artificial Sequence
  Description of Artificial Sequence Synthetic peptide
TTGNLTV

SEQ ID NO: 44 - protein, 7 aa, Artificial Sequence
  Description of Artificial Sequence Synthetic peptide
SPADLTR

SEQ ID NO: 45 - protein, 7 aa, Artificial Sequence
  Description of Artificial Sequence Synthetic peptide
DKKDLTR

SEQ ID NO: 46 - protein, 7 aa, Artificial Sequence
  Description of Artificial Sequence Synthetic peptide
RTDTLRD

SEQ ID NO: 47 - protein, 7 aa, Artificial Sequence
  Description of Artificial Sequence Synthetic peptide
THLDLIR

SEQ ID NO: 48 - protein, 7 aa, Artificial Sequence
  Description of Artificial Sequence Synthetic peptide
QLAHLRA

SEQ ID NO: 49 - protein, 7 aa, Artificial Sequence
  Description of Artificial Sequence Synthetic peptide
RSDHLAE

SEQ ID NO: 50 - protein, 7 aa, Artificial Sequence
  Description of Artificial Sequence Synthetic peptide
HRTTLLN

SEQ ID NO: 51 - protein, 7 aa, Artificial Sequence
  Description of Artificial Sequence Synthetic peptide
QKSSLIA

SEQ ID NO: 52 - protein, 7 aa, Artificial Sequence
  Description of Artificial Sequence Synthetic peptide
RRDALNV

SEQ ID NO: 53 - protein, 7 aa, Artificial Sequence
  Description of Artificial Sequence Synthetic peptide
HKNALQN

SEQ ID NO: 54 - protein, 7 aa, Artificial Sequence
  Description of Artificial Sequence Synthetic peptide
RSDNLSN

SEQ ID NO: 55 - protein, 7 aa, Artificial Sequence
  Description of Artificial Sequence Synthetic peptide
RKDNLKN

SEQ ID NO: 56 - protein, 7 aa, Artificial Sequence
  Description of Artificial Sequence Synthetic peptide
TSGNLLV

SEQ ID NO: 57 - protein, 7 aa, Artificial Sequence
  Description of Artificial Sequence Synthetic peptide
RSDHLTN

SEQ ID NO: 58 - protein, 7 aa, Artificial Sequence
  Description of Artificial Sequence Synthetic peptide
HRTTLTN

SEQ ID NO: 59 - protein, 7 aa, Artificial Sequence
  Description of Artificial Sequence Synthetic peptide
SHSDLVR

SEQ ID NO: 60 - protein, 7 aa, Artificial Sequence
  Description of Artificial Sequence Synthetic peptide
NGGELIR

SEQ ID NO: 61 - protein, 7 aa, Artificial Sequence
  Description of Artificial Sequence Synthetic peptide
STKDLKR

SEQ ID NO: 62 - protein, 7 aa, Artificial Sequence
  Description of Artificial Sequence Synthetic peptide
RRDELNV

SEQ ID NO: 63 - protein, 7 aa, Artificial Sequence
  Description of Artificial Sequence Synthetic peptide
QASSLKA

SEQ ID NO: 64 - protein, 7 aa, Artificial Sequence
  Description of Artificial Sequence Synthetic peptide
TSHGLTT

SEQ ID NO: 65 - protein, 7 aa, Artificial Sequence
  Description of Artificial Sequence Synthetic peptide
QSSHLVR

SEQ ID NO: 66 - protein, 7 aa, Artificial Sequence
  Description of Artificial Sequence Synthetic peptide
QSSNLVR

SEQ ID NO: 67 - protein, 7 aa, Artificial Sequence
  Description of Artificial Sequence Synthetic peptide
DPGALRV

SEQ ID NO: 68 - protein, 7 aa, Artificial Sequence
  Description of Artificial Sequence Synthetic peptide
RSDNLVR

SEQ ID NO: 69 - protein, 7 aa, Artificial Sequence
  Description of Artificial Sequence Synthetic peptide
QSGDLRR

SEQ ID NO: 70 - protein, 7 aa, Artificial Sequence
  Description of Artificial Sequence Synthetic peptide
DCRDLAR

SEQ ID NO: 71 - protein, 7 aa, Artificial Sequence
  Description of Artificial Sequence Synthetic peptide
DPGALIN

SEQ ID NO: 72 - protein, 7 aa, Artificial Sequence
  Description of Artificial Sequence Synthetic peptide
ERSHLRE

SEQ ID NO: 73 - protein, 7 aa, Artificial Sequence
  Description of Artificial Sequence Synthetic peptide
DPGHLTE

SEQ ID NO: 74 - protein, 7 aa, Artificial Sequence
  Description of Artificial Sequence Synthetic peptide
EPGALIN

SEQ ID NO: 75 - protein, 7 aa, Artificial Sequence
  Description of Artificial Sequence Synthetic peptide
DRSHLRE

SEQ ID NO: 76 - protein, 7 aa, Artificial Sequence
  Description of Artificial Sequence Synthetic peptide
EPGHLTE

SEQ ID NO: 77 - protein, 7 aa, Artificial Sequence
  Description of Artificial Sequence Synthetic peptide
ERSLLRE

SEQ ID NO: 78 - protein, 7 aa, Artificial Sequence
  Description of Artificial Sequence Synthetic peptide
DRSKLRE

SEQ ID NO: 79 - protein, 7 aa, Artificial Sequence
  Description of Artificial Sequence Synthetic peptide
DPGKLTE

SEQ ID NO: 80 - protein, 7 aa, Artificial Sequence
  Description of Artificial Sequence Synthetic peptide
EPGKLTE

SEQ ID NO: 81 - protein, 7 aa, Artificial Sequence
  Description of Artificial Sequence Synthetic peptide
DPGWLIN

SEQ ID NO: 82 - protein, 7 aa, Artificial Sequence
  Description of Artificial Sequence Synthetic peptide
DPGTLIN

SEQ ID NO: 83 - protein, 7 aa, Artificial Sequence
  Description of Artificial Sequence Synthetic peptide
DPGHLIN

SEQ ID NO: 84 - protein, 7 aa, Artificial Sequence
  Description of Artificial Sequence Synthetic peptide
ERSWLIN

SEQ ID NO: 85 - protein, 7 aa, Artificial Sequence
  Description of Artificial Sequence Synthetic peptide
ERSTLIN

SEQ ID NO: 86 - protein, 7 aa, Artificial Sequence
  Description of Artificial Sequence Synthetic peptide
DPGWLTE

SEQ ID NO: 87 - protein, 7 aa, Artificial Sequence
  Description of Artificial Sequence Synthetic peptide
DPGTLTE

SEQ ID NO: 88 - protein, 7 aa, Artificial Sequence
  Description of Artificial Sequence Synthetic peptide
EPGWLIN

SEQ ID NO: 89 - protein, 7 aa, Artificial Sequence
  Description of Artificial Sequence Synthetic peptide
EPGTLIN

SEQ ID NO: 90 - protein, 7 aa, Artificial Sequence
  Description of Artificial Sequence Synthetic peptide
EPGHLIN

SEQ ID NO: 91 - protein, 7 aa, Artificial Sequence
  Description of Artificial Sequence Synthetic peptide
DRSWLRE

SEQ ID NO: 92 - protein, 7 aa, Artificial Sequence
  Description of Artificial Sequence Synthetic peptide
DRSTLRE

SEQ ID NO: 93 - protein, 7 aa, Artificial Sequence
  Description of Artificial Sequence Synthetic peptide
EPGWLTE

SEQ ID NO: 94 - protein, 7 aa, Artificial Sequence
  Description of Artificial Sequence Synthetic peptide
EPGTLTE

SEQ ID NO: 95 - protein, 7 aa, Artificial Sequence
  Description of Artificial Sequence Synthetic peptide
ERSWLRE

SEQ ID NO: 96 - protein, 7 aa, Artificial Sequence
  Description of Artificial Sequence Synthetic peptide
ERSTLRE

SEQ ID NO: 97 - protein, 7 aa, Artificial Sequence
  Description of Artificial Sequence Synthetic peptide
DPGALRE

SEQ ID NO: 98 - protein, 7 aa, Artificial Sequence
  Description of Artificial Sequence Synthetic peptide
DPGALTE

SEQ ID NO: 99 - protein, 7 aa, Artificial Sequence
  Description of Artificial Sequence Synthetic peptide
ERSHLIN

SEQ ID NO: 100 - protein, 7 aa, Artificial Sequence
  Description of Artificial Sequence Synthetic peptide
ERSHLTE

SEQ ID NO: 101 - protein, 7 aa, Artificial Sequence
  Description of Artificial Sequence Synthetic peptide
DPGHLIN

SEQ ID NO: 102 - protein, 7 aa, Artificial Sequence
  Description of Artificial Sequence Synthetic peptide
DPGHLRE

SEQ ID NO: 103 - protein, 7 aa, Artificial Sequence
  Description of Artificial Sequence Synthetic peptide
EPGALRE

SEQ ID NO: 104 - protein, 7 aa, Artificial Sequence
  Description of Artificial Sequence Synthetic peptide
EPGALTE

SEQ ID NO: 105 - protein, 7 aa, Artificial Sequence
  Description of Artificial Sequence Synthetic peptide
DRSHLIN

SEQ ID NO: 106 - protein, 7 aa, Artificial Sequence
  Description of Artificial Sequence Synthetic peptide
DRSHLTE

SEQ ID NO: 107 - protein, 7 aa, Artificial Sequence
  Description of Artificial Sequence Synthetic peptide
EPGHLRE

SEQ ID NO: 108 - protein, 7 aa, Artificial Sequence
  Description of Artificial Sequence Synthetic peptide
ERSKLIN

SEQ ID NO: 109 - protein, 7 aa, Artificial Sequence
  Description of Artificial Sequence Synthetic peptide
ERSKLTE

SEQ ID NO: 110 - protein, 7 aa, Artificial Sequence
  Description of Artificial Sequence Synthetic peptide
DRSKLIN

SEQ ID NO: 111 - protein, 7 aa, Artificial Sequence
  Description of Artificial Sequence Synthetic peptide
DRSKLTE

SEQ ID NO: 112 - protein, 7 aa, Artificial Sequence
  Description of Artificial Sequence Synthetic peptide
DPGKLIN

SEQ ID NO: 113 - protein, 7 aa, Artificial Sequence
  Description of Artificial Sequence Synthetic peptide
DPGKLRE

SEQ ID NO: 114 - protein, 7 aa, Artificial Sequence
  Description of Artificial Sequence Synthetic peptide
EPGKLIN

SEQ ID NO: 115 - protein, 7 aa, Artificial Sequence
  Description of Artificial Sequence Synthetic peptide
EPGKLRE

SEQ ID NO: 116 - protein, 7 aa, Artificial Sequence
  Description of Artificial Sequence Synthetic peptide
DPGWLRE

SEQ ID NO: 117 - protein, 7 aa, Artificial Sequence
  Description of Artificial Sequence Synthetic peptide
DPGTLRE

SEQ ID NO: 118 - protein, 7 aa, Artificial Sequence
  Description of Artificial Sequence Synthetic peptide
DPGHLRE

SEQ ID NO: 119 - protein, 7 aa, Artificial Sequence
  Description of Artificial Sequence Synthetic peptide
DPGHLTE

SEQ ID NO: 120 - protein, 7 aa, Artificial Sequence
  Description of Artificial Sequence Synthetic peptide
ERSWLTE

SEQ ID NO: 121 - protein, 7 aa, Artificial Sequence
  Description of Artificial Sequence Synthetic peptide
ERSTLTE

SEQ ID NO: 122 - protein, 7 aa, Artificial Sequence
  Description of Artificial Sequence Synthetic peptide
EPGWLRE

SEQ ID NO: 123 - protein, 7 aa, Artificial Sequence
  Description of Artificial Sequence Synthetic peptide
EPGTLRE

SEQ ID NO: 124 - protein, 7 aa, Artificial Sequence
  Description of Artificial Sequence Synthetic peptide
DRSWLIN

SEQ ID NO: 125 - protein, 7 aa, Artificial Sequence
  Description of Artificial Sequence Synthetic peptide
DRSWLTE

SEQ ID NO: 126 - protein, 7 aa, Artificial Sequence
  Description of Artificial Sequence Synthetic peptide
DRSTLIN

SEQ ID NO: 127 - protein, 7 aa, Artificial Sequence
  Description of Artificial Sequence Synthetic peptide
DRSTLTE

SEQ ID NO: 128 - protein, 7 aa, Artificial Sequence
  Description of Artificial Sequence Synthetic peptide
QRHNLTE

SEQ ID NO: 129 - protein, 7 aa, Artificial Sequence
  Description of Artificial Sequence Synthetic peptide
QSGNLTE

SEQ ID NO: 130 - protein, 7 aa, Artificial Sequence
  Description of Artificial Sequence Synthetic peptide
NLQHLGE

SEQ ID NO: 131 - protein, 7 aa, Artificial Sequence
  Description of Artificial Sequence Synthetic peptide
RADNLTE

SEQ ID NO: 132 - protein, 7 aa, Artificial Sequence
  Description of Artificial Sequence Synthetic peptide
RADNLAI

SEQ ID NO: 133 - protein, 7 aa, Artificial Sequence
  Description of Artificial Sequence Synthetic peptide
NTTHLEH

SEQ ID NO: 134 - protein, 7 aa, Artificial Sequence
  Description of Artificial Sequence Synthetic peptide
SKKHLAE

SEQ ID NO: 135 - protein, 7 aa, Artificial Sequence
  Description of Artificial Sequence Synthetic peptide
RNDTLTE

SEQ ID NO: 136 - protein, 7 aa, Artificial Sequence
  Description of Artificial Sequence Synthetic peptide
RNDTLQA

SEQ ID NO: 137 - protein, 7 aa, Artificial Sequence
  Description of Artificial Sequence Synthetic peptide
QSGHLTE

SEQ ID NO: 138 - protein, 7 aa, Artificial Sequence
  Description of Artificial Sequence Synthetic peptide
QLAHLKE

SEQ ID NO: 139 - protein, 7 aa, Artificial Sequence
  Description of Artificial Sequence Synthetic peptide
QRAHLTE

SEQ ID NO: 140 - protein, 7 aa, Artificial Sequence
  Description of Artificial Sequence Synthetic peptide
HTGHLLE

SEQ ID NO: 141 - protein, 7 aa, Artificial Sequence
  Description of Artificial Sequence Synthetic peptide
RSDHLTE

SEQ ID NO: 142 - protein, 7 aa, Artificial Sequence
  Description of Artificial Sequence Synthetic peptide
RSDKLTE

SEQ ID NO: 143 - protein, 7 aa, Artificial Sequence
  Description of Artificial Sequence Synthetic peptide
RSDHLTD

SEQ ID NO: 144 - protein, 7 aa, Artificial Sequence
  Description of Artificial Sequence Synthetic peptide
RSDHLTN

SEQ ID NO: 145 - protein, 7 aa, Artificial Sequence
  Description of Artificial Sequence Synthetic peptide
SRRTCRA

SEQ ID NO: 146 - protein, 7 aa, Artificial Sequence
  Description of Artificial Sequence Synthetic peptide
QLRHLRE

SEQ ID NO: 147 - protein, 7 aa, Artificial Sequence
  Description of Artificial Sequence Synthetic peptide
QRHSLTE

SEQ ID NO: 148 - protein, 7 aa, Artificial Sequence
  Description of Artificial Sequence Synthetic peptide
QLAHLKR

SEQ ID NO: 149 - protein, 7 aa, Artificial Sequence
  Description of Artificial Sequence Synthetic peptide
NLQHLGE

SEQ ID NO: 150 - protein, 7 aa, Artificial Sequence
  Description of Artificial Sequence Synthetic peptide
RNDALTE

SEQ ID NO: 151 - protein, 7 aa, Artificial Sequence
  Description of Artificial Sequence Synthetic peptide
TKQTLTE

SEQ ID NO: 152 - protein, 7 aa, Artificial Sequence
  Description of Artificial Sequence Synthetic peptide
QSGDLTE

SEQ ID NO: 153 - protein, 7 aa, Artificial Sequence
  Description of Artificial Sequence Synthetic peptide
QSSNLVR

SEQ ID NO: 154 - protein, 7 aa, Artificial Sequence
  Description of Artificial Sequence Synthetic peptide
DPGNLVR

SEQ ID NO: 155 - protein, 7 aa, Artificial Sequence
  Description of Artificial Sequence Synthetic peptide
RSDNLVR

SEQ ID NO: 156 - protein, 7 aa, Artificial Sequence
  Description of Artificial Sequence Synthetic peptide
TSGNLVR

SEQ ID NO: 157 - protein, 7 aa, Artificial Sequence
  Description of Artificial Sequence Synthetic peptide
QSGDLRR

SEQ ID NO: 158 - protein, 7 aa, Artificial Sequence
  Description of Artificial Sequence Synthetic peptide
DCRDLAR

SEQ ID NO: 159 - protein, 7 aa, Artificial Sequence
  Description of Artificial Sequence Synthetic peptide
RSDDLVK

SEQ ID NO: 160 - protein, 7 aa, Artificial Sequence
  Description of Artificial Sequence Synthetic peptide
TSGELVR

SEQ ID NO: 161 - protein, 7 aa, Artificial Sequence
  Description of Artificial Sequence Synthetic peptide
QRAHLER

SEQ ID NO: 162 - protein, 7 aa, Artificial Sequence
  Description of Artificial Sequence Synthetic peptide
DPGHLVR

SEQ ID NO: 163 - protein, 7 aa, Artificial Sequence
  Description of Artificial Sequence Synthetic peptide
RSDKLVR

SEQ ID NO: 164 - protein, 7 aa, Artificial Sequence
  Description of Artificial Sequence Synthetic peptide
TSGHLVR

SEQ ID NO: 165 - protein, 7 aa, Artificial Sequence
  Description of Artificial Sequence Synthetic peptide
QSSSLVR

SEQ ID NO: 166 - protein, 7 aa, Artificial Sequence
  Description of Artificial Sequence Synthetic peptide
DPGALVR

SEQ ID NO: 167 - protein, 7 aa, Artificial Sequence
  Description of Artificial Sequence Synthetic peptide
RSDELVR

SEQ ID NO: 168 - protein, 7 aa, Artificial Sequence
  Description of Artificial Sequence Synthetic peptide
TSGSLVR

SEQ ID NO: 169 - protein, 7 aa, Artificial Sequence
  Description of Artificial Sequence Synthetic peptide
QRSNLVR

SEQ ID NO: 170 - protein, 7 aa, Artificial Sequence
  Description of Artificial Sequence Synthetic peptide
QSGNLVR

SEQ ID NO: 171 - protein, 7 aa, Artificial Sequence
  Description of Artificial Sequence Synthetic peptide
QPGNLVR

SEQ ID NO: 172 - protein, 7 aa, Artificial Sequence
  Description of Artificial Sequence Synthetic peptide
DPGNLKR

SEQ ID NO: 173 - protein, 7 aa, Artificial Sequence
  Description of Artificial Sequence Synthetic peptide
RSDNLRR

SEQ ID NO: 174 - protein, 7 aa, Artificial Sequence
  Description of Artificial Sequence Synthetic peptide
KSANLVR

SEQ ID NO: 175 - protein, 7 aa, Artificial Sequence
  Description of Artificial Sequence Synthetic peptide
RSDNLVK

SEQ ID NO: 176 - protein, 7 aa, Artificial Sequence
  Description of Artificial Sequence Synthetic peptide
KSAQLVR

SEQ ID NO: 177 - protein, 7 aa, Artificial Sequence
  Description of Artificial Sequence Synthetic peptide
QSSTLVR

SEQ ID NO: 178 - protein, 7 aa, Artificial Sequence
  Description of Artificial Sequence Synthetic peptide
QSGTLRR

SEQ ID NO: 179 - protein, 7 aa, Artificial Sequence
  Description of Artificial Sequence Synthetic peptide
QPGDLVR

SEQ ID NO: 180 - protein, 7 aa, Artificial Sequence
  Description of Artificial Sequence Synthetic peptide
QGPDLVR

SEQ ID NO: 181 - protein, 7 aa, Artificial Sequence
  Description of Artificial Sequence Synthetic peptide
QAGTLMR

SEQ ID NO: 182 - protein, 7 aa, Artificial Sequence
  Description of Artificial Sequence Synthetic peptide
QPGTLVR

SEQ ID NO: 183 - protein, 7 aa, Artificial Sequence
  Description of Artificial Sequence Synthetic peptide
QGPELVR

SEQ ID NO: 184 - protein, 7 aa, Artificial Sequence
  Description of Artificial Sequence Synthetic peptide
GCRELSR

SEQ ID NO: 185 - protein, 7 aa, Artificial Sequence
  Description of Artificial Sequence Synthetic peptide
DPSTLKR

SEQ ID NO: 186 - protein, 7 aa, Artificial Sequence
  Description of Artificial Sequence Synthetic peptide
DPSDLKR

SEQ ID NO: 187 - protein, 7 aa, Artificial Sequence
  Description of Artificial Sequence Synthetic peptide
DSGDLVR

SEQ ID NO: 188 - protein, 7 aa, Artificial Sequence
  Description of Artificial Sequence Synthetic peptide
DSGELVR

SEQ ID NO: 189 - protein, 7 aa, Artificial Sequence
  Description of Artificial Sequence Synthetic peptide
DSGELKR

SEQ ID NO: 190 - protein, 7 aa, Artificial Sequence
  Description of Artificial Sequence Synthetic peptide
RLDTLGR

SEQ ID NO: 191 - protein, 7 aa, Artificial Sequence
  Description of Artificial Sequence Synthetic peptide
RPGDLVR

SEQ ID NO: 192 - protein, 7 aa, Artificial Sequence
  Description of Artificial Sequence Synthetic peptide
RSDTLVR

SEQ ID NO: 193 - protein, 7 aa, Artificial Sequence
  Description of Artificial Sequence Synthetic peptide
KSADLKR

SEQ ID NO: 194 - protein, 7 aa, Artificial Sequence
  Description of Artificial Sequence Synthetic peptide
RSDDLVR

SEQ ID NO: 195 - protein, 7 aa, Artificial Sequence
  Description of Artificial Sequence Synthetic peptide
RSDTLVK

SEQ ID NO: 196 - protein, 7 aa, Artificial Sequence
  Description of Artificial Sequence Synthetic peptide
KSAELKR

SEQ ID NO: 197 - protein, 7 aa, Artificial Sequence
  Description of Artificial Sequence Synthetic peptide
KSAELVR

SEQ ID NO: 198 - protein, 7 aa, Artificial Sequence
  Description of Artificial Sequence Synthetic peptide
RGPELVR

SEQ ID NO: 199 - protein, 7 aa, Artificial Sequence
  Description of Artificial Sequence Synthetic peptide
KPGELVR

SEQ ID NO: 200 - protein, 7 aa, Artificial Sequence
  Description of Artificial Sequence Synthetic peptide
SSQTLTR

SEQ ID NO: 201 - protein, 7 aa, Artificial Sequence
  Description of Artificial Sequence Synthetic peptide
TPGELVR

SEQ ID NO: 202 - protein, 7 aa, Artificial Sequence
  Description of Artificial Sequence Synthetic peptide
TSGDLVR

SEQ ID NO: 203 - protein, 7 aa, Artificial Sequence
  Description of Artificial Sequence Synthetic peptide
SSQTLVR

SEQ ID NO: 204 - protein, 7 aa, Artificial Sequence
  Description of Artificial Sequence Synthetic peptide
TSQTLTR

SEQ ID NO: 205 - protein, 7 aa, Artificial Sequence
  Description of Artificial Sequence Synthetic peptide
TSGELKR

SEQ ID NO: 206 - protein, 7 aa, Artificial Sequence
  Description of Artificial Sequence Synthetic peptide
QSSDLVR

SEQ ID NO: 207 - protein, 7 aa, Artificial Sequence
  Description of Artificial Sequence Synthetic peptide
SSGTLVR

SEQ ID NO: 208 - protein, 7 aa, Artificial Sequence
  Description of Artificial Sequence Synthetic peptide
TPGTLVR

SEQ ID NO: 209 - protein, 7 aa, Artificial Sequence
  Description of Artificial Sequence Synthetic peptide
TSQDLKR

SEQ ID NO: 210 - protein, 7 aa, Artificial Sequence
  Description of Artificial Sequence Synthetic peptide
TSGTLVR

SEQ ID NO: 211 - protein, 7 aa, Artificial Sequence
  Description of Artificial Sequence Synthetic peptide
QSSHLVR

SEQ ID NO: 212 - protein, 7 aa, Artificial Sequence
  Description of Artificial Sequence Synthetic peptide
QSGHLVR

SEQ ID NO: 213 - protein, 7 aa, Artificial Sequence
  Description of Artificial Sequence Synthetic peptide
QPGHLVR

SEQ ID NO: 214 - protein, 7 aa, Artificial Sequence
  Description of Artificial Sequence Synthetic peptide
ERSKLAR

SEQ ID NO: 215 - protein, 7 aa, Artificial Sequence
  Description of Artificial Sequence Synthetic peptide
DPGHLAR

SEQ ID NO: 216 - protein, 7 aa, Artificial Sequence
  Description of Artificial Sequence Synthetic peptide
QRAKLER

SEQ ID NO: 217 - protein, 7 aa, Artificial Sequence
  Description of Artificial Sequence Synthetic peptide
QSSKLVR

SEQ ID NO: 218 - protein, 7 aa, Artificial Sequence
  Description of Artificial Sequence Synthetic peptide
DRSKLAR

SEQ ID NO: 219 - protein, 7 aa, Artificial Sequence
  Description of Artificial Sequence Synthetic peptide
DPGKLAR

SEQ ID NO: 220 - protein, 7 aa, Artificial Sequence
  Description of Artificial Sequence Synthetic peptide
RSKDLTR

SEQ ID NO: 221 - protein, 7 aa, Artificial Sequence
  Description of Artificial Sequence Synthetic peptide
RSDHLTR

SEQ ID NO: 222 - protein, 7 aa, Artificial Sequence
  Description of Artificial Sequence Synthetic peptide
KSAKLER

SEQ ID NO: 223 - protein, 7 aa, Artificial Sequence
  Description of Artificial Sequence Synthetic peptide
TADHLSR

SEQ ID NO: 224 - protein, 7 aa, Artificial Sequence
  Description of Artificial Sequence Synthetic peptide
TADKLSR

SEQ ID NO: 225 - protein, 7 aa, Artificial Sequence
  Description of Artificial Sequence Synthetic peptide
TPGHLVR

SEQ ID NO: 226 - protein, 7 aa, Artificial Sequence
  Description of Artificial Sequence Synthetic peptide
TSSHLVR

SEQ ID NO: 227 - protein, 7 aa, Artificial Sequence
  Description of Artificial Sequence Synthetic peptide
TSGKLVR

SEQ ID NO: 228 - protein, 7 aa, Artificial Sequence
  Description of Artificial Sequence Synthetic peptide
QPGELVR

SEQ ID NO: 229 - protein, 7 aa, Artificial Sequence
  Description of Artificial Sequence Synthetic peptide
QSGELVR

SEQ ID NO: 230 - protein, 7 aa, Artificial Sequence
  Description of Artificial Sequence Synthetic peptide
QSGELRR

SEQ ID NO: 231 - protein, 7 aa, Artificial Sequence
  Description of Artificial Sequence Synthetic peptide
DPGSLVR

SEQ ID NO: 232 - protein, 7 aa, Artificial Sequence
  Description of Artificial Sequence Synthetic peptide
RKDSLVR

SEQ ID NO: 233 - protein, 7 aa, Artificial Sequence
  Description of Artificial Sequence Synthetic peptide
RSDVLVR

SEQ ID NO: 234 - protein, 7 aa, Artificial Sequence
  Description of Artificial Sequence Synthetic peptide
RHDSLLR

SEQ ID NO: 235 - protein, 7 aa, Artificial Sequence
  Description of Artificial Sequence Synthetic peptide
RSDALVR

SEQ ID NO: 236 - protein, 7 aa, Artificial Sequence
  Description of Artificial Sequence Synthetic peptide
RSSSLVR

SEQ ID NO: 237 - protein, 7 aa, Artificial Sequence
  Description of Artificial Sequence Synthetic peptide
RSSSHVR

SEQ ID NO: 238 - protein, 7 aa, Artificial Sequence
  Description of Artificial Sequence Synthetic peptide
RSDELVK

SEQ ID NO: 239 - protein, 7 aa, Artificial Sequence
  Description of Artificial Sequence Synthetic peptide
RSDALVK

SEQ ID NO: 240 - protein, 7 aa, Artificial Sequence
  Description of Artificial Sequence Synthetic peptide
RSDVLVK

SEQ ID NO: 241 - protein, 7 aa, Artificial Sequence
  Description of Artificial Sequence Synthetic peptide
RSSALVR

SEQ ID NO: 242 - protein, 7 aa, Artificial Sequence
  Description of Artificial Sequence Synthetic peptide
RKDSLVK

SEQ ID NO: 243 - protein, 7 aa, Artificial Sequence
  Description of Artificial Sequence Synthetic peptide
RSASLVR

SEQ ID NO: 244 - protein, 7 aa, Artificial Sequence
  Description of Artificial Sequence Synthetic peptide
RSDSLVR

SEQ ID NO: 245 - protein, 7 aa, Artificial Sequence
  Description of Artificial Sequence Synthetic peptide
RIHSLVR

SEQ ID NO: 246 - protein, 7 aa, Artificial Sequence
  Description of Artificial Sequence Synthetic peptide
RPGSLVR

SEQ ID NO: 247 - protein, 7 aa, Artificial Sequence
  Description of Artificial Sequence Synthetic peptide
RGPSLVR

SEQ ID NO: 248 - protein, 7 aa, Artificial Sequence
  Description of Artificial Sequence Synthetic peptide
RPGALVR

SEQ ID NO: 249 - protein, 7 aa, Artificial Sequence
  Description of Artificial Sequence Synthetic peptide
KSASKVR

SEQ ID NO: 250 - protein, 7 aa, Artificial Sequence
  Description of Artificial Sequence Synthetic peptide
KSAALVR

SEQ ID NO: 251 - protein, 7 aa, Artificial Sequence
  Description of Artificial Sequence Synthetic peptide
KSAVLVR

SEQ ID NO: 252 - protein, 7 aa, Artificial Sequence
  Description of Artificial Sequence Synthetic peptide
TSGSLTR

SEQ ID NO: 253 - protein, 7 aa, Artificial Sequence
  Description of Artificial Sequence Synthetic peptide
TSQSLVR

SEQ ID NO: 254 - protein, 7 aa, Artificial Sequence
  Description of Artificial Sequence Synthetic peptide
TSSSLVR

SEQ ID NO: 255 - protein, 7 aa, Artificial Sequence
  Description of Artificial Sequence Synthetic peptide
TPGSLVR

SEQ ID NO: 256 - protein, 7 aa, Artificial Sequence
  Description of Artificial Sequence Synthetic peptide
TSGALVR

SEQ ID NO: 257 - protein, 7 aa, Artificial Sequence
  Description of Artificial Sequence Synthetic peptide
TPGALVR

SEQ ID NO: 258 - protein, 7 aa, Artificial Sequence
  Description of Artificial Sequence Synthetic peptide
TGGSLVR

SEQ ID NO: 259 - protein, 7 aa, Artificial Sequence
  Description of Artificial Sequence Synthetic peptide
TSGELVR

SEQ ID NO: 260 - protein, 7 aa, Artificial Sequence
  Description of Artificial Sequence Synthetic peptide
TSGELTR

SEQ ID NO: 261 - protein, 7 aa, Artificial Sequence
  Description of Artificial Sequence Synthetic peptide
TSSALVK

SEQ ID NO: 262 - protein, 7 aa, Artificial Sequence
  Description of Artificial Sequence Synthetic peptide
TSSALVR

SEQ ID NO: 263 - protein, 7 aa, Artificial Sequence
  Description of Artificial Sequence Synthetic peptide
QASNLIS

SEQ ID NO: 264 - protein, 7 aa, Artificial Sequence
  Description of Artificial Sequence Synthetic peptide
SRGNLKS

SEQ ID NO: 265 - protein, 7 aa, Artificial Sequence
  Description of Artificial Sequence Synthetic peptide
RLDNLQT

SEQ ID NO: 266 - protein, 7 aa, Artificial Sequence
  Description of Artificial Sequence Synthetic peptide
ARGNLRT

SEQ ID NO: 267 - protein, 7 aa, Artificial Sequence
  Description of Artificial Sequence Synthetic peptide
RKDALRG

SEQ ID NO: 268 - protein, 7 aa, Artificial Sequence
  Description of Artificial Sequence Synthetic peptide
REDNLHT

SEQ ID NO: 269 - protein, 7 aa, Artificial Sequence
  Description of Artificial Sequence Synthetic peptide
ARGNLKS

SEQ ID NO: 270 - protein, 7 aa, Artificial Sequence
  Description of Artificial Sequence Synthetic peptide
RSDNLTT

SEQ ID NO: 271 - protein, 7 aa, Artificial Sequence
  Description of Artificial Sequence Synthetic peptide
VRGNLKS

SEQ ID NO: 272 - protein, 7 aa, Artificial Sequence
  Description of Artificial Sequence Synthetic peptide
VRGNLRT

SEQ ID NO: 273 - protein, 7 aa, Artificial Sequence
  Description of Artificial Sequence Synthetic peptide
RLRALDR

SEQ ID NO: 274 - protein, 7 aa, Artificial Sequence
  Description of Artificial Sequence Synthetic peptide
DMGALEA

SEQ ID NO: 275 - protein, 7 aa, Artificial Sequence
  Description of Artificial Sequence Synthetic peptide
EKDALRG

SEQ ID NO: 276 - protein, 7 aa, Artificial Sequence
  Description of Artificial Sequence Synthetic peptide
RSDHLTT

SEQ ID NO: 277 - protein, 7 aa, Artificial Sequence
  Description of Artificial Sequence Synthetic peptide
AQQLLMW

SEQ ID NO: 278 - protein, 7 aa, Artificial Sequence
  Description of Artificial Sequence Synthetic peptide
RSDERKR

SEQ ID NO: 279 - protein, 7 aa, Artificial Sequence
  Description of Artificial Sequence Synthetic peptide
DYQSLRQ

SEQ ID NO: 280 - protein, 7 aa, Artificial Sequence
  Description of Artificial Sequence Synthetic peptide
CFSRLVR

SEQ ID NO: 281 - protein, 7 aa, Artificial Sequence
  Description of Artificial Sequence Synthetic peptide
GDGGLWE

SEQ ID NO: 282 - protein, 7 aa, Artificial Sequence
  Description of Artificial Sequence Synthetic peptide
LQRPLRG

SEQ ID NO: 283 - protein, 7 aa, Artificial Sequence
  Description of Artificial Sequence Synthetic peptide
QGLACAA

SEQ ID NO: 284 - protein, 7 aa, Artificial Sequence
  Description of Artificial Sequence Synthetic peptide
WVGWLGS

SEQ ID NO: 285 - protein, 7 aa, Artificial Sequence
  Description of Artificial Sequence Synthetic peptide
RLRDIQF

SEQ ID NO: 286 - protein, 7 aa, Artificial Sequence
  Description of Artificial Sequence Synthetic peptide
GRSQLSC

SEQ ID NO: 287 - protein, 7 aa, Artificial Sequence
  Description of Artificial Sequence Synthetic peptide
GWQRLLT

SEQ ID NO: 288 - protein, 7 aa, Artificial Sequence
  Description of Artificial Sequence Synthetic peptide
SGRPLAS

SEQ ID NO: 289 - protein, 7 aa, Artificial Sequence
  Description of Artificial Sequence Synthetic peptide
APRLLGP

SEQ ID NO: 290 - protein, 7 aa, Artificial Sequence
  Description of Artificial Sequence Synthetic peptide
APKALGW

SEQ ID NO: 291 - protein, 7 aa, Artificial Sequence
  Description of Artificial Sequence Synthetic peptide
SVHELQG

SEQ ID NO: 292 - protein, 7 aa, Artificial Sequence
  Description of Artificial Sequence Synthetic peptide
AQAALSW

SEQ ID NO: 293 - protein, 7 aa, Artificial Sequence
  Description of Artificial Sequence Synthetic peptide
GANALRR

SEQ ID NO: 294 - protein, 7 aa, Artificial Sequence
  Description of Artificial Sequence Synthetic peptide
QSLLLGA

SEQ ID NO: 295 - protein, 7 aa, Artificial Sequence
  Description of Artificial Sequence Synthetic peptide
HRGTLGG

SEQ ID NO: 296 - protein, 7 aa, Artificial Sequence
  Description of Artificial Sequence Synthetic peptide
QVGLLAR

SEQ ID NO: 297 - protein, 7 aa, Artificial Sequence
  Description of Artificial Sequence Synthetic peptide
GARGLRG

SEQ ID NO: 298 - protein, 7 aa, Artificial Sequence
  Description of Artificial Sequence Synthetic peptide
DKHMLDT

SEQ ID NO: 299 - protein, 7 aa, Artificial Sequence
  Description of Artificial Sequence Synthetic peptide
DLGGLRQ

SEQ ID NO: 300 - protein, 7 aa, Artificial Sequence
  Description of Artificial Sequence Synthetic peptide
QCYRLER

SEQ ID NO: 301 - protein, 7 aa, Artificial Sequence
  Description of Artificial Sequence Synthetic peptide
AEAELQR

SEQ ID NO: 302 - protein, 7 aa, Artificial Sequence
  Description of Artificial Sequence Synthetic peptide
QGGVLAA

SEQ ID NO: 303 - protein, 7 aa, Artificial Sequence
  Description of Artificial Sequence Synthetic peptide
QGRCLVT

SEQ ID NO: 304 - protein, 7 aa, Artificial Sequence
  Description of Artificial Sequence Synthetic peptide
HPEALDN

SEQ ID NO: 305 - protein, 7 aa, Artificial Sequence
  Description of Artificial Sequence Synthetic peptide
GRGALQA

SEQ ID NO: 306 - protein, 7 aa, Artificial Sequence
  Description of Artificial Sequence Synthetic peptide
LASRLQQ

SEQ ID NO: 307 - protein, 7 aa, Artificial Sequence
  Description of Artificial Sequence Synthetic peptide
REDNLIS

SEQ ID NO: 308 - protein, 7 aa, Artificial Sequence
  Description of Artificial Sequence Synthetic peptide
RGGWLQA

SEQ ID NO: 309 - protein, 7 aa, Artificial Sequence
  Description of Artificial Sequence Synthetic peptide
DASNLIS

SEQ ID NO: 310 - protein, 7 aa, Artificial Sequence
  Description of Artificial Sequence Synthetic peptide
EASNLIS

SEQ ID NO: 311 - protein, 7 aa, Artificial Sequence
  Description of Artificial Sequence Synthetic peptide
RASNLIS

SEQ ID NO: 312 - protein, 7 aa, Artificial Sequence
  Description of Artificial Sequence Synthetic peptide
TASNLIS

SEQ ID NO: 313 - protein, 7 aa, Artificial Sequence
  Description of Artificial Sequence Synthetic peptide
SASNLIS

SEQ ID NO: 314 - protein, 7 aa, Artificial Sequence
  Description of Artificial Sequence Synthetic peptide
QASTLIS

SEQ ID NO: 315 - protein, 7 aa, Artificial Sequence
  Description of Artificial Sequence Synthetic peptide
QASDLIS

SEQ ID NO: 316 - protein, 7 aa, Artificial Sequence
  Description of Artificial Sequence Synthetic peptide
QASELIS

SEQ ID NO: 317 - protein, 7 aa, Artificial Sequence
  Description of Artificial Sequence Synthetic peptide
QASHLIS

SEQ ID NO: 318 - protein, 7 aa, Artificial Sequence
  Description of Artificial Sequence Synthetic peptide
QASKLIS

SEQ ID NO: 319 - protein, 7 aa, Artificial Sequence
  Description of Artificial Sequence Synthetic peptide
QASSLIS

SEQ ID NO: 320 - protein, 7 aa, Artificial Sequence
  Description of Artificial Sequence Synthetic peptide
QASALIS

SEQ ID NO: 321 - protein, 7 aa, Artificial Sequence
  Description of Artificial Sequence Synthetic peptide
DASTLIS

SEQ ID NO: 322 - protein, 7 aa, Artificial Sequence
  Description of Artificial Sequence Synthetic peptide
DASDLIS

SEQ ID NO: 323 - protein, 7 aa, Artificial Sequence
  Description of Artificial Sequence Synthetic peptide
DASELIS

SEQ ID NO: 324 - protein, 7 aa, Artificial Sequence
  Description of Artificial Sequence Synthetic peptide
DASHLIS

SEQ ID NO: 325 - protein, 7 aa, Artificial Sequence
  Description of Artificial Sequence Synthetic peptide
DASKLIS

SEQ ID NO: 326 - protein, 7 aa, Artificial Sequence
  Description of Artificial Sequence Synthetic peptide
DASSLIS

SEQ ID NO: 327 - protein, 7 aa, Artificial Sequence
  Description of Artificial Sequence Synthetic peptide
DASALIS

SEQ ID NO: 328 - protein, 7 aa, Artificial Sequence
  Description of Artificial Sequence Synthetic peptide
EASTLIS

SEQ ID NO: 329 - protein, 7 aa, Artificial Sequence
  Description of Artificial Sequence Synthetic peptide
EASDLIS

SEQ ID NO: 330 - protein, 7 aa, Artificial Sequence
  Description of Artificial Sequence Synthetic peptide
EASELIS

SEQ ID NO: 331 - protein, 7 aa, Artificial Sequence
  Description of Artificial Sequence Synthetic peptide
EASHLIS

SEQ ID NO: 332 - protein, 7 aa, Artificial Sequence
  Description of Artificial Sequence Synthetic peptide
EASKLIS

SEQ ID NO: 333 - protein, 7 aa, Artificial Sequence
  Description of Artificial Sequence Synthetic peptide
EASSLIS

SEQ ID NO: 334 - protein, 7 aa, Artificial Sequence
  Description of Artificial Sequence Synthetic peptide
EASALIS

SEQ ID NO: 335 - protein, 7 aa, Artificial Sequence
  Description of Artificial Sequence Synthetic peptide
RASTLIS

SEQ ID NO: 336 - protein, 7 aa, Artificial Sequence
  Description of Artificial Sequence Synthetic peptide
RASDLIS

SEQ ID NO: 337 - protein, 7 aa, Artificial Sequence
  Description of Artificial Sequence Synthetic peptide
RASELIS

SEQ ID NO: 338 - protein, 7 aa, Artificial Sequence
  Description of Artificial Sequence Synthetic peptide
RASHLIS

SEQ ID NO: 339 - protein, 7 aa, Artificial Sequence
  Description of Artificial Sequence Synthetic peptide
RASKLIS

SEQ ID NO: 340 - protein, 7 aa, Artificial Sequence
  Description of Artificial Sequence Synthetic peptide
RASSLIS

SEQ ID NO: 341 - protein, 7 aa, Artificial Sequence
  Description of Artificial Sequence Synthetic peptide
RASALIS

SEQ ID NO: 342 - protein, 7 aa, Artificial Sequence
  Description of Artificial Sequence Synthetic peptide
TASTLIS

SEQ ID NO: 343 - protein, 7 aa, Artificial Sequence
  Description of Artificial Sequence Synthetic peptide
TASDLIS

SEQ ID NO: 344 - protein, 7 aa, Artificial Sequence
  Description of Artificial Sequence Synthetic peptide
TASELIS

SEQ ID NO: 345 - protein, 7 aa, Artificial Sequence
  Description of Artificial Sequence Synthetic peptide
TASHLIS

SEQ ID NO: 346 - protein, 7 aa, Artificial Sequence
  Description of Artificial Sequence Synthetic peptide
TASKLIS

SEQ ID NO: 347 - protein, 7 aa, Artificial Sequence
  Description of Artificial Sequence Synthetic peptide
TASSLIS

SEQ ID NO: 348 - protein, 7 aa, Artificial Sequence
  Description of Artificial Sequence Synthetic peptide
TASALIS

SEQ ID NO: 349 - protein, 7 aa, Artificial Sequence
  Description of Artificial Sequence Synthetic peptide
SASTLIS

SEQ ID NO: 350 - protein, 7 aa, Artificial Sequence
  Description of Artificial Sequence Synthetic peptide
SASDLIS

SEQ ID NO: 351 - protein, 7 aa, Artificial Sequence
  Description of Artificial Sequence Synthetic peptide
SASELIS

SEQ ID NO: 352 - protein, 7 aa, Artificial Sequence
  Description of Artificial Sequence Synthetic peptide
SASHLIS

SEQ ID NO: 353 - protein, 7 aa, Artificial Sequence
  Description of Artificial Sequence Synthetic peptide
SASKLIS

SEQ ID NO: 354 - protein, 7 aa, Artificial Sequence
  Description of Artificial Sequence Synthetic peptide
SASSLIS

SEQ ID NO: 355 - protein, 7 aa, Artificial Sequence
  Description of Artificial Sequence Synthetic peptide
SASALIS

SEQ ID NO: 356 - protein, 7 aa, Artificial Sequence
  Description of Artificial Sequence Synthetic peptide
QLDNLQT

SEQ ID NO: 357 - protein, 7 aa, Artificial Sequence
  Description of Artificial Sequence Synthetic peptide
DLDNLQT

SEQ ID NO: 358 - protein, 7 aa, Artificial Sequence
  Description of Artificial Sequence Synthetic peptide
ELDNLQT

SEQ ID NO: 359 - protein, 7 aa, Artificial Sequence
  Description of Artificial Sequence Synthetic peptide
TLDNLQT

SEQ ID NO: 360 - protein, 7 aa, Artificial Sequence
  Description of Artificial Sequence Synthetic peptide
SLDNLQT

SEQ ID NO: 361 - protein, 7 aa, Artificial Sequence
  Description of Artificial Sequence Synthetic peptide
RLDTLQT

SEQ ID NO: 362 - protein, 7 aa, Artificial Sequence
  Description of Artificial Sequence Synthetic peptide
RLDDLQT

SEQ ID NO: 363 - protein, 7 aa, Artificial Sequence
  Description of Artificial Sequence Synthetic peptide
RLDELQT

SEQ ID NO: 364 - protein, 7 aa, Artificial Sequence
  Description of Artificial Sequence Synthetic peptide
RLDHLQT

SEQ ID NO: 365 - protein, 7 aa, Artificial Sequence
  Description of Artificial Sequence Synthetic peptide
RLDKLQT

SEQ ID NO: 366 - protein, 7 aa, Artificial Sequence
  Description of Artificial Sequence Synthetic peptide
RLDSLQT

SEQ ID NO: 367 - protein, 7 aa, Artificial Sequence
  Description of Artificial Sequence Synthetic peptide
RLDALQT

SEQ ID NO: 368 - protein, 7 aa, Artificial Sequence
  Description of Artificial Sequence Synthetic peptide
QLDTLQT

SEQ ID NO: 369 - protein, 7 aa, Artificial Sequence
  Description of Artificial Sequence Synthetic peptide
QLDDLQT

SEQ ID NO: 370 - protein, 7 aa, Artificial Sequence
  Description of Artificial Sequence Synthetic peptide
QLDELQT

SEQ ID NO: 371 - protein, 7 aa, Artificial Sequence
  Description of Artificial Sequence Synthetic peptide
QLDHLQT

SEQ ID NO: 372 - protein, 7 aa, Artificial Sequence
  Description of Artificial Sequence Synthetic peptide
QLDKLQT

SEQ ID NO: 373 - protein, 7 aa, Artificial Sequence
  Description of Artificial Sequence Synthetic peptide
QLDSLQT

SEQ ID NO: 374 - protein, 7 aa, Artificial Sequence
  Description of Artificial Sequence Synthetic peptide
QLDALQT

SEQ ID NO: 375 - protein, 7 aa, Artificial Sequence
  Description of Artificial Sequence Synthetic peptide
DLDTLQT

SEQ ID NO: 376 - protein, 7 aa, Artificial Sequence
  Description of Artificial Sequence Synthetic peptide
DLDDLQT

SEQ ID NO: 377 - protein, 7 aa, Artificial Sequence
  Description of Artificial Sequence Synthetic peptide
DLDELQT

SEQ ID NO: 378 - protein, 7 aa, Artificial Sequence
  Description of Artificial Sequence Synthetic peptide
DLDHLQT

SEQ ID NO: 379 - protein, 7 aa, Artificial Sequence
  Description of Artificial Sequence Synthetic peptide
DLDKLQT

SEQ ID NO: 380 - protein, 7 aa, Artificial Sequence
  Description of Artificial Sequence Synthetic peptide
DLDSLQT

SEQ ID NO: 381 - protein, 7 aa, Artificial Sequence
  Description of Artificial Sequence Synthetic peptide
DLDALQT

SEQ ID NO: 382 - protein, 7 aa, Artificial Sequence
  Description of Artificial Sequence Synthetic peptide
ELDTLQT

SEQ ID NO: 383 - protein, 7 aa, Artificial Sequence
  Description of Artificial Sequence Synthetic peptide
ELDDLQT

SEQ ID NO: 384 - protein, 7 aa, Artificial Sequence
  Description of Artificial Sequence Synthetic peptide
ELDELQT

SEQ ID NO: 385 - protein, 7 aa, Artificial Sequence
  Description of Artificial Sequence Synthetic peptide
ELDHLQT

SEQ ID NO: 386 - protein, 7 aa, Artificial Sequence
  Description of Artificial Sequence Synthetic peptide
ELDKLQT

SEQ ID NO: 387 - protein, 7 aa, Artificial Sequence
  Description of Artificial Sequence Synthetic peptide
ELDSLQT

SEQ ID NO: 388 - protein, 7 aa, Artificial Sequence
  Description of Artificial Sequence Synthetic peptide
ELDALQT

SEQ ID NO: 389 - protein, 7 aa, Artificial Sequence
  Description of Artificial Sequence Synthetic peptide
TLDTLQT

SEQ ID NO: 390 - protein, 7 aa, Artificial Sequence
  Description of Artificial Sequence Synthetic peptide
TLDDLQT

SEQ ID NO: 391 - protein, 7 aa, Artificial Sequence
  Description of Artificial Sequence Synthetic peptide
TLDELQT

SEQ ID NO: 392 - protein, 7 aa, Artificial Sequence
  Description of Artificial Sequence Synthetic peptide
TLDHLQT

SEQ ID NO: 393 - protein, 7 aa, Artificial Sequence
  Description of Artificial Sequence Synthetic peptide
TLDKLQT

SEQ ID NO: 394 - protein, 7 aa, Artificial Sequence
  Description of Artificial Sequence Synthetic peptide
TLDSLQT

SEQ ID NO: 395 - protein, 7 aa, Artificial Sequence
  Description of Artificial Sequence Synthetic peptide
TLDALQT

SEQ ID NO: 396 - protein, 7 aa, Artificial Sequence
  Description of Artificial Sequence Synthetic peptide
SLDTLQT

SEQ ID NO: 397 - protein, 7 aa, Artificial Sequence
  Description of Artificial Sequence Synthetic peptide
SLDDLQT

SEQ ID NO: 398 - protein, 7 aa, Artificial Sequence
  Description of Artificial Sequence Synthetic peptide
SLDELQT

SEQ ID NO: 399 - protein, 7 aa, Artificial Sequence
  Description of Artificial Sequence Synthetic peptide
SLDHLQT

SEQ ID NO: 400 - protein, 7 aa, Artificial Sequence
  Description of Artificial Sequence Synthetic peptide
SLDKLQT

SEQ ID NO: 401 - protein, 7 aa, Artificial Sequence
  Description of Artificial Sequence Synthetic peptide
SLDSLQT

SEQ ID NO: 402 - protein, 7 aa, Artificial Sequence
  Description of Artificial Sequence Synthetic peptide
SLDALQT

SEQ ID NO: 403 - protein, 7 aa, Artificial Sequence
  Description of Artificial Sequence Synthetic peptide
ARGTLRT

SEQ ID NO: 404 - protein, 7 aa, Artificial Sequence
  Description of Artificial Sequence Synthetic peptide
ARGDLRT

SEQ ID NO: 405 - protein, 7 aa, Artificial Sequence
  Description of Artificial Sequence Synthetic peptide
ARGELRT

SEQ ID NO: 406 - protein, 7 aa, Artificial Sequence
  Description of Artificial Sequence Synthetic peptide
ARGHLRT

SEQ ID NO: 407 - protein, 7 aa, Artificial Sequence
  Description of Artificial Sequence Synthetic peptide
ARGKLRT

SEQ ID NO: 408 - protein, 7 aa, Artificial Sequence
  Description of Artificial Sequence Synthetic peptide
ARGSLRT

SEQ ID NO: 409 - protein, 7 aa, Artificial Sequence
  Description of Artificial Sequence Synthetic peptide
ARGALRT

SEQ ID NO: 410 - protein, 7 aa, Artificial Sequence
  Description of Artificial Sequence Synthetic peptide
SRGTLRT

SEQ ID NO: 411 - protein, 7 aa, Artificial Sequence
  Description of Artificial Sequence Synthetic peptide
SRGDLRT

SEQ ID NO: 412 - protein, 7 aa, Artificial Sequence
  Description of Artificial Sequence Synthetic peptide
SRGELRT

SEQ ID NO: 413 - protein, 7 aa, Artificial Sequence
  Description of Artificial Sequence Synthetic peptide
SRGHLRT

SEQ ID NO: 414 - protein, 7 aa, Artificial Sequence
  Description of Artificial Sequence Synthetic peptide
SRGKLRT

SEQ ID NO: 415 - protein, 7 aa, Artificial Sequence
  Description of Artificial Sequence Synthetic peptide
SRGSLRT

SEQ ID NO: 416 - protein, 7 aa, Artificial Sequence
  Description of Artificial Sequence Synthetic peptide
SRGALRT

SEQ ID NO: 417 - protein, 7 aa, Artificial Sequence
  Description of Artificial Sequence Synthetic peptide
QKDALRG

SEQ ID NO: 418 - protein, 7 aa, Artificial Sequence
  Description of Artificial Sequence Synthetic peptide
DKDALRG

SEQ ID NO: 419 - protein, 7 aa, Artificial Sequence
  Description of Artificial Sequence Synthetic peptide
EKDALRG

SEQ ID NO: 420 - protein, 7 aa, Artificial Sequence
  Description of Artificial Sequence Synthetic peptide
TKDALRG

SEQ ID NO: 421 - protein, 7 aa, Artificial Sequence
  Description of Artificial Sequence Synthetic peptide
SKDALRG

SEQ ID NO: 422 - protein, 7 aa, Artificial Sequence
  Description of Artificial Sequence Synthetic peptide
RKDNLRG

SEQ ID NO: 423 - protein, 7 aa, Artificial Sequence
  Description of Artificial Sequence Synthetic peptide
RKDTLRG

SEQ ID NO: 424 - protein, 7 aa, Artificial Sequence
  Description of Artificial Sequence Synthetic peptide
RKDDLRG

SEQ ID NO: 425 - protein, 7 aa, Artificial Sequence
  Description of Artificial Sequence Synthetic peptide
RKDELRG

SEQ ID NO: 426 - protein, 7 aa, Artificial Sequence
  Description of Artificial Sequence Synthetic peptide
RKDHLRG

SEQ ID NO: 427 - protein, 7 aa, Artificial Sequence
  Description of Artificial Sequence Synthetic peptide
RKDKLRG

SEQ ID NO: 428 - protein, 7 aa, Artificial Sequence
  Description of Artificial Sequence Synthetic peptide
RKDSLRG

SEQ ID NO: 429 - protein, 7 aa, Artificial Sequence
  Description of Artificial Sequence Synthetic peptide
QKDNLRG

SEQ ID NO: 430 - protein, 7 aa, Artificial Sequence
  Description of Artificial Sequence Synthetic peptide
QKDTLRG

SEQ ID NO: 431 - protein, 7 aa, Artificial Sequence
  Description of Artificial Sequence Synthetic peptide
QKDDLRG

SEQ ID NO: 432 - protein, 7 aa, Artificial Sequence
  Description of Artificial Sequence Synthetic peptide
QKDELRG

SEQ ID NO: 433 - protein, 7 aa, Artificial Sequence
  Description of Artificial Sequence Synthetic peptide
QKDHLRG

SEQ ID NO: 434 - protein, 7 aa, Artificial Sequence
  Description of Artificial Sequence Synthetic peptide
QKDKLRG

SEQ ID NO: 435 - protein, 7 aa, Artificial Sequence
  Description of Artificial Sequence Synthetic peptide
QKDSLRG

SEQ ID NO: 436 - protein, 7 aa, Artificial Sequence
  Description of Artificial Sequence Synthetic peptide
DKDNLRG

SEQ ID NO: 437 - protein, 7 aa, Artificial Sequence
  Description of Artificial Sequence Synthetic peptide
DKDTLRG

SEQ ID NO: 438 - protein, 7 aa, Artificial Sequence
  Description of Artificial Sequence Synthetic peptide
DKDDLRG

SEQ ID NO: 439 - protein, 7 aa, Artificial Sequence
  Description of Artificial Sequence Synthetic peptide
DKDELRG

SEQ ID NO: 440 - protein, 7 aa, Artificial Sequence
  Description of Artificial Sequence Synthetic peptide
DKDHLRG

SEQ ID NO: 441 - protein, 7 aa, Artificial Sequence
  Description of Artificial Sequence Synthetic peptide
DKDKLRG

SEQ ID NO: 442 - protein, 7 aa, Artificial Sequence
  Description of Artificial Sequence Synthetic peptide
DKDSLRG

SEQ ID NO: 443 - protein, 7 aa, Artificial Sequence
  Description of Artificial Sequence Synthetic peptide
EKDNLRG

SEQ ID NO: 444 - protein, 7 aa, Artificial Sequence
  Description of Artificial Sequence Synthetic peptide
EKDTLRG

SEQ ID NO: 445 - protein, 7 aa, Artificial Sequence
  Description of Artificial Sequence Synthetic peptide
EKDDLRG

SEQ ID NO: 446 - protein, 7 aa, Artificial Sequence
  Description of Artificial Sequence Synthetic peptide
EKDELRG

SEQ ID NO: 447 - protein, 7 aa, Artificial Sequence
  Description of Artificial Sequence Synthetic peptide
EKDHLRG

SEQ ID NO: 448 - protein, 7 aa, Artificial Sequence
  Description of Artificial Sequence Synthetic peptide
EKDKLRG

SEQ ID NO: 449 - protein, 7 aa, Artificial Sequence
  Description of Artificial Sequence Synthetic peptide
EKDSLRG

SEQ ID NO: 450 - protein, 7 aa, Artificial Sequence
  Description of Artificial Sequence Synthetic peptide
TKDNLRG

SEQ ID NO: 451 - protein, 7 aa, Artificial Sequence
  Description of Artificial Sequence Synthetic peptide
TKDTLRG

SEQ ID NO: 452 - protein, 7 aa, Artificial Sequence
  Description of Artificial Sequence Synthetic peptide
TKDDLRG

SEQ ID NO: 453 - protein, 7 aa, Artificial Sequence
  Description of Artificial Sequence Synthetic peptide
TKDELRG

SEQ ID NO: 454 - protein, 7 aa, Artificial Sequence
  Description of Artificial Sequence Synthetic peptide
TKDHLRG

SEQ ID NO: 455 - protein, 7 aa, Artificial Sequence
  Description of Artificial Sequence Synthetic peptide
TKDKLRG

SEQ ID NO: 456 - protein, 7 aa, Artificial Sequence
  Description of Artificial Sequence Synthetic peptide
TKDSLRG

SEQ ID NO: 457 - protein, 7 aa, Artificial Sequence
  Description of Artificial Sequence Synthetic peptide
SKDNLRG

SEQ ID NO: 458 - protein, 7 aa, Artificial Sequence
  Description of Artificial Sequence Synthetic peptide
SKDTLRG

SEQ ID NO: 459 - protein, 7 aa, Artificial Sequence
  Description of Artificial Sequence Synthetic peptide
SKDDLRG

SEQ ID NO: 460 - protein, 7 aa, Artificial Sequence
  Description of Artificial Sequence Synthetic peptide
SKDELRG

SEQ ID NO: 461 - protein, 7 aa, Artificial Sequence
  Description of Artificial Sequence Synthetic peptide
SKDHLRG

SEQ ID NO: 462 - protein, 7 aa, Artificial Sequence
  Description of Artificial Sequence Synthetic peptide
SKDKLRG

SEQ ID NO: 463 - protein, 7 aa, Artificial Sequence
  Description of Artificial Sequence Synthetic peptide
SKDSLRG

SEQ ID NO: 464 - protein, 7 aa, Artificial Sequence
  Description of Artificial Sequence Synthetic peptide
VRGTLRT

SEQ ID NO: 465 - protein, 7 aa, Artificial Sequence
  Description of Artificial Sequence Synthetic peptide
VRGDLRT

SEQ ID NO: 466 - protein, 7 aa, Artificial Sequence
  Description of Artificial Sequence Synthetic peptide
VRGELRT

SEQ ID NO: 467 - protein, 7 aa, Artificial Sequence
  Description of Artificial Sequence Synthetic peptide
VRGHLRT

SEQ ID NO: 468 - protein, 7 aa, Artificial Sequence
  Description of Artificial Sequence Synthetic peptide
VRGKLRT

SEQ ID NO: 469 - protein, 7 aa, Artificial Sequence
  Description of Artificial Sequence Synthetic peptide
VRGSLRT

SEQ ID NO: 470 - protein, 7 aa, Artificial Sequence
  Description of Artificial Sequence Synthetic peptide
VRGTLRT

SEQ ID NO: 471 - protein, 7 aa, Artificial Sequence
  Description of Artificial Sequence Synthetic peptide
QLRALDR

SEQ ID NO: 472 - protein, 7 aa, Artificial Sequence
  Description of Artificial Sequence Synthetic peptide
DLRALDR

SEQ ID NO: 473 - protein, 7 aa, Artificial Sequence
  Description of Artificial Sequence Synthetic peptide
ELRALDR

SEQ ID NO: 474 - protein, 7 aa, Artificial Sequence
  Description of Artificial Sequence Synthetic peptide
TLRALDR

SEQ ID NO: 475 - protein, 7 aa, Artificial Sequence
  Description of Artificial Sequence Synthetic peptide
SLRALDR

SEQ ID NO: 476 - protein, 7 aa, Artificial Sequence
  Description of Artificial Sequence Synthetic peptide
RSDNRKR

SEQ ID NO: 477 - protein, 7 aa, Artificial Sequence
  Description of Artificial Sequence Synthetic peptide
RSDTRKR

SEQ ID NO: 478 - protein, 7 aa, Artificial Sequence
  Description of Artificial Sequence Synthetic peptide
RSDDRKR

SEQ ID NO: 479 - protein, 7 aa, Artificial Sequence
  Description of Artificial Sequence Synthetic peptide
RSDHRKR

SEQ ID NO: 480 - protein, 7 aa, Artificial Sequence
  Description of Artificial Sequence Synthetic peptide
RSDKRKR

SEQ ID NO: 481 - protein, 7 aa, Artificial Sequence
  Description of Artificial Sequence Synthetic peptide
RSDSRKR

SEQ ID NO: 482 - protein, 7 aa, Artificial Sequence
  Description of Artificial Sequence Synthetic peptide
RSDARKR

SEQ ID NO: 483 - protein, 7 aa, Artificial Sequence
  Description of Artificial Sequence Synthetic peptide
QYQSLRQ

SEQ ID NO: 484 - protein, 7 aa, Artificial Sequence
  Description of Artificial Sequence Synthetic peptide
EYQSLRQ

SEQ ID NO: 485 - protein, 7 aa, Artificial Sequence
  Description of Artificial Sequence Synthetic peptide
RYQSLRQ

SEQ ID NO: 486 - protein, 7 aa, Artificial Sequence
  Description of Artificial Sequence Synthetic peptide
TYQSLRQ

SEQ ID NO: 487 - protein, 7 aa, Artificial Sequence
  Description of Artificial Sequence Synthetic peptide
SYQSLRQ

SEQ ID NO: 488 - protein, 7 aa, Artificial Sequence
  Description of Artificial Sequence Synthetic peptide
RLRNIQF

SEQ ID NO: 489 - protein, 7 aa, Artificial Sequence
  Description of Artificial Sequence Synthetic peptide
RLRTIQF

SEQ ID NO: 490 - protein, 7 aa, Artificial Sequence
  Description of Artificial Sequence Synthetic peptide
RLREIQF

SEQ ID NO: 491 - protein, 7 aa, Artificial Sequence
  Description of Artificial Sequence Synthetic peptide
RLRHIQF

SEQ ID NO: 492 - protein, 7 aa, Artificial Sequence
  Description of Artificial Sequence Synthetic peptide
RLRKIQF

SEQ ID NO: 493 - protein, 7 aa, Artificial Sequence
  Description of Artificial Sequence Synthetic peptide
RLRSIQF

SEQ ID NO: 494 - protein, 7 aa, Artificial Sequence
  Description of Artificial Sequence Synthetic peptide
RLRAIQF

SEQ ID NO: 495 - protein, 7 aa, Artificial Sequence
  Description of Artificial Sequence Synthetic peptide
DSLLLGA

SEQ ID NO: 496 - protein, 7 aa, Artificial Sequence
  Description of Artificial Sequence Synthetic peptide
ESLLLGA

SEQ ID NO: 497 - protein, 7 aa, Artificial Sequence
  Description of Artificial Sequence Synthetic peptide
RSLLLGA

SEQ ID NO: 498 - protein, 7 aa, Artificial Sequence
  Description of Artificial Sequence Synthetic peptide
TSLLLGA

SEQ ID NO: 499 - protein, 7 aa, Artificial Sequence
  Description of Artificial Sequence Synthetic peptide
SSLLLGA

SEQ ID NO: 500 - protein, 7 aa, Artificial Sequence
  Description of Artificial Sequence Synthetic peptide
HRGNLGG

SEQ ID NO: 501 - protein, 7 aa, Artificial Sequence
  Description of Artificial Sequence Synthetic peptide
HRGDLGG

SEQ ID NO: 502 - protein, 7 aa, Artificial Sequence
  Description of Artificial Sequence Synthetic peptide
HRGELGG

SEQ ID NO: 503 - protein, 7 aa, Artificial Sequence
  Description of Artificial Sequence Synthetic peptide
HRGHLGG

SEQ ID NO: 504 - protein, 7 aa, Artificial Sequence
  Description of Artificial Sequence Synthetic peptide
HRGKLGG

SEQ ID NO: 505 - protein, 7 aa, Artificial Sequence
  Description of Artificial Sequence Synthetic peptide
HRGSLGG

SEQ ID NO: 506 - protein, 7 aa, Artificial Sequence
  Description of Artificial Sequence Synthetic peptide
HRGALGG

SEQ ID NO: 507 - protein, 7 aa, Artificial Sequence
  Description of Artificial Sequence Synthetic peptide
QKHMLDT

SEQ ID NO: 508 - protein, 7 aa, Artificial Sequence
  Description of Artificial Sequence Synthetic peptide
EKHMLDT

SEQ ID NO: 509 - protein, 7 aa, Artificial Sequence
  Description of Artificial Sequence Synthetic peptide
RKHMLDT

SEQ ID NO: 510 - protein, 7 aa, Artificial Sequence
  Description of Artificial Sequence Synthetic peptide
TKHMLDT

SEQ ID NO: 511 - protein, 7 aa, Artificial Sequence
  Description of Artificial Sequence Synthetic peptide
SKHMLDT

SEQ ID NO: 512 - protein, 7 aa, Artificial Sequence
  Description of Artificial Sequence Synthetic peptide
QLGGLRQ

SEQ ID NO: 513 - protein, 7 aa, Artificial Sequence
  Description of Artificial Sequence Synthetic peptide
ELGGLRQ

SEQ ID NO: 514 - protein, 7 aa, Artificial Sequence
  Description of Artificial Sequence Synthetic peptide
RLGGLRQ

SEQ ID NO: 515 - protein, 7 aa, Artificial Sequence
  Description of Artificial Sequence Synthetic peptide
TLGGLRQ

SEQ ID NO: 516 - protein, 7 aa, Artificial Sequence
  Description of Artificial Sequence Synthetic peptide
SLGGLRQ

SEQ ID NO: 517 - protein, 7 aa, Artificial Sequence
  Description of Artificial Sequence Synthetic peptide
AEANLQR

SEQ ID NO: 518 - protein, 7 aa, Artificial Sequence
  Description of Artificial Sequence Synthetic peptide
AEATLQR

SEQ ID NO: 519 - protein, 7 aa, Artificial Sequence
  Description of Artificial Sequence Synthetic peptide
AEADLQR

SEQ ID NO: 520 - protein, 7 aa, Artificial Sequence
  Description of Artificial Sequence Synthetic peptide
AEAHLQR

SEQ ID NO: 521 - protein, 7 aa, Artificial Sequence
  Description of Artificial Sequence Synthetic peptide
AEAKLQR

SEQ ID NO: 522 - protein, 7 aa, Artificial Sequence
  Description of Artificial Sequence Synthetic peptide
AEASLQR

SEQ ID NO: 523 - protein, 7 aa, Artificial Sequence
  Description of Artificial Sequence Synthetic peptide
AEAALQR

SEQ ID NO: 524 - protein, 7 aa, Artificial Sequence
  Description of Artificial Sequence Synthetic peptide
DGRCLVT

SEQ ID NO: 525 - protein, 7 aa, Artificial Sequence
  Description of Artificial Sequence Synthetic peptide
EGRCLVT

SEQ ID NO: 526 - protein, 7 aa, Artificial Sequence
  Description of Artificial Sequence Synthetic peptide
RGRCLVT

SEQ ID NO: 527 - protein, 7 aa, Artificial Sequence
  Description of Artificial Sequence Synthetic peptide
TGRCLVT

SEQ ID NO: 528 - protein, 7 aa, Artificial Sequence
  Description of Artificial Sequence Synthetic peptide
SGRCLVT

SEQ ID NO: 529 - protein, 7 aa, Artificial Sequence
  Description of Artificial Sequence Synthetic peptide
QEDNLHT

SEQ ID NO: 530 - protein, 7 aa, Artificial Sequence
  Description of Artificial Sequence Synthetic peptide
DEDNLHT

SEQ ID NO: 531 - protein, 7 aa, Artificial Sequence
  Description of Artificial Sequence Synthetic peptide
EEDNLHT

SEQ ID NO: 532 - protein, 7 aa, Artificial Sequence
  Description of Artificial Sequence Synthetic peptide
SEDNLHT

SEQ ID NO: 533 - protein, 7 aa, Artificial Sequence
  Description of Artificial Sequence Synthetic peptide
REDTLHT

SEQ ID NO: 534 - protein, 7 aa, Artificial Sequence
  Description of Artificial Sequence Synthetic peptide
REDDLHT

SEQ ID NO: 535 - protein, 7 aa, Artificial Sequence
  Description of Artificial Sequence Synthetic peptide
REDELHT

SEQ ID NO: 536 - protein, 7 aa, Artificial Sequence
  Description of Artificial Sequence Synthetic peptide
REDHLHT

SEQ ID NO: 537 - protein, 7 aa, Artificial Sequence
  Description of Artificial Sequence Synthetic peptide
REDKLHT

SEQ ID NO: 538 - protein, 7 aa, Artificial Sequence
  Description of Artificial Sequence Synthetic peptide
REDSLHT

SEQ ID NO: 539 - protein, 7 aa, Artificial Sequence
  Description of Artificial Sequence Synthetic peptide
REDALHT

SEQ ID NO: 540 - protein, 7 aa, Artificial Sequence
  Description of Artificial Sequence Synthetic peptide
QEDTLHT

SEQ ID NO: 541 - protein, 7 aa, Artificial Sequence
  Description of Artificial Sequence Synthetic peptide
QEDDLHT

SEQ ID NO: 542 - protein, 7 aa, Artificial Sequence
  Description of Artificial Sequence Synthetic peptide
QEDELHT

SEQ ID NO: 543 - protein, 7 aa, Artificial Sequence
  Description of Artificial Sequence Synthetic peptide
QEDHLHT

SEQ ID NO: 544 - protein, 7 aa, Artificial Sequence
  Description of Artificial Sequence Synthetic peptide
QEDKLHT

SEQ ID NO: 545 - protein, 7 aa, Artificial Sequence
  Description of Artificial Sequence Synthetic peptide
QEDSLHT

SEQ ID NO: 546 - protein, 7 aa, Artificial Sequence
  Description of Artificial Sequence Synthetic peptide
QEDALHT

SEQ ID NO: 547 - protein, 7 aa, Artificial Sequence
  Description of Artificial Sequence Synthetic peptide
DEDTLHT

SEQ ID NO: 548 - protein, 7 aa, Artificial Sequence
  Description of Artificial Sequence Synthetic peptide
DEDDLHT

SEQ ID NO: 549 - protein, 7 aa, Artificial Sequence
  Description of Artificial Sequence Synthetic peptide
DEDELHT

SEQ ID NO: 550 - protein, 7 aa, Artificial Sequence
  Description of Artificial Sequence Synthetic peptide
DEDHLHT

SEQ ID NO: 551 - protein, 7 aa, Artificial Sequence
  Description of Artificial Sequence Synthetic peptide
DEDKLHT

SEQ ID NO: 552 - protein, 7 aa, Artificial Sequence
  Description of Artificial Sequence Synthetic peptide
DEDSLHT

SEQ ID NO: 553 - protein, 7 aa, Artificial Sequence
  Description of Artificial Sequence Synthetic peptide
DEDALHT

SEQ ID NO: 554 - protein, 7 aa, Artificial Sequence
  Description of Artificial Sequence Synthetic peptide
EEDTLHT

SEQ ID NO: 555 - protein, 7 aa, Artificial Sequence
  Description of Artificial Sequence Synthetic peptide
EEDDLHT

SEQ ID NO: 556 - protein, 7 aa, Artificial Sequence
  Description of Artificial Sequence Synthetic peptide
EEDELHT

SEQ ID NO: 557 - protein, 7 aa, Artificial Sequence
  Description of Artificial Sequence Synthetic peptide
EEDHLHT

SEQ ID NO: 558 - protein, 7 aa, Artificial Sequence
  Description of Artificial Sequence Synthetic peptide
EEDKLHT

SEQ ID NO: 559 - protein, 7 aa, Artificial Sequence
  Description of Artificial Sequence Synthetic peptide
EEDSLHT

SEQ ID NO: 560 - protein, 7 aa, Artificial Sequence
  Description of Artificial Sequence Synthetic peptide
EEDALHT

SEQ ID NO: 561 - protein, 7 aa, Artificial Sequence
  Description of Artificial Sequence Synthetic peptide
TEDTLHT

SEQ ID NO: 562 - protein, 7 aa, Artificial Sequence
  Description of Artificial Sequence Synthetic peptide
TEDDLHT

SEQ ID NO: 563 - protein, 7 aa, Artificial Sequence
  Description of Artificial Sequence Synthetic peptide
TEDELHT

SEQ ID NO: 564 - protein, 7 aa, Artificial Sequence
  Description of Artificial Sequence Synthetic peptide
TEDHLHT

SEQ ID NO: 565 - protein, 7 aa, Artificial Sequence
  Description of Artificial Sequence Synthetic peptide
TEDKLHT

SEQ ID NO: 566 - protein, 7 aa, Artificial Sequence
  Description of Artificial Sequence Synthetic peptide
TEDSLHT

SEQ ID NO: 567 - protein, 7 aa, Artificial Sequence
  Description of Artificial Sequence Synthetic peptide
TEDALHT

SEQ ID NO: 568 - protein, 7 aa, Artificial Sequence
  Description of Artificial Sequence Synthetic peptide
SEDTLHT

SEQ ID NO: 569 - protein, 7 aa, Artificial Sequence
  Description of Artificial Sequence Synthetic peptide
SEDDLHT

SEQ ID NO: 570 - protein, 7 aa, Artificial Sequence
  Description of Artificial Sequence Synthetic peptide
SEDELHT

SEQ ID NO: 571 - protein, 7 aa, Artificial Sequence
  Description of Artificial Sequence Synthetic peptide
SEDHLHT

SEQ ID NO: 572 - protein, 7 aa, Artificial Sequence
  Description of Artificial Sequence Synthetic peptide
SEDKLHT

SEQ ID NO: 573 - protein, 7 aa, Artificial Sequence
  Description of Artificial Sequence Synthetic peptide
SEDSLHT

SEQ ID NO: 574 - protein, 7 aa, Artificial Sequence
  Description of Artificial Sequence Synthetic peptide
SEDALHT

SEQ ID NO: 575 - protein, 7 aa, Artificial Sequence
  Description of Artificial Sequence Synthetic peptide
QEDNLIS

SEQ ID NO: 576 - protein, 7 aa, Artificial Sequence
  Description of Artificial Sequence Synthetic peptide
DEDNLIS

SEQ ID NO: 577 - protein, 7 aa, Artificial Sequence
  Description of Artificial Sequence Synthetic peptide
EEDNLIS

SEQ ID NO: 578 - protein, 7 aa, Artificial Sequence
  Description of Artificial Sequence Synthetic peptide
SEDNLIS

SEQ ID NO: 579 - protein, 7 aa, Artificial Sequence
  Description of Artificial Sequence Synthetic peptide
REDTLIS

SEQ ID NO: 580 - protein, 7 aa, Artificial Sequence
  Description of Artificial Sequence Synthetic peptide
REDDLIS

SEQ ID NO: 581 - protein, 7 aa, Artificial Sequence
  Description of Artificial Sequence Synthetic peptide
REDELIS

SEQ ID NO: 582 - protein, 7 aa, Artificial Sequence
  Description of Artificial Sequence Synthetic peptide
REDHLIS

SEQ ID NO: 583 - protein, 7 aa, Artificial Sequence
  Description of Artificial Sequence Synthetic peptide
REDKLIS

SEQ ID NO: 584 - protein, 7 aa, Artificial Sequence
  Description of Artificial Sequence Synthetic peptide
REDSLIS

SEQ ID NO: 585 - protein, 7 aa, Artificial Sequence
  Description of Artificial Sequence Synthetic peptide
REDALIS

SEQ ID NO: 586 - protein, 7 aa, Artificial Sequence
  Description of Artificial Sequence Synthetic peptide
QEDTLIS

SEQ ID NO: 587 - protein, 7 aa, Artificial Sequence
  Description of Artificial Sequence Synthetic peptide
QEDDLIS

SEQ ID NO: 588 - protein, 7 aa, Artificial Sequence
  Description of Artificial Sequence Synthetic peptide
QEDELIS

SEQ ID NO: 589 - protein, 7 aa, Artificial Sequence
  Description of Artificial Sequence Synthetic peptide
QEDHLIS

SEQ ID NO: 590 - protein, 7 aa, Artificial Sequence
  Description of Artificial Sequence Synthetic peptide
QEDKLIS

SEQ ID NO: 591 - protein, 7 aa, Artificial Sequence
  Description of Artificial Sequence Synthetic peptide
QEDSLIS

SEQ ID NO: 592 - protein, 7 aa, Artificial Sequence
  Description of Artificial Sequence Synthetic peptide
QEDALIS

SEQ ID NO: 593 - protein, 7 aa, Artificial Sequence
  Description of Artificial Sequence Synthetic peptide
DEDTLIS

SEQ ID NO: 594 - protein, 7 aa, Artificial Sequence
  Description of Artificial Sequence Synthetic peptide
DEDDLIS

SEQ ID NO: 595 - protein, 7 aa, Artificial Sequence
  Description of Artificial Sequence Synthetic peptide
DEDELIS

SEQ ID NO: 596 - protein, 7 aa, Artificial Sequence
  Description of Artificial Sequence Synthetic peptide
DEDHLIS

SEQ ID NO: 597 - protein, 7 aa, Artificial Sequence
  Description of Artificial Sequence Synthetic peptide
DEDKLIS

SEQ ID NO: 598 - protein, 7 aa, Artificial Sequence
  Description of Artificial Sequence Synthetic peptide
DEDSLIS

SEQ ID NO: 599 - protein, 7 aa, Artificial Sequence
  Description of Artificial Sequence Synthetic peptide
DEDALIS

SEQ ID NO: 600 - protein, 7 aa, Artificial Sequence
  Description of Artificial Sequence Synthetic peptide
EEDTLIS

SEQ ID NO: 601 - protein, 7 aa, Artificial Sequence
  Description of Artificial Sequence Synthetic peptide
EEDDLIS

SEQ ID NO: 602 - protein, 7 aa, Artificial Sequence
  Description of Artificial Sequence Synthetic peptide
EEDELIS

SEQ ID NO: 603 - protein, 7 aa, Artificial Sequence
  Description of Artificial Sequence Synthetic peptide
EEDHLIS

SEQ ID NO: 604 - protein, 7 aa, Artificial Sequence
  Description of Artificial Sequence Synthetic peptide
EEDKLIS

SEQ ID NO: 605 - protein, 7 aa, Artificial Sequence
  Description of Artificial Sequence Synthetic peptide
EEDSLIS

SEQ ID NO: 606 - protein, 7 aa, Artificial Sequence
  Description of Artificial Sequence Synthetic peptide
EEDALIS

SEQ ID NO: 607 - protein, 7 aa, Artificial Sequence
  Description of Artificial Sequence Synthetic peptide
TEDTLIS

SEQ ID NO: 608 - protein, 7 aa, Artificial Sequence
  Description of Artificial Sequence Synthetic peptide
TEDDLIS

SEQ ID NO: 609 - protein, 7 aa, Artificial Sequence
  Description of Artificial Sequence Synthetic peptide
TEDELIS

SEQ ID NO: 610 - protein, 7 aa, Artificial Sequence
  Description of Artificial Sequence Synthetic peptide
TEDHLIS

SEQ ID NO: 611 - protein, 7 aa, Artificial Sequence
  Description of Artificial Sequence Synthetic peptide
TEDKLIS

SEQ ID NO: 612 - protein, 7 aa, Artificial Sequence
  Description of Artificial Sequence Synthetic peptide
TEDSLIS

SEQ ID NO: 613 - protein, 7 aa, Artificial Sequence
  Description of Artificial Sequence Synthetic peptide
TEDALIS

SEQ ID NO: 614 - protein, 7 aa, Artificial Sequence
  Description of Artificial Sequence Synthetic peptide
SEDTLIS

SEQ ID NO: 615 - protein, 7 aa, Artificial Sequence
  Description of Artificial Sequence Synthetic peptide
SEDDLIS

SEQ ID NO: 616 - protein, 7 aa, Artificial Sequence
  Description of Artificial Sequence Synthetic peptide
SEDELIS

SEQ ID NO: 617 - protein, 7 aa, Artificial Sequence
  Description of Artificial Sequence Synthetic peptide
SEDHLIS

SEQ ID NO: 618 - protein, 7 aa, Artificial Sequence
  Description of Artificial Sequence Synthetic peptide
SEDKLIS

SEQ ID NO: 619 - protein, 7 aa, Artificial Sequence
  Description of Artificial Sequence Synthetic peptide
SEDSLIS

SEQ ID NO: 620 - protein, 7 aa, Artificial Sequence
  Description of Artificial Sequence Synthetic peptide
SEDALIS

SEQ ID NO: 621 - protein, 7 aa, Artificial Sequence
  Description of Artificial Sequence Synthetic peptide
TGGWLQA

SEQ ID NO: 622 - protein, 7 aa, Artificial Sequence
  Description of Artificial Sequence Synthetic peptide
SGGWLQA

SEQ ID NO: 623 - protein, 7 aa, Artificial Sequence
  Description of Artificial Sequence Synthetic peptide
DGGWLQA

SEQ ID NO: 624 - protein, 7 aa, Artificial Sequence
  Description of Artificial Sequence Synthetic peptide
EGGWLQA

SEQ ID NO: 625 - protein, 7 aa, Artificial Sequence
  Description of Artificial Sequence Synthetic peptide
QGGWLQA

SEQ ID NO: 626 - protein, 7 aa, Artificial Sequence
  Description of Artificial Sequence Synthetic peptide
RGGTLQA

SEQ ID NO: 627 - protein, 7 aa, Artificial Sequence
  Description of Artificial Sequence Synthetic peptide
RGGDLQA

SEQ ID NO: 628 - protein, 7 aa, Artificial Sequence
  Description of Artificial Sequence Synthetic peptide
RGGELQA

SEQ ID NO: 629 - protein, 7 aa, Artificial Sequence
  Description of Artificial Sequence Synthetic peptide
RGGNLQA

SEQ ID NO: 630 - protein, 7 aa, Artificial Sequence
  Description of Artificial Sequence Synthetic peptide
RGGHLQA

SEQ ID NO: 631 - protein, 7 aa, Artificial Sequence
  Description of Artificial Sequence Synthetic peptide
RGGKLQA

SEQ ID NO: 632 - protein, 7 aa, Artificial Sequence
  Description of Artificial Sequence Synthetic peptide
RGGSLQA

SEQ ID NO: 633 - protein, 7 aa, Artificial Sequence
  Description of Artificial Sequence Synthetic peptide
RGGALQA

SEQ ID NO: 634 - protein, 7 aa, Artificial Sequence
  Description of Artificial Sequence Synthetic peptide
TGGTLQA

SEQ ID NO: 635 - protein, 7 aa, Artificial Sequence
  Description of Artificial Sequence Synthetic peptide
TGGDLQA

SEQ ID NO: 636 - protein, 7 aa, Artificial Sequence
  Description of Artificial Sequence Synthetic peptide
TGGELQA

SEQ ID NO: 637 - protein, 7 aa, Artificial Sequence
  Description of Artificial Sequence Synthetic peptide
TGGNLQA

SEQ ID NO: 638 - protein, 7 aa, Artificial Sequence
  Description of Artificial Sequence Synthetic peptide
TGGHLQA

SEQ ID NO: 639 - protein, 7 aa, Artificial Sequence
  Description of Artificial Sequence Synthetic peptide
TGGKLQA

SEQ ID NO: 640 - protein, 7 aa, Artificial Sequence
  Description of Artificial Sequence Synthetic peptide
TGGSLQA

SEQ ID NO: 641 - protein, 7 aa, Artificial Sequence
  Description of Artificial Sequence Synthetic peptide
TGGALQA

SEQ ID NO: 642 - protein, 7 aa, Artificial Sequence
  Description of Artificial Sequence Synthetic peptide
SGGTLQA

SEQ ID NO: 643 - protein, 7 aa, Artificial Sequence
  Description of Artificial Sequence Synthetic peptide
SGGDLQA

SEQ ID NO: 644 - protein, 7 aa, Artificial Sequence
  Description of Artificial Sequence Synthetic peptide
SGGELQA

SEQ ID NO: 645 - protein, 7 aa, Artificial Sequence
  Description of Artificial Sequence Synthetic peptide
SGGNLQA

SEQ ID NO: 646 - protein, 7 aa, Artificial Sequence
  Description of Artificial Sequence Synthetic peptide
SGGHLQA

SEQ ID NO: 647 - protein, 7 aa, Artificial Sequence
  Description of Artificial Sequence Synthetic peptide
SGGKLQA

SEQ ID NO: 648 - protein, 7 aa, Artificial Sequence
  Description of Artificial Sequence Synthetic peptide
SGGSLQA

SEQ ID NO: 649 - protein, 7 aa, Artificial Sequence
  Description of Artificial Sequence Synthetic peptide
SGGALQA

SEQ ID NO: 650 - protein, 7 aa, Artificial Sequence
  Description of Artificial Sequence Synthetic peptide
DGGTLQA

SEQ ID NO: 651 - protein, 7 aa, Artificial Sequence
  Description of Artificial Sequence Synthetic peptide
DGGDLQA

SEQ ID NO: 652 - protein, 7 aa, Artificial Sequence
  Description of Artificial Sequence Synthetic peptide
DGGELQA

SEQ ID NO: 653 - protein, 7 aa, Artificial Sequence
  Description of Artificial Sequence Synthetic peptide
DGGNLQA

SEQ ID NO: 654 - protein, 7 aa, Artificial Sequence
  Description of Artificial Sequence Synthetic peptide
DGGHLQA

SEQ ID NO: 655 - protein, 7 aa, Artificial Sequence
  Description of Artificial Sequence Synthetic peptide
DGGKLQA

SEQ ID NO: 656 - protein, 7 aa, Artificial Sequence
  Description of Artificial Sequence Synthetic peptide
DGGSLQA

SEQ ID NO: 657 - protein, 7 aa, Artificial Sequence
  Description of Artificial Sequence Synthetic peptide
DGGALQA

SEQ ID NO: 658 - protein, 7 aa, Artificial Sequence
  Description of Artificial Sequence Synthetic peptide
EGGTLQA

SEQ ID NO: 659 - protein, 7 aa, Artificial Sequence
  Description of Artificial Sequence Synthetic peptide
EGGDLQA

SEQ ID NO: 660 - protein, 7 aa, Artificial Sequence
  Description of Artificial Sequence Synthetic peptide
EGGELQA

SEQ ID NO: 661 - protein, 7 aa, Artificial Sequence
  Description of Artificial Sequence Synthetic peptide
EGGNLQA

SEQ ID NO: 662 - protein, 7 aa, Artificial Sequence
  Description of Artificial Sequence Synthetic peptide
EGGHLQA

SEQ ID NO: 663 - protein, 7 aa, Artificial Sequence
  Description of Artificial Sequence Synthetic peptide
EGGKLQA

SEQ ID NO: 664 - protein, 7 aa, Artificial Sequence
  Description of Artificial Sequence Synthetic peptide
EGGSLQA

SEQ ID NO: 665 - protein, 7 aa, Artificial Sequence
  Description of Artificial Sequence Synthetic peptide
EGGALQA

SEQ ID NO: 666 - protein, 7 aa, Artificial Sequence
  Description of Artificial Sequence Synthetic peptide
QGGTLQA

SEQ ID NO: 667 - protein, 7 aa, Artificial Sequence
  Description of Artificial Sequence Synthetic peptide
QGGDLQA

SEQ ID NO: 668 - protein, 7 aa, Artificial Sequence
  Description of Artificial Sequence Synthetic peptide
QGGELQA

SEQ ID NO: 669 - protein, 7 aa, Artificial Sequence
  Description of Artificial Sequence Synthetic peptide
QGGNLQA

SEQ ID NO: 670 - protein, 7 aa, Artificial Sequence
  Description of Artificial Sequence Synthetic peptide
QGGHLQA

SEQ ID NO: 671 - protein, 7 aa, Artificial Sequence
  Description of Artificial Sequence Synthetic peptide
QGGKLQA

SEQ ID NO: 672 - protein, 7 aa, Artificial Sequence
  Description of Artificial Sequence Synthetic peptide
QGGSLQA

SEQ ID NO: 673 - protein, 7 aa, Artificial Sequence
  Description of Artificial Sequence Synthetic peptide
QGGALQA

SEQ ID NO: 674 - protein, 5 aa, Artificial Sequence
  Description of Artificial Sequence Synthetic peptide
TGEKP

SEQ ID NO: 675 - protein, 15 aa, Artificial Sequence
  Description of Artificial Sequence Synthetic peptide
TGGGGSGGGGTGEKP

SEQ ID NO: 676 - protein, 12 aa, Artificial Sequence
  Description of Artificial Sequence Synthetic peptide
LRQKDGGGSERP

SEQ ID NO: 677 - protein, 9 aa, Artificial Sequence
  Description of Artificial Sequence Synthetic peptide
LRQKDGERP

SEQ ID NO: 678 - protein, 10 aa, Artificial Sequence
  Description of Artificial Sequence Synthetic peptide
GGRGRGRGRQ

SEQ ID NO: 679 - protein, 16 aa, Artificial Sequence
  Description of Artificial Sequence Synthetic peptide
QNKKGGSGDGKKKQHI

SEQ ID NO: 680 - protein, 6 aa, Artificial Sequence
  Description of Artificial Sequence Synthetic peptide
TGGERP

SEQ ID NO: 681 - protein, 6 aa, Artificial Sequence
  Description of Artificial Sequence Synthetic peptide
ATGEKP

SEQ ID NO: 682 - protein, 11 aa, Artificial Sequence
  Description of Artificial Sequence Synthetic peptide
GGGSGGGGEGP

SEQ ID NO: 683 - protein, 7 aa, Artificial Sequence
  Description of Artificial Sequence Synthetic peptide
  MOD_RES (4)..(4): variable amino acid
RSDXLVR

SEQ ID NO: 684 - DNA, 9 bp, Artificial Sequence
  Description of Artificial Sequence Synthetic peptide
gcgtgggcg

SEQ ID NO: 685 - DNA, 9 bp, Artificial Sequence
  Description of Artificial Sequence Synthetic peptide
  modified_base (4)..(6): a, c, g, t, unknown or other
gcgnnngcg

SEQ ID NO: 686 - protein, 7 aa, Artificial Sequence
  Description of Artificial Sequence Synthetic peptide
RSDELKR

SEQ ID NO: 687 - DNA, 9 bp, Artificial Sequence
  Description of Artificial Sequence Synthetic peptide
  modified_base (5)..(6): a, c, g, t, unknown or other
gatcnngcg

SEQ ID NO: 688 - protein, 7 aa, Artificial Sequence
  Description of Artificial Sequence Synthetic peptide
SPADLTN

SEQ ID NO: 689 - protein, 7 aa, Artificial Sequence
  Description of Artificial Sequence Synthetic peptide
HISNFCR

SEQ ID NO: 690 - DNA, 9 bp, Artificial Sequence
  Description of Artificial Sequence Synthetic peptide
gcgtgggcg

SEQ ID NO: 691 - DNA, 9 bp, Artificial Sequence
  Description of Artificial Sequence Synthetic peptide
  modified_base (5)..(6): a, c, g, t, unknown or other
gatanngcg

SEQ ID NO: 692 - protein, 7 aa, Artificial Sequence
  Description of Artificial Sequence Synthetic peptide
ERSKLRA

SEQ ID NO: 693 - protein, 7 aa, Artificial Sequence
  Description of Artificial Sequence Synthetic peptide
DPGHLRV

SEQ ID NO: 694 - protein, 7 aa, Artificial Sequence
  Description of Artificial Sequence Synthetic peptide
DPGSLRV

SEQ ID NO: 695 - protein, 7 aa, Artificial Sequence
  Description of Artificial Sequence Synthetic peptide
RSDNLKN

SEQ ID NO: 696 - protein, 7 aa, Artificial Sequence
  Description of Artificial Sequence Synthetic peptide
SRDALNV

SEQ ID NO: 697 - protein, 7 aa, Artificial Sequence
  Description of Artificial Sequence Synthetic peptide
VKDYLTK

SEQ ID NO: 698 - protein, 7 aa, Artificial Sequence
  Description of Artificial Sequence Synthetic peptide
KNWKLQA

SEQ ID NO: 699 - protein, 7 aa, Artificial Sequence
  Description of Artificial Sequence Synthetic peptide
AQYMLVV

SEQ ID NO: 700 - protein, 7 aa, Artificial Sequence
  Description of Artificial Sequence Synthetic peptide
QSTNLKS

SEQ ID NO: 701 - protein, 7 aa, Artificial Sequence
  Description of Artificial Sequence Synthetic peptide
LDFNLRT

SEQ ID NO: 702 - protein, 7 aa, Artificial Sequence
  Description of Artificial Sequence Synthetic peptide
RKDNMTA

SEQ ID NO: 703 - protein, 7 aa, Artificial Sequence
  Description of Artificial Sequence Synthetic peptide
QSSNLIT

SEQ ID NO: 704 - protein, 7 aa, Artificial Sequence
  Description of Artificial Sequence Synthetic peptide
QRSALTV

SEQ ID NO: 705 - protein, 7 aa, Artificial Sequence
  Description of Artificial Sequence Synthetic peptide
QSGSLTR

SEQ ID NO: 706 - RNA, 7 nt, Artificial Sequence
  Description of Artificial Sequence Synthetic peptide
aggaggu

SEQ ID NO: 707 - DNA, 20 bp, Artificial Sequence
  Description of Artificial Sequence Synthetic peptide
tcagaactcacctgttagac

SEQ ID NO: 708 - DNA, 37 bp, Artificial Sequence
  Description of Artificial Sequence Synthetic peptide
  modified_base (10)..(12): a, c, g, t, unknown or other
tatatagcgnnngcgtatatatcaagtcaatcggtcc

SEQ ID NO: 709 - DNA, 16 bp, Artificial Sequence
  Description of Artificial Sequence Synthetic peptide
ggaccgattgacttga

SEQ ID NO: 710 - DNA, 34 bp, Artificial Sequence
  Description of Artificial Sequence Synthetic peptide
  modified_base (4)..(12): a, c, g, t, unknown or other
  modified_base (23)..(31): a, c, g, t, unknown or other
ggannnnnnnnngggttttcccnnnnnnnnntcc

SEQ ID NO: 711 - DNA, 49 bp, Artificial Sequence
  Description of Artificial Sequence Synthetic peptide
  modified_base (22)..(31): a, c, g, t, unknown or other
gagctcatggaagtaccatagnnnnnnnnnngaacgtcgatcactcgag

SEQ ID NO: 712 - DNA, 51 bp, Artificial Sequence
  Description of Artificial Sequence Synthetic peptide
  modified_base (22)..(33): a, c, g, t, unknown or other
gagctcatggaagtaccatagnnnnnnnnnnnngaacgtcgatcactcgag

SEQ ID NO: 713 - DNA, 60 bp, Artificial Sequence
  Description of Artificial Sequence Synthetic peptide
  modified_base (22)..(42): a, c, g, t, unknown or other
gagctcatggaagtaccatagnnnnnnnnnnnnnnnnnnnnngaacgtcgatcactcgag

SEQ ID NO: 714 - DNA, 21 bp, Artificial Sequence
  Description of Artificial Sequence Synthetic peptide
gagctcatggaagtaccatag

SEQ ID NO: 715 - DNA, 18 bp, Artificial Sequence
  Description of Artificial Sequence Synthetic peptide
ctcgagtgatcgacgttc

SEQ ID NO: 716 - DNA, 18 bp, Artificial Sequence
  Description of Artificial Sequence Synthetic peptide
  modified_base (2)..(3): a, c, g, t, unknown or other
  modified_base (5)..(6): a, c, g, t, unknown or other
  modified_base (8)..(9): a, c, g, t, unknown or other
  modified_base (11)..(12): a, c, g, t, unknown or other
  modified_base (14)..(15): a, c, g, t, unknown or other
  modified_base (17)..(18): a, c, g, t, unknown or other
gnngnngnngnngnngnn

(19) United States
(12) Patent Application Publication
(10) Pub. No.: US 2007/0178499 A1
(43) Pub. Date: Aug. 2, 2007

(54) Specific Labeling of Protein with Zinc Finger Tags and Use of Zinc-Finger-Tagged Proteins for Analysis

(75) Inventor: Carlos F. Barbas, III, Solana Beach, CA (US)

Correspondence Address: CATALYST LAW GROUP, APC, 9710 SCRANTON ROAD, SUITE S-170, SAN DIEGO, CA, 92121, US

(73) Assignee: The Scripps Research Institute, La Jolla, CA (US)

(21) Appl. No.: 11/620,585

(22) Filed: Jan. 5, 2007

Related U.S. Application Data
(60) Provisional application No. 60/756,936, filed on Jan. 6, 2006.

(51) Int. Cl. C12Q 1/68; C12M 1/34

(52) U.S. Cl. 435/6; 435/287.2

(57) ABSTRACT

Fusion proteins including zinc finger tags that bind in a sequence-specific manner and a peptide, polypeptide, or protein can be prepared and expressed. Such fusion proteins can be used to generate protein arrays by binding the zinc finger tags to a DNA array. The fusion proteins can also be used to label cell surfaces with DNA tags. Fusion proteins according to the invention can be used to localize the peptide, polypeptide, or protein that is incorporated into the fusion protein by using a labeled DNA, such as a fluorescent DNA. The invention further includes vectors and host cells, as well as a method of analyzing double-stranded DNA.

### CROSS-REFERENCES

This application claims priority from U.S. Provisional Application Ser. No. 60/756,936 by Carlos F. Barbas, III, entitled “Specific Labeling of Proteins with Zinc Finger Tags and Use of Zinc-Finger Tagged Proteins for Analysis,” filed on Jan. 6, 2006, the contents of which are incorporated herein in their entirety by this reference.

### FIELD OF THE INVENTION

This invention is directed to methods and compositions for the specific labeling of proteins with zinc finger tags and methods for the use of zinc-finger-tagged proteins for analysis.

### BACKGROUND OF THE INVENTION

With the completion of the Human Genome Project, increased attention has turned to the structure and function of proteins encoded by the genes of the genome. The complete collection of proteins encoded by a genome is defined as the “proteome,” and the study of the properties of these proteins, including their primary structure, secondary structure, tertiary structure, quaternary structure, function, and interactions with other proteins, nucleic acids, and small molecules, is defined as “proteomics,” by analogy with “genomics.” The quantity of information required to gain an understanding of these properties for all or substantially all of the proteins in a particular organism is orders of magnitude greater than the quantity of information required to gain an understanding of the structure of the genome of that organism. That is because there is as yet no generally applicable way to predict the secondary, tertiary, or quaternary structure of proteins to the degree of precision required for this analysis, much less to analyze the function of these proteins or their interactions with other proteins, nucleic acids, or small molecules. This is because the additional information, in addition to the primary sequence, required to predict these structures or activities is far greater for proteins than it is for nucleic acids, and the range of interaction with other molecules is far greater

Therefore much of this information can, at present, be acquired by detailed studies of each protein on a protein-by-protein basis. Even for relatively intensively studied model organisms such as Escherichia coli and Saccharomyces cerevisiae, functions have been assigned to only about half of their proteins. For mammals, which have considerably greater complexity, the task is slower.

In recent years, several approaches to proteomics have been developed that allow high-throughput protein analysis. Several of these approaches are two-dimensional gel electrophoresis, affinity chromatography combined with mass spectroscopy, the yeast two-hybrid system, and a computational approach called “Rosetta Stone” that is based on the analysis of genomic DNA sequences. These techniques are described, for example, in J. Pevsner, “Bioinformatics and Functional Genomics” (Wiley-Liss, Hoboken, N.J., 2003), pp. 247-258, incorporated herein by this reference.

Additional techniques include protein microarrays and tissue microarrays. However, the latter techniques suffer from the problem of the inherent difficulty of maintaining the native three-dimensional structure and function of proteins immobilized in such microarrays. The failure of proteins in these microarrays to maintain their native three-dimensional structure and function means that information obtained from these microarrays frequently needs to be verified by other, slower techniques to ensure that the information reflects the native conformation of the proteins.

Additionally, none of these techniques allows the tracking of proteins in living cells. Although techniques for the labeling and tracking of proteins in living cells are known, such as the use of Green Fluorescent Protein (GFP) (B. A. Griffin et al., “Specific Covalent Labeling of Recombinant Protein Molecules Inside Live Cells,” Science 281: 269-272 (1998)), there is a need for additional techniques that can both allow the tracking of proteins in living cells and allow the assembly of proteins into arrays to study proteomics without the risk of disturbing the native conformation of the proteins.

### SUMMARY OF THE INVENTION

The development of fusion proteins incorporating a peptide, polypeptide, or protein of interest and a zinc finger tag that binds in a sequence-specific manner to a defined nucleotide sequence provides a means for the tracking of proteins in living cells and for the assembly of proteins into arrays.

Accordingly, one aspect of the present invention is an array comprising:

(1) a solid support;

(2) a plurality of nucleotide sequences attached to the solid support; and

(3) a plurality of fusion proteins specifically and noncovalently bound to the plurality of nucleotide sequences, each fusion protein comprising: (a) a protein, peptide, or polypeptide of interest; and (b) a zinc protein finger tag, wherein each zinc finger protein tag has specific binding affinity for only one of the nucleotide sequences attached to the solid support.

Another aspect of the present invention is a method for assaying activity of a peptide, polypeptide, or protein of interest comprising the steps of:

(1) providing an array as described above;

(2) contacting the array with a reagent that reacts with a peptide, polypeptide, or protein of interest that may or not be present in the array to produce a detectable product; and

(3) determining the location of a peptide, polypeptide, or protein in the array by determining the location of the detectable product in order to identify the location of a peptide, polypeptide, or protein that has a defined activity associated with the production of the detectable product.

Still another aspect of the present invention is a fusion protein comprising:

(1) a protein, polypeptide, or peptide of interest; and

(2) at least one zinc finger tag in a single polypeptide; such that the protein, polypeptide, or protein of interest substantially maintains its three-dimensional conformation and activity, and the zinc finger tag substantially maintains its sequence-specific nucleotide sequence binding activity.

Additional aspects of the invention are polynucleotides encoding the fusion proteins. The polynucleotides can be DNA, and the invention further includes vectors including the DNA. The invention further includes host cells transformed or transfected by the vectors.

Accordingly, another aspect of the invention is a method of expressing a fusion protein comprising the steps of:

(1) introducing a vector according to the present invention as described above into a compatible host cell; and

(2) causing the fusion protein to be expressed in the host cell; and

(3) isolating the expressed fusion protein.

In accordance with the need for improved in vivo localization of a target protein in a cell, another aspect of the invention is a method for in vivo localization of a target protein in a cell comprising the steps of:

(1) expressing a fusion protein according to the present invention as described above in a cell, the target protein being incorporated in the fusion protein;

(2) introducing a DNA molecule into the cell that is specifically bound by the zinc finger tag of the fusion protein, wherein the DNA molecule is covalently labeled with a fluorescent indicator molecule;

(3) incubating the cell so that the DNA molecule binds to the fusion protein; and

(4) localizing the target protein in the cell by locating the fluorescent indicator molecule.

Similarly, another aspect of the invention is a method for labeling the cell membrane of a cell comprising the steps of:

(1) transforming or transfecting a host cell with a nucleic acid sequence that encodes a fusion protein that is a fusion of a membrane protein with a zinc finger tag such that the cell expresses the fusion protein;

(2) culturing the transformed or transfected cell under conditions such that the fusion protein is expressed and is incorporated in the cell membrane of the cell;

(3) contacting the cell expressing the fusion protein incorporated in the membrane with a labeled DNA molecule that binds the zinc finger tag of the fusion protein in a sequence-specific manner; and

(4) detecting the label of the labeled DNA molecule on the cell surface.

Another aspect of the invention is a cell including therein a fusion protein according to the present invention wherein the fusion protein includes therein a membrane protein, such that the fusion protein is incorporated into the cell membrane. This cell can be used in a method of cross-linking cells comprising the steps of:

(1) providing the cells;

(2) labeling the cells with DNA;

(3) arraying the cells on DNA surfaces; and

(4) cross-linking the cells on the DNA surfaces.

Another aspect of the invention is a method of analyzing double-stranded DNA comprising the steps of:

(1) providing a plurality of fusion proteins according to the present invention as described above;

(2) binding the fusion proteins to a solid support, each fusion protein being attached at a defined nonoverlapping location on the solid support, to produce a fusion protein microarray;

(3) exposing the fusion protein to a sample containing one or more double-stranded DNA molecules so that any double-stranded DNA molecule possessing a defined nucleotide sequence bound by a zinc finger tag incorporated in a fusion protein is bound; and

(4) analyzing the binding of DNA molecules to the fusion proteins in order to determine whether DNA molecules possessing any of the defined nucleotide sequences are present in the sample.

### BRIEF DESCRIPTION OF THE DRAWINGS

The following invention will become better understood with reference to the specification, appended claims, and accompanying drawings, where:

FIG. 1 is a schematic depiction of a fusion protein according to the present invention.

FIG. 2 is a schematic depiction of a protein array according to the present invention.

FIG. 3 is a schematic depiction of the process of preparing fusion proteins from a cDNA library.

FIG. 4 is a schematic depiction of fusion proteins incorporating scFv antibody molecules for the preparation of an antibody array.

FIG. 5 is a schematic depiction of double-stranded DNA analysis using fusion proteins according to the present invention.

FIG. 6 is a diagram of representations of zinc finger-DNA interactions, based on the structure of the naturally-occurring zinc finger protein Zif268.

FIG. 7 shows the specificity of 80 zinc finger proteins based on the multi-target ELISA assay.

FIG. 8 shows an overview of the CAST assay: (A) A flow diagram describing the steps of the CAST assay. (B) Raw data from the CAST analysis of B3-HS2(S).

FIG. 9 is a series of graphs showing results of the CAST assay (FIG. 8) on a number of constructed zinc finger proteins.

### DETAILED DESCRIPTION OF THE INVENTION

The construction and use of proteins tagged with zinc finger domains provides a way of meeting these needs. This allows the tracking of the tagged proteins in a living cell. It also allows the assembly of such proteins into arrays based on the affinity between the zinc finger tags and the corresponding nucleic acid segments recognized specifically by the zinc finger tags.

Zinc fingers are motifs of proteins that have the property of specifically binding defined nucleic acid sequences. Such zinc fingers are utilized in cells as part of transcription factors and other proteins that are required to specifically bind DNA as part of their function. There are several types of zinc fingers, but the most significant one is the Cys₂-His₂ zinc finger. As used herein, the term “zinc finger” refers to a motif containing one or more Cys₂-His₂ zinc fingers, as well as to other types of zinc fingers described below. These Cys₂-His₂ zinc fingers are described, for example, in U.S. Pat. No. 7,101,972 to Barbas, U.S. Pat. No. 7,067,617 to Barbas et al., U.S. Pat. No. 6,790,941 to Barbas et al., U.S. Pat. No. 6,610,512 to Barbas, U.S. Pat. No. 6,242,568 to Barbas et al., U.S. Pat. No. 6,140,466 to Barbas et al., U.S. Pat. No. 6,140,081 to Barbas, United States Patent Application Publication No. 20060223757 by Barbas, United States Patent Application Publication No. 20060211846 by Barbas et al., United States Patent Application Publication No. 20060078880 by Barbas et al., United States Patent Application Publication No. 20050148075 by Barbas, United States Patent Application Publication No. 20050084885 by Barbas et al., United States Patent Application Publication No. 20040224385 by Barbas et al., United States Patent Application Publication No. 20030059767 by Barbas et al., and United States Patent Application Publication No. 20020165356 by Barbas et al., all of which are incorporated herein by this reference.

The Cys₂-His₂ zinc finger motif, identified first in the DNA and RNA binding transcription factor TFIIIA (Miller, J., McLachlan, A. D. & Klug, A. (1985) Embo J 4, 1609-14), is perhaps the ideal structural scaffold on which a sequence specific protein might be constructed. A single zinc finger domain consists of approximately 30 amino acids folded into a ββα structure stabilized by hydrophobic interactions and the chelation of a single zinc ion (Miller, J., McLachlan, A. D. & Klug, A. (1985) Embo J 4, 1609-14, Lee, M. S., Cippert, G. P., Soman, K. V., Case, D. A. & Wright, P. E. (1989) Science 245, 635-7). Presentation of the α-helix of this domain into the major groove of DNA allows for sequence specific base contacts. Each zinc finger domain typically recognizes three base pairs of DNA (Pavletich, N. P. & Pabo, C. O. (1991) Science (Washington, D.C., 1883-) 252, 809-17, Elrod-Erickson, M., Rould, M. A., Nekludova, L. & Pabo, C. O. (1996) Structure (London) 4, 1171-1180, Elrod-Erickson, M., Benson, T. E. & Pabo, C. O. (1998) Structure (London) 6, 451464, Kim, C. A. & Berg, J. M. (1996) Nature Structural Biology 3, 940-945), though variation in helical presentation can allow for recognition of a more extended site (Pavletich, N. P. & Pabo, C. O. (1993) Science (Washington, D.C., 1883-) 261, 1701-7, Houbaviy, H. B., Usheva, A., Shenk, T. & Burley, S. K. (1996) Proc Natl Acad Sci USA 93, 13577-82, Fairall, L., Schwabe, J. W. R., Chapman, L., Finch, J. T. & Rhodes, D. (1993) Nature (London) 366, 483-7, Wuttke, D. S., Foster, M. P., Case, D. A., Gottesfeld, J. M. & Wright, P. E. (1997) J. Mol. Biol. 273, 183-206). In contrast to most transcription factors that rely on dimerization of protein domains for extending protein-DNA contacts to longer DNA sequences or addresses, simple covalent tandem repeats of the zinc finger domain allow for the recognition of longer asymmetric sequences of DNA by this motif. Polydactyl zinc finger proteins that contain 6 zinc finger domains and bind 18 base pairs of contiguous DNA sequence were described (Liu, Q., Segal, D. J., Ghiara, J. B. & Barbas III, C. F. (1997) PNAS 94, 5525-5530). Recognition of 18 base pairs of DNA is sufficient to describe a unique DNA address within all known genomes, a requirement for using polydactyl proteins as highly specific gene switches. Indeed, control of both gene activation and repression has been shown using these polydactyl proteins in a model system (Liu, Q., Segal, D. J., Ghiara, J. B. & Barbas III, C. F. (1997) PNAS 94, 5525-5530).

Since each zinc finger domain typically binds three base pairs of sequence, a complete recognition alphabet requires the characterization of 64 domains. Existing information which could guide the construction of these domains has come from three types of studies: structure determination (Pavletich, N. P. & Pabo, C. O. (1991) Science (Washington, D.C., 1883) 252, 809-17, Elrod-Erickson, M., Rould, M. A., Nekludova, L. & Pabo, C. O. (1996) Structure (London) 4, 1171-1180, Elrod-Erickson, M., Benson, T. E. & Pabo, C. O. (1998) Structure (London) 6, 451-464, Kim, C. A. & Berg, J. M. (1996) Nature Structural Biology 3, 940-945, Pavletich, N. P. & Pabo, C. O. (1993) Science (Washington, D.C., 1883-) 261, 1701-7, Houbaviy, H. B., Usheva, A., Shenk, T. & Burley, S. K. (1996) Proc Natl Acad Sci USA 93, 13577-82, Fairall, L., Schwabe, J. W. R., Chapman, L., Finch, J. T. & Rhodes, D. (1993) Nature (London) 366, 483-7., 11, Wuttke, D. S., Foster, M. P., Case, D. A., Gottesfeld, J. M. & Wright, P. E. (1997) J. Mol. Biol. 273, 183-206., Nolte, R. T., Conlin, R. M., Harrison, S. C. & Brown, R. S. (1998) Proc. Natl. Acad. Sci. U.S.A. 95, 2938-2943, Narayan, V. A., Kriwacki, R. W. & Caradonna, J. P. (1997) J. Biol. Chem. 272, 7801-7809., site-directed mutagenesis (Isalan, M., Choo, Y. & Klug, A. (1997) Proc. Natl. Acad. Sci. U.S.A. 94, 5617-5621, Nardelli, J., Gibson, T. J., Vesque, C. & Charnay, P. (1991) Nature 349, 175-178, Nardelli, J., Gibson, T. & Charnay, P. (1992) Nucleic Acids Res. 20, 413744, Taylor, W. E., Suruki, H. K., Lin, A. H. T., Naraghi-Arani, P., Igarashi, R. Y., Younessian, M., Katkus, P. & Vo, N. V. (1995) Biochemistry 34, 3222-3230, Desjarlais, J. R. & Berg, J. M. (1992) Proteins: Struct., Funct., Genet. 12, 1014, Desjarlais, J. R. & Berg, J. M. (1992) Proc Natl Acad Sci USA 89, 7345-9), and phage-display selections (Choo, Y. & Klug, A. (1994) Proc Natl Acad Sci USA 91, 11163-7, Greisman, H. A. & Pabo, C. O. (1997) Science (Washington, D.C.) 275, 657-661.23, Rebar, E. J. & Pabo, C. O. (1994) Science (Washington, D.C., 1883-) 263, 671-3, Jamieson, A. C., Kim, S.-H. & Wells, J. A. (1994) Biochemistry 33, 5689-5695, Jamieson, A. C., Wang, H. & Kim, S.-H. (1996) PNAS 93, 12834-12839, Isalan, M., Klug, A. & Choo, Y. (1998) Biochemistry 37, 12026-33, Wu, H., Yang, W.-P. & Barbas III, C. F. (1995) PNAS 92, 344-348). All have contributed significantly to understanding of zinc finger/DNA recognition, but each has its limitations. Structural studies have identified a diverse spectrum of protein/DNA interactions but do not explain if alternative interactions might be more optimal. Further, while interactions that allow for sequence specific recognition are observed, little information is provided on how alternate sequences are excluded from binding. These questions have been partially addressed by mutagenesis of existing proteins, but the data is always limited by the number of mutants that can be characterized. Phage-display and selection of randomized libraries overcomes certain numerical limitations, but providing the appropriate selective pressure to ensure that both specificity and affinity drive the selection is difficult. Experimental studies from several laboratories (Choo, Y. & Klug, A. (1994) Proc Natl Acad Sci USA 91, 11163-7, Greisman, H. A. & Pabo, C. O. (1997) Science (Washington, D.C.) 275, 657-661, Rebar, E. J. & Pabo, C. O. (1994) Science (Washington, D.C., 1883-) 263, 671-3, Jamieson, A. C., Kim, S.-H. & Wells, J. A. (1994) Biochemistry 33, 5689-5695.25, Jamieson, A. C., Wang, H. & Kim, S.-H. (1996) PNAS 93, 12834-12839, Isalan, M., Klug, A. & Choo, Y. (1998) Biochemistry 37, 12026-33; Wu, H., Yang, W.-P. & Barbas III, C. F. (1995) PNAS 92, 344-348), have demonstrated that it is possible to design or select a few members of this recognition alphabet. However, the specificity and affinity of these domains for their target DNA were rarely investigated in a rigorous and systematic fashion in these early studies.

I. Fusion Proteins

A. Fusion Proteins with Polypeptides and Zinc Finger Tag

One aspect of the invention is a fusion protein that incorporates: (1) a protein, polypeptide, or peptide of interest (referred to hereinafter for convenience as a “protein of interest”); and (2) at least one zinc finger tag in a single polypeptide. In a fusion protein according to the invention, the protein of interest substantially maintains its three-dimensional conformation and activity, and the zinc finger tag substantially maintains its sequence-specific DNA binding activity.

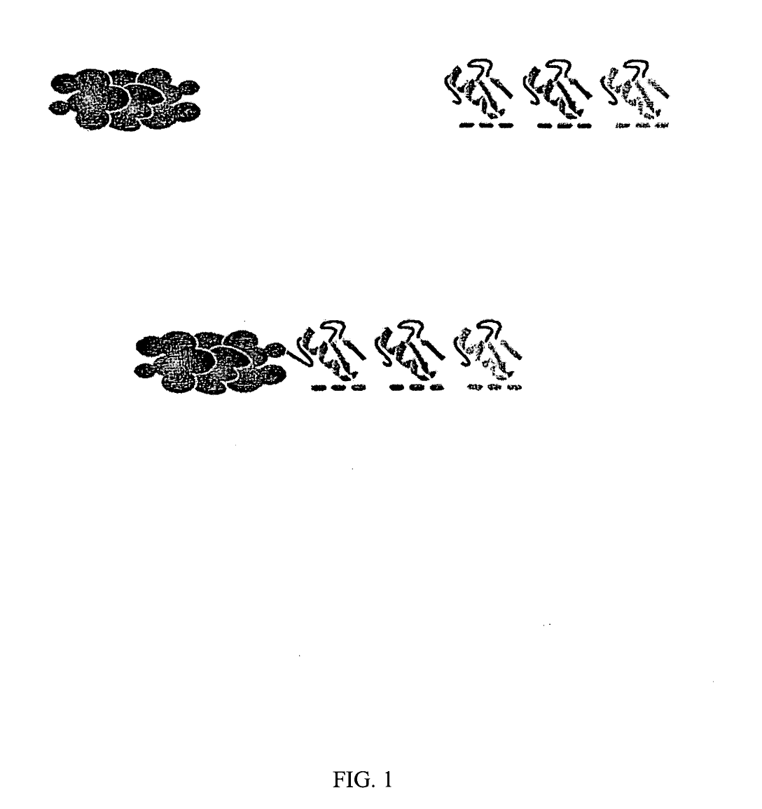

Fusion proteins according to the present invention are depicted schematically in FIG. 1.

In fusion proteins according to the invention, the zinc finger tag can be selected so that it specifically binds a nucleotide sequence that is 3, 6, 9, 12, 15, or 18 bases long. Typically, the nucleotide sequence is 9, 12, 15, or 18 bases long. In many applications, for maximum specificity, the nucleotide sequence is 18 bases long.

The fusion protein can include more than one protein of interest, but typically includes only one protein of interest. Within the fusion protein, the protein of interest and the zinc finger tag can be joined end-to-end in a single reading frame, or can be joined via a linker so that the protein of interest, the linker sequence, and the zinc finger tag are expressed in a single polypeptide that is the translation product of a single open reading frame. Suitable linkers include linkers such as TGEKP (SEQ ID NO: 674) and the longer linker TGGGGSGGGGTGEKP (SEQ ID NO: 675). This longer linker can be used when it is desired to have the two halves of a longer plurality of zinc finger binding polypeptides operate in a substantially independent manner in a fusion protein according to this invention. Modifications of this longer linker can also be used. For example, the polyglycine runs of four glycine (C) residues each can be of greater or lesser length (i.e., 3 or 5 glycine residues each). The serine residue (S) between the polyglycine runs can be replaced with threonine (T). The TGEKP (SEQ ID NO: 674) moiety that comprises part of the linker TGGGGSGGGGTGEKP (SEQ ID NO: 675) can be modified as described above for the TGEKP (SEQ ID NO: 674) linker alone. Still other linkers are known in the art and can alternatively be used. These include the linkers LRQKDGGGSERP (SEQ ID NO: 676), LRQKDGERP (SEQ ID NO: 677), GGRGRGRGRQ (SEQ ID NO: 678), QNKKGGSGDGKKKQHI (SEQ ID NO: 679), TGGERP (SEQ ID NO: 680), ATGEKP (SEQ ID NO: 681), and GGGSGGGGEGP (SEQ ID NO: 682), as well as derivatives of those linkers in which amino acid substitutions are made as described above for TGEKP (SEQ ID NO: 674) and TGGGGSGGGGTGEKP (SEQ ID NO: 675). For example, in these linkers, the serine (S) residue between the diglycine or polyglycine runs in QNKKGGSGDGKKKQHI (SEQ ID NO: 679) or GGGSGGGGEGP (SEQ ID NO: 682) can be replaced with threonine (T). In GGGSGGGGEGP (SEQ ID NO: 682), the glutamic acid (E) at position 9 can be replaced with aspartic acid (D). Other linkers such as glycine or serine repeats are well known in the art to link peptides (e.g., single chain antibody domains) and can be used in fusion proteins according to this invention. The use of a linker is not required for all purposes and can optionally be omitted. Additional suitable linkers for fusion proteins are well known in the art and need not be described further here; some suitable linkers are described, for example in U.S. Pat. No. 6,936,439 to Mann et al., incorporated herein by this reference. Such linkers typically comprise short oligopeptide regions that typically assume a random coil conformation. The linker typically consists of less than about 15 amino acid residues, more typically about 4 to 10 amino acid residues. For some applications, it might be desirable that the linker be cleavable. Cleavable linkers are known for a variety of applications.

The fusion protein can, if desired, further include conventional purification tags, such as polyhistidine or FLAG, or detectable protein moieties such as β-galactosidase, alkaline phosphatase, glutathione S-transferase, Protein A, or maltose-binding protein. The use of such tags and proteins as part of fusion proteins is described, for example, in J. Sambrook & D. W. Russell, “Molecular Cloning: A Laboratory Manual” (3rd ed., Cold Spring Harbor Laboratory Press, Cold Spring Harbor, N.Y., 2001), v. 3., ch. 15, pp 15.4-15.8, incorporated herein by this reference.

1. Components of Fusion Protein

a. Proteins of Interest

The protein of interest that is incorporated into a fusion protein according to the present invention can be virtually any protein whose properties need to be studied. This includes, but is not limited to, an antibody, an enzyme, a reporter protein, a receptor protein, a ligand for a receptor protein, a regulatory protein, or a membrane protein. The protein or polypeptide can be prokaryotic, eukaryotic, or viral in origin. If the protein is an antibody, it is typically in the form of a scFv or Fab′ fragment. The term “antibody” is used herein to refer to all protein molecules having affinity and cross-reactivity substantially equivalent to native antibodies having a four-chained L₂H₂ structure, whether monomeric or multimeric, and thus includes scFv or Fab′ fragments unless such fragments are specifically excluded. The term “antibody” as used herein further encompasses catalytic antibodies.

Additionally, as indicated above, a peptide can be linked to the zinc finger in a fusion protein. This can be done for virtually any peptide of physiological interest, including neurotransmitters, hormones, and other peptides.

Typically, the protein is monomeric, homodimeric, or homomultimeric; however, as discussed below, it is possible to express heterodimeric or heteromultimeric proteins, such as native antibodies, by the use of several fusion protein constructs, each engineered to express one chain of the heterodimer or heteromultimer. For example, the protein can be a chain of an antibody molecule, such as a heavy chain or a light chain, which can then reassemble to form an intact native antibody molecule. However, it is generally preferred that the protein is monomeric.

Typically, the protein of interest that is incorporated into a fusion protein according to the present invention is between about 80 and about 100,000 daltons in size, and has an isoelectric point of between about 4.5 and about 8.5. These parameters can vary depending on whether a peptide, a polypeptide, or a protein is incorporated into the fusion protein.

b. Zinc Finger Tags

Typically, a fusion protein according to the present invention includes a zinc finger tag that specifically binds a nucleotide sequence that is 3, 6, 9, 12, 15, or 18 bases long. Typically, the nucleotide sequence is 9, 12, 15, or 18 bases long. In many applications, for maximum specificity, the nucleotide sequence is 18 bases long.

Zinc finger tags, also referred to herein as zinc finger modules when incorporated into a fusion protein according to the present invention, that are suitable for use in fusion proteins according to the present invention have been described. For example, zinc finger modules that bind to nucleotide sequences of the general sequence 5′-ANN-3′ are disclosed in United States Patent Application Publication No. 2002/0165356, by Barbas et al., incorporated herein by this reference. Zinc finger modules that bind to nucleotide sequences of the general sequence 5′-GNN-3′ are disclosed in United States Patent Application Publication No. 2005/0148075 by Barbas, incorporated herein by this reference. Zinc finger modules that bind to nucleotide sequences of the general sequence 5′-CNN-3′ are disclosed in United States Patent Application Publication No. 2004/024385 by Barbas et al., incorporated herein by this reference. These zinc finger modules are all of the Cys₂-His₂ type, as described above. As used herein, the term “zinc finger module” means a segment of amino acids that has sequence-specific binding affinity for a defined segment of nucleotides, typically a 3-nucleotide segment. The zinc finger module can be incorporated into a larger molecule that is capable of sequence-specifically binding a longer defined segment of nucleotides, either as an independent zinc finger protein molecule or as a domain within a larger protein, such as a fusion protein. The term “zinc finger tag” as used herein refers specifically to a zinc finger module that is incorporated within a fusion protein.

In using zinc finger modules that bind these triplets, typically, longer zinc finger modules are assembled in tandem to form a domain that binds a nucleotide sequence that is 3, 6, 9, 12, 15, or 18 bases long. Typically, the nucleotide sequence is 9, 12, 15, or 18 bases long. In many applications, for maximum specificity, the nucleotide sequence is 18 bases long.

The nucleotide sequence that is bound is selected such that it is found in a DNA molecule that is utilized in various ways according to the method in which the fusion protein is employed. For example, the DNA molecule can be bound to a solid support and incorporated into an array. In another alternative, the DNA molecule can be covalently linked to a fluorescent moiety and used to label the protein of interest.

As used herein, the amino acids, which occur in the various amino acid sequences appearing herein, are identified according to their well-known, three-letter or one-letter abbreviations. The nucleotides, which occur in the various DNA fragments, are designated with the standard single-letter designations used routinely in the art.

In a peptide or protein, suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g. J. D. Watson et al., “Molecular Biology of the Gene” (4th Edition, 1987, Benjamin/Cummings, Palo Alto), p. 224). Specifically, in particular, the conservative amino acid substitutions can be any of the following: (1) any of isoleucine for leucine or valine, leucine for isoleucine, and valine for leucine or isoleucine; (2) aspartic acid for glutamic acid and glutamic acid for aspartic acid; (3) glutamine for asparagine and asparagine for glutamine; and (4) serine for threonine and threonine for serine. Other substitutions can also be considered conservative, depending upon the environment of the particular amino acid. For example, glycine (G) and alanine (A) can frequently be interchangeable, as can be alanine and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the different pK's of these two amino acid residues or their different sizes are not significant. Still other changes can be considered “conservative” in particular environments. For example, if an amino acid on the surface of a protein is not involved in a hydrogen bond or salt bridge interaction with another molecule, such as another protein subunit or a ligand bound by the protein, negatively charged amino acids such as glutamic acid and aspartic acid can be substituted for by positively charged amino acids such as lysine or arginine and vice versa. Histidine (H), which is more weakly basic than arginine or lysine, and is partially charged at neutral pH, can sometimes be substituted for these more basic amino acids. Additionally, the amides glutamine (Q) and asparagine (N) can sometimes be substituted for their carboxylic acid homologues, glutamic acid and aspartic acid.

As used herein, “expression vector” refers to a plasmid, virus or other vehicle known in the art that has been manipulated by insertion or incorporation of heterologous DNA, such as nucleic acid encoding the fusion proteins herein or expression cassettes provided herein. Such expression vectors contain a promoter sequence for efficient transcription of the inserted nucleic acid in a cell. The expression vector typically contains an origin of replication, and a promoter, as well as specific genes that permit phenotypic selection of transformed cells.

As used herein, “host cells” are cells in which a vector can be propagated and its DNA expressed. The term also includes any progeny of the subject host cell. It is understood that all progeny may not be identical to the parental cell since there may be mutations that occur during replication. Such progeny are included when the term “host cell” is used. Methods of stable transfer where the foreign DNA is continuously maintained in the host are known in the art.

As used herein, an expression or delivery vector refers to any plasmid or virus into which a foreign or heterologous DNA may be inserted for expression in a suitable host cell—i.e., the protein or polypeptide encoded by the DNA is synthesized in the host cell's system. Vectors capable of directing the expression of DNA segments (genes) encoding one or more proteins are referred to herein as “expression vectors”. Also included are vectors that allow cloning of cDNA (complementary DNA) from mRNAs produced using reverse transcriptase.

As used herein, a gene refers to a nucleic acid molecule whose nucleotide sequence encodes an RNA or polypeptide. A gene can be either RNA or DNA. Genes may include regions preceding and following the coding region (leader and trailer) as well as intervening sequences (introns) between individual coding segments (exons).

As used herein, “isolated,” with reference to a nucleic acid molecule or polypeptide or other biomolecule means that the nucleic acid or polypeptide has separated from the genetic environment from which the polypeptide or nucleic acid were obtained. It may also mean altered from the natural state. For example, a polynucleotide or a polypeptide naturally present in a living animal is not “isolated”, but the same polynucleotide or polypeptide separated from the coexisting materials of its natural state is “isolated”, as the term is employed herein. Thus, a polypeptide or polynucleotide produced and/or contained within a recombinant host cell is considered isolated. Also intended as an “isolated polypeptide” or an “isolated polynucleotide” are polypeptides or polynucleotides that have been purified, partially or substantially, from a recombinant host cell or from a native source. For example, a recombinantly produced version of a compound can be substantially purified by the one-step method described in Smith et al. (1988) Gene 67:3140. The terms isolated and purified are sometimes used interchangeably.

Thus, by “isolated” the nucleic acid is free of the coding sequences of those genes that, in a naturally-occurring genome immediately flank the gene encoding the nucleic acid of interest Isolated DNA may be single-stranded or double-stranded, and may be genomic DNA, cDNA, recombinant hybrid DNA, or synthetic DNA. It may be identical to a native DNA sequence, or may differ from such sequence by the deletion, addition, or substitution of one or more nucleotides.

Isolated or purified as it refers to preparations made from biological cells or hosts means any cell extract containing the indicated DNA or protein including a crude extract of the DNA or protein of interest. For example, in the case of a protein, a purified preparation can be obtained following an individual technique or a series of preparative or biochemical techniques and the DNA or protein of interest can be present at various degrees of purity in these preparations. The procedures may include for example, but are not limited to, ammonium sulfate fractionation, gel filtration, ion exchange chromatography, affinity chromatography, density gradient centrifugation, electrophoresis, electrofocusing, chromatofocusing, or other protein purification techniques known in the art.

A preparation of DNA or protein that is “substantially pure” or “isolated” should be understood to mean a preparation free from naturally occurring materials with which such DNA or protein is normally associated in nature. “Essentially pure” should be understood to mean a “highly” purified preparation that contains at least 95% of the DNA or protein of interest.

A cell extract that contains the DNA or protein of interest should be understood to mean a homogenate preparation or cell-free preparation obtained from cells that express the protein or contain the DNA of interest. The term “cell extract” is intended to include culture media, especially spent culture media from which the cells have been removed.

As used herein, “truncated” refers to a zinc finger-nucleotide binding polypeptide derivative that contains less than the full number of zinc fingers found in the native zinc finger binding protein or that has been deleted of non-desired sequences. For example, truncation of the zinc finger-nucleotide binding protein TFIIIA, which naturally contains nine zinc fingers, might be a polypeptide with only zinc fingers one through three. Expansion refers to a zinc finger polypeptide to which additional zinc finger modules have been added. For example, TFIIIA can be extended to 12 fingers by adding 3 zinc finger domains. In addition, a truncated zinc finger-nucleotide binding polypeptide may include zinc finger modules from more than one wild type polypeptide, thus resulting in a “hybrid” zinc finger-nucleotide binding polypeptide.

As used herein, “mutagenized” refers to a zinc finger derived-nucleotide binding polypeptide that has been obtained by performing any of the known methods for accomplishing random or site-directed mutagenesis of the DNA encoding the protein. For instance, in TFIIIA, mutagenesis can be performed to replace nonconserved residues in one or more of the repeats of the consensus sequence. Truncated zinc finger-nucleotide binding proteins can also be mutagenized. Techniques for mutagenesis are known in the art, and include, but are not limited to, site-directed mutagenesis, linker-scanning mutagenesis, and other techniques.

As used herein, a polypeptide “variant” or “derivative” refers to a polypeptide that is a mutagenized form of a polypeptide or one produced through recombination but that still retains a desired activity, such as the ability to bind to a ligand or a nucleic acid molecule or to modulate transcription.

As used herein, a zinc finger-nucleotide binding polypeptide “variant” or “derivative” refers to a polypeptide that is a mutagenized form of a zinc finger protein or one produced through recombination. A variant may be a hybrid that contains zinc finger domain(s) from one protein linked to zinc finger domain(s) of a second protein, for example. The domains may be wild type or mutagenized. A “variant” or “derivative” includes a truncated form of a wild type zinc finger protein, which contains less than the original number of fingers in the wild type protein. Examples of zinc finger-nucleotide binding polypeptides from which a derivative or variant may be produced include SP1C, TFIIIA and Zif268, as well as C7 (a derivative of Zif268) and other zinc finger proteins known in the art. These zinc finger proteins from which other zinc finger proteins are derived are referred to herein as “backbones.”

As used herein a “zinc finger-nucleotide binding target or motif” refers to any two or three-dimensional feature of a nucleotide segment to which a zinc finger-nucleotide binding derivative polypeptide binds with specificity. Included within this definition are nucleotide sequences, generally of five nucleotides or less, as well as the three dimensional aspects of the DNA double helix, such as, but are not limited to, the major and minor grooves and the face of the helix. The motif is typically any sequence of suitable length to which the zinc finger polypeptide can bind. For example, a three finger polypeptide binds to a motif typically having about 9 to about 14 base pairs. Preferably, the recognition sequence is at least about 16 base pairs, more preferably 18 bases, to ensure specificity within the genome. Therefore, zinc finger-nucleotide binding polypeptides of any specificity are provided. The zinc finger binding motif can be any sequence designed empirically or to which the zinc finger protein binds. The motif may be found in any DNA or RNA sequence, including regulatory sequences, exons, introns, or any non-coding sequence. As detailed further below, the motif can be selected for binding to an array.

As used herein, the term “vector” refers to a nucleic acid molecule capable of transporting between different genetic environments another nucleic acid to which it has been operably linked. Preferred vectors are those capable of autonomous replication and expression of structural gene products present in the DNA segments to which they are operably linked. Vectors, therefore, preferably contain the replicons and selectable markers described earlier.

As used herein with regard to nucleic acid molecules, including DNA fragments, the phrase “operably linked” means the sequences or segments have been covalently joined, preferably by conventional phosphodiester bonds, into one strand of DNA, whether in single or double-stranded form such that the operably linked portions function as intended. If the DNA fragments are not originally in one strand of DNA, they can be joined by ligation, such as blunt-ended ligation or ligation employing cohesive ends, as is well known in the art. The choice of vector to which transcription unit or a cassette provided herein is operably linked depends directly, as is well known in the art, on the functional properties desired, e.g., vector replication and protein expression, and the host cell to be transformed, these being limitations inherent in the art of constructing recombinant DNA molecules. As used herein, the term “operably linked” includes both DNA segments that are joined directly end-to-end and DNA segments that are joined through one or more intervening DNA segments, such as linkers or other functional domains in a fusion protein.

The zinc finger tag that forms part of a fusion protein according to this invention typically contains a nucleotide binding region of from 5 to 10 amino acid residues, preferably about 7 amino acid residues, for each triplet of bases that is specifically bound.

A zinc finger tag incorporated into a fusion protein of this invention can be a non-naturally occurring variant. As used herein, the term “non-naturally occurring” means, for example, one or more of the following: (a) a peptide comprised of a non-naturally occurring amino acid sequence; (b) a peptide having a non-naturally occurring secondary structure not associated with the peptide as it occurs in nature; (c) a peptide which includes one or more amino acids not normally associated with the species of organism in which that peptide occurs in nature; (d) a peptide which includes a stereoisomer of one or more of the amino acids comprising the peptide, which stereoisomer is not associated with the peptide as it occurs in nature; (e) a peptide which includes one or more chemical moieties other than one of the natural amino acids; or (f) an isolated portion of a naturally occurring amino acid sequence (e.g., a truncated sequence). A fusion protein of this invention exists in an isolated form and purified to be substantially free of contaminating substances. A zinc finger tag in a fusion protein according to the present invention can refer to a polypeptide that is, preferably, a mutagenized form of a zinc finger protein or one produced through recombination. The zinc finger tag can be a hybrid which contains zinc finger domain(s) from one protein linked to zinc finger domain(s) of a second protein, for example. The domains may be wild type or mutagenized. The zinc finger tag can be a truncated form of a wild type zinc finger protein. Examples of zinc finger proteins from which a zinc finger tag can be produced include TFIIIA and zif268.

A zinc finger tag incorporated into a fusion protein according to this invention can comprise a unique heptamer (contiguous sequence of 7 amino acid residues) within the α-helical domain of the zinc finger tag, which heptameric sequence determines binding specificity to a target nucleotide. That heptameric sequence can be located anywhere within the α-helical domain but it is preferred that the heptamer extend from position −1 to position 6 as the residues are conventionally numbered in the art. A zinc finger tag incorporated into a fusion protein according to this invention can include any β-sheet and framework sequences known in the art to function as part of a zinc finger protein.

The zinc finger tag can be derived or produced from a wild type zinc finger protein by truncation or expansion, or as a variant of a wild type-derived polypeptide by a process of site directed mutagenesis, or by a combination of the procedures. The term “truncated” refers to a zinc finger tag that contains less that the full number of zinc fingers found in the native zinc finger binding protein or that has been deleted of non-desired sequences. For example, truncation of the zinc finger-nucleotide binding protein TFIIIA, which naturally contains nine zinc fingers, might be a polypeptide with only zinc fingers one through three. Expansion refers to a zinc finger polypeptide to which additional zinc finger modules have been added. For example, TFIIIA may be extended to 12 fingers by adding 3 zinc finger domains. In addition, a truncated zinc finger-nucleotide binding polypeptide may include zinc finger modules from more than one wild type polypeptide, thus resulting in a “hybrid” zinc finger-nucleotide binding polypeptide.

The term “mutagenized” refers to a zinc finger tag incorporated into a fusion protein according to the present invention that has been obtained by performing any of the known methods for accomplishing random or site-directed mutagenesis of the DNA encoding the protein. For instance, in TFIIIA, mutagenesis can be performed to replace nonconserved residues in one or more of the repeats of the consensus sequence. Truncated zinc finger-nucleotide binding proteins can also be mutagenized. Examples of known zinc finger-nucleotide binding polypeptides that can be truncated, expanded, and/or mutagenized according to the present invention in order to alter the function of a nucleotide sequence containing a zinc finger-nucleotide binding motif includes TFIIIA, Zif268, and Sp1C. Those of skill in the art know other zinc finger-nucleotide binding proteins that can be truncated, expanded, and/or mutagenized as described above.

Specific zinc finger modules that have a specific binding affinity for nucleotide sequences of the form 5′-ANN-3′ are disclosed, for example, in United States Patent Application Publication No. 2002/0165356 by Barbas et al., particularly those sequences that are identified as SEQ ID NO: 7 through SEQ ID NO: 70 and SEQ ID NO: 107 through SEQ ID NO: 112 therein. Specific zinc finger modules that have a specific binding affinity for nucleotide sequences of the form 5′-CNN-3′ are disclosed in United States Patent Application Publication No. 2004/024385 by Barbas et al., particularly those sequences that are identified as SEQ ID NO: 1 through SEQ ID NO: 25 therein. Specific zinc finger modules that have a specific binding affinity for nucleotide sequences of the form 5′-GNN-3′ are disclosed in United States Patent Application Publication No. 2005/0148075 by Barbas, particularly those sequences that are identified as SEQ ID NO: 17-SEQ ID NO:110 therein. Specific zinc finger modules that have a specific binding affinity for nucleotide sequences of the form 5′-AGC-3′ are described further below in terms of the sequences of the zinc finger modules. Specific zinc finger modules that have a specific binding affinity for nucleotide sequences of the form 5′-TNN-3′ are described further below in terms of the sequences of the zinc finger modules. These zinc finger modules or zinc finger tags are all of the Cys₂-His₂ type; however, other alternatives are described below. These zinc finger modules can be combined as needed and used as zinc finger tags in fusion proteins according to the present invention; other zinc finger modules are also known in the art. As used herein, the terms “zinc finger modules” and “zinc finger DNA binding domains” are used interchangeably and equivalently.

Methods for isolating, selecting, and screening these zinc finger modules are disclosed, for example, in United States Patent Application Publication No. 2002/0165356 by Barbas et al., United States Patent Application Publication No. 2004/024385 by Barbas et al. and United States Patent Application Publication No. 2005/0148075 by Barbas. These methods can use, for example, the production and screening of phagemid libraries. These methods are described farther in D. J. Segal et al., “Toward Controling Gene Expression at Will: Selection and Design of Zinc Finger Domains Recognizing Each of the 5′-GNN-3′ DNA Target Sequences,” Proc. Natl. Acad. Sci. USA 96: 2758-2765 (1999); B. Dreier et al., “Insights into the Molecular Recognition of the 5′-GNN-3' Family of DNA Sequences by Zinc Finger Domains,” J. Mol. Biol. 303: 489-502 (2000); P. Blancafort et al., “Designing Transcription Factor Architectures for Drug Discovery,” Mol. Pharmacol. 66: 1361-1371 (2004); and B. Dreier et al., “Development of Zinc Finger Domains for Recognition of the 5′-ANN-3′ Family of DNA Sequences and Their Use in the Construction of Artificial Transcription Factors,” J. Biol. Chem. 276: 29466-29478 (2001), all of which are incorporated herein by this reference.

For example, for the determination of zinc finger modules capable of specifically binding 5′-GNN-3′, a striking conservation of all three of the primary DNA contact positions (−1, 3, and 6) was observed for virtually all the clones of a given target. Although many of these residues were observed previously at these positions following selections with much less complete libraries, the extent of conservation observed here represents a dramatic improvement over earlier studies (Choo, Y. & Klug, A. (1994) Proc Natl Acad Sci USA 91, 11163-7, Greisman, H. A. & Pabo, C. O. (1997) Science (Washington, D.C.) 275, 657-661, Rebar, E. J. & Pabo, C. O. (1994) Science (Washington, D.C., 1883-) 263, 671-3, Jamieson, A. C., Kim, S.-H. & Wells, J. A. (1994) Biochemistry 33, 5689-5695, Jamieson, A. C., Wang, H. & Kim, S.-H. (1996) PNAS 93, 12834-12839., Wu, H., Yang, W.-P. & Barbas III, C. F. (1995) PNAS 92, 344-348). These results establish that the teachings of the prior art that the three helical positions −1, 3, and 6 of a zinc finger domain are sufficient to allow for the detailed description of the DNA binding specificity of the domain are incorrect.

Typically, phage selections have shown a consensus selection in only one or two of these positions. The greatest sequence variation occurred at the residues in positions 1 and 5, which do not make bases contacts in the Zif268/DNA structure and were expected not to contribute significantly to recognition (Pavletich, N. P. & Pabo, C. O. (1991) Science (Washington, D.C., 1883-) 252, 809-17, Elrod-Erickson, M., Rould, M. A., Nekludova, L. & Pabo, C. O. (1996) Structure (London) 4, 1171-1180). Variation in positions 1 and 5 also implied that the conservation in the other positions was due to their interaction with the DNA and not simply the fortuitous amplification of a single clone due to other reasons. Conservation of residue identity at position 2 was also observed. The conservation of position −2 is somewhat artifactual; the NNK library had this residue fixed as serine. This residue makes contacts with the DNA backbone in the Zif268 structure. Both libraries contained an invariant leucine at position 4, a critical residue in the hydrophobic core that stabilizes folding of this domain.

Impressive amino acid conservation was observed for recognition of the same nucleotide in different targets. For example, Asn in position 3 (Asn³) was virtually always selected to recognize adenine in the middle position, whether in the context of GAG, GM, GAT, or GAC. Gln⁻¹ and Arg⁻¹ were always selected to recognize adenine or guanine, respectively, in the 3′ position regardless of context. Amide side chain based recognition of adenine by Gln or Asn is well documented in structural studies as is the Arg guanidinium side chain to guanine contact with a 3′ or 5′ guanine (Elrod-Erickson, M., Benson, T. E. & Pabo, C. O. (1998) Structure (London) 6, 451-464, Kim, C. A. & Berg, J. M. (1996) Nature Structural Biology 3, 940-945., Fairall, L., Schwabe, J. W. R., Chapman, L., Finch, J. T. & Rhodes, D. (1993) Nature (London) 366, 483-7). More often, however, two or three amino acids were selected for nucleotide recognition. His³ or Lys³ (and to a lesser extent, Gly³) were selected for the recognition of a middle guanine. Ser and Ala³ were selected to recognize a middle thymine. Thr³, Asp³, and Glu³ were selected to recognize a middle cytosine. Asp and Glu were also selected in position −1 to recognize a 3′ cytosine, while Thr-1 and Ser-1 were selected to recognize a 3′ thymine. Accordingly, these findings, and analogous findings, can be used to design suitable sequences for zinc finger tags incorporated into fusion proteins according to the present invention.

Selected Zif268 variants were subcloned into a bacterial expression vector, and the proteins overexpressed (finger-2 proteins, hereafter referred to by the subsite for which they were panned). It is important to study soluble proteins rather than phage-fusions since it is known that the two may differ significantly in their binding characteristics (Crameri, A., Cwirla, S. & Stemmer, W. P. (1996) Nat. Med. 2, 100-102). The proteins were tested for their ability to recognize each of the 16 5′-GNN-3′ finger-2 subsites using a multi-target ELISA assay. This assay provided an extremely rigorous test for specificity since there were always six “non-specific” sites which differed from the “specific” site by only a single nucleotide out of a nine-nucleotide target. Many of the phage-selected finger-2 proteins showed exquisite specificity, while others demonstrated varying degrees of crossreactivity. Some polypeptides actually bound better to subsites other than those for which they were selected.

Attempts were made to improve binding specificity by modifying the recognition helix using site-directed mutagenesis. Data from selections and structural information guided mutant design. As the most exhaustive study performed to date, over 100 mutant proteins were characterized in an effort to expand understanding of the rules of recognition. Although helix positions 1 and 5 are not expected to play a direct role in DNA recognition, the best improvements in specificity always involved modifications in these positions. These residues have been observed to make phosphate backbone contacts, which contribute to affinity in a non-sequence specific manner. Removal of non-specific contacts increases the importance of the specific contacts to the overall stability of the complex, thereby enhancing specificity. For example, the specificity of polypeptides for target triplets GAC, GAA, and GAG were improved simply by replacing atypical, charged residues in positions 1 and 5 with smaller, uncharged residues. Again, these findings can be used to design suitable sequences for zinc finger tags incorporated into fusion proteins according to the present invention.

Another class of modifications involved changes to both binding and non-binding residues. The crossreactivity of polypeptides for GGG and the finger-2 subsite GAG was abolished by the modifications His³→Lys and Thr⁵→Val. It is interesting to note that His³ was unanimously selected during panning to recognize the middle guanine, although Lys³ provided better discrimination of A and G. This suggests that panning conditions for this protein may have favored selection by a parameter such as affinity over that of specificity. In the Zif268 structure, His³ donates a hydrogen bond to the N7 of the middle guanine (Pavletich, N. P. & Pabo, C. O. (1991) Science (Washington, D.C., 1883-) 252, 809-17, Elrod-Erickson, M., Rould, M. A., Nekludova, L. & Pabo, C. O. (1996) Structure (London) 4, 1171-1180). This bond could also be made with N7 of adenine, and in fact Zif268 does not discriminate between G and A in this position (Swimoff, A. H. & Milbrandt, J. (1995) Mol. Cell. Biol. 15, 2275-87). His³ was found to specify only a middle guanine in polypeptides targeted to GGA, GGC, and GGT, even though Lys³ was selected during panning for GGC and GGT. Similarly, the multiple crossreactivities of polypeptides targeted to GTG were attenuated by modifications Lys¹→Ser and Ser³→Glu, resulting in a 5-fold loss in affinity. Glu³ has been shown to be very specific for cytosine in binding site selection studies of Zif268 (Swimoff, A. H. & Milbrandt, J. (1995) Mol. Cell. Biol. 15, 2275-87). No structural studies show an interaction of Glu³ with the middle thymine and Glu³ was never selected to recognize a middle thymine in this study or any others (Choo, Y. & Klug, A. (1994) Proc Natl Acad Sci USA 91, 11163-7, Greisman, H. A. & Pabo, C. O. (1997) Science (Washington, D.C.) 275, 657-661, Rebar, E. J. & Pabo, C. O. (1994) Science (Washington, D.C., 1883-) 263, 671-3, Jamieson, A. C., Kim, S.-H. & Wells, J. A. (1994) Biochemistry 33, 5689-5695, Jamieson, A. C., Wang, H. & Kim, S.-H. (1996) PNAS 93, 12834-12839, Isalan, M., Klug, A. & Choo, Y. (1998) Biochemistry 37, 12026-33, Wu, H., Yang, W.-P. & Barbas III, C. F. (1995) PNAS 92, 344-348). Despite this, the Ser³→Glu modification favored the recognition of a middle thymine over cytosine. These examples illustrate the limitations of relying on previous structures and selection data to understand the structural elements underlying specificity. It should also be emphasized that improvements by modifications involving positions 1 and 5 could not have been predicted by existing “recognition codes” (Desjarlais, J. R. & Berg, J. M. (1992) Proc Natl Acad Sci USA 89, 7345-9. Suzuki, M., Gerstein, M. & Yagi, N. (1994) Nucleic Acids Res. 22, 3397-405, Choo, Y. & Klug, A. (1994) Proc. Natl. Acad. Sci. U.S.A. 91, 11168-72, Choo, Y. & Klug, A. (1997) Curr. Opin. Struct. Biol. 7, 117-125), which typically only consider positions −1, 2, 3, and 6. Only by the combination of selection and site-directed mutagenesis can the intricacies of zinc finger/DNA recognition be fully understood.

From the combined selection and mutagenesis data it emerged that specific recognition of many nucleotides could be best accomplished using motifs, rather than a single amino acid. For example, the best specification of a 3′ guanine was achieved using the combination of Arg⁻¹, Ser¹, and Asp² (the RSD motif. By using Val⁵ and Arg⁶ to specify a 5′ guanine, recognition of subsites GGG, GAG, GTG, and GCG could be accomplished using a common helix structure (RSD-X-LVR) (SEQ ID NO: 683) differing only in the position 3 residue (Lys³ for GGG, Asn³ for GAG, Glu³ for GTG, and Asp³ for GCG). Similarly, 3′ thymine was specified using Thr⁻¹, Ser¹, and Gly² in the final clones (the TSG motif). Further, a 3′ cytosine could be specified using Asp⁻¹, Pro¹, and Gly² in (the DPG motif) except when the subsite was GCC; Pro¹ was not tolerated by this subsite. Specification of a 3′ adenine was with Gln⁻¹, Ser¹, Ser² in two clones (QSS motif). Residues of positions 1 and 2 of the motifs were studied for each of the 3′ bases and found to provide optimal specificity for a given 3′ base as described here. These motifs can be used to construct appropriate zinc finger tags.

The multi-target ELISA assay assumed that all the proteins preferred guanine in the 5′ position since all proteins contained Arg⁶ and this residue is known from structural studies to contact guanine at this position (Pavletich, N. P. & Pabo, C. O. (1991) Science (Washington, D.C., 1883-) 252, 809-17, Elrod-Erickson, M., Rould, M. A., Nekludova, L. & Pabo, C. O. (1996) Structure (London) 4, 1171-1180, Elrod-Erickson, M., Benson, T. E. & Pabo, C. O. (1998) Structure (London) 6, 451-464, Kim, C. A. & Berg, J. M. (1996) Nature Structural Biology 3, 940-945, Pavletich, N. P. & Pabo, C. O. (1993) Science (Washington, D.C., 1883-) 261, 1701-7, Houbaviy, H. B., Usheva, A., Shenk, T. & Burley, S. K. (1996) Proc Nat Acad Sci USA 93, 13577-82, Fairall, L., Schwabe, J. W. R., Chapman, L., Finch, J. T. & Rhodes, D. (1993) Nature (London) 366, 483-7, Wuttke, D. S., Foster, M. P., Case, D. A., Gottesfeld, J. M. & Wright, P. E. (1997) J. Mol. Biol. 273, 183-206, Nolte, R. T., Conlin, R. M., Harrison, S. C. & Brown, R. S. (1998) Proc. Natl. Acad. Sci. U.S.A. 95, 2938-2943). This interaction was demonstrated using the 5′ binding site signature assay ((Choo, Y. & Klug, A. (1994) Proc. Natl. Acad. Sci. U.S.A. 91, 11168-72). Each protein was applied to pools of 16 oligonucleotide targets in which the 5′ nucleotide of the finger-2 subsite was fixed as G, A, T, or C and the middle and 3′ nucleotides were randomized. All proteins preferred the GNN pool with essentially no crossreactivity.

The results of the multi-target ELISA assay were confirmed by affinity studies of purified proteins. In cases where crossreactivity was minimal in the ELISA assay, a single nucleotide mismatch typically resulted in a greater than 100-fold loss in affinity. This degree of specificity had yet to be demonstrated with zinc finger proteins. In general, proteins selected or designed to bind subsites with G or A in the middle and 3′ position had the highest affinity, followed by those which had only one C or A in the middle or 3′ position, followed by those which contained only T or C. The former group typically bound their targets with a higher affinity than Zif268 (10 nM), the latter with somewhat lower affinity, and almost all the proteins had an affinity lower than that of the parental C7 protein. There was no correlation between binding affinity and binding specificity suggesting that specificity can result not only from specific protein-DNA contacts, but also from interactions which exclude all but the correct nucleotide. These findings can be used to design suitable sequences for zinc finger tags incorporated into fusion proteins according to the present invention.

Asp² was always co-selected with Arg⁻¹ in all proteins for which the target subsite was GNG. It is now understood that there are two reasons for this. From structural studies of Zif268 (Pavletich, N. P. & Pabo, C. O. (1991) Science (Washington, D.C., 1883-) 252, 809-17, Elrod-Erickson, M., Rould, M. A., Nekludova, L. & Pabo, C. O. (1996) Structure (London) 4, 1171-1180), it is known that Asp² of finger 2 makes a pair of buttressing hydrogen bonds with Arg⁻¹ which stabilize the Arg⁻¹/3′ guanine interaction, as well as some water-mediated contacts. However, the carboxylate of Asp² also accepts a hydrogen bond from the N4 of a cytosine that is base-paired to a 5′ guanine of the finger-1 subsite. Adenine base paired to T in this position can make an analogous contact to that seen with cytosine. This interaction is particularly important because it extends the recognition subsite of finger 2 from three nucleotides (GNG) to four (GNG(G/T)) (Isalan, M., Choo, Y. & Kug, A. (1997) Proc. Nat. Acad. Sci. U.S.A. 94, 5617-5621., Jamieson, A. C., Wang, H. & Kim, S.-H. (1996) PNAS 93, 12834-12839, Isalan, M., Klug, A. & Choo, Y. (1998) Biochemistry 37, 12026-33). This phenomenon is referred to as “target site overlap”, and has three important ramifications. First, Asp² was favored for selection by the library when the finger-2 subsite was GNG because the finger-1 subsite contained a 5′ guanine. Second, it may limit the utility of the libraries used in this study to selection on GNN or TNN finger-2 subsites because finger 3 of these libraries contains an Asp², which may help specify the 5′ nucleotide of the finger-2 subsite to be G or T. In Zif268 and C7, which have Thr⁶ in finger 2, Asp² of finger 3 enforces G or T recognition in the 5′ position (T/G)GG. This interaction may also explain why previous phage display studies, which all used Zif268-based libraries, have found selection limited primarily to GNN recognition (Choo, Y. & Klug, A. (1994) Proc Natl Acad Sci USA 91, 11163-7., Rebar, E. J. & Pabo, C. O. (1994) Science (Washington, D.C., 1883-) 263, 671-3, Jamieson, A. C., Kim, S.-H. & Wells, J. A. (1994) Biochemistry 33, 5689-5695, Jamieson, A. C., Wang, H. & Kim, S.-H. (1996) PNAS 93, 12834-12839, Isalan, M., Klug, A. & Choo, Y. (1998) Biochemistry 37, 12026-33, Wu, H., Yang, W.-P. & Barbas III, C. F. (1995) PNAS 92, 344-348).

Finally, target site overlap potentially limits the use of these zinc fingers as modular building blocks. From structural data it is known that there are some zinc fingers in which target site overlap is quite extensive, such as those in GL1 and YY1, and others which are similar to Zif268 and display only modest overlap. In the final set of proteins, Asp² is found in polypeptides that bind GGG, GAG, GTG, and GCG. The overlap potential of other residues found at position 2 is largely unknown, however structural studies reveal that many other residues found at this position may participate in such cross-subsite contacts. Fingers containing Asp² may limit modularity, since they would require that each GNG subsite be followed by a T or G. However, this is relatively rare. Accordingly, it is typically preferred that zinc finger tags incorporated into fusion proteins according to the present invention do not include modules with target site overlap.

A zinc finger tag incorporated into a fusion protein according to this invention can be made using a variety of standard techniques well known in the art (See, e.g., U.S. patent application Ser. No. 08/676,318, filed Jan. 18, 1995, the entire disclosure of which is incorporated herein by reference). Phage display libraries of zinc finger proteins were created and selected under conditions that favored enrichment of sequence specific proteins. Zinc finger domains recognizing a number of sequences required refinement by site-directed mutagenesis that was guided by both phage selection data and structural information.

The murine Cys₂-His₂ zinc finger protein Zif268 can be used for construction of phage display libraries (Wu, H, Yang, W.-P. & Barbas III, C. F. (1995) PNAS 92, 344-348) for the generation of zinc finger tags incorporated into fusion proteins according to this invention. Zif268 is structurally the most well characterized of the zinc-finger proteins (Pavletich, N. P. & Pabo, C. O. (1991) Science (Washington, D.C., 1883-) 252, 809-17, Elrod-Erickson, M., Rould, M. A., Nekludova, L. & Pabo, C. O. (1996) Structure (London) 4, 1171-1180, Swimoff, A. H. & Milbrandt, J. (1995) Mol. Cell. Biol. 15, 2275-87). DNA recognition in each of the three zinc finger domains of this protein is mediated by residues in the N-terminus of the α-helix contacting primarily three nucleotides on a single strand of the DNA. The operator binding site for this three finger protein is 5′-GCGTGGGCG-3′. (SEQ ID NO: 684). Structural studies of Zif268 and other related zinc finger-DNA complexes (Elrod-Erickson, M., Benson, T. E. & Pabo, C. O. (1998) Structure (London) 6, 451-464, Kim, C. A. & Berg, J. M. (1996) Nature Structural Biology 3, 940-945, Pavletich, N. P. & Pabo, C. O. (1993) Science Washington, D.C., 1883-) 261, 1701-7, Houbaviy, H. B., Usheva, A., Shenk, T. & Burley, S. K. (1996) Proc Natl Acad Sci USA 93, 13577-82, Fairall, L., Schwabe, J. W. R., Chapman, L., Finch, J. T. & Rhodes, D. (1993) Nature (London) 366, 483-7, Wuttke, D. S., Foster, M. P., Case, D. A., Gottesfeld, J. M. & Wright, P. E. (1997) J. Mol. Biol. 273, 183-206., Nolte, R. T., Conlin, R. M., Harrison, S. C. & Brown, R. S. (1998) Proc. Natl. Acad. Sci. U.S.A. 95, 2938-2943, Narayan, V. A., Kriwacki, R. W. & Caradonna, J. P. (1997) J. Biol. Chem. 272, 7801-7809) have shown that residues from primarily three positions on the α-helix, −1, 3, and 6, are involved in specific base contacts. Typically, the residue at position −1 of the α-helix contacts the 3′ base of that finger's subsite while positions 3 and 6 contact the middle base and the 5′ base, respectively.

In order to select a family of zinc finger domains recognizing the 5′-GNN-3′ subset of sequences, two highly diverse zinc finger libraries were constructed in the phage display vector pComb3H (Barbas III, C. F., Kang, A. S., Lerner, R. A. & Benkovic, S. J. (1991) Proc. Natl. Acad. Sci. USA 88, 7978-7982., Rader, C. & Barbas III, C. F. (1997) Curr. Opin. Biotechnol. 8, 503-508). Both libraries involved randomization of residues within the α-helix of finger 2 of C7, a variant of Zif268 (Wu, H., Yang, W.-P. & Barbas III, C. F. (1995) PNAS 92, 344-348). Library 1 was constructed by randomization of positions −1, 1, 2, 3, 5, 6 using a NNK doping strategy while library 2 was constructed using a VNS doping strategy with randomization of positions −2, −1, 1, 2, 3, 5, 6. The NNK doping strategy allows for all amino acid combinations within 32 codons while VNS precludes Tyr, Phe, Cys and all stop codons in its 24 codon set. The libraries consisted of 4.4×10⁹ and 3.5×10⁹ members, respectively, each capable of recognizing sequences of the 5′ GCGNNNGCG-3′ (SEQ ID NO: 685) type. The size of the NNK library ensured that it could be surveyed with 99% confidence while the VNS library was highly diverse but somewhat incomplete. These libraries are, however, significantly larger than previously reported zinc finger libraries (Choo, Y. & Klug, A. (1994) Proc Natl Acad Sci USA 91, 11163-7, Greisman, H. A. & Pabo, C. O. (1997) Science (Washington, D.C.) 275, 657-661, Rebar, E. J. & Pabo, C. O. (1994) Science (Washington, D.C., 1883-) 263, 671-3, Jamieson, A. C., Kim, S.-H. & Wells, J. A. (1994) Biochemistry 33, 5689-5695, Jamieson, A. C., Wang, H. & Kim, S.-H. (1996) PNAS 93, 12834-12839, Isalan, M., Klug, A. & Choo, Y. (1998) Biochemistry 37, 12026-33). Seven rounds of selection were performed on the zinc finger displaying-phage with each of the 16 5′-GCGNNNGCG-3′ (SEQ ID NO: 685) biotinylated hairpin DNAs targets using a solution binding protocol. Stringency was increased in each round by the addition of competitor DNA. Sheared herring sperm DNA was provided for selection against phage that bound non-specifically to DNA. Stringent selective pressure for sequence specificity was obtained by providing DNAs of the 5′-GCGNNNGCG-3′ (SEQ ID NO: 685) types as specific competitors. Excess DNA of the 5′-GCGGNNGCG-3′ (SEQ ID NO: 685) type was added to provide even more stringent selection against binding to DNAs with single or double base changes as compared to the biotinylated target. Phage binding to the single biotinylated DNA target sequence were recovered using streptavidin coated beads. In some cases the selection process was repeated. The present data show that these domains are functionally modular and can be recombined with one another to create polydactyl proteins capable of binding 18-bp sequences with subnanomolar affinity. The family of zinc finger domains described herein is sufficient for the construction of 17 million novel proteins that bind the 5′-(GNN)₆-3′ family of DNA sequences. These domains can be used for the construction of zinc finger tags in fusion proteins according to the present invention.

Similarly, for the determination of zinc finger modules capable of specifically binding 5′-CNN-3′, methods known in the art are again employed. Typically, phage display libraries of zinc finger proteins were created and selected under conditions that favored enrichment of sequence specific proteins. Zinc finger domains recognizing a number of sequences required refinement by site-directed mutagenesis that was guided by both phage selection data and structural information.

Previously the characterization of 16 zinc finger domains specifically recognizing each of the 5′-GNN-3′ type of DNA sequences, that were isolated by phage display selections based on C7, a variant of the mouse transcription factor Zif268 and refined by site-directed mutagenesis was reported [Segal et al., (1999) Proc Natl Acad Sci USA 96(6), 2758-2763; Dreier et al., (2000) J. Mol. Biol. 303, 489-502; and U.S. Pat. No. 6,140,081, the disclosures of which are incorporated herein by reference]. In general, the specific DNA recognition of zinc finger domains of the Cys₂-His₂ type is mediated by the amino acid residues −1, 3, and 6 of each α-helix, although not in every case are all three residues contacting a DNA base. One dominant cross-subsite interaction has been observed from position 2 of the recognition helix. Asp² has been shown to stabilize the binding of zinc finger domains by directly contacting the complementary adenine or cytosine of the 5′ thymine or guanine, respectively, of the following 3 bp subsite. These non-modular interactions have been described as target site overlap. In addition, other interactions of amino acids with nucleotides outside the 3 bp subsites creating extended binding sites have been reported [Pavletich et al., (1991) Science 252(5007), 809-817; Elrod-Erickson et al., (1996) Structure 4(10), 1171-1180; Isalan et al., (1997) Proc Natl Acad Sci USA 94(11), 5617-5621].

Selection of the previously reported phage display library for zinc finger domains binding to 5′ nucleotides other than guanine or thymine met with no success, due to the cross-subsite interaction from aspartate in position 2 of the finger-3 recognition helix RSD-E-LKR (SEQ ID NO: 686). To extend the availability of zinc finger domains for the construction of artificial transcription factors, domains specifically recognizing the 5′-ANN-3′ type of DNA sequences were selected (U.S. patent application Ser. No. 09/791,106, filed Feb. 21, 2001, the disclosure of which is incorporated herein by reference). Other groups have described a sequential selection method which led to the characterization of domains recognizing four 5′-ANN-3′ subsites, 5′-AAA-3′, 5′-AAG-3′, 5′-ACA-3′, and 5′-ATA-3′ (Greisman et al., (1997) Science 275(5300), 657-661; Wolfe et al., (1999) J Mol Biol 285(5), 1917-1934). As indicated above, it is generally preferred to use an approach to select zinc finger domains recognizing CNN sites by eliminating the target site overlap. First, finger 3 of C7 (RSD-E-RKR) (SEQ ID NO: 278) binding to the subsite 5′-GCG-3′ was exchanged with a domain which did not contain aspartate in position 2. The helix TSG-N-LVR (SEQ ID NO: 156), previously characterized in finger 2 position to bind with high specificity to the triplet 5′-GAT-3′, seemed a good candidate. This 3-finger protein (C7.GAT), containing finger 1 and 2 of C7 and the 5′-GAT-3′-recognition helix in finger-3 position, was analyzed for DNA-binding specificity on targets with different finger-2 subsites by multi-target ELISA in comparison with the original C7 protein (C7.GCG). Both proteins bound to the 5′-TGG-3′ subsite (note that C7.GCG binds also to 5′-GGG-3′ due to the 5′ specification of thymine or guanine by Asp² of finger 3 which has been reported earlier. The recognition of the 5′ nucleotide of the finger-2 subsite was evaluated using a mixture of all 16 5′-XNN-3′ target sites (X=adenine, guanine, cytosine or thymine). Indeed, while the original C7.GCG protein specified a guanine or thymine in the 5′ position of finger 2, C7.GAT did not specify a base, indicating that the cross-subsite interaction to the adenine complementary to the 5′ thymine was abolished. A similar effect has previously been reported for variants of Zif268 where Asp was replaced by Ala² by site-directed mutagenesis [Isalan et al., (1997) Proc Natl Acad Sci USA 94(11), 5617-5621; Dreier et al., (2000) J. Mol. Biol. 303, 489-502]. The affinity of C7.GAT, measured by gel mobility shift analysis, was found to be relatively low, about 400 nM compared to 0.5 nM for C7.GCG [Segal et al., (1999) Proc Natl Acad Sci USA 96(6), 2758-2763], which may in part be due to the lack of the Asp² in finger 3.

Based on the 3-finger protein C7.GAT, a library was constructed in the phage display vector pComb3H [Barbas et al., (1991) Proc. Natl. Acad. Sci. USA 88, 7978-7982; Rader et al., (1997) Curr. Opin. Biotechnol. 8(4), 503-508]. Randomization involved positions −1, 1, 2, 3, 5, and 6 of the α-helix of finger 2 using a VNS codon doping strategy (V=adenine, cytosine or guanine, N=adenine, cytosine, guanine or thymine, S=cytosine or guanine). This allowed 24 possibilities for each randomized amino acid position, whereas the aromatic amino acids Trp, Phe, and Tyr, as well as stop codons, were excluded in this strategy. Because Leu is predominately found in position 4 of the recognition helices of zinc finger domains of the type Cys₂-His₂ this position was not randomized. After transformation of the library into ER2537 cells (New England Biolabs) the library contained 1.5×10⁹ members. This exceeded the necessary library size by 60-fold and was sufficient to contain all amino acid combinations.

Six rounds of selection of zinc finger-displaying phage were performed binding to each of the sixteen 5′-GAT-CNN-GCG-3′ (SEQ ID NO: 687) biotinylated hairpin target oligonucleotides, respectively, in the presence of non-biotinylated competitor DNA. Stringency of the selection was increased in each round by decreasing the amount of biotinylated target oligonucleotide and increasing amounts of the competitor oligonucleotide mixtures. In the sixth round the target concentration was usually 18 nM, 5′-ANN-3′, 5′-GNN-3′, and 5′-TNN-3′ competitor mixtures were in 5-fold excess for each oligonucleotide pool, respectively, and the specific 5′-CNN-3′ mixture (excluding the target sequence) in 10-fold excess. Phage binding to the biotinylated target oligonucleotide was recovered by capture to streptavidin-coated magnetic beads. Clones were usually analyzed after the sixth round of selection. The amino acid sequences of selected finger-2 helices were determined and generally showed good conservation in positions −1 and 3, consistent with previously observed amino acid residues in these positions [Segal et al., (1999) Proc Natl Acad Sci USA 96(6), 2758-2763]. Position −1 was Gln when the 3′ nucleotide was adenine, with the exception of domains binding 5′-ACA-3′ (SPA-D-LTN) (SEQ ID NO: 688) where a Ser was strongly selected. Triplets containing a 3′ cytosine selected Asp⁻¹ (exceptions were domains binding 5′-AGC-3′ and 5′-ATC-3′), a 3′ guanine Arg-1, and a 5′ thymine Thr⁻¹ and His⁻¹. The recognition of a 3′ thymine by His¹ has also been observed in finger 1 of TKK binding to 5′-GAT-3′ (HIS-N-FCR) (SEQ ID NO: 689); [Fairall et al., (1993) Nature (London) 366(6454), 483-7]). For the recognition of a middle adenine, Asp and Thr were selected in position 3 of the recognition helix. For binding to a middle cytosine, an Asp³ or Thr³ was selected, for a middle guanine, His³ (an exception was recognition of 5′-AGT-3′, which may have a different binding mechanism due to the unusual amino acid residue His⁻¹) and for a middle thymine, Ser³ and Ala³. Note also that the domains binding to 5′-ANG-3′ subsites contain Asp² which likely stabilizes the interaction of the 3-finger protein by contacting the complementary cytosine of the 5′ guanine in the finger-1 subsite. Even though there was a predominant selection of Arg and Thr in position 5 of the recognition helices, positions 1, 2 and 5 were variable.

Again, similarly, for the determination of zinc finger modules capable of specifically binding 5′-ANN-3′, methods known in the art are again employed, specifically, phage display libraries of zinc finger proteins were created and selected under conditions that favored enrichment of sequence specific proteins. Zinc finger domains recognizing a number of sequences required refinement by site-directed mutagenesis that was guided by both phage selection data and structural information. Previously the characterization of 16 zinc finger domains specifically recognizing each of the 5′-GNN-3′ type of DNA sequences, that were isolated by phage display selections based on C7, a variant of the mouse transcription factor Zif268 and refined by site-directed mutagenesis was reported [Segal et al., (1999) Proc Natl Acad Sci USA 96(6), 2758-2763; Dreier et al., (2000) J. Mol. Biol. 303, 489-502]. The molecular interaction of Zif268 with its target DNA 5′-GCG TGG GCG-3′ (SEQ ID NO: 690) has been characterized in great detail. In general, the specific DNA recognition of zinc finger domains of the Cys₂-His₂ type is mediated by the amino acid residues −1, 3, and 6 of each α-helix, although not in every case are all three residues contacting a DNA base. One dominant cross-subsite interaction has been observed from position 2 of the recognition helix. Asp² has been shown to stabilize the binding of zinc finger domains by directly contacting the complementary adenine or cytosine of the 5′ thymine or guanine, respectively, of the following 3 bp subsite. These non-modular interactions have been described as target site overlap. In addition, other interactions of amino acids with nucleotides outside the 3 bp subsites creating extended binding sites have been reported [Pavletich et al., (1991) Science 252(5007), 809-817; Elrod-Erickson et al., (1996) Structure 4(10), 1171-180; Isalan et al., (1997) Proc Natl Acad Sci USA 94(11), 5617-5621].

In general, methods analogous to those described above for the selection of zinc finger modules specifically binding 5′-GNN-3′ subsites were used for the selection of zinc finger modules specifically binding 5′-ANN-3′ subsites. These methods can be used for the generation of zinc finger tags incorporated into fusion proteins according to the present invention.

Selection of the previously reported phage display library for zinc finger domains binding to 5′ nucleotides other than guanine or thymine met with no success, due to the cross-subsite interaction from aspartate in position 2 of the finger-3 recognition helix RSD-E-LKR (SEQ ID NO: 686). To extend the availability of zinc finger domains for the construction of zinc finger tags incorporated into fusion proteins according to the present invention, domains specifically recognizing the 5′-ANN-3′ type of DNA sequences were selected. Other groups have described a sequential selection method which led to the characterization of domains recognizing four 5′-ANN-3′ subsites, 5′-AAA-3′, 5′-AAG-3′, 5′-ACA3′, and 5′-ATA-3′ [Greisman et al., (1997) Science 275(5300), 657-661; Wolfe et al., (1999) J Mol Biol 285(5), 1917-1934]. The present disclosure uses a different approach to select zinc finger domains recognizing such sites by eliminating the target site overlap. First, finger 3 of C7 (RSD-E-RKR) (SEQ ID NO: 278) binding to the subsite 5′-GCG-3′ was exchanged with a domain which did not contain aspartate in position 2. The helix TSG-N-LVR (SEQ ID NO: 156), previously characterized in finger 2 position to bind with high specificity to the triplet 5′-GAT-3′, seemed a good candidate. This 3-finger protein (C7.GAT), containing finger 1 and 2 of C7 and the 5′-GAT-3′-recognition helix in finger-3 position, was analyzed for DNA-binding specificity on targets with different finger-2 subsites by multi-target ELISA in comparison with the original C7 protein (C7.GCG). Both proteins bound to the 5′-TGG-3′ subsite (note that C7.GCG binds also to 5′-GGG-3′ due to the 5′ specification of thymine or guanine by Asp² of finger 3 which has been reported earlier.

The amino acid sequences of selected finger-2 helices were determined and generally showed good conservation in positions −1 and 3, consistent with previously observed amino acid residues in these positions [Segal et al., (1999) Proc Natl Acad Sci USA 96(6), 2758-2763]. Position −1 was Gln when the 3′ nucleotide was adenine, with the exception of domains binding 5′-ACA-3′ (SPA-D-LTN) (SEQ ID NO: 688) where a Ser was strongly selected. Triplets containing a 3′ cytosine selected Asp⁻¹ (exceptions were domains binding 5′-AGC-3′ and 5′-ATC-3′), a 3′ guanine Arg⁻¹, and a 5′ thymine Thr⁻¹ and His⁻¹. The recognition of a 3′ thymine by His⁻¹ has also been observed in finger 1 of TKK binding to 5′-GAT-3′ (HIS-N-FCR) (SEQ ID NO: 689); [Fairall et al., (1993) Nature (London) 366(6454), 483-7]). For the recognition of a middle adenine, Asp and Thr were selected in position 3 of the recognition helix. For binding to a middle cytosine, an Asp³ or Thr3 was selected, for a middle guanine, His³ (an exception was recognition of 5′-AGT-3′, which may have a different binding mechanism due to the unusual amino acid residue His⁻¹) and for a middle thymine, Ser³ and Ala³. Note also that the domains binding to 5′-ANG-3′ subsites contain Asp² which likely stabilizes the interaction of the 3-finger protein by contacting the complementary cytosine of the 5′ guanine in the finger-1 subsite. Even though there was a predominant selection of Arg and Thr in position 5 of the recognition helices, positions 1, 2 and 5 were variable.

The most interesting observation was the selection of amino acid residues in position 6 of the α-helices that determines binding to the 5′ nucleotide of a 3 bp subsite. In contrast to the recognition of a 5′ guanine, where the direct base contact is achieved by Arg or Lys in position 6 of the helix, no direct interaction has been observed in protein/DNA complexes for any other nucleotide in the 5′ position [Elrod-Erickson et al., (1996) Structure 4(10), 1171-1180; Pavletich et al., (1993) Science (Washington, D.C., 1883-) 261(5129), 1701-7; Kim et al., (1996) Nat Struct Biol 3(11), 940-945; Fairall et al., (1993) Nature (London) 366(6454), 483-7; Houbaviy et al., (1996) Proc Natl Acad Sci USA 93(24), 13577-82; Wuttke et al., (1997) J Mol Biol 273(1), 183-206; Nolte et al., (1998) Proc Natl Acad Sci USA 95(6), 2938-2943]. Selection of domains against finger-2 subsites of the type 5′-GNN-3′ had previously generated domains containing only Arg⁶ which directly contacts the 5′ guanine [Segal et al., (1999) Proc Natl Acad Sci USA 96(6), 2758-2763]. However, unlike the results for 5′-GNN-3′ zinc finger domains, selections of the phage display library against finger-2 subsites of the type 5′-ANN-3′ identified domains containing various amino acid residues: Ala⁶, Arg⁶, Asn⁶, Asp⁶, Gln⁶, Glu⁶, Thr⁶ or Val⁶. In addition, one domain recognizing 5′-TAG-3′ was selected from this library with the amino acid sequence RED-N-LHT (SEQ ID NO: 268). Thr⁶ is also present in finger 2 of Zif268 (RSD-H-LTT) (SEQ ID NO: 276) binding 5′-TGG-3 for which no direct contact was observed in the Zif268/DNA complex.

Finger-2 variants of C7.GAT were subcloned into bacterial expression vector as fusion with maltose-binding protein (MBP) and proteins were expressed by induction with 1 mM IPTG (proteins (p) are given the name of the finger-2 subsite against which they were selected). Proteins were tested by enzyme-linked immunosorbent assay (ELISA) against each of the 16 finger-2 subsites of the type 5′-GAT ANN GCG-3′ (SEQ ID NO: 691) to investigate their DNA-binding specificity. In addition, the 5′-nucleotide recognition was analyzed by exposing zinc finger proteins to the specific target oligonucleotide and three subsites which differed only in the 5′-nucleotide of the middle triplet. For example, pAAA was tested on 5′-AAA-3′, 5′-CAA-3′, 5′-GAA-3′, and 5′-TAA-3′ subsites. Many of the tested 3-finger proteins showed exquisite DNA-binding specificity for the finger-2 subsite against which they were selected. The most promising helix for pAGC (DAS-H-LHT) (SEQ ID NO: 18) which contained the expected amino acid Asp⁻¹ and His³ specifying a 3′ cytosine and middle guanine, but also a Thr⁶ not selected in any other case for a 5′ adenine, was analyzed without detectable DNA binding.

To analyze a larger set, the pool of coding sequences for pAGC was subcloned into the plasmid pMa1 after the sixth round of selection and 18 individual clones were tested for DNA-binding specificity, of which none showed measurable DNA-binding in ELISA. In the case of pATC, two helices (RRS-S-CRK and RRS-A-CRR) (SEQ ID NOs: 23, 22) were selected containing a Leu⁴ to Cys⁴ mutation, for which no DNA binding was detectable. Rational design was applied to find domains binding to 5′-AGC-3′ or 5′-ATC-3′, since no proteins binding these finger-2 subsites were generated by phage display. Finger-2 mutants were constructed based on the recognition helices which were previously demonstrated to bind specifically to 5′-GGC-3′ (ERS-K-LAR (SEQ ID NO: 214), DPG-H-LVR (SEQ ID NO: 162)) and 5′-GTC-3′ (DPG-A-LVR) (SEQ ID NO: 166) [Segal et al., (1999) Proc Natl Acad Sci USA 96(6), 2758-2763]. For pAGC two proteins were constructed (ERS-K-LRA (SEQ ID NO: 692), DPG-H-LRV (SEQ ID NO: 693)) by simply exchanging position 5 and 6 to a 5′ adenine recognition motif RA or RV. DNA binding of these proteins was below detection level. In the case of pATC two finger-2 mutants containing a RV motif were constructed (DPG-A-LRV (SEQ ID NO: 67), DPG-S-LRV (SEQ ID NO: 694)). Both proteins bound DNA with extremely low affinity regardless if position 3 was Ala or Ser.

Analysis of the 3-finger proteins on the sixteen finger-2 subsites by ELISA revealed that some finger-2 domains bound best to a target they were not selected against. First, the predominantly selected helix for 5′-AGA-3′ was RSD-H-LTN (SEQ ID NO: 11), which in fact bound 5′-AGG-3′. This can be explained by the Arg in position −1. In addition, this protein showed a better discrimination of a 5′ adenine compared to the predominantly selected helix pAGG (RSD-H-LAE (SEQ ID NO: 28)). Second, a helix binding specifically to 5′-AAG-3′ (RSD-N-LKN (SEQ ID NO: 695) was actually selected against 5′-AAC-3′, and bound more specifically to the finger-2 subsite 5′-AAG-3′ than pAAG (RSD-T-LSN (SEQ ID NO: 24), which had been selected in the 5′-AAG-3′ set. In addition, proteins directed to target sites of the type 5′-ANG-3′ showed cross reactivity with all four target sites of the type 5′-ANG-3′, except for pAGG. The recognition of a middle purine seems more restrictive than of a middle pyrimidine, because also pAAG (RSD-N-LKN (SEQ ID NO: 25) had only moderate cross-reactivity.

In comparison, the proteins pACG (RTD-T-LRD (SEQ ID NO: 46)) and pATG (RRD-A-LNV (SEQ ID NO: 29) show cross-reactivity with all 5′-ANG-3′ subsites. The recognition of a middle pyrimidine has been reported to be difficult in previous studies for domains binding to 5′-GNG-3′ DNA sequences [Segal et al., (1999) Proc Natl Acad Sci USA 96(6), 2758-2763; Dreier et al., (2000) J. Mol. Biol. 303, 489-502]. To improve the recognition of the middle nucleotide, finger-2 mutants containing different amino acid residues in position 3 were generated by site-directed mutagenesis. Binding of pAAG (RSD-T-LSN (SEQ ID NO: 24) was more specific for a middle adenine after a Thr³ to Asn³ mutation. The binding to 5′-ATG-3′ (SRD-A-LNV (SEQ ID NO: 696)) was improved by a single amino acid exchange Ala³ to Gln³, while a Thr³ to Asp³ or Gln³ mutation for pACG (RSD-T-LRD (SEQ ID NO: 26) abolished DNA binding. In addition, the recognition helix pAGT (HRT-T-LLN (SEQ ID NO: 50) showed cross-reactivity for the middle nucleotide which was reduced by a Leu⁵ to Thr⁵ substitution. Surprisingly, improved discrimination for the middle nucleotide was often associated with some loss of specificity for the recognition of the 5′ adenine.

Selection of zinc finger domains binding to subsites containing a 5′ adenine or cytosine from the previously described finger-2 library based on the 3-finger protein C7 [Segal et al., (1999) Proc Natl Acad Sci USA 96(6), 2758-2763] was not suitable for the selection of zinc-finger domains due to the limitation of aspartate in position 2 of finger 3 which makes a cross-subsite contact to the nucleotide complementary of the 5′ position of the finger-2 subsite. This contact was eliminated by exchanging finger 3 with a domain lacking Asp². Finger 2 of C7.GAT was randomized and a phage display library constructed. In most cases, novel 3-finger proteins were selected binding to finger-2 subsites of the type 5′-ANN-3′. For the subsites 5′-AGC-3′ and 5′-ATC-3′ no tight binders were identified. This was not expected, because the domains binding to the subsite 5′-GGC-3′ and 5′-GTC-3′ previously selected from the C7-based phage display library showed excellent DNA-binding specificity and affinity of 40 nM to their target site [Segal et al., (1999) Proc Natl Acad Sci USA 96(6), 2758-2763]. One simple explanation would be the limiting randomization strategy by the usage of VNS codons which do not include the aromatic amino acid residues. These were not included in the library, because for the domains binding to 5′-GNN-3′ subsites no aromatic amino acid residues were selected, even though they were included in the randomization strategy [Segal et al., (1999) Proc Natl Acad Sci USA 96(6), 2758-2763]. However, there have been zinc finger domains reported containing aromatic residues, like finger 2 of CFII2 (VKD-Y-LTK (SEQ ID NO: 697); [Gogos et al., (1996) PNAS 93, 2159-2164]), finger 1 of TFIIIA (KNW-K-LQA (SEQ ID NO: 698); [Wuttke et al., (1997) J Mol Biol 273(1), 183-206]), finger 1 of TTK (HIS-N-FCR (SEQ ID NO: 689; [Fairall et al., (1993) Nature (London) 366(6454), 483-7]) and finger 2 of GLI (AQY-M-LVV (SEQ ID NO: 699); [Pavletich et al., (1993) Science (Washington, D.C., 1883-) 261(5129), 1701-7]). Aromatic amino acid residues might be important for the recognition of the subsites 5′-AGC-3′ and 5′-ATC-3′.

In recent years it has become clear that the recognition helix of Cys₂-His₂ zinc finger domains can adopt different orientations relative to the DNA in order to achieve optimal binding [Pabo et al., (2000) J. Mol. Biol. 301, 597-624]. However, the orientation of the helix in this region may be partially restricted by the frequently observed interaction involving the zinc ion, His⁷, and the phosphate backbone. Furthermore, comparison of binding properties of interactions in protein/DNA complexes have led to the conclusion that the Ca atom of position 6 is usually 8.8±0.8 Å apart from the nearest heavy atom of the 5′ nucleotide in the DNA subsite, which favors only the recognition of a 5′ guanine by Arg⁶ or Lys⁶ [Pabo et al., (2000) J. Mol. Biol. 301, 597-624]. To date, no interaction of any other position 6 residue with a base other than guanine has been observed in protein/DNA complexes. For example, finger 4 of YY1 (QST-N-LKS) (SEQ ID NO: 700) recognizes 5′-CAA-3′ but there was no contact observed between Ser⁶ and the 5′ cytosine [Houbaviy et al., (1996) Proc Natl Acad Sci USA 93(24), 13577-82]. Further, in the case of Thr⁶ in finger 3 of YY1 (LDF-N-LRT) (SEQ ID NO: 701), recognizing 5′-ATT-3′, and in finger 2 of Zif268 (RSD-H-LTT) (SEQ ID NO: 276), specifying 5′-T/GGG-3′, no contact with the 5′ nucleotide was observed [Houbaviy et al., (1996) Proc Natl Acad Sci USA 93(24), 13577-82; Elrod-Erickson et al., (1996) Structure 4(10), 1171-1180]. Finally, Ala⁶ of finger 2 of Tramtrack (RKD-N-MTA) (SEQ ID NO: 702) binding to the subsite 5′-AAG-3′ does not contact the 5′ adenine [Fairall et al., (1993) Nature (London) 366(6454), 483-7].

Amino acid residues Ala⁶, Val⁶, Asn⁶ and even Arg⁶, which in a different context was demonstrated to bind a 5′ guanine efficiently [Segal et al., (1999) Proc Natl Acad Sci USA 96(6), 2758-2763], were predominantly selected from the C7.GAT library for DNA subsites of the type 5′-ANN-3′. In addition, position 6 was selected as Thr, Glu and Asp depending on the finger-2 target site. This is consistent with early studies from other groups where positions of adjacent fingers were randomized [Jamieson et al., (1996) Proc Natl Acad Sci USA 93, 12834-12839; Isalan et al., (1998) Biochemistry 37(35), 12026-12033]. Screening of phage display libraries had resulted in selection of amino acid residues Tyr, Val, Thr, Asn, Lys, Glu and Leu, as well as Gly, Ser and Arg, but not Ala, for the recognition of a 5′ adenine. In addition, using a sequential phage display selection strategy several domains binding to 5′-ANN-3′ subsites were identified and specificity evaluated by target site selections. Arg, Ala and Thr in position 6 of the helix were demonstrated to recognize predominantly a 5′ adenine [Wolfe et al., (1999) Annu. Rev. Biophys. Biomol. Struct. 3, 183-212].

In addition, Thr⁶ specifies a 5′ adenine as shown by target site selection for finger 5 of Gfi-1 (QSS-N-LIT) (SEQ ID NO: 703) binding to the subside 5′-AAA-3′ [Zweidler-McKay et al., (1996) Mol. Cell. Biol. 16(8), 4024-4034]. These examples, including the present results, indicate that there is likely a relation between amino acid residue in position 6 and the 5′ adenine, because they are frequently selected. This is at odds with data from crystallographic studies, that never showed interaction of position 6 of the α-helix with a 5′ nucleotide except guanine. One simple explanation might be that short amino acid residues, like Ala, Val, Thr, or Asn do not give rise to steric hindrance in the binding mode of domains recognizing 5′-ANN-3′ subsites. This is supported by results gathered by site-directed mutagenesis in position 6 for a helix (QRS-A-LTV) (SEQ ID NO: 704) binding to a 5′-G/ATA-3′ subsite [Gogos et al., (1996) PNAS 93, 2159-2164]. Replacement of Val⁶ with Ala⁶, which were also found for domains described here, or Lys⁶, had no effect on the binding specificity or affinity.

Computer modeling was used to investigate possible interactions of the frequently selected Ala⁶, Asn⁶ and Arg⁶ with a 5′ adenine. Analysis of the interaction from Ala⁶ in the helix binding to 5′-AAA-3′ (QRA-N-LRA) (SEQ ID NO: 4) with a 5′ adenine was based on the coordinates of the protein/DNA complex of finger 1 (QSG-S-LTR) (SEQ ID NO: 705) from a Zif268 variant. If Gln⁻¹ and Asn³ of QRA-N-LRA (SEQ ID NO: 4) hydrogen bond with their respective adenine bases in the canonical way, these interactions should fix a distance of about 8 Å between the methyl group of Ala⁶ and the 5′ adenine and more than 11 Å between the methyl groups of Ala⁶ and the thymine base-paired to the adenine, suggesting also that no direct contact can be proposed for Val⁶ and Thr⁶.

Interestingly, the expected lack of 5′ specificity by short amino acids in position 6 of the α-helix is only partially supported by the binding data. Helices such as RR)-A-LNV (SEQ ID NO: 29) and the finger-2 helix RSD-H-LTT (SEQ ID NO: 276) of C7.GAT did indeed show essentially no 5′ specificity. However, helix DSG-N-LRV (SEQ ID NO: 15) displayed excellent specificity for a 5′ adenine, while TSH-G-LTT (SEQ ID NO: 38) was specific for 5′ adenine or guanine. Other helices with short position −6 residues displayed varying degrees of 5′ specificity, with the only obvious consistency being that 5′ thymine was usually excluded. Since it is unlikely that the position −6 residue can make a direct contribution to specificity, the observed binding patterns must derive from another source. Possibilities include local sequence-specific DNA structure and overlapping interactions from neighboring domains. The latter possibility is disfavored, however, because the residue in position 2 of finger 3 (which is frequently observed to contact the neighboring site) is glycine in the parental protein C7.GAT, and because 5′ thymine was not excluded by the two helices mentioned above.

Asparagine was also frequently selected in position 6. Helix HRT-T-LTN (SEQ ID NO: 58) and RSD-T-LSN (SEQ ID NO: 24) displayed excellent specificity for 5′ adenine. However, Asn⁶ also seemed to impart specificity for both adenine and guanine, suggesting an interaction with the N7 common to both nucleotides. Computer modeling of the helix binding to 5′-AGG-3′ (RSD-H-LTN (SEQ ID NO: 10), based on the coordinates of finger 2, binding to 5′-TGG-3′, in the Zif268/DNA crystal structure (RSD-H-LTT (SEQ ID NO: 276); [Elrod-Erickson et al., (1996) Structure 4(10), 1171-1180]), suggested that the Nd of Asn would be approximately 4.5 Å from N7 of the 5′ adenine. A modest reorientation of the α-helix which is considered within the range of canonical docking orientations [Pabo et al., (2000) J. Mol. Biol. 301, 597-624], could plausibly bring the Nd within hydrogen bonding distance, analogous to the reorientation observed when glutamate rather than arginine appears in position −1. However, it is interesting to speculate why Asn⁶ was selected in this 5′-ANN-3′ recognition set while the longer Gln⁶ was not. Gln⁶, being more flexible, may have been able to stabilize other interactions that were selected against during phage display. Alternatively, the shorter side chain of Asn⁶ might accommodate an ordered water molecule that could contact the 5′ nucleotide without reorientation of the helix.

The final residue to be considered is Arg⁶. It was somewhat surprising that Arg⁶ was selected so frequently on 5′-ANN-3′ targets because in previous studies, it was unanimously selected to recognize a 5′ guanine with high specificity [Segal et al., (1999) Proc Natl Acad Sci USA 96(6), 2758-2763]. However, in the current study, Arg⁶ primarily specified 5′ adenine, in some cases in addition to recognition of a 5′ guanine. Computer modeling of helix binding to 5′-ACA-3′ (SPA-D-LTR (SEQ ID NO: 5), based on the coordinates of finger 1 QSG-S-LTR (SEQ ID NO: 705) of a Zif268 variant binding 5′-GCA-3′ [Elrod-Erickson et al., (1998) Structure 6(4), 451-464], suggested that Arg⁶ could easily adopt a configuration that allowed it to make a cross-strand hydrogen bond to O4 of a thymine base-paired to 5′ adenine. In fact, Arg⁶ could bind with good geometry to both the O4 of thymine and O6 of a guanine base-paired to a middle cytosine Such an interaction is consistent with the fact that Arg⁶ was selected almost unanimously when the target sequence was 5′-ACN-3′. The expectation for arginine to facilitate multiple interactions is compelling. Several lysines in TFIIIA were observed by NMR to be conformationally flexible [Foster et al., (1997) Nat. Struct. Biol. 4(8), 605-608], and Gln⁻¹ behaves in a manner which suggests flexibility [Dreier et al., (2000) J. Mol. Biol. 303, 489-502]. Arginine has more rotatable bonds and more hydrogen bonding potential than lysine or glutamine and it is attractive to speculate that Arg⁶ is not limited to recognition of 5′ guanine.

Amino acid residues in positions −1 and 3 were generally selected in analogy to their 5′-GNN-3 counterparts with two exceptions. His⁻¹ was selected for pAGT and pATT, recognizing a 3′ thymine, and Ser⁻¹ for pACA, recognizing a 3′ adenine. While Gln³ was frequently used to specify a 3′ adenine in subsites of the type 5′-GNN-3′, a new element of 3′ adenine recognition was suggested from this study involving Ser⁻¹ selected for domains recognizing the 5′-ACA-3′ subsite which can make a hydrogen bond with the 3′ adenine. Computer modeling demonstrates that Ala², co-selected in the helix SPA-D-LTR (SEQ ID NO: 5), can potentially make a van der Waals contact with the methyl group of the thymine based-paired to 3′ adenine. The best evidence that Ala² might be involved is that helix SPA-D-LTR (SEQ ID NO: 5) is strongly specific for 3′ adenine while SHS-D-LVR (SEQ ID NO: 6) is not. Gln⁻¹ is often sufficient for 3′ adenine recognition. However, data from previous studies suggested that the side chain of Gln⁻¹ can adopt multiple conformations, enabling, for example, recognition of 3′ thymine [Nardelli et al., (1992) Nucleic Acids Res. 20(16), 4137-44; Elrod-Erickson et al., (1998) Structure 6(4), 451-464; Dreier et al., (2000) J. Mol. Biol. 303, 489-502]. Ala² in combination with Ser⁻¹ may be an alternative means to specificity a 3′ adenine.

Another interaction not observed in the 5′-GNN-3′ study is the cooperative recognition of 3′ thymine by His⁻¹ and the residue at position 2. In finger 1 of the crystal structure of the Tramtrack/DNA complex, helix HIS-N-FCR (SEQ ID NO: 689) binds the subsite 5′-GAT-3′ [Fairall et al., (1993) Nature (London) 366(6454), 483-7]. The His⁻¹ ring is perpendicular to the plane of the 3′ thymine base and is approximately 4 Å from the methyl group. Ser² additionally makes a hydrogen bond with 04 of 3′ thymine. A similar set of contacts can be envisioned by computer modeling for the recognition of 5′-ATT-3′ by helix HKN-A-LQN (SEQ ID NO: 39). Asn² in this helix has the potential not only to hydrogen bond with 3′ thymine but also with the adenine base-paired to thymine. His⁻¹ was also found for the helix binding 5′-AGT-3′ (HRT-T-LLN (SEQ ID NO: 50) in combination with a Thr². Thr is structurally similar to Ser and might be involved in a similar recognition mechanism.

In conclusion, the results of the characterization of zinc finger domains described above binding 5′-ANN-3′ DNA subsites is consistent with the overall view that there is no general recognition code, which makes rational design of additional domains difficult. However, phage display selections can be applied and pre-defined zinc finger domains can serve as modules for the construction of fusion proteins according to the present invention. The domains characterized here enables targeting of DNA sequences other than 5′-(GNN)₆-3′. This is an important supplement to existing domains, since G/C-rich sequences often contain binding sites for cellular proteins and 5′-(GNN)₆-3′ sequences may not be found in all promoters. These results also enable the construction of zinc finger tags that have the desired specificity and can be incorporated into fusion proteins according to the present invention.

With respect to zinc finger tags that recognize a triplet for which the 5′-base is A, one conclusion that can be drawn is that a variety of amino acid residues at position 6 of the heptapeptide can specify an adenine at the 5′-position of the triplet subsite. These residues include alanine (A), arginine (R), asparagine (N), aspartate (D), glutamine (Q), glutamate (E), threonine (T), and valine (V).

Accordingly, in view of these results, rational design was performed to develop additional zinc fingers that bound the 5′-(AGC)-3′ subsite with a substantial degree of affinity and specificity. This was done by studying the binding profiles of many mutant proteins and made mutations based on proteins that seemed to have favorable interactions with the 5′-(AGC)-3′ subsite as a target sequence. Site-directed mutagenesis was carried out to develop these additional zinc fingers. The fingers developed by this strategy include: DPG-A-LIN (SEQ ID NO: 71), ERS-H-LRE (SEQ ID NO: 72); and DPG-H-LTE (SEQ ID NO: 73).

Notwithstanding the lack of a general recognition code, these results provide a number of guidelines for the determination of sequences within the present invention to one of ordinary skill in the art. Some of these guidelines are also useful for selection of zinc finger domains specifically binding sequences of the form 5′-(AGC)-3′. These guidelines include the following: (1) For subsites containing a 3′-cytosine, Gln, Asn, Ser, Gly, His, or Asp are typically preferred in position −1. (2) For the target site 5′-AGN-3′, His is preferred at position 3. (3) For the target site 5′-AGC-3′ Trp and Thr are typically preferred at position 3; His is also possible. (4) Positions 1, 2, and 5 can vary widely. These are only guidelines, and the secondary or tertiary structure of a protein or polypeptide incorporating a zinc finger domain according to the present invention can lead to different amino acids being preferred for recognition of particular subsites or particular nucleotides at a defined position of such subsites. Additionally, the conformation of a particular zinc finger moiety within a protein having a plurality of zinc finger moieties can affect the binding.

Other amino acid residues are also subject to mutation or substitution. For example, leucine is often located in position 4 of the seven-amino acid domain and packs into the hydrophobic core of the protein. Accordingly, the leucine in position 4 can be replaced with other relatively small hydrophobic residues, such as valine and isoleucine, without disturbing the three-dimensional structure or function of the protein. Alternatively, the leucine in position 4 can also be replaced with other hydrophobic residues such as phenylalanine or tryptophan.

Other amino acid substitutions are possible. When G is in the middle position of the triplet, His is a possibility for position 3 of the helix and can replace another amino acid there. When the last two bases of the triplet are GC, Trp and Thr are alternatives at position 3 and can replace another amino acid there. Cys is also an alternative for position 4, particularly when Leu was present there.

One general substitution pattern for amino acids in these zinc finger tags is shown in Table 1, below.

TABLE 1
Protein/DNA-Interactions of Zinc finger domains (D. J. Segal, B. Dreier,
R. R. Beerli, C. F. Barbas III, Proc. Natl. Acad. Sci. USA 1999,
96, 2758-2763.)

| | Position within the | | |
|---|---|---|---|
| | triplet Nucleotide | | |
| | 5′ | Middle | 3′ |
| Adenine | nd | Asn | Gln |
| Cytosine | nd | Thr, Asp, Glu | Asp, Glu |
| Guanine | Arg | His, Lys | Arg |
| Thymine | nd | Ser, Ala | Thr, Ser |

In addition, the following table (Table 2) describes a potentially useful range of amino acid substitutions assuming that the 5′-base is A, as would be the case in the triplet 5′-(AGC)-3′.

TABLE 2

| Middle | 3′ | Zinc Finger Amino | Amino Acid |
|---|---|---|---|
| Base | Base | Acid Position | Alternatives |
| A | A | −1 | Q, N, S |
| C | A | −1 | S |
| N | G | −1 | R, N, Q, H, S, T, I |
| N | G | 2 | D |
| N | T | −1 | R, N, Q, H, S, T, A, C |
| N | C | −1 | Q, N, S, G, H, D |
| A | N | 3 | H, N, G, V, P, I, K |
| C | N | 3 | T, D, H, K, R, N |
| C | C | 3 | N, H, S, D, T, Q, G |
| C | G | 3 | T, H, S, D, N, Q, G |
| G | N | 3 | H |
| G | G/T | 3 | S, D, T, N, Q, G, H |
| G | C | 3 | W, T, H |
| G | N | 3 | H |
| T | A/G | 3 | S, A |
| T | C/T | 3 | H |
| N | A | −1 | R |
| N | T | −1 | S, T, H |
| N | N | 4 | L, V, I, C |

In Table 2, particularly preferred amino acids are underlined.
“N” is any of the four possible naturally-occuring nucleotides (A, C, G, or T).

Additionally, inspection of the domains binding nucleotide sequences of the form 5′-(AGC)-3′ reveals that residues 4, 5, and 6 can be selected from LIN, LRE, and LTE, and that these three-amino-acid partial sequences can be interchanged when the 3′-residue of the nucleic acid subsite to be recognized is A. This finding can be used to generate additional zinc finger domains.

Accordingly, preferred zinc finger domains included in fusion proteins according to the present invention and binding sequences of the form 5′-(AGC)-3′ include the following: SEQ ID NO: 71 through SEQ ID NO: 127.

Of these, SEQ ID NO: 71 through SEQ ID NO: 80 are particularly preferred; SEQ ID NO: 71, SEQ ID NO: 72, and SEQ ID NO: 73 are more particularly preferred.

SEQ ID NO: 74 through SEQ ID NO: 127 are derived from the sequences of SEQ ID NO: 71, SEQ ID NO: 72, or SEQ ID NO: 73 by the rules of general applicability for substitution of amino acids set forth above in Tables 1 and 2 or by the interchangeability of the partial motifs LIN, LRE, and LTE at positions 4, 5, and 6, respectively, of these domains. SEQ ID NO: 74 through SEQ ID NO: 80 are derived by the rules set forth in Table 1. SEQ ID NO: 81 through SEQ ID NO: 96 are derived by the rules set forth in Table 2. SEQ ID NO: 97 through SEQ ID NO: 127 are derived by the interchangeability of the partial motifs LIN, LRE, and LTE at positions 4, 5, and 6, respectively, of these domains. Accordingly, these sequences can be incorporated in zinc finger tags that are within the scope of the invention. The specific sequences are set forth below.

A similar procedure was followed to develop zinc finger tags that incorporate TNN-specific sequences. Table 2 can also be used to specify the middle and 3′-bases, assuming that the 5′-base is T. The specific sequences for these zinc finger tags are set forth below.

In addition, additional zinc finger tags that include TNN-specific sequences can incorporate the following TNN-specific zinc finger domains: (1) a zinc finger nucleotide binding domain specifically binding the nucleotide sequence 5′-(TAA)-3′, wherein the amino acid residue of the domain numbered −1 is selected from the group consisting of Q, N, and S; (2) a zinc finger nucleotide binding domain specifically binding the nucleotide sequence 5′-(TCA)-3′, wherein the amino acid residue of the domain numbered −1 is S; (3) a zinc finger nucleotide binding domain specifically binding the nucleotide sequence 5′-(TNG)-3′, wherein N is any of A, C, G, or T, wherein the amino acid residue of the domain numbered −1 is selected from the group consisting of R, N, Q, H, S, T, and I; (4) a zinc finger nucleotide binding domain specifically binding the nucleotide sequence 5′-(TNG)-3′, wherein N is any of A, C, G, or T, wherein the amino acid residue numbered 2 of the domain is D; (5) a zinc finger nucleotide binding domain specifically binding the nucleotide sequence 5′-(TNT)-3′, wherein N is any of A, C, G, or T, wherein the amino acid residue of the domain numbered −1 is selected from the group consisting of R, N, Q, H, S, T, A, and C; (6) a zinc finger nucleotide binding domain specifically binding the nucleotide sequence 5′-(TNC)-3′, wherein N is any of A, C, G, or T, wherein the amino acid residue of the domain numbered −1 is selected from the group consisting of Q, N, S, C, H, and D; (7) a zinc finger nucleotide binding domain specifically binding the nucleotide sequence 5′-(TAN)-3′, wherein N is any of A, C, G, or T, wherein the amino acid residue of the domain numbered 3 is selected from the group consisting of H, N, G, V, P, I, and K; (8) a zinc finger nucleotide binding domain specifically binding the nucleotide sequence 5′-(TCN)-3′, wherein N is any of A, C, G, or T, wherein the amino acid residue of the domain numbered 3 is selected from the group consisting of T, D, H, K, R, and N; (9) a zinc finger nucleotide binding domain specifically binding the nucleotide sequence 5′-(TCC)-3′, wherein the amino acid residue of the domain numbered 3 is selected from the group consisting of N, H, S, D, T, Q, and G; (10) a zinc finger nucleotide binding domain specifically binding the nucleotide sequence 5′-(TCG)-3′, wherein the amino acid residue of the domain numbered 3 is selected from the group consisting of T, H, S, D, N, Q, and G; (11) a zinc finger nucleotide binding domain specifically binding the nucleotide sequence 5′-(TGN)-3′, wherein N is any of A, C, G, or T, wherein the amino acid residue of the domain numbered 3 is H; (12) a zinc finger nucleotide binding domain specifically binding a nucleotide sequence selected from the group consisting of 5′-(TGG)-3′ and 5′-(TGT)-3′, wherein the amino acid residue of the domain numbered 3 is selected from the group consisting of S, D, T, N, Q, G, and H; (13) a zinc finger nucleotide binding domain specifically binding the nucleotide sequence 5′-(TGC)-3′, wherein the amino acid residue of the domain numbered 3 is selected from the group consisting of W, T, and H; (14) a zinc finger nucleotide binding domain specifically binding the nucleotide sequence 5′-(TGN)-3′, wherein N is any of A, C, C, or T, wherein the amino acid residue of the domain numbered 3 is H; (15) a zinc finger nucleotide binding domain specifically binding a nucleotide sequence selected from the group consisting of 5′-(TTA)-3′ and 5′-(TTG)-3′, wherein the amino acid residue of the domain numbered 3 is selected from the group consisting of S and A; (16) a zinc finger nucleotide binding domain specifically binding a nucleotide sequence selected from the group consisting of 5′-(TTC)-3′ and 5′-(TTT)-3′, wherein the amino acid residue of the domain numbered 3 is H; (17) a zinc finger nucleotide binding domain specifically binding the nucleotide sequence 5′-(TNA)-3′, wherein N is any of A, C, G, or T, wherein the amino acid residue of the domain numbered −1 is R; (18) a zinc finger nucleotide binding domain specifically binding the nucleotide sequence 5′-(TNT)-3′, wherein N is any of A, C, G, or T, wherein the amino acid residue of the domain numbered −1 is selected from the group consisting of S, T, and H; and (19) a zinc finger nucleotide binding domain specifically binding the nucleotide sequence 5′-(TNN)-3′, wherein N is any of A, C, G, or T, wherein the amino acid residue of the domain numbered 4 is selected from the group consisting of L, V, I, and C.

The following zinc finger nucleotide binding domains, therefore, can be included in zinc finger tags that are incorporated into fusion proteins according to the present invention:

Preferred binding domains for ANN include: STNTKLHA (SEQ ID NO: 1); SSDRTLRR (SEQ ID NO: 2); STKERLKT (SEQ ID NO: 3); SQRANLRA (SEQ ID NO: 4); SSPADLTR (SEQ ID NO: 5); SSHSDLVR (SEQ ID NO: 6); SNGGELIR (SEQ ID NO: 7); SNQLILLK (SEQ ID NO: 8); SSRMDLKR (SEQ ID NO: 9); SRSDHLTN (SEQ ID NO: 10); SQLAHLRA (SEQ ID NO: 1); SQASSLKA (SEQ ID NO: 12); SQKSSLIA (SEQ ID NO: 13); SRKDNLKN (SEQ ID NO: 14); SDSGNLRV (SEQ ID NO: 15); SDRRNLRR (SEQ ID NO: 16); SDKKDLSR (SEQ ID NO: 17); SDASHLHT (SEQ ID NO: 18); STNSGLKN (SEQ ID NO: 19); STRMSLST (SEQ ID NO: 20); SNHDALRA (SEQ ID NO: 21); SRRSACRR (SEQ ID NO: 22); SRRSSCRK (SEQ ID NO: 23); SRSDTLSN (SEQ ID NO: 24); SRMGNLIR (SEQ ID NO: 25); SRSDTLRD (SEQ ID NO:26); SRAHDLVR (SEQ ID NO: 27); SRSDHLAE (SEQ ID NO: 28); SRRDALNV (SEQ ID NO: 29); STTGNLTV (SEQ ID NO: 30); STSGNLLV (SEQ ID NO: 31); STLTILKN (SEQ ID NO: 32); SRMSTLRH (SEQ ID NO: 33); STRSDLLR (SEQ ID NO: 34); STKTDLKR (SEQ ID NO: 35); STHIDLIR (SEQ ID NO: 36); SHRSTLLN (SEQ ID NO: 37); STSHGLTT (SEQ ID NO: 38); SHKNALQN (SEQ ID NO: 39); QRANLRA (SEQ ID NO: 40); DSGNLRV (SEQ ID NO: 41); RSDTLSN (SEQ ID NO: 42); TTGNLTV (SEQ ID NO: 43); SPADLTR (SEQ ID NO: 44); DKKDLTR (SEQ ID NO: 45); RTDTLRD (SEQ ID NO: 46); THLDLIR (SEQ ID NO: 47); QLAHLRA (SEQ ID NO: 48); RSDHLAE (SEQ ID NO: 49); HRTTLLN (SEQ ID NO: 50); QKSSLIA (SEQ ID NO: 51); RRDALNV (SEQ ID NO: 52); HKNALQN (SE ID NO: 53); RSDNLSN (SEQ ID NO: 54); RKDNLKN (SEQ ID NO: 55); TSGNLLV (SEQ ID NO: 56); RSDHLTN (SEQ ID NO: 57); HRTTLTN (SEQ ID NO: 58); SHSDLVR (SEQ ID NO: 59); NGGELIR (SEQ ID NO: 60); STKDLKR (SEQ ID NO: 61); RRDELNV (SEQ ID NO: 62); QASSLKA (SEQ ID NO: 63); TSHGLTT (SEQ ID NO: 64); QSSHLVR (SEQ ID NO: 65); QSSNLVR (SEQ ID NO: 66); DPGALRV (SEQ ID NO: 67); RSDNLVR (SEQ ID NO: 68); QSGDLRR (SEQ ID NO: 69); and DCRDLAR (SEQ ID NO: 70).

Particularly preferred DNA binding domains for ANN include: SEQ ID NOs: 40-49.

For SEQ ID NO: 1 through SEQ ID NO: 39, eight amino acids are shown. In these sequences, the first amino acid, S (serine), is derived from the framework and can, optionally, be omitted These sequences can be used as zinc finger DNA domains with or without the initial serine.

Preferred additional domains for AGC include: DPGALIN (SEQ ID NO: 71); ERSHLRE (SEQ ID NO: 72); DPGHLTE (SEQ ID NO: 73); EPGALIN (SEQ ID NO: 74); DRSIILRE (SEQ ID NO: 75); EPGHLTE (SEQ ID NO: 76); ERSLLRE (SEQ ID NO: 77); DRSKLRE (SEQ ID NO: 78); DPGKLTE (SEQ ID NO: 79); EPGKLTE (SEQ ID NO: 80); DPGWLIN (SEQ ID NO: 81); DPGTLIN (SEQ ID NO: 82); DPGHLIN (SEQ ID NO: 83); ERSWLIN (SEQ ID NO: 84); ERSTLIN (SEQ ID NO: 85); DPGWLTE (SEQ ID NO: 86); DPGTLTE (SEQ ID NO: 87); EPGWLIN (SEQ ID NO: 88); EPGTLIN (SEQ ID NO: 89); EPGHLIN (SEQ ID NO: 90); DRSWLRE (SEQ ID NO: 91); DRSTLRE (SEQ ID NO: 92); EPGWLTE (SEQ ID NO: 93); EPGTLTE (SEQ ID NO: 94); ERSWLRE (SEQ ID NO: 95); ERSTLRE (SEQ ID NO: 96); DPGALRE (SEQ ID NO: 97); DPGALTE (SEQ ID NO: 98); ERSHLIN (SEQ ID NO: 99); ERSHLTE (SEQ ID NO: 100); DPGHLIN (SEQ ID NO: 101); DPGHLRE (SEQ ID NO: 102); EPGALRE (SEQ ID NO: 103); EPGALTE (SEQ ID NO: 104); DRSHLIN (SEQ ID NO: 105); DRSHLTE (SEQ ID NO: 106); EPGHLRE (SEQ ID NO: 107); ERSKLIN (SEQ ID NO: 108); ERSKLTE (SEQ ID NO: 109); DRSKLIN (SEQ ID NO: 110); DRSKLTE (SEQ ID NO: 111); DPGKLIN (SEQ ID NO: 112); DPGKLRE (SEQ ID NO: 113); EPGKLIN (SEQ ID NO: 114); EPGKLRE (SEQ ID NO: 115); DPGWLRE (SEQ ID NO: 116); DPGTLRE (SEQ ID NO: 117); DPGHLRE (SEQ ID NO: 118); DPGHLTE (SEQ ID NO: 119); ERSWLTE (SEQ ID NO: 120); ERSTLTE (SEQ ID NO: 121); EPGWLRE (SEQ ID NO: 122); EPGTLRE (SEQ ID NO: 123); DRSWLIN (SEQ ID NO: 124); DRSWLTE (SEQ ID NO: 125); DRSTLIN (SEQ ID NO: 126); and DRSTLTE (SEQ ID NO: 127).

Particularly preferred binding domains for AGC include SEQ NOs: 71-80.

Preferred binding domains for CNN include: QRHNLTE (SEQ ID NO: 128); QSGNLTE (SEQ ID NO: 129); NLQHLGE (SEQ ID NO: 130); RADNLTE (SEQ ID NO: 131); RADNLAI (SEQ ID NO: 132); NTTHLEH (SEQ ID NO: 133); SKKHLAE (SEQ ID NO: 134); RNDTLTE (SEQ ID NO: 135); RNDTLQA (SEQ ID NO: 136); QSGHLTE (SEQ ID NO: 137); QLAHLKE (SEQ ID NO: 138); QRAHLTE (SEQ ID NO: 139); HTGHLLE (SEQ ID NO: 140); RSDHLTE (SEQ ID NO: 141); RSDKLTE (SEQ ID NO: 142); RSDHLTD (SEQ ID NO: 143); RSDHLTN (SEQ ID NO: 144); SRRTCRA (SEQ ID NO: 145); QLRHLRE (SEQ ID NO: 146); QRHSLTE (SEQ ID NO: 147); QLAHLKR (SEQ ID NO: 148); NLQHLGE (SEQ ID NO: 149); RNDALTE (SEQ ID NO: 150); TKQTLTE (SEQ ID NO: 151); and QSGDLTE (SEQ ID NO: 152).

Preferred binding domains for GNN include: QSSNLVR (SEQ ID NO: 153); DPGNLVR (SEQ ID NO: 154); RSDNLVR (SEQ ID NO: 155); TSGNLVR (SEQ ID NO: 156); QSGDLRR (SEQ ID NO: 157); DCRDLAR (SEQ ID NO: 158); RSDDLVK (SEQ ID NO: 159); TSGELVR (SEQ ID NO: 160); QRAHLER (SEQ ID NO: 161); DPGHLVR (SEQ ID NO: 162); RSDKLVR (SEQ ID NO: 163); TSGHLVR (SEQ ID NO: 164); QSSSLVR (SEQ ID NO: 165); DPGALVR (SEQ ID NO: 166); RSDELVR (SEQ ID NO: 167); TSGSLVR (SEQ ID NO: 168); QRSNLVR (SEQ ID NO: 169); QSGNLVR (SEQ ID NO: 170); QPGNLVR (SEQ ID NO: 171); DPGNLKR (SEQ ID NO: 172); RSDNLRR (SEQ ID NO: 173); KSANLVR (SEQ ID NO: 174); RSDNLVK (SEQ ID NO: 175); KSAQLVR (SEQ ID NO: 176); QSSTLVR (SEQ ID NO: 177); QSGTLRR (SEQ ID NO: 178); QPGDLVR (SEQ ID NO: 179); QGPDLVR (SEQ ID NO: 180); QAGTLMR (SEQ ID NO: 181); QPGTLVR (SEQ ID NO: 182); QGPELVR (SEQ ID NO: 183); GCRELSR (SEQ ID NO: 184); DPSTLKR (SEQ ID NO: 185); DPSDLKR (SEQ ID NO: 186); DSGDLVR (SEQ ID NO: 187); DSGELVR (SEQ ID NO: 188); DSGELKR (SEQ ID NO: 189); RLDTLGR (SEQ ID NO: 190); RPGDLVR (SEQ ID NO: 191); RSDTLVR (SEQ ID NO: 192); KSADLKR (SEQ ID NO: 193); RSDDLVR (SEQ ID NO: 194); RSDTLVK (SEQ ID NO: 195); KSAELKR (SEQ ID NO: 196); KSAELVR (SEQ ID NO: 197); RGPELVR (SEQ ID NO: 198); KPGELVR (SEQ ID NO: 199); SSQTLTR (SEQ ID NO: 200); TPGELVR (SEQ ID NO: 201); TSGDLVR (SEQ ID NO: 202); SSQTLVR (SEQ ID NO: 203); TSQTLTR (SEQ ID NO: 204); TSGELKR (SEQ ID NO: 205); QSSDLVR (SEQ ID NO: 206); SSGTLVR (SEQ ID NO: 207); TPGTLVR (SEQ ID NO: 208); TSQDLKR (SEQ ID NO: 209); TSGTLVR (SEQ ID NO: 210); QSSHLVR (SEQ ID NO: 211); QSGHLVR (SEQ ID NO: 212); QPGHLVR (SEQ ID NO: 213); ERSKLAR (SEQ ID NO: 214); DPGHLAR (SEQ ID NO: 215); QRAKLER (SEQ ID NO: 216); QSSKLVR (SEQ ID NO: 217); DRSKLAR (SEQ ID NO: 218); DPGKLAR (SEQ ID NO: 219); RSKDLTR (SEQ ID NO: 220); RSDHLTR (SEQ ID NO: 221); KSAKLER (SEQ ID NO: 222); TADHLSR (SEQ ID NO: 223); TADKLSR (SEQ ID NO: 224); TPGHLVR (SEQ ID NO: 225); TSSHLVR (SEQ ID NO: 226); TSGKLVR (SEQ ID NO: 227); QPGELVR (SEQ ID NO: 228); QSGELVR (SEQ ID NO: 229); QSGELRR (SEQ ID NO: 230); DPGSLVR (SEQ ID NO: 231); RKDSLVR (SEQ ID NO: 232); RSDVLVR (SEQ ID NO: 233); RHDSLLR (SEQ ID NO: 234); RSDALVR (SEQ ID NO: 235); RSSSLVR (SEQ ID NO: 236); RSSSHVR (SEQ ID NO: 237); RSDELVK (SEQ ID NO: 238); RSDALVK (SEQ ID NO: 239); RSDVLVK (SEQ ID NO: 240); RSSALVR (SEQ ID NO: 241); RKDSLVK (SEQ ID NO: 242); RSASLVR (SEQ ID NO: 243); RSDSLVR (SEQ ID NO: 244); RIHSLVR (SEQ ID NO: 245); RPGSLVR (SEQ ID NO: 246); RGPSLVR (SEQ ID NO: 247); RPGALVR (SEQ ID NO: 248); KSASKVR (SEQ ID NO: 249); KSAALVR (SEQ ID NO: 250); KSAVLVR (SEQ ID NO: 251); TSGSLTR (SEQ ID NO: 252); TSQSLVR (SEQ ID NO: 253); TSSSLVR (SEQ ID NO: 254); TPGSLVR (SEQ ID NO: 255); TSGALVR (SEQ ID NO: 256); TPGALVR (SEQ ID NO: 257); TGGSLVR (SEQ ID NO: 258); TSGELVR (SEQ ID NO: 259); TSGELTR (SEQ ID NO: 260); TSSALVK (SEQ ID NO: 261); and TSSALVR (SEQ ID NO: 262).

Particularly preferred binding domains for CNN include SEQ ID NOs: 153-168.

Preferred binding domains for TNN include: QASNLIS (SEQ ID NO: 263); SRGNLKS (SEQ ID NO: 264); RLDNLQT (SEQ ID NO: 265); ARGNLRT (SEQ ID NO: 266); RKDALRG (SEQ ID NO: 267); REDNLHT (SEQ ID NO: 268); ARGNLKS (SEQ ID NO: 269); RSDNLTT (SEQ ID NO: 270); VRGNLKS (SEQ ID NO: 271); VRGNLRT (SEQ ID NO: 272); RLRALDR (SEQ ID NO: 273); DMGALEA (SEQ ID NO: 274); EKDALRG (SEQ ID NO: 275); RSDHLTT (SEQ ID NO: 276); AQQLLMW (SEQ ID NO: 277); RSDERKR (SEQ ID NO: 278); DYQSLRQ (SEQ ID NO: 279); CFSRLVR (SEQ ID NO: 280); CDGGLWE (SEQ ID NO: 281); LQRPLRG (SEQ ID NO: 282); QGLACAA (SEQ ID NO: 283); WVGWLGS (SEQ ID NO: 284); RLRDIQF (SEQ ID NO: 285); GRSQLSC (SEQ ID NO: 286); GWQRLLT (SEQ ID NO: 287); SGRPLAS (SEQ ID NO: 288); APRLLGP (SEQ ID NO: 289); APKALGW (SEQ ID NO: 290); SVHELQG (SEQ ID NO: 291); AQAALSW (SEQ ID NO: 292); GANALRR (SEQ ID NO: 293); QSLLLGA (SEQ ID NO: 294); HRGTLGG (SEQ ID NO: 295); QVGLLAR (SEQ ID NO: 296); GARGLRG (SEQ ID NO: 297); DKHMLDT (SEQ ID NO: 298); DLGGLRQ (SEQ ID NO: 299); QCYRLER (SEQ ID NO: 300); AEAELQR (SEQ ID NO: 301); QGGVLAA (SEQ ID NO: 302); QGRCLVT (SEQ ID NO: 303); HPEALDN (SEQ ID NO: 304); GRGALQA (SEQ ID NO: 305); LASRLQQ (SEQ ID NO: 306); REDNLIS (SEQ ID NO: 307); RGGWLQA (SEQ ID NO: 308); DASNLIS (SEQ ID NO: 309); EASNLIS (SEQ ID NO: 310); RASNLIS (SEQ ID NO: 311); TASNLIS (SEQ ID NO: 312); SASNLIS (SEQ ID NO: 313); QASTLIS (SEQ ID NO: 314); QASDLIS (SEQ ID NO: 315); QASELIS (SEQ ID NO: 316); QASHLIS (SEQ ID NO: 317); QASKLIS (SEQ ID NO: 318); QASSLIS (SEQ ID NO: 319); QASALIS (SEQ ID NO: 320); DASTLIS (SEQ ID NO: 321); DASDLIS (SEQ ID NO: 322); DASELIS (SEQ ID NO: 323); DASHLIS (SEQ ID NO: 324); DASKLIS (SEQ ID NO: 325); DASSLIS (SEQ ID NO: 326); DASALIS (SEQ ID NO: 327); EASTLIS (SEQ ID NO: 328); EASDLIS (SEQ ID NO: 329); EASELIS (SEQ ID NO: 330); EASHLIS (SEQ ID NO: 331); EASKLIS (SEQ ID NO: 332); EASSLIS (SEQ ID NO: 333); EASALIS (SEQ ID NO: 334); RASTLIS (SEQ ID NO: 335); RASDLIS (SEQ ID NO: 336); RASELIS (SEQ ID NO: 337); RASHLIS (SEQ ID NO: 338); RASKLIS (SEQ ID NO: 339); RASSLIS (SEQ ID NO: 340); RASALIS (SEQ ID NO: 341); TASTLIS (SEQ ID NO: 342); TASDLIS (SEQ ID NO: 343); TASELIS (SEQ ID NO: 344); TASHLIS (SEQ ID NO: 345); TASKLIS (SEQ ID NO: 346); TASSLIS (SEQ ID NO: 347); TASALIS (SEQ ID NO: 348); SASTLIS (SEQ ID NO: 349); SASDLIS (SEQ ID NO: 350); SASELIS (SEQ ID NO: 351); SASHLIS (SEQ ID NO: 352); SASKLIS (SEQ ID NO: 353); SASSLIS (SEQ ID NO: 354); SASALIS (SEQ ID NO: 355); QLDNLQT (SEQ ID NO: 356); DLDNLQT (SEQ ID NO: 357); ELDNLQT (SEQ ID NO: 358); TLDNLQT (SEQ ID NO: 359); SLDNLQT (SEQ ID NO: 360); RLDTLQT (SEQ ID NO: 361); RLDDLQT (SEQ ID NO: 362); RLDELQT (SEQ ID NO: 363); RLDHLQT (SEQ ID NO: 364); RLDKLQT (SEQ ID NO: 365); RLDSLQT (SEQ ID NO: 366); RLDALQT (SEQ ID NO: 367); QLDTLQT (SEQ ID NO: 368); QLDDLQT (SEQ ID NO: 369); QLDELQT (SEQ ID NO: 370); QLDHLQT (SEQ ID NO: 371); QLDKLQT (SEQ ID NO: 372); QLDSLQT (SEQ ID NO: 373); QLDALQT (SEQ ID NO: 374); DLDTLQT (SEQ ID NO: 375); DLDDLQT (SEQ ID NO: 376); DLDELQT (SEQ ID NO: 377); DLDHLQT (SEQ ID NO: 378); DLDKLQT (SEQ ID NO: 379); DLDSLQT (SEQ ID NO: 380); DLDALQT (SEQ ID NO: 381); ELDTLQT (SEQ ID NO: 382); ELDDLQT (SEQ ID NO: 383); ELDELQT (SEQ ID NO: 384); ELDHLQT (SEQ ID NO: 385); ELDKLQT (SEQ ID NO: 386); ELDSLQT (SEQ ID NO: 387); ELDALQT (SEQ ID NO: 388); TLDTLQT (SEQ ID NO: 389); TLDDLQT (SEQ ID NO: 390); TLDELQT (SEQ ID NO: 391); TLDHLQT (SEQ ID NO: 392); TLDKLQT (SEQ ID NO: 393); TLDSLQT (SEQ ID NO: 394); TLDALQT (SEQ ID NO: 395); SLDTLQT (SEQ ID NO: 396); SLDDLQT (SEQ ID NO: 397); SLDELQT (SEQ ID NO: 398); SLDHLQT (SEQ ID NO: 399); SLDKLQT (SEQ ID NO: 400); SLDSLQT (SEQ ID NO: 401); SLDALQT (SEQ ID NO: 402); ARGTLRT (SEQ ID NO: 403); ARGDLRT (SEQ ID NO: 404); ARGELRT (SEQ ID NO: 405); ARGHLRT (SEQ ID NO: 406); ARGKLRT (SEQ ID NO: 407); ARGSLRT (SEQ ID NO: 408); ARGALRT (SEQ ID NO: 409); SRGTLRT (SEQ ID NO: 410); SRGDLRT (SEQ ID NO: 411); SRGELRT (SEQ ID NO: 412); SRGHLRT (SEQ ID NO: 413); SRGKLRT (SEQ ID NO: 414); SRGSLRT (SEQ ID NO: 415); SRGALRT (SEQ ID NO: 416); QKDALRG (SEQ ID NO: 417); DKDALRG (SEQ ID NO: 418); EKDALRG (SEQ ID NO: 419); TKDALRG (SEQ ID NO: 420); SKDALRG (SEQ ID NO: 421); RKDNLRG (SEQ ID NO: 422); RKDTLRG (SEQ ID NO: 423); RKDDLRG (SEQ ID NO: 424); RKDELRG (SEQ ID NO: 425); RKDHLRG (SEQ ID NO: 426); RKDKLRG (SEQ ID NO: 427); REDSLRG (SEQ ID NO: 428); QKDNLRG (SEQ ID NO: 429); QKDTLRG (SEQ ID NO: 430); QKDDLRG (SEQ ID NO: 431); QKDELRG (SEQ ID NO: 432); QKDHLRG (SEQ ID NO: 433); QKDKLRG (SEQ ID NO: 434); QKDSLRG (SEQ ID NO: 435); DKDNLRG (SEQ ID NO: 436); DKDTLRG (SEQ ID NO: 437); DKDDLRG (SEQ ID NO: 438); DKDELRG (SEQ ID NO: 439); DKDHLRG (SEQ ID NO: 440); DKDKLRG (SEQ ID NO: 441); DKDSLRG (SEQ ID NO: 442); EKDNLRG (SEQ ID NO: 443); EKDTLRG (SEQ ID NO: 444); EKDDLRG (SEQ ID NO: 445); EKDELRG (SEQ ID NO: 446); EKDHLRG (SEQ ID NO: 447); EKDKLRG (SEQ ID NO: 448); EKDSLRG (SEQ ID NO: 449); TKDNLRG (SEQ ID NO: 450); TKDTLRG (SEQ ID NO: 451); TKDDLRG (SEQ ID NO: 452); TKDELRG (SEQ ID NO: 453); TKDHLRG (SEQ ID NO: 454); TKDKLRG (SEQ ID NO: 455); TKDSLRG (SEQ ID NO: 456); SKDNLRG (SEQ ID NO: 457); SKDTLRG (SEQ ID NO: 458); SKDDLRG (SEQ ID NO: 459); SKDELRG (SEQ ID NO: 460); SKDHLRG (SEQ ID NO: 461); SKDKLRG (SEQ ID NO: 462); SKDSLRG (SEQ ID NO: 463); VRGTLRT (SEQ ID NO: 464); VRGDLRT (SEQ ID NO: 465); VRGELRT (SEQ ID NO: 466); VRGHLRT (SEQ ID NO: 467); VRCKLRT (SEQ ID NO: 468); VRGSLRT (SEQ ID NO: 469); VRGTLRT (SEQ ID NO: 470); QLRALDR (SEQ ID NO: 471); DLRALDR (SEQ ID NO: 472); ELRALDR (SEQ ID NO: 473); TLRALDR (SEQ ID NO: 474); SLRALDR (SEQ ID NO: 475); RSDNRKR (SEQ ID NO: 476); RSDTRKR (SEQ ID NO: 477); RSDDRKR (SEQ ID NO: 478); RSDHRKR (SEQ ID NO: 479); RSDKRKR (SEQ ID NO: 480); RSDSRKR (SEQ ID NO: 481); RSDARKR (SEQ ID NO: 482); QYQSLRQ (SEQ ID NO: 483); EYQSLRQ (SEQ ID NO: 484); RYQSLRQ (SEQ ID NO: 485); TYQSLRQ (SEQ ID NO: 486); SYQSLRQ (SEQ ID NO: 487); RLRNIQF (SEQ ID NO: 488); RLRTIQF (SEQ ID NO: 489); RLREIQF (SEQ ID NO: 490); RLRHIQF (SEQ ID NO: 491); RLRKIQF (SEQ ID NO: 492); RLRSIQF (SEQ ID NO: 493); RLRAIQF (SEQ ID NO: 494); DSLLLGA (SEQ ID NO: 495); ESLLLGA (SEQ ID NO: 496); RSLLLGA (SEQ ID NO: 497); TSLLLGA (SEQ ID NO: 498); SSLLLGA (SEQ ID NO: 499); HRGNLGG (SEQ ID NO: 500); HRGDLGG (SEQ ID NO: 501); HRGELGG (SEQ ID NO: 502); HRGHLGG (SEQ ID NO: 503); HRGKLGG (SEQ ID NO: 504); HRGSLGG (SEQ ID NO: 505); HRGALGG (SEQ ID NO: 506); QKHMLDT (SEQ ID NO: 507); EKHMLDT (SEQ ID NO: 508); RKHMLDT (SEQ ID NO: 509); TKHMLDT (SEQ ID NO: 510); SKHMLDT (SEQ ID NO: 511); QLGGLRQ (SEQ ID NO: 512); ELGGLRQ (SEQ ID NO: 513); RLGGLRQ (SEQ ID NO: 514); TLGGLRQ (SEQ ID NO: 515); SLGGLRQ (SEQ ID NO: 516); AEANLQR (SEQ ID NO: 517); AEATLQR (SEQ ID NO: 518); AEADLQR (SEQ ID NO: 519); AEAHLQR (SEQ ID NO: 520); AEAKLQR (SEQ ID NO: 521); AEASLQR (SEQ ID NO: 522); AEAALQR (SEQ ID NO: 523); DGRCLVT (SEQ ID NO: 524); EGRCLVT (SEQ ID NO: 525); RGRCLVT (SEQ ID NO: 526); TGRCLVT (SEQ ID NO: 527); SGRCLVT (SEQ ID NO: 528); QEDNLHT (SEQ ID NO: 529); DEDNLHT (SEQ ID NO: 530); EEDNLHT (SEQ ID NO: 531); SEDNLHT (SEQ ID NO: 532); REDTLHT (SEQ ID NO: 533); REDDLHT (SEQ ID NO: 534); REDELHT (SEQ ID NO: 535); REDHLHT (SEQ ID NO: 536); REDKLHT (SEQ ID NO: 537); REDSLHT (SEQ ID NO: 538); REDALHT (SEQ ID NO: 539); QEDTLHT (SEQ ID NO: 540); QEDDLHT (SEQ ID NO: 541); QEDELHT (SEQ ID NO: 542); QEDHLHT (SEQ ID NO: 543); QEDKLHT (SEQ ID NO: 544); QEDSLHT (SEQ ID NO: 545); QEDALHT (SEQ ID NO: 546); DEDTLHT (SEQ ID NO: 547); DEDDLHT (SEQ ID NO: 548); DEDELHT (SEQ ID NO: 549); DEDHLHT (SEQ ID NO: 550); DEDKLHT (SEQ ID NO: 551); DEDSLHT (SEQ ID NO: 552); DEDALHT (SEQ ID NO: 553); EEDTLHT (SEQ ID NO: 554); EEDDLHT (SEQ ID NO: 555); EEDELHT (SEQ ID NO: 556); EEDHLHT (SEQ ID NO: 557); EEDKLHT (SEQ ID NO: 558); EEDSLHT (SEQ ID NO: 559); EEDALHT (SEQ ID NO: 560); TEDTLHT (SEQ ID NO: 561); TEDDLHT (SEQ ID NO: 562); TEDELHT (SEQ ID NO: 563); TEDHLHT (SEQ ID NO: 564); TEDKLHT (SEQ ID NO: 565); TEDSLHT (SEQ ID NO: 566); TEDALHT (SEQ ID NO: 567); SEDTLHT (SEQ ID NO: 568); SEDDLHT (SEQ ID NO: 569); SEDELHT (SEQ ID NO: 570); SEDHLHT (SEQ ID NO: 571); SEDKLHT (SEQ ID NO: 572); SEDSLHT (SEQ ID NO: 573); SEDALHT (SEQ ID NO: 574); QEDNLIS (SEQ ID NO: 575); DEDNLIS (SEQ ID NO: 576); EEDNLIS (SEQ ID NO: 577); SEDNLIS (SEQ ID NO: 578); REDTLIS (SEQ ID NO: 579); REDDLIS (SEQ ID NO: 580); REDELIS (SEQ ID NO: 581); REDHLIS; (SEQ ID NO: 582); REDKLIS (SEQ ID NO: 583); REDSLIS (SEQ ID NO: 584); REDALIS (SEQ ID NO: 585); QEDTLIS (SEQ ID NO: 586); QEDDLIS (SEQ ID NO: 587); QEDELIS (SEQ ID NO: 588); QEDHLIS (SEQ ID NO: 589); QEDKLIS (SEQ ID NO: 590); QEDSLIS (SEQ ID NO: 591); QEDALIS (SEQ ID NO: 592); DEDTLIS (SEQ ID NO: 593); DEDDLIS (SEQ ID NO: 594); DEDELIS (SEQ ID NO: 595); DEDHLIS (SEQ ID NO: 596); DEDKLIS (SEQ ID NO: 597); DEDSLIS (SEQ ID NO: 598); DEDALIS (SEQ ID NO: 599); EEDTLIS (SEQ ID NO: 600); EEDDLIS (SEQ ID NO: 601); EEDELIS (SEQ ID NO: 602); EEDHLIS (SEQ ID NO: 603); EEDKLIS (SEQ ID NO: 604); EEDSLIS (SEQ ID NO: 605); EEDALIS (SEQ ID NO: 606); TEDTLIS (SEQ ID NO: 607); TEDDLIS (SEQ ID NO: 608); TEDELIS (SEQ ID NO: 609); TEDHLIS (SEQ ID NO: 610); TEDKLIS (SEQ ID NO: 611); TEDSLIS (SEQ ID NO: 612); TEDALIS (SEQ ID NO: 613); SEDTLIS (SEQ ID NO: 614); SEDDLIS (SEQ ID NO: 615); SEDELIS (SEQ ID NO: 616); SEDHLIS (SEQ ID NO: 617); SEDKLIS (SEQ ID NO: 618); SEDSLIS (SEQ ID NO: 619); SEDALIS (SEQ ID NO: 620); TGGWLQA (SEQ ID NO: 621); SGGWLQA (SEQ ID NO: 622); DGGWLQA (SEQ ID NO: 623); EGGWLQA (SEQ ID NO: 624); QGGWLQA (SEQ ID NO: 625); RGGTLQA (SEQ ID NO: 626); RGGDLQA (SEQ ID NO: 627); RGGELQA (SEQ ID NO: 628); RGGNLQA (SEQ ID NO: 629); RGGHLQA (SEQ ID NO: 630); RGGKLQA (SEQ ID NO: 631); RGGSLQA (SEQ ID NO: 632); RGGALQA (SEQ ID NO: 633); TGGTLQA (SEQ ID NO: 634); TGGDLQA (SEQ ID NO: 635); TGGELQA (SEQ ID NO: 636); TGGNLQA (SEQ ID NO: 637); TGGHLQA (SEQ ID NO: 638); TGGKLQA (SEQ ID NO: 639); TGGSLQA (SEQ ID NO: 640); TGGALQA (SEQ ID NO: 641); SGGTLQA (SEQ ID NO: 642); SGGDLQA (SEQ ID NO: 643); SGGELQA (SEQ ID NO: 644); SGGNLQA (SEQ ID NO: 645); SGGHLQA (SEQ ID NO: 646); SGGKLQA (SEQ ID NO: 647); SGGSLQA (SEQ ID NO: 648); SGGALQA (SEQ ID NO: 649); DGGTLQA (SEQ ID NO: 650); DGGDLQA (SEQ ID NO: 651); DGGELQA (SEQ ID NO: 652); DGGNLQA (SEQ ID NO: 653); DGGHLQA (SEQ ID NO: 654); DGGKLQA (SEQ ID NO: 655); DGGSLQA (SEQ ID NO: 656); DGGALQA (SEQ ID NO: 657); EGGTLQA (SEQ ID NO: 658); EGGDLQA (SEQ ID NO: 659); EGGELQA (SEQ ID NO: 660); EGGNLQA (SEQ ID NO: 661); EGGHLQA (SEQ ID NO: 662); EGGKLQA (SEQ ID NO: 663); EGGSLQA (SEQ ID NO: 664); EGGALQA (SEQ ID NO: 665); QGGTLQA (SEQ ID NO: 666); QGGDLQA (SEQ ID NO: 667); QGGELQA (SEQ ID NO: 668); QGGNLQA (SEQ ID NO: 669); QGGHLQA (SEQ ID NO: 670); QGGKLQA (SEQ ID NO: 671); QGGSLQA (SEQ ID NO: 672); and QGGALQA (SEQ ID NO: 673).

Particularly preferred binding domains for TNN include SEQ ID NOs: 263-308. More particularly preferred binding domains for TNN include SEQ ID NOs: 263-268.

Accordingly, in one alternative, at least one of the zinc finger protein tags of the fusion protein has at least one zinc finger DNA binding domain therein specifically binding at least one DNA subsite of the structure 5′-ANN-3′ and at least one zinc finger DNA binding domain therein specifically binding at least one DNA subsite of a structure selected from the group consisting of 5′-CNN-3′, 5′-GNN-3′, and 5′-TNN-3′. In another alternative, at least one of the zinc finger protein tags of the fusion protein has at least one zinc finger DNA binding domain therein specifically binding at least one DNA subsite of the structure 5′-CNN-3′ and at least one zinc finger DNA binding domain therein specifically binding at least one DNA subsite of a structure selected from the group consisting of 5′-ANN-3′, 5′-GNN-3′, and 5′-TNN-3′. In yet another alternative, at least one of the zinc finger protein tags of the fusion protein has at least one zinc finger DNA binding domain therein specifically binding at least one DNA subsite of the structure 5′-GNN-3′ and at least one zinc finger DNA binding domain therein specifically binding at least one DNA subsite of a structure selected from the group consisting of 5′-ANN-3′, 5′-CNN-3′, and 5′-TNN-3′. In still another alternative, at least one of the zinc finger protein tags of the fusion protein has at least three zinc finger DNA binding domains therein, each zinc finger DNA binding domain binding a DNA subsite of a different structure wherein the structures are selected from the group consisting of 5′-ANN-3′, 5′-CNN-3′, 5′-GNN-3′, and 5′-TNN-3′. In this alternative, at least one of the zinc finger protein tags of the fusion protein can have at least four zinc finger DNA binding domains therein, each zinc finger DNA binding domain binding a DNA subsite of a different structure wherein the structures are selected from the group consisting of 5′-ANN-3′, 5′-CNN-3′, 5′-GNN-3′, and 5′-TNN-3′.

Other zinc finger modules or zinc finger DNA binding domains are known in the art. For example, zinc finger modules or zinc finger DNA binding domains are described in: U.S. Pat. No. 7,067,317 to Rebar et al.; U.S. Pat. No. 7,030,215 to Liu et al.; U.S. Pat. No. 7,026,462 to Rebar et al.; U.S. Pat. No. 7,013,219 to Case et al.; U.S. Pat. No. 6,979,539 to Cox III et al.; U.S. Pat. No. 6,933,113 to Case et al.; U.S. Pat. No. 6,824,978 to Cox III et al.; U.S. Pat. No. 6,794,136 to Eisenberg et al.; U.S. Pat. No. 6,785,613 to Eisenberg et al.; U.S. Pat. No. 6,777,185 to Case et al.; U.S. Pat. No. 6,706,470 to Choo et al.; U.S. Pat. No. 6,607,882 to Cox III et al.; U.S. Pat. No. 6,599,692 to Case et al.; U.S. Pat. No. 6,534,261 to Cox III et al.; U.S. Pat. No. 6,503,717 to Case et al.; U.S. Pat. No. 6,453,242 to Eisenberg et al.; United States Patent Application Publication No. 2006/0246588 to Rebar et al.; United States Patent Application Publication No. 2006/0246567 to Rebar et al.; United States Patent Application Publication No. 2006/0166263 to Case et al.; United States Patent Application Publication No. 2006/0078878 to Cox III et al.; United States Patent Application Publication No. 2005/0257062 to Rebar et al.; United States Patent Application Publication No. 2005/0215502 to Cox III et al.; United States Patent Application Publication No. 2005/0130304 to Cox III et al.; United States Patent Application Publication No. 2004/0203064 to Case et al.; United States Patent Application Publication No. 2003/0166141 to Case et al.; United States Patent Application Publication No. 2003/0134318 to Case et al.; United States Patent Application Publication No. 2003/0105593 to Eisenberg et al.; United States Patent Application Publication No. 2003/0087817 to Cox III et al.; United States Patent Application Publication No. 2003/0021776 to Rebar et al.; and U.S. Pat. No. Application Publication No. 2002/0081614 to Case et al., all of which are incorporated herein by this reference. These zinc finger modules or zinc finger DNA binding domains described in these patents and patent publications can be incorporated in fusion proteins according to the present invention. For example, one alternative described in these patents and patent publications involves the use of so-called “D-able sites” and zinc finger modules or zinc finger DNA binding domains that can bind to such sites. A “D-able” site is a region of a target site that allows an appropriately designed zinc finger module or zinc finger DNA binding domain to bind to four bases rather than three of the target strand. Such a zinc finger module or zinc finger DNA binding domain binds to a triplet of three bases on one strand of a double-stranded DNA target segment (target strand) and a fourth base on the other, complementary, strand. Binding of a single zinc finger to a four base target segment imposes constraints both on the sequence of the target strand and on the amino acid sequence of the zinc finger. The target site within the target strand should include the “D-able” site motif 5′ NNGK 3′, in which N and K are conventional IUPAC-IUB ambiguity codes. A zinc finger for binding to such a site should include an arginine residue at position −1 and an aspartic acid, (or less preferably a glutamic acid) at position +2. The arginine residues at position −1 interacts with the G residue in the D-able site. The aspartic acid (or glutamic acid) residue at position +2 of the zinc finger interacts with the opposite strand base complementary to the K base in the D-able site. It is the interaction between aspartic acid (symbol D) and the opposite strand base (fourth base) that confers the name D-able site. As is apparent from the D-able site formula, there are two subtypes of D-able sites: 5′ NNGG 3′ and 5′ NNGT 3′. For the former site, the aspartic acid or glutamic acid at position +2 of a zinc finger interacts with a C in the opposite strand to the D-able site. In the latter site, the aspartic acid or glutamic acid at position +2 of a zinc finger interacts with an A in the opposite strand to the D-able site. In general, NNGG is preferred over NNGT. In the design of a ZFP with three fingers, a target site should be selected in which at least one finger of the protein, and optionally, two or all three fingers have the potential to bind a D-able site. Such can be achieved by selecting a target site from within a larger target gene having the formula 5′-NNx aNy bNzc-3′, wherein each of the sets (x,a), (y,b) and (z,c) is either (N,N) or (G,K); at least one of (x,a), (y,b) and (z,e) is (G,K), and N and K are IUPAC-IUB ambiguity codes. In other words, at least one of the three sets (x,a), (y,b) and (z,c) is the set (G,K), meaning that the first position of the set is G and the second position is G or T. Those of the three sets (if any) which are not (G,K) are (N,N), meaning that the first position of the set can be occupied by any nucleotide and the second position of the set can be occupied by any nucleotide. As an example, the set (x,a) can be (G,K) and the sets (y,b) and (z,c) can both be (N,N). In the formula 5′-NNx aNy bNzc-3′, the triplets of NNx aNy and bNzc represent the triplets of bases on the target strand bound by the three fingers in a ZFP. If only one of x, y and z is a G, and this G is followed by a K, the target site includes a single D-able subsite. These can be incorporated into fusion proteins according to the present invention.

However, as defined above, the terms “zinc finger,” “zinc finger “zinc finger tag,” zinc finger module,” “zinc finger nucleotide binding domain,” and the like do not require that the amino acid sequence specified thereby originate from an actual zinc finger or necessarily have substantial homology with a naturally-occurring or constructed zinc finger protein. They are used to describe the general nature of the protein domains involved and do not necessarily require the participation of a zinc ion in the protein structure.

Zinc finger nucleotide binding domains that are included in chimeric recombinases according to the present invention comprise two subdomains.

The first of these subdomains is the DNA binding subdomain. As described above, typically this subdomain comprises from about 7 to about 10 amino acids, most commonly 7 or 8 amino acids, and possesses the specific DNA binding capacity described above. The DNA binding subdomain can alternatively be referred to as a domain and is so referred to herein; however, it is so referred to with the understanding that the framework subdomain, referred to below, is typically required for the maintenance of optimal secondary and tertiary structure.

The second of these subdomains is the framework subdomain. In one alternative, based on the structure of naturally-occurring zinc finger proteins, the framework subdomain is split into two halves, a first half that is located such that the amino-terminus of the DNA binding subdomain is located at the carboxyl terminus of the first half of the framework subdomain, and the second located such that the carboxyl-terminus of the DNA binding subdomain is located at the amino-terminus of the second half of the framework subdomain.

In this alternative, the framework subdomain can include two cysteine residues and two histidine residues, as is commonly found in wild-type zinc finger proteins. This arrangement is designated herein as C₂H₂. In wild-type zinc finger proteins in the C₂H₂ arrangement, the two cysteine residues are located to the amino-terminal side of the DNA binding subdomain, and the two histidine residues are located to the carboxyl-terminal side of the DNA binding subdomain. The cysteine and histidine residues bind the zinc ion in the zinc finger protein.

Although wild-type zinc finger proteins generally, but not exclusively have the C₂H₂ arrangement, it is possible to interchange the cysteine and histidine residues in the framework subdomain in order to generate framework domains with three cysteine residues and one histidine residue (C₃H), or with four cysteine residues (C₄), which are known for a few naturally-occurring zinc finger proteins. Additionally, mutagenesis has been employed to generate H₄ and CH₃ arrangements of these framework subdomains. In the CH₃ arrangements, any of the four relevant residues can be cysteine; the other three are all histidine. These mutated zinc finger proteins are disclosed in S. Neri et al., “Creation and Characteristics of Unnatural CysHis₃-Type Zinc Finger Protein,” Biochem. Biophys. Res. Commun. 325: 421-425 (2004), incorporated herein by this reference. Similar mutated zinc finger proteins are also disclosed in Y. Hori et al., “The Engineering, Structure and DNA Binding Properties of a Novel His₄-Type Zinc Finger Peptide,” Nucleic Acids Symp. 44: 295-296 (2000), incorporated herein by this reference.

Additionally, there exist zinc finger proteins with a C₆ (six cysteine residues) arrangement, and that arrangement can be incorporated into framework subdomains that form part of zinc finger nucleotide binding domains in fusion proteins according to the present invention (Y. Hori et al., “The Engineering, Structure, and DNA Binding Properties of a Novel His₄-Type Zinc Finger Peptide,” Nucleic Acids Symp. 44: 295-296 (2000)).

An additional framework subdomain is that based on the protein avian pancreatic polypeptide (aPP). The small protein aPP has a solvent-exposed α-helical face and a solvent-exposed Type II polyproline helical face. In zinc finger nucleotide binding domains based on aPP, the DNA binding subdomains from zinc finger nucleotide binding domains, as described above, are grafted onto either the solvent-exposed α-helical face or the solvent-exposed Type II polyproline helical face of aPP. Residues can be mutated to provide tighter or more specific DNA binding. This approach is described in L. Yang & A. Schepartz, “Relationship Between Folding and Function in a Sequence-Specific Miniature DNA-Binding Protein,” Biochemistry 44: 7469-7478 (2005), and in N. J. Zondlo & A. Schepartz, “Highly Specific DNA Recognition by a Designed Miniature Protein,” J. Am. Chem. Soc. 121: 6938-6939 (1999), both incorporated herein by this reference. Typically, the residues are grafted onto the solvent-exposed α-helical face of aPP. In this approach, the DNA binding subdomains can be interspersed with α-helical residues. These framework domains can, therefore, be incorporated into fusion proteins according to the present invention.

In summary, the preparation of zinc finger tags for incorporation into fusion proteins according to the present invention involves: (1) selection of the nucleotide sequence to be specifically bound by the zinc finger tag; (2) determination of how many zinc finger modules are required in 3-base pair units, each module considered to bind 3 base pairs; (3) selection of the appropriate background (i.e., Zif268); (4) selection of appropriate sequence specificity-conferring heptapeptide or octapeptide sequences for each module considering the information provided above, including the 5′-nucleotide of the triplet (A, C, G, or T), and the information presented herein or otherwise available regarding the correspondence between particular amino acids in the amino acid sequence of the heptapeptide or octapeptide and the particular nucleotide interacting with that amino acid and general rules for such correspondence, so that cross-subsite interactions are minimized; (5) construction and testing of the zinc finger module; and (6) modification of the heptapeptide or octapeptide sequence or of the background to optimize specificity, such as by site-specific mutagenesis if required. The process can also include consideration of an appropriate framework subdomain, as described above, as the conformational constraints imposed by the framework subdomain chosen can modify the binding pattern of the zinc finger module to the nucleic acid sequence.

Additionally, fusion proteins according to the present invention can include conservative amino acid substitutions, in the protein of interest, in the at least one zinc finger tag, and where appropriate, in the framework subdomain. In the zinc finger tag, fusion proteins according to the present invention include zinc finger tags that that differ from the zinc finger tags disclosed above or included herein by this reference by no more than two conservative amino acid substitutions that have a binding affinity for the desired subsite or target region of at least 80% as great as the zinc finger tag before the substitutions are made. In terms of dissociation constants, this is equivalent to a dissociation constant no greater than 125% of that of the zinc finger tag before the substitutions are made. In this context, the term “conservative amino acid substitution” is defined as one of the following substitutions: Ala/Gly or Ser; Arg/Lys; Asn/Gln or His; Asp/Glu; Cys/Ser; Gln/Asn; Gly/Asp; Gly/Ala or Pro; His/Asn or Gln; Ile/Leu or Val; Leu/Ile or Val; Lys/Arg or Gln or Glu; Met/Leu or Tyr or Ile; Phe/Met or Leu or Tyr; Ser/Thr; Thr/Ser; Trp/Tyr; Tyr/Trp or Phe; Val/Ile or Leu. Preferably, the zinc finger tag differs from the zinc finger tag described above or included herein by this reference by no more than one conservative amino acid substitution. In the protein of interest, conservative amino acid substitutions according to the guidelines given above can include up to about 10% of the residues of the protein of interest, subject to the proviso that the substituted protein of interest substantially retains its original activity. If a quantitative measurement is available for the activity of the protein of interest, “substantially retains” is defined herein to mean that the protein of interest retains at least 80% of its activity before substitution, such as a dissociation constant no more than 125% of the original dissociation constant for binding a ligand or a maximum rate of enzymatic catalysis no less than 80% of the original rate. Preferably, conservative amino acid substitutions include no more than about 5% of the residues of the protein of interest. More preferably, conservative amino acid substitutions include no more than about 2.5% of the residues of the protein of interest.

II. Polynucleotides, Expression Vectors, Transformed Cells and Processes of Expression of Fusion Proteins

Another aspect of the invention is polynucleotides that encode fusion proteins according to the present invention, expression vectors that incorporate such polynucleotides, and cells that are transformed or transfected with such expression vectors.

Polynucleotides that encode fusion proteins according to the present invention are within the scope of the invention. As used herein, the terms “polynucleotide,” “nucleotide sequence,” “nucleic acid sequence,” “nucleic acid construct,” and terms of similar import include both DNA, DNA complements and RNA unless otherwise specified, and, unless otherwise specified, includes both double-stranded and single-stranded nucleic acids. Also included are hybrids such as DNA-RNA hybrids. In particular, a reference to DNA includes RNA that has either the equivalent base sequence except for the substitution of uracil and RNA for thymine in DNA, or has a complementary base sequence except for the substitution of uracil for thymine, complementarity being determined according to the Watson-Crick base pairing rules. Reference to nucleic acid sequences can also include modified bases as long as the modifications do not significantly interfere either with binding of a ligand such as a protein by the nucleic acid or with Watson-Crick base pairing.

Additionally, unless specifically excluded, all nucleic acid sequences that encode a specific fusion protein of the present invention according to the generally-accepted triplet code are within the scope of the invention. The recitation of one nucleic acid sequence that encodes a particular fusion protein according to the present invention is therefore not to be interpreted as an exclusion of any other nucleic acid sequence that can encode the fusion protein. Once the sequence of the fusion protein is determined, all nucleic acid sequences that can encode that fusion protein can be readily be determined by one of ordinary skill in the art by using the generally-accepted triplet code, such as that recited at B. Lewin, “Genes VIII” (Pearson/Prentice-Hall, Upper Saddle River, N.J., 2004), p. 168, incorporated herein by this reference.

Additionally, in view of the existence of conservative amino acid substitutions as described above, unless specifically excluded, all nucleic acid sequences that encode a variant of a fusion protein according to the present invention differing by one or more conservative amino acid substitutions, as defined above, while retaining appropriate functioning in all domains of the fusion protein, are within the scope of the present invention. Such nucleic acid sequences can again be readily determined by one of ordinary skill in the art using the triplet code once the protein sequence of the variant of the fusion protein is specified.

DNA sequences encoding fusion proteins according to the present invention can be obtained by several methods. For example, the DNA can be isolated using hybridization procedures which are well known in the art. These include, but are not limited to: (1) hybridization of probes to genomic or cDNA libraries to detect shared nucleotide sequences; (2) antibody screening of expression libraries to detect shared structural features; and (3) synthesis by the polymerase chain reaction (PCR). RNA sequences of the invention can be obtained by methods known in the art (See, for example, Current Protocols in Molecular Biology, Ausubel, et al., eds., 1989).

The development of specific DNA sequences encoding fusion proteins according to the present invention can be obtained by: (1) isolation of a double-stranded DNA sequence from the genomic DNA, typically the genomic DNA of a genetically-engineered organism as described in further detail below; (2) chemical manufacture of a DNA sequence to provide the necessary codons for the fusion protein; and (3) in vitro synthesis of a double-stranded DNA sequence by reverse transcription of mRNA isolated from a eukaryotic donor cell, typically a genetically-engineered cell. In the latter case, a double-stranded DNA complement of mRNA is eventually formed which is generally referred to as cDNA. Of these three methods for developing specific DNA sequences for use in recombinant procedures, the isolation of genomic DNA is the least common. This is especially true when it is desirable to obtain the microbial expression of mammalian polypeptides due to the presence of introns.

For obtaining DNA sequences that encode fusion proteins according to the present invention, the synthesis of DNA sequences is frequently the method of choice when the entire sequence of amino acid residues of the desired polypeptide product is known. When the entire sequence of amino acid residues of the desired polypeptide is not known, the direct synthesis of DNA sequences is not possible and the method of choice is the formation of cDNA sequences. Among the standard procedures for isolating cDNA sequences of interest is the formation of plasmid-carrying cDNA libraries which are derived from reverse transcription of mRNA which is abundant in donor cells that have a high level of genetic expression. When used in combination with polymerase chain reaction technology, even rare expression products can be clones. In those eases where significant portions of the amino acid sequence of the polypeptide are known, the production of labeled single or double-stranded DNA or RNA probe sequences duplicating a sequence putatively present in the target cDNA may be employed in DNA/DNA hybridization procedures which are carried out on cloned copies of the cDNA which have been denatured into a single-stranded form (Jay, et al., Nucleic Acid Research 11:2325, 1983).

Nucleic acid constructs encoding fusion proteins according to the present invention can be constructed by standard molecular cloning techniques, as described, for example, in J. Sambrook & D. W. Russell, “Molecular Cloning: A Laboratory Manual” (3rd ed., Cold Spring Harbor Laboratory Press, Cold Spring Harbor, N.Y., 2001). In general, a single nucleic acid construct includes regions encoding the protein of interest and encoding the zinc finger tag as described above. These regions can be contiguous or can be separated by one or more spacers. The nucleic acid construct encoding the fusion protein can be constructed such that the zinc finger tag is either at the N-terminal end or at the C-terminal end of the expressed protein. As indicated above, nucleic acid constructs encoding the fusion protein can also encode additional domains such as purification tags, enzyme domains, or other domains, without significantly altering the specific DNA-binding activity of the zinc finger tag or the activity of the protein of interest. In one example, the polypeptides can be incorporated into two halves of a split enzyme like a β-lactamase to allow the sequences to be sensed in cells or in vivo. Binding of two halves of such a split enzyme then allows for assembly of the split enzyme (J. M. Spotts et al. “Time-Lapse Imaging of a Dynamic Phosphorylation Protein-Protein Interaction in Mammalian Cells,” Proc. Natl. Acad. Sci. USA 99: 15142-15147 (2002)).

Construction of nucleic acid sequences according to the present invention can be accomplished by techniques well known in the art, including solid-phase nucleotide synthesis, the polymerase chain reaction (PCR) technique, reverse transcription of DNA from RNA, the use of DNA polymerases and ligases, and other techniques. If an amino acid sequence is known, the corresponding nucleic acid sequence can be constructed according to the genetic code.

Hybridization procedures are useful for the screening of recombinant clones by using labeled mixed synthetic oligonucleotide probes where each probe is potentially the complete complement of a specific DNA sequence in the hybridization sample which includes a heterogeneous mixture of denatured double-stranded DNA. For such screening, hybridization is preferably performed on either single-stranded DNA or denatured double-stranded DNA. Hybridization is particularly useful in the detection of cDNA clones derived from sources where an extremely low amount of mRNA sequences encoding a fusion protein according to the present invention interest are present. By using stringent hybridization conditions directed to avoid non-specific binding, it is possible, for example, to allow the autoradiographic visualization of a specific cDNA clone by the hybridization of the target DNA to that single probe in the mixture which is its complete complement (Wallace, et al., Nucleic Acid Research, 9:879, 1981; Maniatis, et al., Molecular Cloning: A Laboratory Manual, Cold Spring Harbor Laboratory, 1982).

Screening procedures which rely on nucleic acid hybridization make it possible to isolate any gene sequence from any organism, provided the appropriate probe is available. Oligonucleotide probes, which correspond to a part of the sequence encoding the protein in question, can be synthesized chemically. This requires that short, oligopeptide stretches of amino acid sequence must be known. The DNA sequence encoding the protein can be deduced from the genetic code, however, the degeneracy of the code must be taken into account. It is possible to perform a mixed addition reaction when the sequence is degenerate. This includes a heterogeneous mixture of denatured double-stranded DNA. For such screening, hybridization is preferably performed on either single-stranded DNA or denatured double-stranded DNA.

Since the DNA sequences of the invention encode essentially all or part of an zinc finger-nucleotide binding protein as part of the zinc finger tag that forms part of a fusion protein according to the present invention, it is now a routine matter to prepare, subclone, and express truncated polypeptide fragments of DNA from this or corresponding DNA sequences. Alternatively, by utilizing the DNA fragments disclosed herein which encode fragments of fusion proteins according to the present invention it is possible, in conjunction with known techniques, to determine the DNA sequences encoding the entire fusion protein. Such techniques are described in U.S. Pat. Nos. 4,394,443 and 4,446,235 which are incorporated herein by reference.

A cDNA expression library, such as λgt11, can be screened indirectly for nucleic acid sequences encoding fusion proteins according to the present invention, using antibodies specific for the fusion protein. Such antibodies can be either polyclonally or monoclonally derived and used to detect expression product indicative of cDNA encoding the fusion protein. Alternatively, binding of the derived polypeptides to DNA targets can be assayed by incorporated radiolabeled DNA into the target site and testing for retardation of electrophoretic mobility as compared with unbound target site. Such assays are well known in the art and are described, for example, in D. J. Segal et al., “Toward Controlling Gene Expression at Will: Selection and Design of Zinc Finger Domains Recognizing Each of the 5′-GNN-3′ DNA Target Sequences,” Proc. Natl. Acad. Sci. USA 96: 2758-2765 (1999). Other suitable methods for determining the binding of the polypeptides to DNA targets are known in the art.

Another aspect of the present invention is vectors incorporating nucleic acid sequences or nucleic acid constructs according to the present invention. Typically, the vector includes at least one additional sequence that enable it to be used to transform or transfect a prokaryotic cell or a eukaryotic cell. The prokaryotic cell can be a bacterial cell, such as Escherichia coli or Salmonella typhimurium. The eukaryotic cell can be a mammalian cell, such as a murine cell, a Chinese hamster cell, or a human cell, or, alternatively, a yeast cell, a plant cell, or an insect cell. The vector can also include a reporter gene to monitor the transformation or transfection of an appropriate prokaryotic or eukaryotic cell, or to monitor the expression of the nucleic acid construct. Reporter genes are well known in the art, and are described, for example, in U.S. Pat. No. 6,858,773 to Zhang, incorporated herein by this reference. A variety of reporter genes may be used in the practice of the present invention. Preferred are those that produce a protein product which is easily measured in a routine assay. Suitable reporter genes include, but are not limited to chloramphenicol acetyl transferase (CAT), light generating proteins (e.g., luciferase), and β-galactosidase. Convenient assays include, but are not limited to colorimetric, fluorometric and enzymatic assays. In one aspect, reporter genes may be employed that are expressed within the cell and whose extracellular products are directly measured in the intracellular medium, or in an extract of the intracellular medium of a cultured cell line. This provides advantages over using a reporter gene whose product is secreted, since the rate and efficiency of the secretion introduces additional variables that may complicate interpretation of the assay. In one preferred embodiment, the reporter gene is a light generating protein. When using the light generating reporter proteins described herein, expression can be evaluated accurately and non-invasively as described above (see, for example, Contag, P. R., et al., (1998) Nature Med. 4:245-7; Contag, C. H., et al., (1997) Photochem Photobiol. 66:523-31; Contag, C. H., et al., (1995) Mol. Microbiol. 18:593-603).

In one aspect of the invention, the light generating protein is luciferase. Luciferase coding sequences useful in the practice of the present invention include sequences obtained from lux genes (procaryotic genes encoding a luciferase activity) and luc genes (eucaryotic genes encoding a luciferase activity). A variety of luciferase encoding genes have been identified including, but not limited to, the following: B. A. Sherf and K. V. Wood, U.S. Pat. No. 5,670,356, issued 23 Sep. 1997; Kazami, J., et al., U.S. Pat. No. 5,604,123, issued 18 Feb. 1997; S. Zenno, et al, U.S. Pat. No. 5,618,722; K. V. Wood, U.S. Pat. No. 5,650,289, issued 22 Jul. 1997; K. V. Wood, U.S. Pat. No. 5,641,641, issued 24 Jun. 1997; N. Kajiyama and E. Nakano, U.S. Pat. No. 5,229,285, issued 20 Jul. 1993; M. J. Cormier and W. W. Lorenz, U.S. Pat. No. 5,292,658, issued 8 Mar. 1994; M. J. Cormier and W. W. Lorenz, U.S. Pat. No. 5,418,155, issued 23 May 1995; de Wet, J. R., et al, Molec. Cell Biol. 7:725-737, 1987; Tatsumi, H. N., et al, Biochim. Biophys. Acta 1131:161-165, 1992; and Wood, K. V., et al, Science 244:700-702, 1989; all herein incorporated by reference. Another group of bioluminescent proteins includes light-generating proteins of the acquorin family (Prasher, D. C., et al., Biochem. 26:1326-1332 (1987)). Luciferases, as well as aequorin-like molecules, require a source of energy, such as ATP, NAD(P)H, and the like, and a substrate, such as luciferin or coelentrizine and oxygen. Wild-type firefly luciferases typically have emission maxima at about 550 nm. Numerous variants with distinct emission maxima have also been studied. For example, Kajiyama and Nakano (Protein Eng. 4(6):691-693, 1991; U.S. Pat. No. 5,330,906, issued 19 Jul. 1994, herein incorporated by reference) teach five variant firefly luciferases generated by single amino acid changes to the Luciola cruciata luciferase coding sequence. The variants have emission peaks of 558 nm, 595 nm, 607 nm, 609 nm and 612 nm. A yellow-green luciferase with an emission peak of about 540 nm is commercially available from Promega, Madison, Wis. under the name pGL3. A red luciferase with an emission peak of about 610 nm is described, for example, in Contag et al. (1998) Nat. Med. 4:245-247 and Kajiyama et al. (1991) Port Eng. 4:691-693. The coding sequence of a luciferase derived from Renilla muelleri has also been described (mRNA, GENBANK Accession No. AY015988, protein Accession AAG54094).

In another aspect of the present invention, the light-generating protein is a fluorescent protein, for example, blue, cyan, green, yellow, and red fluorescent proteins. Several light-generating protein coding sequences are commercially available, including, but not limited to, the following. Clontech (Palo Alto, Calif.) provides coding sequences for luciferase and a variety of fluorescent proteins, including, blue, cyan, green, yellow, and red fluorescent proteins. Enhanced green fluorescent protein (EGFP) variants are well expressed in mammalian systems and tend to exhibit brighter fluorescence than wild-type GFP. Enhanced fluorescent proteins include enhanced green fluorescent protein (EGFP), enhanced cyan fluorescent protein (ECFP), and enhanced yellow fluorescent protein (EYFP). Further, Clontech provides destabilized enhanced fluorescent proteins (dEFP) variants that feature rapid turn over rates. The shorter half life of the dEFP variants makes them useful in kinetic studies and as quantitative reporters. DsRed coding sequences are available from Clontech DsRed is a red fluorescent protein useful in expression studies. Further, Fradkov, A. F., et. al., described a novel fluorescent protein from Discosoma coral and its mutants which possesses a unique far-red fluorescence (FEBS Lett. 479 (3), 127-130 (2000)) (mRNA sequence, GENBANK Accession No. AF272711, protein sequence, GENBANK Accession No. AAG16224). Promega (Madison, Wis.) also provides coding sequences for firefly luciferase (for example, as contained in the pGL3 vectors). Further, coding sequences for a number of fluorescent proteins are available from GENBANK, for example, accession numbers AY015995, AF322221, AF080431, AF292560, AF292559, AF292558, AF292557, AF139645, U47298, U47297, AY015988, AY015994, and AF292556. Modified lux coding sequences have also been described, e.g., WO 01/18195, published 15 Mar. 2001, Xenogen Corporation. In addition, further light generating systems may be employed, for example, when evaluating expression in cells. Such systems include, but are not limited to, Luminescent β-galactosidase Genetic Reporter System (Clontech).

The vector can also include a positive selection marker. Positive selection markers are well known in the art. Positive selection markers include any gene which a product that can be readily assayed. Examples include, but are not limited to, an HPRT gene (Littlefield, J. W., Science 145:709-710 (1964), herein incorporated by reference), a xanthine-guanine phosphoribosyltransferase (GPT) gene, or an adenosine phosphoribosyltransferase (APRT) gene (J. Sambrook & D. W. Russell, “Molecular Cloning: A Laboratory Manual” (3^{rd} ed., Cold Spring Harbor Laboratory Press, Cold Spring Harbor, N.Y., 2001), a thymidine kinase gene (i.e. “TK”) and especially the TK gene of the herpes simplex virus (Giphart-Gassler, M. et al., Mutat. Res. 214:223-232 (1989) herein incorporated by reference), a nptII gene (Thomas, K. R. et al., Cell 51:503-512 (1987); Mansour, S. L. et al., Nature 336:348-352 (1988), both references herein incorporated by reference), or other genes which confer resistance to amino acid or nucleoside analogues, or antibiotics, etc., for example, gene sequences which encode enzymes such as dihydrofolate reductase (DHFR) enzyme, adenosine deaminase (ADA), asparagine synthetase (AS), hygromycin B phosphotransferase, or a CAD enzyme (carbamyl phosphate synthetase, aspartate transcarbamylase, and dihydroorotase). Addition of the appropriate substrate of the positive selection marker can be used to determine if the product of the positive selection marker is expressed, for example cells which do not express the positive selection marker nptII, are killed when exposed to the substrate G418 (Gibco BRL Life Technology, Gaithersburg, Md.). Appropriate positive selection markers can be chosen depending on the prokaryotic cell or eukaryotic cell used.

The vector typically contains insertion sites for inserting polynucleotide sequences of interest, e.g., the nucleic acid constructs of the present invention. In one suitable alternative, these insertion sites are preferably included such that there are two sites, one site on either side of the sequences encoding the positive selection marker, luciferase and the promoter. Insertion sites are, for example, restriction endonuclease recognition sites, and can, for example, represent unique restriction sites. In this way, the vector can be digested with the appropriate enzymes and the sequences of interest ligated into the vector.

Optionally, the vector construct can contain a polynucleotide encoding a negative selection marker. Suitable negative selection markers include, but are not limited to, HSV-tk (see, e.g., Majzoub et al. (1996) New Engl. J. Med. 334:904-907 and U.S. Pat. No. 5,464,764), as well as genes encoding various toxins including the diphtheria toxin, the tetanus toxin, the cholera toxin and the pertussis toxin. A further negative selection marker gene is the hypoxanthine-guanine phosphoribosyl transferase (HPRT) gene for negative selection in 6-thioguanine.

The vectors described herein can be constructed utilizing methodologies known in the art of molecular biology (see, for example, F. M. Ausubel et al., “Short Protocols in Molecular Biology (2^{nd} ed., John Wiley & Sons, New York, 1992) and J. Sambrook & D. W. Russell., “Molecular Cloning: A Laboratory Manual” (3^{rd} ed., Cold Spring Harbor Laboratory Press, Cold Spring Harbor, N.Y., 2001)) in view of the teachings of the specification.

A preferred vector used for incorporating nucleic acid constructs encoding fusion proteins according to the present invention is a recombinant DNA (rDNA) molecule containing a nucleotide sequence that codes for and is capable of expressing a fusion polypeptide containing, in the direction of amino- to carboxy-terminus, (1) a prokaryotic secretion signal domain, (2) a heterologous polypeptide, and (3) a filamentous phage membrane anchor domain. The vector includes DNA expression control sequences for expressing the fusion polypeptide, preferably prokaryotic control sequences. The heterologous polypeptide includes at least the fusion protein according to the present invention and can optionally include additional sequences at its N- or C-terminus.

The filamentous phage membrane anchor is preferably a domain of the cpIII or cpVIII coat protein capable of associating with the matrix of a filamentous phage particle, thereby incorporating the fusion polypeptide onto the phage surface.

The secretion signal is a leader peptide domain of a protein that targets the protein to the periplasmic membrane of gram negative bacteria. A preferred secretion signal is a pelB secretion signal. The predicted amino acid residue sequences of the secretion signal domain from two pelB gene product variants from Erwinia carotovora are described in Lei, et al. (Nature, 331:543-546, 1988).

The leader sequence of the pelB protein has previously been used as a secretion signal for fusion proteins (Better, et al., Science, 240:1041-1043, 1988; Sastry, et al., Proc. Natl. Acad. Sci. USA, 86:5728-5732, 1989; and Mullinax, et al., Proc. Natl. Acad. Sci. USA, 87:8095-8099, 1990). Amino acid residue sequences for other secretion signal polypeptide domains from E. coli useful in this invention can be found in Oliver, In Neidhard, F. C. (ed.), Escherichia coli and Salmonella typhimurium, American Society for Microbiology, Washington, D.C., 1:56-69 (1987).

Preferred membrane anchors for the vector are obtainable from filamentous phage M13, fl, fd, and equivalent filamentous phage. Preferred membrane anchor domains are found in the coat proteins encoded by gene III and gene VII. The membrane anchor domain of a filamentous phage coat protein is a portion of the carboxy terminal region of the coat protein and includes a region of hydrophobic amino acid residues for spanning a lipid bilayer membrane, and a region of charged amino acid residues normally found at the cytoplasmic face of the membrane and extending away from the membrane. In the phage fl, gene VIII coat protein's membrane spanning region comprises residue Trp-26 through Lys-40, and the cytoplasmic region comprises the carboxy-terminal 11 residues from 41 to 52 (Ohkawa, et al., J. Biol. Chem., 256:9951-9958, 1981). Thus, the amino acid residue sequence of a preferred membrane anchor domain is derived from the M13 filamentous phage gene VIII coat protein (also designated cpVIII or CP 8). Gene VIII coat protein is present on a mature filamentous phage over the majority of the phage particle with typically about 2500 to 3000 copies of the coat protein.

In addition, the amino acid residue sequence of another preferred membrane anchor domain is derived from the M13 filamentous phage gene III coat protein (also designated cpIII). Gene III coat protein is present on a mature filamentous phage at one end of the phage particle with typically about 4 to 6 copies of the coat protein. For detailed descriptions of the structure of filamentous phage particles, their coat proteins and particle assembly, see the reviews by Rached, et al. (Microbiol Rev., 50:401-427 1986; and Model, et al., in “The Bacteriophages: Vol. 2”, R. Calendar, ed. Plenum Publishing Co., pp. 375-456, 1988).

DNA expression control sequences comprise a set of DNA expression signals for expressing a structural gene product and include both 5′ and 3′ elements, as is well known, operably linked to the cistron such that the cistron is able to express a structural gene product. The 5′ control sequences define a promoter for initiating transcription and a ribosome binding site operably linked at the 5′ terminus of the upstream translatable DNA sequence.

To achieve high levels of gene expression in E. coli, it is necessary to use not only strong promoters to generate large quantities of mRNA, but also ribosome binding sites to ensure that the mRNA is efficiently translated. In E. coli, the ribosome binding site includes an initiation codon (AUG) and a sequence 3-9 nucleotides long located 3-11 nucleotides upstream from the initiation codon (Shine, et al., Nature, 254:34, 1975). The sequence, AGGAGGU (SEQ ID NO: 706), which is called the Shine-Dalgarno (SD) sequence, is complementary to the 3′ end of E. coli 16S rRNA. Binding of the ribosome to mRNA and the sequence at the 3′ end of the mRNA-can be affected by several factors: (1) The degree of complementarity between the SD sequence and 3′ end of the 16S rRNA. (2) The spacing and possibly the RNA sequence lying between the SD sequence and the AUG (Roberts, et al., Proc. Natl. Acad. Sci. USA, 76:760, 1979a; Roberts, et al., Proc. Natl. Acad. Sci. USA, 76:5596, 1979b; Guarente, et al., Science, 209:1428, 1980; and Guarente, et al., Cell, 20:543, 1980). Optimization is achieved by measuring the level of expression of genes in plasmids in which this spacing is systematically altered. Comparison of different mRNAs shows that there are statistically preferred sequences from positions −20 to +13 (where the A of the AUG is position 0) (Gold, et al., Annu. Rev. Microbiol., 35:365, 1981). Leader sequences have been shown to influence translation dramatically (Roberts, et al., 1979 a, b supra). (3) The nucleotide sequence following the AUG, which affects ribosome binding (Taniguchi, et al., J. Mol. Biol., 118:533, 1978).

The 3′ control sequences define at least one termination (stop) codon in frame with and operably linked to the heterologous fusion polypeptide.

In preferred embodiments, the vector utilized includes a prokaryotic origin of replication or replicon, i.e., a DNA sequence having the ability to direct autonomous replication and maintenance of the recombinant DNA molecule extra-chromosomally in a prokaryotic host cell, such as a bacterial host cell, transformed therewith. Such origins of replication are well known in the art. Preferred origins of replication are those that are efficient in the host organism. A preferred host cell is E. coli. For use of a vector in E. coli, a preferred origin of replication is ColE1 found in pBR322 and a variety of other common plasmids. Also preferred is the p15A origin of replication found on pACYC and its derivatives. The ColE1 and p15A replicon have been extensively utilized in molecular biology, are available on a variety of plasmids and are described at least by Sambrook, et al., Molecular Cloning: a Laboratory Manual, 2nd edition, Cold Spring Harbor Laboratory Press, 1989).

The ColE1 and p15A replicons are particularly preferred for use in the present invention because they each have the ability to direct the replication of a plasmid in E. coli while the other replicon is present in a second plasmid in the same E. coli cell. In other words, ColE1 and p15A are non-interfering replicons that allow the maintenance of two plasmids in the same host.

In addition, those embodiments that include a prokaryotic replicon also include a gene whose expression confers a selective advantage, such as drug resistance, to a bacterial host transformed therewith. Typical bacterial drug resistance genes are those that confer resistance to ampicillin, tetracycline, neomycin/kanamycin or chloramphenicol. Vectors typically also contain convenient restriction sites for insertion of translatable DNA sequences. Exemplary vectors are the plasmids pUC8, pUC9, pBR322, and pBR329 available from BioRad Laboratories, (Richmond, Calif.) and pPL and pKK223 available from Pharmacia (Piscataway, N.J.) and pBS (Stratagene, La Jolla, Calif.).

The vector comprises a first cassette that includes upstream and downstream translatable DNA sequences operably linked via a sequence of nucleotides adapted for directional ligation to an insert DNA. The upstream translatable sequence encodes the secretion signal as defined herein. The downstream translatable sequence encodes the filamentous phage membrane anchor as defined herein. The cassette preferably includes DNA expression control sequences for expressing the heterologous polypeptide, including a fusion protein according to the present invention, that is produced when an insert translatable DNA sequence (insert DNA) is directionally inserted into the cassette via the sequence of nucleotides adapted for directional ligation. The filamentous phage membrane anchor is preferably a domain of the cpIII or cpVIII coat protein capable of binding the matrix of a filamentous phage particle, thereby incorporating the fusion polypeptide onto the phage surface

The zinc finger derived polypeptide expression vector also contains a second cassette for expressing a second receptor polypeptide. The second cassette includes a second translatable DNA sequence that encodes a secretion signal, as defined herein, operably linked at its 3′ terminus via a sequence of nucleotides adapted for directional ligation to a downstream DNA sequence of the vector that typically defines at least one stop codon in the reading frame of the cassette. The second translatable DNA sequence is operably linked at its 5′ terminus to DNA expression control sequences forming the 5′ elements. The second cassette is capable, upon insertion of a translatable DNA sequence (insert DNA), of expressing the second fusion polypeptide comprising a receptor of the secretion signal with a polypeptide coded by the insert DNA. For purposes of this invention, the second cassette sequences have been deleted.

As used herein, the term “vector” refers to a nucleic acid molecule capable of transporting between different genetic environments another nucleic acid to which it has been operably liked. Preferred vectors are those capable of autonomous replication and expression of structural gene products present in the DNA segments to which they are operably linked. Vectors, therefore, preferably contain the replicons and selectable markers described earlier.

As used herein with regard to DNA sequences or segments, the phrase “operably linked” means the sequences or segments have been covalently joined, preferably by conventional phosphodiester bonds, into one strand of DNA, whether in single or double stranded form. The choice of vector to which transcription unit or a cassette of this invention is operably linked depends directly as is well known in the art, on the functional properties desired, e.g., vector replication and protein expression, and the host cell to be transformed, these being limitations inherent in the art of constructing recombinant DNA molecules. The phrase “operably linked” or equivalent phraseology, when applied to DNA sequences or segments, does not necessarily imply that the DNA sequences or segments are adjacent to one another in the single strand of DNA or that the DNA sequences or segments are translated into a single protein molecule.

A sequence of nucleotides adapted for directional ligation, i.e., a polylinker, is a region of the DNA expression vector that (1) operatively links for replication and transport the upstream and downstream translatable DNA sequences and (2) provides a site or means for directional ligation of a DNA sequence into the vector. Typically, a directional polylinker is a sequence of nucleotides that defines two or more restriction endonuclease recognition sequences, or restriction sites. Upon restriction cleavage, the two sites yield cohesive termini to which a translatable DNA sequence can be ligated to the DNA expression vector. Preferably, the two restriction sites provide, upon restriction cleavage, cohesive termini that are non-complementary and thereby permit directional insertion of a translatable DNA sequence into the cassette. In one embodiment, the directional ligation means is provided by nucleotides present in the upstream translatable DNA sequence, downstream translatable DNA sequence, or both. In another embodiment, the sequence of nucleotides adapted for directional ligation comprises a sequence of nucleotides that defines multiple directional cloning means. Where the sequence of nucleotides adapted for directional ligation defines numerous restriction sites, it is referred to as a multiple cloning site.

In a preferred embodiment, a DNA expression vector is designed for convenient manipulation in the form of a filamentous phage particle encapsulating DNA encoding a fusion protein according to the present invention. In this embodiment, a DNA expression vector further contains a nucleotide sequence that defines a filamentous phage origin of replication such that the vector, upon presentation of the appropriate genetic complementation, can replicate as a filamentous phage in single stranded replicative form and be packaged into filamentous phage particles. This feature provides the ability of the DNA expression vector to be packaged into phage particles for subsequent segregation of the particle, and vector contained therein, away from other particles that comprise a population of phage particles using screening technique well known in the art.

A filamentous phage origin of replication is a region of the phage genome, as is well known, that defines sites for initiation of replication, termination of replication and packaging of the replicative form produced by replication (see, for example, Rasched, et al., Microbiol Rev., 50:401427, 1986; and Horiuchi, J. Mol. Biol., 188:215-223, 1986).

A preferred filamentous phage origin of replication for use in the present invention is an M13, fl or fd phage origin of replication (Short, et al. (Nucl. Acids Res., 16:7583-7600, 1988). Preferred DNA expression vectors are the expression vectors modified pCOMB3 and specifically pCOMB3.5.

The production of a DNA sequence encoding a fusion protein according to the present invention can be accomplished by oligonucleotide(s) which are primers for amplification of the genomic polynucleotide encoding an zinc finger-nucleotide binding polypeptide. These unique oligonucleotide primers can be produced based upon identification of the flanking regions contiguous with the polynucleotide encoding the fusion protein according to the present invention. These oligonucleotide primers comprise sequences which are capable of hybridizing with the flanking nucleotide sequence encoding a fusion protein according to the present invention and sequences complementary thereto and can be used to introduce point mutations into the amplification products.

The primers of the invention include oligonucleotides of sufficient length and appropriate sequence so as to provide specific initiation of polymerization on a significant number of nucleic acids in the polynucleotide encoding the fusion protein according to the present invention. Specifically, the term “primer” as used herein refers to a sequence comprising two or more deoxyribonucleotides or ribonucleotides, preferably more than three, which sequence is capable of initiating synthesis of a primer extension product, which is substantially complementary to a zinc finger-nucleotide binding protein strand, but can also introduce mutations into the amplification products at selected residue sites. Experimental conditions conducive to synthesis include the presence of nucleoside triphosphates and an agent for polymerization and extension, such as DNA polymerase, and a suitable buffer, temperature and pH. The primer is preferably single stranded for maximum efficiency in amplification, but may be double stranded. If double stranded, the primer is first treated to separate the two strands before being used to prepare extension products. Preferably, the primer is an oligodeoxyribonucleotide. The primer must be sufficiently long to prime the synthesis of extension products in the presence of the inducing agent for polymerization and extension of the nucleotides. The exact length of primer will depend on many factors, including temperature, buffer, and nucleotide composition. The oligonucleotide primer typically contains 15-22 or more nucleotides, although it may contain fewer nucleotides. Alternatively, as is well known in the art, the mixture of nucleoside triphosphates can be biased to influence the formation of mutations to obtain a library of cDNAs encoding putative fusion proteins according to the present invention that can be screened in a functional assay for binding to a zinc finger-nucleotide binding motif, such as one in a promoter in which the binding inhibits transcriptional activation.

Primers of the invention are designed to be “substantially” complementary to a segment of each strand of polynucleotide encoding the fusion protein to be amplified. This means that the primers must be sufficiently complementary to hybridize with their respective strands under conditions which allow the agent for polymerization and nucleotide extension to act. In other words, the primers should have sufficient complementarity with the flanking sequences to hybridize therewith and permit amplification of the polynucleotide encoding the fusion protein. Preferably, the primers have exact complementarity with the flanking sequence strand.

Oligonucleotide primers of the invention are employed in the amplification process which is an enzymatic chain reaction that produces exponential quantities of polynucleotide encoding the fusion protein relative to the number of reaction steps involved. Typically, one primer is complementary to the negative (−) strand of the polynucleotide encoding the fusion protein and the other is complementary to the positive (+) strand. Annealing the primers to denatured nucleic acid followed by extension with an enzyme, such as the large fragment of DNA Polymerase I (Klenow) and nucleotides, results in newly synthesized (+) and (−) strands containing the zinc finger-nucleotide binding protein sequence. Because these newly synthesized sequences are also templates, repeated cycles of denaturing, primer annealing, and extension results in exponential production of the sequence (i.e., the fusion protein sequence) defined by the primer. The product of the chain reaction is a discrete nucleic acid duplex with termini corresponding to the ends of the specific primers employed. Those of skill in the art will know of other amplification methodologies which can also be utilized to increase the copy number of target nucleic acid. These may include for example, ligation activated transcription (LAT), ligase chain reaction (LCR), and strand displacement activation (SDA), although PCR is the preferred method.

The oligonucleotide primers of the invention may be prepared using any suitable method, such as conventional phosphotriester and phosphodiester methods or automated embodiments thereof. In one such automated embodiment, diethylphosphoramidites are used as starting materials and may be synthesized as described by Beaucage, et al (Tetrahedron Letters, 22:1859-1862, 1981). One method for synthesizing oligonucleotides on a modified solid support is described in U.S. Pat. No. 4,458,066. One method of amplification which can be used according to this invention is the polymerase chain reaction (PCR) described in U.S. Pat. Nos. 4,683,202 and 4,683,195.

Methods for utilizing filamentous phage libraries to obtain mutations of peptide sequences are disclosed in U.S. Pat. No. 5,223,409 to Ladner et al., which is incorporated by reference herein in its entirety.

In one embodiment of the invention, randomized nucleotide substitutions can be performed on the DNA encoding one or more fingers of a known zinc finger tag to obtain a derived polypeptide that modifies gene expression upon binding to a site on the DNA containing the gene, such as a transcriptional control element. In addition to modifications in the amino acids making up the zinc finger tag, the mutated zinc finger tag can contain more or fewer than the full amount of fingers contained in the wild type protein from which it is derived.

While any method of site directed mutagenesis can be used to perform the mutagenesis, preferably the method used to randomize the segment of the zinc finger protein to be modified utilizes a pool of degenerate oligonucleotide primers containing a plurality of triplet codons having the formula NNS or NNK (and its complement NNM), wherein S is either G or C, K is either G or T, M is either C or A (the complement of NNK) and N can be A, C, G or T. In addition to the degenerate triplet codons, the degenerate oligonucleotide primers also contain at least one segment designed to hybridize to the DNA encoding the wild type zinc finger protein on at least one end, and are utilized in successive rounds of PCR amplification known in the art as overlap extension PCR so as to create a specified region of degeneracy bracketed by the non-degenerate regions of the primers in the primer pool.

The methods of overlap PCR as used to randomize specific regions of a cDNA are well known in the art. The degenerate products of the overlap PCR reactions are pooled and gel purified, preferably by size exclusion chromatography or gel electrophoresis, prior to ligation into a surface display phage expression vector to form a library for subsequent screening against a known or putative zinc finger-nucleotide binding motif

The degenerate primers are utilized in successive rounds of PCR amplification known in the art as overlap extension PCR so as to create a library of cDNA sequences encoding putative zinc finger-derived DNA binding polypeptides. Usually the derived polypeptides contain a region of degeneracy corresponding to the region of the finger that binds to DNA (usually in the tip of the finger and in the α-helix region) bracketed by non-degenerate regions corresponding to the conserved regions of the finger necessary to maintain the three dimensional structure of the finger.

Any nucleic acid specimen, in purified or nonpurified form, can be utilized as the starting nucleic acid for the above procedures, provided it contains, or is suspected of containing, the specific nucleic acid sequence of a fusion protein of the invention. Thus, the process may employ, for example, DNA or RNA, including messenger RNA, wherein DNA or RNA may be single stranded or double stranded. In the event that RNA is to be used as a template, enzymes, and/or conditions optimal for reverse transcribing the template to DNA would be utilized. In addition, a DNA-RNA hybrid which contains one strand of each may be utilized. A mixture of nucleic acids may also be employed, or the nucleic acids produced in a previous amplification reaction herein, using the same or different primers may be so utilized. The specific nucleic acid sequence to be amplified, i.e., a nucleic acid sequence encoding a fusion protein of the present invention, can be a fraction of a larger molecule or can be present initially as a discrete molecule, so that the specific sequence constitutes the entire nucleic acid. It is not necessary that the sequence to be amplified be present initially in a pure form; it may be a minor fraction of a complex mixture, such as contained in whole human DNA or the DNA of any organism. For example, the source of DNA includes prokaryotes, eukaryotes, viruses and plants.

Where the target nucleic acid sequence of the sample contains two strands, it is necessary to separate the strands of the nucleic acid before it can be used as the template. Strand separation can be effected either as a separate step or simultaneously with the synthesis of the primer extension products. This strand separation can be accomplished using various suitable denaturing conditions, including physical, chemical, or enzymatic means, the word “denaturing” includes all such means. One physical method of separating nucleic acid strands involves heating the nucleic acid until it is denatured. Typical heat denaturation may involve temperatures ranging from about 80° C. to 105° C. for times ranging from about 1 to 10 minutes. Strand separation may also be induced by an enzyme from the class of enzymes known as helicases or by the enzyme RecA, which has helicase activity, and in the presence of riboATP, is known to denature DNA. The reaction conditions suitable for strand separation of nucleic acids with helicases are described by Kuhn Hoffmann-Berling (CSH-Quantitative Biology, 43:63, 1978) and techniques for using RecA are reviewed in C. Radding (Ann. Rev. Genetics, 16:405-437, 1982).

If the nucleic acid containing the sequence to be amplified is single stranded, its complement is synthesized by adding one or two oligonucleotide primers. If a single primer is utilized, a primer extension product is synthesized in the presence of primer, an agent for polymerization, and the four nucleoside triphosphates described below. The product will be partially complementary to the single-stranded nucleic acid and will hybridize with a single-stranded nucleic acid to form a duplex of unequal length strands that may then be separated into single strands to produce two single separated complementary strands. Alternatively, two primers may be added to the single-stranded nucleic acid and the reaction carried out as described.

When complementary strands of nucleic acid or acids are separated, regardless of whether the nucleic acid was originally double or single stranded, the separated strands are ready to be used as a template for the synthesis of additional nucleic acid strands. This synthesis is performed under conditions allowing hybridization of primers to templates to occur. Generally synthesis occurs in a buffered aqueous solution, preferably at a pH of 7-9, most preferably about 8. Preferably, a molar excess (for genomic nucleic acid, usually about 10⁸:1 primer:template) of the two oligonucleotide primers is added to the buffer containing the separated template strands. It is understood, however, that the amount of complementary strand may not be known if the process of the invention is used for diagnostic applications, so that the amount of primer relative to the amount of complementary strand cannot be determined with certainty. As a practical matter, however, the amount of primer added will generally be in molar excess over the amount of complementary strand (template) when the sequence to be amplified is contained in a mixture of complicated long-chain nucleic acid strands. A large molar excess is preferred to improve the efficiency of the process.

The deoxyribonucleotide triphosphates dATP, dCTP, dGTP, and dTTP are added to the synthesis mixture, either separately or together with the primers, in adequate amounts and the resulting solution is heated to about 90° C.-100° C. from about 1 to 10 minutes, preferably from 1 to 4 minutes. After this heating period, the solution is allowed to cool to a temperature that is preferable for the primer hybridization. To the cooled mixture is added an appropriate agent for effecting the primer extension reaction (called herein ‘agent for polymerization’), and the reaction is allowed to occur under conditions known in the art. The agent for polymerization may also be added together with the other reagents if it is heat stable. This synthesis (or amplification) reaction may occur at room temperature up to a temperature above which the agent for polymerization no longer functions. Most conveniently the reaction occurs at room temperature.

The agent for polymerization may be any compound or system which will function to accomplish the synthesis of primer extension products, including enzymes. Suitable enzymes for this purpose include, for example, E. coli DNA polymerase I, Klenow fragment of E. coli DNA polymerase I, T4 DNA polymerase, other available DNA polymerases, polymerase muteins, reverse transcriptase, and other enzymes, including heat-stable enzymes (i.e., those enzymes which perform primer extension after being subjected to temperatures sufficiently elevated to cause denaturation). Suitable enzymes will facilitate combination of the nucleotides in the proper manner to form the primer extension products which are complementary to each zinc finger-nucleotide binding protein nucleic acid strand. Generally, the synthesis will be initiated at the 3′ end of each primer and proceed in the 5′ direction along the template strand, until synthesis terminates, producing molecules of different lengths. There may be agents for polymerization, however, which initiate synthesis at the 5′ end and proceed in the other direction, using the same process as described above.

The newly synthesized fusion protein nucleic acid strand and its complementary nucleic acid strand will form a double-stranded molecule under hybridizing conditions described above and this hybrid is used in subsequent steps of the process. In the next step, the newly synthesized double-stranded molecule is subjected to denaturing conditions using any of the procedures described above to provide single-stranded molecules.

The above process is repeated on the single-stranded molecules. Additional agent for polymerization, nucleotides, and primers may be added, if necessary, for the reaction to proceed under the conditions prescribed above. Again, the synthesis will be initiated at one end of each of the oligonucleotide primers and will proceed along the single strands of the template to produce additional nucleic acid. After this step, half of the extension product will consist of the specific nucleic acid sequence bounded by the two primers.

The steps of denaturing and extension product synthesis can be repeated as often as needed to amplify the zinc finger-nucleotide binding protein nucleic acid sequence to the extent necessary for detection. The amount of the specific nucleic acid sequence produced will accumulate in an exponential fashion.

Sequences amplified by the methods of the invention can be further evaluated, detected, cloned, sequenced, and the like, either in solution or after binding to a solid support, by any method usually applied to the detection of a specific DNA sequence such as PCR, oligomer restriction (Saiki, et al., Bio/Technology, 3:1008-1012, 1985), allele-specific oligonucleotide (ASO) probe analysis (Conner, et al., Proc. Natl. Acad. Sci. USA, 80:278, 1983), oligonucleotide ligation assays (OLAs) (Landegren, et al., Science, 241:1077, 1988), and the like. Molecular techniques for DNA analysis have been reviewed (Landegren, et al., Science, 242:229-237, 1988). Preferably, novel fusion proteins of the invention can be isolated utilizing the above techniques wherein the primers allow modification, such as substitution, of nucleotides such that unique zinc fingers are produced (See Examples for further detail).

In the present invention, the fusion protein encoding nucleotide sequences may be inserted into a recombinant expression vector. The term “recombinant expression vector” refers to a plasmid, virus or other vehicle known in the art that has been manipulated by insertion or incorporation of zinc finger derived-nucleotide binding protein genetic sequences. Such expression vectors contain a promoter sequence which facilitates the efficient transcription of the inserted genetic sequence in the host. The expression vector typically contains an origin of replication, a promoter, as well as specific genes which allow phenotypic selection of the transformed cells. Vectors suitable for use in the present invention include, but are not limited to the T7-based expression vector for expression in bacteria (Rosenberg, et al., Gene 56:125, 1987), the pMSXND expression vector for expression in mammalian cells (Lee and Nathans, J. Biol. Chem. 263:3521, 1988) and baculovirus-derived vectors for expression in insect cells. The DNA segment can be present in the vector operably linked to regulatory elements, for example, a promoter (e.g., T7, metallothionein I, or polyhedrin promoters).

Sequences encoding novel fusion proteins of the invention can be expressed in vitro by DNA transfer into a suitable host cell. “Host cells” are cells in which a vector can be propagated and its DNA expressed. The term also includes any progeny of the subject host cell. It is understood that all progeny may not be identical to the parental cell since there may be mutations that occur during replication. However, such progeny are included when the term “host cell” is used. Methods of stable transfer, in other words when the foreign DNA is continuously maintained in the host, are known in the art.

A preferred method of obtaining polynucleotides containing suitable regulatory sequences (e.g., promoters) is PCR. General procedures for PCR as taught in MacPherson et al., PCR: A PRACTICAL APPROACH, (IRL Press at Oxford University Press, (1991)). PCR conditions for each application reaction may be empirically determined. A number of parameters influence the success of a reaction. Among these parameters are annealing temperature and time, extension time, Mg²⁺ and ATP concentration, pH, and the relative concentration of primers, templates and deoxyribonucleotides. After amplification, the resulting fragments can be detected by agarose gel electrophoresis followed by visualization with ethidium bromide staining and ultraviolet illumination.

In one embodiment, PCR can be used to amplify fragments from genomic libraries. Many genomic libraries are commercially available. Alternatively, libraries can be produced by any method known in the art. The purified DNA is then introduced into a suitable expression system, for example a λ phage. Another method for obtaining polynucleotides, for example, short, random nucleotide sequences, is by enzymatic digestion.

Polynucleotides are inserted into vector backbones using methods known in the art. For example, insert and vector DNA can be contacted, under suitable conditions, with a restriction enzyme to create complementary or blunt ends on each molecule that can pair with each other and be joined with a ligase. Alternatively, synthetic nucleic acid linkers can be ligated to the termini of a polynucleotide. These synthetic linkers can contain nucleic acid sequences that correspond to a particular restriction site in the vector DNA. Other means are known and, in view of the teachings herein, can be used.

The vector backbone may comprise components functional in more than one selected organism in order to provide a shuffle vector, for example, a bacterial origin of replication and a eukaryotic promoter. Alternately, the vector backbone may comprise an integrating vector, i.e., a vector that is used for random or site-directed integration into a target genome.

The final constructs can be used immediately (e.g., for introduction into ES cells), or stored frozen (e.g., at −20° C.) until use. In some embodiments, the constructs are linearized prior to use, for example by digestion with suitable restriction endonucleases. The selection of appropriate restriction endonucleases is made based on the restriction endonuclease sites in the construct.

Among particularly suitable vectors are phagemid vectors, whose use is described, for example, in U.S. Pat. No. 6,790,941 to Barbas et al., incorporated herein by this reference.

Expression of nucleic acid constructs according to the present invention can be performed by standard techniques, either in eukaryotic cells or in prokaryotic cells. For example, expression can be performed in bacterial cells, in mammalian cells, in yeast cells, in insect cells, or in other eukaryotic cells. Such techniques are described, for example, in U.S. Pat. No. 6,790,941 to Barbas et al., incorporated herein.

Transformation of a host cell with recombinant DNA may be carried out by conventional techniques as are well known to those skilled in the art. Where the host is prokaryotic, such as E. coli, competent cells which are capable of DNA uptake can be prepared from cells harvested after exponential growth phase and subsequently treated by the CaCl₂ method by procedures well known in the art. Alternatively, MgCl₂ or RbCl can be used. Transformation can also be performed after forming a protoplast of the host cell or by electroporation.

When the host is a eukaryote, such methods of transfection of DNA as calcium phosphate coprecipitation, conventional mechanical procedures such as microinjection, electroporation, insertion of a plasmid encased in liposomes, or virus vectors may be used.

A variety of host-expression vector systems may be utilized to express the fusion protein coding sequence. These include but are not limited to microorganisms such as bacteria transformed with recombinant bacteriophage DNA, plasmid DNA or cosmid DNA expression vectors containing a zinc finger derived-nucleotide binding polypeptide coding sequence; yeast transformed with recombinant yeast expression vectors containing the zinc finger-nucleotide binding coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing a zinc finger derived-DNA binding coding sequence; insect cell systems infected with recombinant virus expression vectors (e.g., baculovirus) containing a zinc finger-nucleotide binding coding sequence; or animal cell systems infected with recombinant virus expression vectors (e.g., retroviruses, adenovirus, vaccinia virus) containing a zinc finger derived-nucleotide binding coding sequence, or transformed animal cell systems engineered for stable expression. In such cases where glycosylation may be important, expression systems that provide for translational and post-translational modifications may be used; e.g., mammalian, insect, yeast or plant expression systems.

Depending on the host/vector system utilized, any of a number of suitable transcription and translation elements, including constitutive and inducible promoters, transcription enhancer elements, transcription terminators, etc. may be used in the expression vector (see e.g., Bitter, et al., Methods in Enzymology, 153:516-544, 1987). For example when cloning in bacterial systems, inducible promoters such as pL of bacteriophage λ, plac, ptrp, ptac (ptrp-lac hybrid promoter) and the like may be used. When cloning in mammalian cell systems, promoters derived from the genome of mammalian cells (e.g., metallothionein promoter) or from mammalian viruses (e.g., the retrovirus long terminal repeat; the adenovirus late promoter; the vaccinia virus 7.5K promoter) may be used. Promoters produced by recombinant DNA or synthetic techniques may also be used to provide for transcription of the fusion protein.

In bacterial systems a number of expression vectors may be advantageously selected depending upon the use intended for the fusion protein expressed. For example, when large quantities are to be produced, vectors which direct the expression of high levels of fusion protein products that are readily purified may be desirable. Those which are engineered to contain a cleavage site to aid in recovering the protein are preferred. Such vectors include but are not limited to the E. coli expression vector pUR278 (Ruther, et al., EMBO J., 2:1791, 1983), in which the fusion protein coding sequence may be ligated into the vector in frame with the lac Z coding region so that a hybrid zinc finger-containing fusion protein-lac Z protein is produced; pIN vectors (Inouye & Inouye, Nucleic Acids Res. 13:3101-3109, 1985; Van Heeke & Schuster, J. Biol. Chem. 264:5503-5509, 1989); and the like.

In yeast, a number of vectors containing constitutive or inducible promoters may be used. For a review see, Current Protocols in Molecular Biology, Vol. 2, 1988, Ed. Ausubel, et al., Greene Publish. Assoc. & Wiley Interscience, Ch. 13; Grant, et al., 1987, Expression and Secretion Vectors for Yeast, in Methods in Enzymology, Eds. Wu & Grossman, 31987, Acad. Press, N.Y., Vol. 153, pp. 516-544; Glover, 1986, DNA Cloning, Vol. 11, IRL Press, Wash., D.C., Ch. 3; and Bitter, 1987, Heterologous Gene Expression in Yeast, Methods in Enzymology, Eds. Berger & Kimmel, Acad. Press, N.Y., Vol. 152, pp. 673-684; and The Molecular Biology of the Yeast Saccharomyces, 1982, Eds. Strathern et al., Cold Spring Harbor Press, Vols. I and II. A constitutive yeast promoter such as ADH or LEU2 or an inducible promoter such as GAL may be used (Cloning in Yeast, Ch. 3, R. Rothstein In: DNA Cloning Vol. 11, A Practical Approach, Ed. D M Glover, 1986, IRL Press, Wash., D.C.). Alternatively, vectors may be used which promote integration of foreign DNA sequences into the yeast chromosome.

In cases where plant expression vectors are used, the expression of a fusion protein coding sequence may be driven by any of a number of promoters. For example, viral promoters such as the 35S RNA and 19S RNA promoters of CaMV (Brisson, et al., Nature, 310:511-514, 1984), or the coat protein promoter to TMV (Takamatsu, et al., EMBO J., 6:307-311, 1987) may be used; alternatively, plant promoters such as the small subunit of RUBISCO (Coruzzi, et al., EMBO J. 3:1671-1680, 1984; Broglie, et al., Science 224:838-843, 1984); or heat shock promoters, e.g., soybean hsp17.5-E or hsp17.3-B (Gurley, et al., Mol. Cell. Biol., 6:559-565, 1986) may be used. These constructs can be introduced into plant cells using Ti plasmids, Ri plasmids, plant virus vectors, direct DNA transformation, microinjection, electroporation, etc. For reviews of such techniques see, for example, Weissbach & Weissbach, Methods for Plant Molecular Biology, Academic Press, NY, Section VIII, pp. 421-463, 1988; and Grierson & Corey, Plant Molecular Biology, 2d Ed., Blackie, London, Ch. 7-9, 1988.

An alternative expression system that can be used to express a protein of the invention is an insect system. In one such system, Autographa californica nuclear polyhedrosis virus (AcNPV) is used as a vector to express foreign genes. The virus grows in Spodoptera frugiperda cells. The fusion protein coding sequence may be cloned into non-essential regions (Spodoptera frugiperda for example the polyhedrin gene) of the virus and placed under control of an AcNPV promoter (for example the polyhedrin promoter). Successful insertion of the fusion protein coding sequence will result in inactivation of the polyhedrin gene and production of non-occluded recombinant virus (i.e., virus lacking the proteinaceous coat coded for by the polyhedrin gene). These recombinant viruses are then used to infect cells in which the inserted gene is expressed. (E.g., see Smith, et al., J. Biol. 46:584, 1983; Smith, U.S. Pat. No. 4,215,051).

Eukaryotic systems, and preferably mammalian expression systems, allow for proper post-translational modifications of expressed mammalian proteins to occur. Therefore, eukaryotic cells, such as mammalian cells that possess the cellular machinery for proper processing of the primary transcript, glycosylation, phosphorylation, and, advantageously secretion of the gene product, are the preferred host cells for the expression of a fusion protein according to the present invention. Such host cell lines may include but are not limited to CHO, VERO, BHK, HeLa, COS, MDCK, 293, and WI38.

Mammalian cell systems that utilize recombinant viruses or viral elements to direct expression may be engineered. For example, when using adenovirus expression vectors, the coding sequence of a fusion protein according to the present invention may be ligated to an adenovirus transcription/translation control complex, e.g., the late promoter and tripartite leader sequence. This chimeric gene may then be inserted into the adenovirus genome by in vitro or in vivo recombination. Insertion in a non-essential region of the viral genome (e.g., region E1 or E3) will result in a recombinant virus that is viable and capable of expressing the zinc finger polypeptide in infected hosts (e.g., see Logan & Shenk, Proc. Natl. Acad. Sci. USA 81:3655-3659, 1984). Alternatively, the vaccinia virus 7.5K promoter may be used. (e.g., see, Mackett, et al., Proc. Natl. Acad. Sci. USA, 79:7415-7419, 1982; Mackett, et al., J. Virol. 49:857-864, 1984; Panicali, et al., Proc. Natl. Acad. Sci. USA, 79:4927-4931, 1982). Of particular interest are vectors based on bovine papilloma virus which have the ability to replicate as extrachromosomal elements (Sarver, et al., Mol. Cell. Biol. 1:486, 1981). Shortly after entry of this DNA into mouse cells, the plasmid replicates to about 100 to 200 copies per cell. Transcription of the inserted cDNA does not require integration of the plasmid into the host's chromosome, thereby yielding a high level of expression. These vectors can be used for stable expression by including a selectable marker in the plasmid, such as the neo gene. Alternatively, the retroviral genome can be modified for use as a vector capable of introducing and directing the expression of the fusion protein gene in host cells (Cone & Mulligan, Proc. Natl. Acad. Sci. USA 81:6349-6353, 1984). High level expression may also be achieved using inducible promoters, including, but not limited to, the metallothionein IIA promoter and heat shock promoters.

For long-term, high-yield production of recombinant proteins, stable expression is preferred. Rather than using expression vectors which contain viral origins of replication, host cells can be transformed with a cDNA controlled by appropriate expression control elements (e.g., promoter, enhancer, sequences, transcription terminators, polyadenylation sites, etc.), and a selectable marker. The selectable marker in the recombinant plasmid confers resistance to the selection and allows cells to stably integrate the plasmid into their chromosomes and grow to form foci which in turn can be cloned and expanded into cell lines. For example, following the introduction of foreign DNA, engineered cells may be allowed to grow for 1-2 days in an enriched media, and then are switched to a selective medium. A number of selection systems may be used, including but not limited to the herpes simplex virus thymidine kinase (Wigler, et al., Cell 11:223, 1977), hypoxanthine-guanine phosphoribosyltransferase (Szybalska & Szybalski, Proc. Natl. Acad. Sci. USA, 48:2026, 1962), and adenine phosphoribosyltransferase (Lowy, et al., Cell, 22:817, 1980) genes, which can be employed in tk⁻, hgprt⁻ or aprt⁻ cells respectively. Also, antimetabolite resistance-conferring genes can be used as the basis of selection; for example, the genes for dhfr, which confers resistance to methotrexate (Wigler, et al., Natl. Acad. Sci. USA, 77:3567, 1980; O'Hare, et al., Proc. Natl. Acad. Sci. USA, 78:1527, 1981); gpt, which confers resistance to mycophenolic acid (Mulligan & Berg, Proc. Natl. Acad. Sci. USA, 78:2072, 1981; neo, which confers resistance to the aminoglycoside G418 (Colberre-Garapin, et al., J. Mol. Biol., 150:1, 1981); and hygro, which confers resistance to hygromycin (Santerre, et al., Gene, 30:147, 1984). Recently, additional selectable genes have been described, namely trpB, which allows cells to utilize indole in place of tryptophan; hisD, which allows cells to utilize histinol in place of histidine (Hartman & Mulligan, Proc. Natl. Acad. Sci. USA, 85:804, 1988); and ODC (ornithine decarboxylase) which confers resistance to the ornithine decarboxylase inhibitor, 2-(difluoromethyl)-DL-ornithine, DFMO (McConlogue L., In: Current Communications in Molecular Biology, Cold Spring Harbor Laboratory ed., 1987).

Isolation and purification of microbially expressed protein or protein expressed in eukaryotic cells can be carried out by conventional means including preparative chromatography and immunological separations involving monoclonal or polyclonal antibodies. Antibodies can be prepared by standard techniques that are immunoreactive with the zinc finger tag incorporated into the fusion protein of the invention. Antibodies can also be prepared to other portions of the fusion protein. Antibodies which consist essentially of pooled monoclonal antibodies with different epitopic specificities, as well as distinct monoclonal antibody preparations are provided. Monoclonal antibodies are made by methods well known in the art (Kohler, et al., Nature, 256:495, 1975; Current Protocols in Molecular Biology, Ausubel, et al., ed., 1989).

Accordingly, another aspect of the present invention is a method of expressing a fusion protein according to the present invention comprising:

(1) introducing a vector encoding a fusion protein according to the present invention into a compatible host cell; and

(2) causing the fusion protein to be expressed in the host cell; and

(3) isolating the expressed fusion protein.

As indicated above, the compatible host cell can be a eukaryotic or a prokaryotic cell.

III. Applications

A. Localization of Proteins

Accordingly, an embodiment of the invention is a method for in vivo localization of a target protein in a cell comprising the steps of:

(1) expressing a fusion protein according to the present invention in a cell, the target protein being incorporated in the fusion protein;

(2) introducing a DNA molecule into the cell that is specifically bound by the zinc finger tag of the fusion protein, wherein the DNA molecule is covalently labeled with a fluorescent indicator molecule;

(3) incubating the cell so that the DNA molecule binds to the fusion protein; and

(4) localizing the target protein in the cell by locating the fluorescent indicator molecule.

Typically, the fluorescent indicator molecule is selected from the group consisting of 4-acetamido-4′-isothiocyanatostilbene-2,2′-disulfonic acid, diethylaminocoumarin, 7-amino-4-methylcoumarin, Cascade Blue, Oregon Green 488, Alexa 488, fluorescein isothiocyanate, BODIPY FL, B phycoerythrin, tetramethyl rhodamine isothiocyanate, cyanine 3.18, R phycoerythrin, lissamine rhodamine sulfonylchloride, rhodamine X isothiocyanate, Alexa 594, Texas Red, and BODIPY TR. Other fluorescent indicators are known in the art.

The protein can be localized by techniques known in the art, such as those described in L. C. Javois, “Immunocytochemistry” in Molecular Biomethods Handbook (R. Rapley & J. M. Walker, eds., Humana Press, Totowa, N.J., 1998), pp. 631-651, incorporated herein by this reference, which describes various immunocytochemical procedures for localization of proteins in cells, such as the use of paraffin-embedded and sectioned-tissue preparations, frozen sections and touch preparations, and the use of cell suspensions and culture preparations. Fluorescent microscopy can be used to determine the in vivo localization of these DNA-labeled proteins. Cells containing the protein can also be isolated by flow cytometry, as described in R. E. Cunningham, “Flow Cytometry” in Molecular Biomethods Handbook (R. Rapley & J. M. Walker, eds., Humana Press, Totowa, N.J., 1998), pp. 653-667, incorporated herein by this reference. Flow cytometry can be used in an analytical or a preparative manner.

The DNA molecule is one that binds specifically to the zinc finger tag as described above; i.e., one that includes the sequence of 18 base pairs that binds in a sequence-specific manner to the zinc finger tag. Typically, the DNA molecule is single-stranded. Typically, the DNA molecule is in a hairpin conformation with a stem and loop in which the stem is double-stranded and the loop has unpaired bases; however, DNA molecules suitable for use in methods according to the present invention do not require the presence of a hairpin structure. All that is required is a secondary structure that permits sequence-specific binding by the zinc finger tag. Preferably, the fluorescent indicator molecule is covalently bound to the DNA molecule, such as at its 3′-terminus. Conjugation reactions for covalently labeling DNA are known in the art and are described, for example, in G. T. Hermanson, “Bioconjugate Techniques (Academic Press, San Diego, 1996), pp. 639-671. Typically, the DNA is first derivatized to contain a suitable functional group for conjugation with the fluorescent indicator molecule, such as an amine or sulfhydryl moiety. Alternatively, the terminal transferase reaction is used to add a modified nucleoside triphosphate to the 3′-terminus, which is then reacted with the fluorescent indicator molecule. For example, the DNA can be modified with a diamine compound to contain terminal primary amines, which can then be coupled with an amine-reactive fluorescent label. Alternatively, the label can be attached via an avidin-biotin link.

The fusion protein expressed in the cell and used in this method can include therein the zinc finger tags or modules described above. For example, the zinc finger tags or modules can include framework subdomains derived from C₂—H₂ zinc finger proteins, C₃H zinc finger proteins, C₄ zinc finger proteins, H₄ zinc finger proteins, CH₃ zinc finger proteins, C₆ zinc finger proteins, or, alternatively, derived from avian pancreatic polypeptide (aPP). The zinc finger tags or modules can include DNA binding subdomains that bind sequences of the form ANN, AGC, CNN, GNN, or TNN, including the DNA binding subdomains described above, or can include a combination of DNA binding subdomains that bind sequences of these forms. The DNA binding subdomains can be chosen to bind a sequence that is specific to the DNA molecule that is introduced into the cell.

The target protein to be localized can be localized in a particular cellular organelle, such as the nucleus, the nucleolus, the endoplasmic reticulum, the nuclear membrane, the cell membrane, the Golgi apparatus, the mitochondria, the chloroplast, the peroxisome, or any other organelle.

The protein to be localized can be any protein of interest as described above.

This approach is an alternative and a complement to the use of Green Fluorescent Protein (GFP) to label proteins for in vivo localization, such as described in B. A. Griffin et al., “Specific Covalent Labeling of Recombinant Protein Molecules Inside Live Cells, Science 281: 269-272 (1998), incorporated herein by this reference.

B. Assembly of Protein Arrays

Another embodiment of the invention is a protein array that is assembled by the interaction of the zinc finger tag with a DNA sequence to which it specifically binds.

In general, an array according to the present invention comprises:

(1) a solid support;

(2) a plurality of nucleotide sequences, each nucleotide sequence being attached at a defined nonoverlapping location on the solid support, each DNA molecule including a sequence that is specifically bound by a zinc finger tag; and

(3) a plurality of fusion proteins, each fusion protein comprising: (a) a protein of interest as defined above; and (b) a zinc finger tag specifically binding a sequence within a nucleotide sequence attached to the solid support.

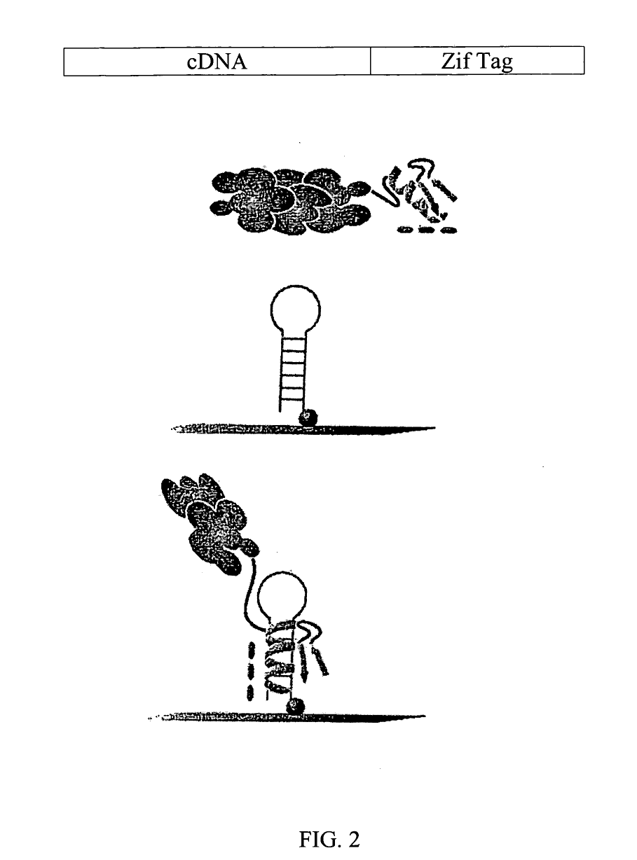

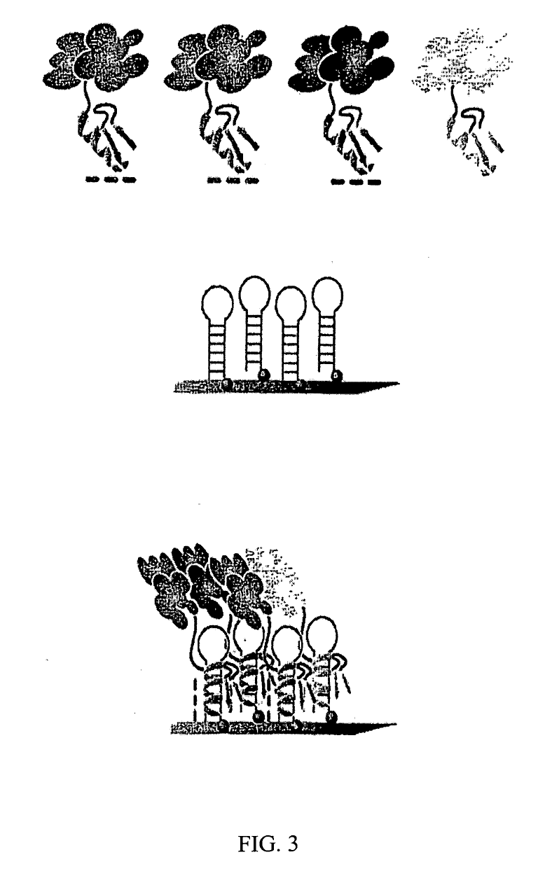

Typically, the nucleotide sequences are DNA sequences, such as cDNA sequences. The construction of these arrays is shown schematically in FIG. 2. Such arrays, when incorporating cDNA sequences, can be referred to as “cDNA biochips.”

The protein attached to the array can be any protein of interest as defined above. One protein that is significant is an antibody molecule, typically in the form of a scFv fragment

Various arrangements of the array are possible. In one variation, all of the nucleotide sequences and zinc finger tags are identical. In another variation, a plurality of different nucleotide sequences is attached to the solid support in defined locations, and different zinc finger tags are used, each zinc finger tag used specifically binding a particular nucleotide sequence. This provides a way of directing a particular subpopulation of proteins to a particular portion of the array.

Each of the plurality of nucleotide sequences can be of a length selected from the group consisting of 3 base pairs, 6 base pairs, 9 base pairs, 12 base pairs, 15 base pairs, and 18 base pairs; typically, the length is selected from the group consisting of 9 base pairs, 12 base pairs, 15 base pairs, and 18 base pairs; preferably, to provide optimal specificity, the length is 18 base pairs.

In one alternative, each of the proteins, peptides, or polypeptides of interest in the fusion proteins is from the same organism. In one application of this alternative, each of the proteins, peptides, or polypeptides of interest in the fusion proteins is from the same organelle or subcellular structure of the same organism. The organelle or subcellular structure is typically selected from the group consisting of the nucleus, the nucleolus, the endoplasmic reticulum, the Golgi apparatus, and the cell membrane.

In another alternative, each fusion protein can include the same peptide, polypeptide, or protein of interest. In still another alternative, all of the nucleotide sequences and zinc finger tags are identical. In still another alternative, a plurality of different nucleotide sequences are attached to the solid support in defined locations, and a plurality of different zinc finger tags is used, each zinc finger tag used specifically binding a particular nucleotide sequence.

The fusion protein or proteins used in these arrays can include therein the zinc finger tags or modules described above. For example, the zinc finger tags or modules can include framework subdomains derived from C₂—H₂ zinc finger proteins, C₃H zinc finger proteins, C₄ zinc finger proteins, H₄ zinc finger proteins, CH₃ zinc finger proteins, C₆ zinc finger proteins, or, alternatively, derived from avian pancreatic polypeptide (aPP). The zinc finger tags or modules can include DNA binding subdomains that bind sequences of the form ANN, AGC, CNN, GNN, or TNN, including the DNA binding subdomains described above, or can include a combination of DNA binding subdomains that bind sequences of these forms, as described above with respect to the construction of the individual fusion proteins. The DNA binding subdomains can be chosen to bind a sequence that is specific to one or more of the nucleotide sequences attached to the solid support, as described above.

Arrays of DNA molecules and methods of attaching DNA molecules to such arrays are well known in the art and need not be described further in detail. Such arrays and methods are described, for example, in D. Stekel, “Microarray Bioinformatics” (Cambridge University Press, 2003), pp. 1-18, incorporated herein by this reference. Solid supports can include, but are not necessarily limited to, glass. The DNA molecules can be presynthesized and affixed to the glass, typically covalently. Alternatively, the DNA molecules can be synthesized in situ and built up base-by-base on the surface of the array.

Various additional techniques for the preparation of DNA arrays have been described For example, in M. L. Bulyk et al., “Exploring the DNA-Binding Specificities of Zinc Fingers with DNA Microarrays,” Proc. Natl. Acad. Sci. USA 98: 7158-7163 (2001), incorporated herein by this reference, DNA microarrays were prepared by silanizing glass slides with aminopropyl methyl diethoxysilane and then activating the surface of the slides with 1,4-diphenylene-diisothiocyanate for binding to DNA molecules. Typically, the DNA molecules bound to the arrays are first prepared as single-stranded molecules and then converted to double-stranded molecules by primer extension. Alternative techniques are further described in M. L. Bulyk et al., “Quantifying DNA-Protein Interactions by Double-Stranded DNA Arrays,” Nature Biotechnol. 17: 573-577 (1999). These techniques involving synthesizing single DNA strands on glass supports, with the DNA being attached to the glass surface with either one or two hexaethylene glycol synthesis linkers, and with the second strand then being synthesized by extension of complementary primers.

In an array according to the present invention, the plurality of fusion proteins can be a result of the expression of a nucleic acid construct that is formed from a cDNA library such that each member of the plurality of fusion proteins comprises a protein that is encoded within the cDNA library together with the zinc finger tag. Techniques for preparing cDNA libraries from isolated mRNA, cloning cDNA libraries into an appropriate vector, and manipulating members of the cDNA libraries such that the cDNA is expressed as a fusion protein are well known in the art and are described, for example, in J. Sambrook & D. W. Russell, “Molecular Cloning: A Laboratory Manual” (3^{rd} ed., Cold Spring Harbor Laboratory Press, Cold Spring Harbor, N.Y., 2001), vol., 2, ch. 11, and in other portions of this reference manual. Typically, the cDNA libraries are cloned into a vector such that the cloning of cDNA into the vector generates a fusion protein such that the protein product of the cDNA and the zinc finger tag are expressed in a single open reading frame, with or without a linker. This process is shown schematically in FIG. 3.

The protein of interest in the fusion protein bound to the array retains its biological activity, such as, but not limited to, enzymatic activity, antibody activity, or receptor activity.

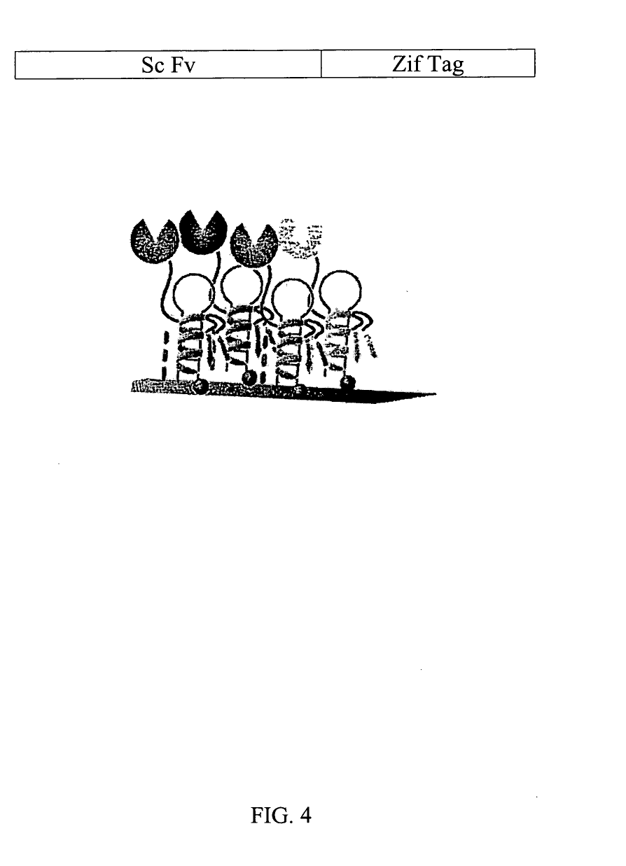

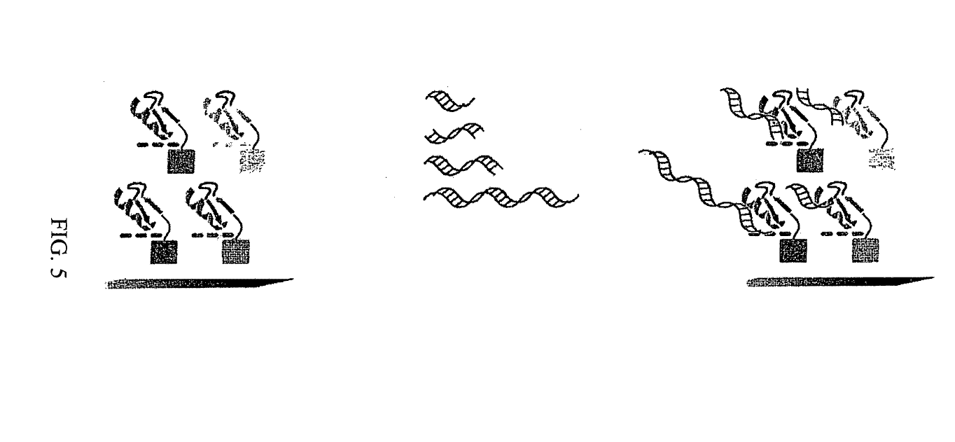

Accordingly, the protein array can be an antibody array, particularly an array of scFv antibody molecules incorporated into fusion proteins, as is shown in FIG. 4.

Accordingly, another aspect of the invention comprises a method for assaying activity of a protein of interest incorporated in a fusion protein bound to an array according to the present invention, the method comprising the steps of:

(1) providing an array according to the present invention as described above;

(2) contacting the array with a reagent that reacts with a protein of interest that may or not be present in the array to produce a detectable product; and

(3) determining the location of a protein in the array by determining the location of the detectable product in order to identify the location of a protein that has a defined activity associated with the production of the detectable product.

The assay can be any assay that can be used to detect the activity of a protein, such as an enzymatic assay, a binding assay, or an assay that measures regulatory activity. For example, if the activity is an enzymatic assay, the assay can measure hydrolysis of a substrate, formation of a bond such as a peptide bond or a phosphodiester bond or any other reaction susceptible to measurement by the production of a detectable product. If the activity is that of an antibody, the assay can measure, for example, inactivation of a molecule specifically bound by the antibody.

This provides a method for analysis of the proteome in terms of function.

This provides for the expression of large arrays of proteins en masse and their self-assembly onto DNA arrays, allowing for the rapid construction of protein arrays without the need for independent protein expression and purification.

C. Labeling of Cells

In another embodiment of the invention, cells can be labeled on their surface to express a fusion protein that is a fusion of a membrane protein with a zinc finger tag. The cells can be labeled with DNA that is specifically bound by the zinc finger tag.

Accordingly, another method according to the present invention comprises:

(1) transforming or transfecting a host cell with a nucleic acid sequence that encodes a fusion protein that is a fusion of a membrane protein with a zinc finger tag such that the cell expresses the fusion protein;

(2) culturing the transformed or transfected cell under conditions such that the fusion protein is expressed and is incorporated in the cell membrane of the cell;

(3) contacting the cell expressing the fusion protein incorporated in the membrane with a labeled DNA molecule that binds the zinc finger tag of the fusion protein in a sequence-specific manner; and

(4) detecting the label of the labeled DNA molecule on the cell surface.

The membrane protein is typically a transmembrane protein that includes an extracellular domain, a transmembrane domain, and an intracellular domain. When the membrane protein is a transmembrane protein, the zinc finger tag is typically positioned in the fusion protein such that the zinc finger tag is adjacent to the extracellular domain and so that it is accessible for binding by the labeled DNA molecule.

The labeled DNA molecule is as described above.

The fusion protein expressed in the cell and used in this method can include therein the zinc finger tags or modules described above. For example, the zinc finger tags or modules can include framework subdomains derived from C₂—H₂ zinc finger proteins, C₃H zinc finger proteins, C₄ zinc finger proteins, H₄ zinc finger proteins, CH₃ zinc finger proteins, C₆ zinc finger proteins, or, alternatively, derived from avian pancreatic polypeptide (aPP). The zinc finger tags or modules can include DNA binding subdomains that bind sequences of the form ANN, AGC, CNN, GNN, or TNN, including the DNA binding subdomains described above, or can include a combination of DNA binding subdomains that bind sequences of these forms. The DNA binding subdomains can be chosen to bind a sequence that is specific to the labeled DNA molecule.

Therefore, yet another aspect of the invention is a cell including therein a fusion protein that is a fusion of a membrane protein with a zinc finger tag such that the fusion protein is incorporated into the cell membrane.

In another aspect of the invention involving cells tagged with a fusion protein that is a fusion of a membrane protein with a zinc finger tag, the cells can be labeled with DNA, the cells arrayed on DNA surfaces by specific base pairing, and then cross-linked on the DNA surfaces. The specific base pairing involved is between the DNA used to label the cells and the DNA on the DNA surfaces; such base pairing occurs by standard Watson-Crick complementarity. The cells cross-linked on the DNA surfaces can then be contacted with a probe to study cell-surface interactions, such as a labeled antibody, a labeled receptor ligand, or other molecule capable of binding to cell surfaces.

D. Double-Stranded DNA Analysis

Yet another aspect of the invention is a method of analysis of double-stranded DNA. In general, this method comprises the steps of:

(1) providing a plurality of fusion proteins, each fusion protein comprising (a) a protein of interest as defined above; and (b) a zinc finger tag specifically binding a defined nucleotide sequence within a DNA molecule;

(2) binding the fusion proteins to a solid support, each fusion protein being attached at a defined nonoverlapping location on the solid support, to produce a fusion protein microarray;

(3) exposing the fusion proteins to a sample containing one or more double-stranded DNA molecules so that any double-stranded DNA molecules possessing a defined nucleotide sequence bound by a zinc finger tag incorporated in a fusion protein is bound; and

(4) analyzing the binding of DNA molecules to the fusion proteins in order to determine whether DNA molecules possessing any of the defined nucleotide sequences are present in the sample.

This process is shown schematically in FIG. 5.

The fusion proteins can be bound to the solid support either covalently or noncovalently. For example, they can be bound via an avidin-biotin link, as is known in the art. Alternatively, they can be bound noncovalently to a plastic surface as is commonly done for ELISA assays. Other methods are known in the art.

Accordingly, yet another aspect of the invention is an array comprising: (1) a solid support;

(2) a plurality of fusion proteins, each fusion protein comprising: (a) a protein of interest as defined above; and (b) a zinc finger tag specifically binding a defined nucleotide sequence within a DNA molecule, the fusion proteins being attached to the solid support.

The fusion protein used in this array can include therein the zinc finger tags or modules described above. For example, the zinc finger tags or modules can include framework subdomains derived from C₂—H₂ zinc finger proteins, C₃H zinc finger proteins, C₄ zinc finger proteins, H₄ zinc finger proteins, C₁H₃ zinc finger proteins, C₆ zinc finger proteins, or, alternatively, derived from avian pancreatic polypeptide (aPP). The zinc finger tags or modules can include DNA binding subdomains that bind sequences of the form ANN, AGC, CNN, GNN, or TNN, including the DNA binding subdomains described above, or can include a combination of DNA binding subdomains that bind sequences of these forms. The DNA binding subdomains can be chosen to bind a sequence that is specific to one or more DNA molecules that are in the sample or are expected to be in the sample.

The invention is described by the following Examples. These Examples are for illustrative purposes only and are not intended to limit the invention.

### EXAMPLE 1

### Use of DNA Microarrays to Explore DNA-Binding Specificities of Zinc Fingers

This Example is based on the work reported in the publication M. L. Bulyk et al., “Exploring the DNA-Binding Specificities of Zinc Fingers with DNA Microarrays,” Proc. Natl. Acad. Sci. 98: 7158-7163 (2001). This Example is provided to demonstrate a method of providing arrays of nucleotide sequences that can be bound specifically by zinc finger proteins. For use in methods according to the present invention, such arrays can be bound by fusion proteins as described above

Materials and Methods

Synthesis of DNA Microarrays. Cy3-labeled oligonucleotide is spotted for alignment purposes. The set of 64 oligonucleotides, synthesized to represent all possible 3-nt central-finger sites for Zif268 zinc fingers, is combined with a 5′ amino-tagged universal primer in a 2:1 molar ratio in a Sequenase (United States Biochemical) reaction. The completed extension reactions are exchanged into 150 mM K₂HPO₄, pH 9.0, by using CentriSpin-10 spin columns (Princeton Separations, Adelphia, N.J.).

The following Cy3-labeled oligonucleotide (Operon Technologies, Alameda, Calif.) is spotted at 10 μM in 150 mM K₂HPO₄, pH 9.0, for alignment purposes: 5′-TCAGAACTCACCTGTTAGAC-3′ (SEQ ID NO: 707). The following set of 64 oligonucleotides 37 nt in length is synthesized (Operon) so as to represent all possible 3 nt central finger sites for Zif268 zinc fingers: 5′-TATATAGCGNNNGCGTATATATCAAGTCAATCGGTCC-3′ (SEQ ID NO: 708) (the three sites for fingers 1 through 3 are underlined; bold letters show the position of the 64 possible 3-nt sites for the central finger). The following 16-mer is synthesized with a 5′ amino linker (Operon) and used as a universal primer: 5′-GGACCGATTGACTTGA-3′ (SEQ ID NO: 709). Each of the 64 unmodified 37-mer is combined with the amino-tagged 16-mer in a 2:1 molar ratio in a Sequenase reaction using 20 μM 16-mer. The completed extension reactions are exchanged into 150 mM K₂HPO₄, pH 9.0, by using CentriSpin-10 spin columns (Princeton Separations, Adelphia, N.J.). The resulting samples are transferred to a 384-well plate for arraying.

Phage ELISAs To determine apparent dissociation constants (K_{d}^{app}s), phage ELISAs are carried out at least in triplicate, essentially as described (4), with some modifications. Exact methods and oligonucleotides are described below. Because these measurements provide apparent, not actual, K_{d}s, all final observed K_{d}^{app} values are scaled by the same constant so that the K_{d}^{app} for wild-type Zif268 with the sequence containing the 3-bp finger 2 binding-site TGG was equal to 3.0 nM.

Phage Library Construction. Construction of the phage display library of the three fingers of Zif268 has been described previously [Choo, Y. & Klug, A. (1994) Proc. Natl. Acad. Sci. USA 91, 11163-11167]. Briefly, the seven positions of the second finger's α-helix that are the primary and secondary putative base recognition positions were randomized. In addition, position +9 (relative to the first residue in the α-helix, +1), was allowed to be either Arg or Lys, the two most frequently occurring residues at that position. This design was intended to direct the randomized finger to the variant DNA triplets, since the overall register of protein-DNA contacts should be fixed by the first and third fingers.

Microarray Protein Binding. For production of Zif phage, overnight bacterial cultures of TG1 (or JM109) cells, each producing a particular zinc-finger phage or pool of phages, are grown at 30° C. in 2×TY medium containing 50 mM zinc acetate and 15 mg/ml tetracycline (2×TY/Zn/Tet). Culture supernatants containing phage are diluted 2-fold by addition of PBS/Zn containing 4% (wt/vol) nonfat dried milk, 2% (vol/vol) Tween 20, and 100 mg/ml salmon testes DNA (Sigma). The slides are blocked with 2% milk in PBS/Zn for 1 h, then washed once with PBS/Zn/0.1% Tween 20, then once with PBS/Zn/0.01% Triton X-100. The diluted phage solutions are then added to the slides, and binding was allowed to proceed for 1 h. The slides are then washed five times with PBS/Zn/1% Tween 20, and then three times with PBS/Zn/0.01% Triton X-100. Mouse anti-(M13) antibody (Amersham Pharmacia) is diluted in PBS/Zn containing 2% milk, preincubated for at least 1 h, and added to the slide. After incubation for 1 h at room temperature, the slides are washed three times with PBS/Zn/0.05% Tween 20, and three times with PBS/Zn/0.01% Triton X-100. R-phycoerythrin-conjugated goat anti-(mouse IgG) (Sigma) is diluted in PBS/Zn containing 2% milk, preincubated for at least 1 h, and added to the slides. After incubation for 1 h at room temperature, the slides are washed three times with PBS/Zn/0.05% Tween 20, three times with PBS/Zn/0.01% Triton X-100, and once with PBS/Zn, and then scanned. This basic protocol can be used for phages expressing fusion proteins according to the present invention for binding to microarrays that have nucleotide sequences for which zinc finger tags in the fusion proteins are specific.

To ensure that all the binding affinity data are calculated with fluorescence intensities below the saturation level of the microarray scanner, the microarrays are scanned at multiple laser power settings. The relative fluorescence intensities for each scan are were normalized relative to a sequence with one of the highest fluorescence intensities on the respective scans. These ratios are then multiplied to calculate all the fluorescence intensities as a fraction of the sequence with the overall highest fluorescence intensity.

Formally, the microarray binding experiments only indicate which sequences are bound. However, in the case of the well studied Zif268-like zinc fingers, it is possible to deduce the binding sites within these DNA sequences. The AT-rich sequences flanking the 9-bp binding sites for the Zif phage serve as an attempt to confine zinc finger binding to within the GC-rich portion.

Microarray Data Analysis

Microarrays are scanned essentially as described (M. Schena et al., “Quantitative Monitoring of Gene Expression Patterns with a Complementary DNA Microarray,” Science 270: 467-470 (1995)). The signal intensities of each of the spots in the scanned images are quantified by using IMAGENE Version 3.0 software (BioDiscovery, Los Angeles, Calif.). Subsequent analyses are performed with PERL scripts. After background subtraction, the relative signal intensity of each of the spots within a replicate is calculated as a fraction of the highest signal intensity for a spot containing one of the 64 different 37-bp sequences. To normalize for possible variability in the DNA concentrations of the different DNA samples that are spotted onto the microarrays, each of the average relative signal intensities from zinc-finger phage binding is divided by each of the respective average relative signal intensities from SybrGreen I staining.

Microarrays are scanned by using a GSI Lumonics ScanArray 5000 microarray scanner. Images are scanned at a resolution of 10 μm per pixel. Fluorescent signals are detected with a helium neon laser with an excitation of 543.5 nm and a 570-nm bandpass filter for R-phycoerythrin and Cy3, and an argon laser with an excitation of 488 nm and a 522-nm bandpass filter for SybrGreen I. The signal intensities of each of the spots in the scanned images are quantified by using IMAGENE ver. 3.0 software (BioDiscovery, Los Angeles, Calif.). Subsequent analyses are performed with PERL scripts.

Background signal intensities are calculated individually for each spot as the area of the spot multiplied by the median signal intensity in a 5-pixel-thick perimeter at a distance of 5 pixels outside of each spot. After background subtraction, the relative signal intensity of each of the spots within a replicate is calculated as a fraction of the highest signal intensity for a spot containing one of the 64 different 37-bp sequences. The relative intensities are calculated individually within each replicate before averaging over all the replicates on the microarray so as to control for any overall variation in the binding and antibody reactions. Each of these relative signal intensities is then averaged over the nine replicates present on each slide. To normalize for possible variability in the DNA concentrations of the different DNA samples that were spotted onto the microarrays, separate microarrays manufactured in the same print run are quantified by SybrGreen I staining. Each of the average relative signal intensities from zinc finger phage binding are divided by each of the respective average relative signal intensities from SybrGreen I staining. The fluorescence intensities of spots at or below background are set to be the standard deviation of the spot with the lowest quantifiable fluorescence intensity on the respective microarrays. For the microarray binding experiment using wild-type Zif268, the highest relative signal intensity observed is expected to be 1 for the triplet TGG, and the lowest relative fluorescence intensity observed is expected to be 0.0305 for the triplet AGA.

### EXAMPLE 2

### Construction of Polydactyl Zinc Finger Tags

This Example is intended to describe one method for the design and construction of polydactyl zinc finger tags for inclusion in fusion proteins according to the present invention. This Example is not intended to limit fusion proteins according to the present invention to those including polydactyl zinc finger tags designed and constructed according to the method of this Example.

Introduction

In recent years, advances in the area of protein engineering and in understanding of protein-DNA interactions have enabled the creation of novel DNA-binding proteins that are capable of recognizing virtually any desired DNA sequence (1-3). Such proteins have enabled the development of artificial transcription factors, which have been shown to up- or down-regulate a growing list of specific endogenous genes (4-6). Successful transgenic plants (7, 8) and pre-clinical studies (9) have validated the utility of novel DNA-binding proteins to produce targeted gene regulators and therapeutics. New sequence-specific tools such as targeted endonucleases and integrases are nearing functional readiness (10, 11).

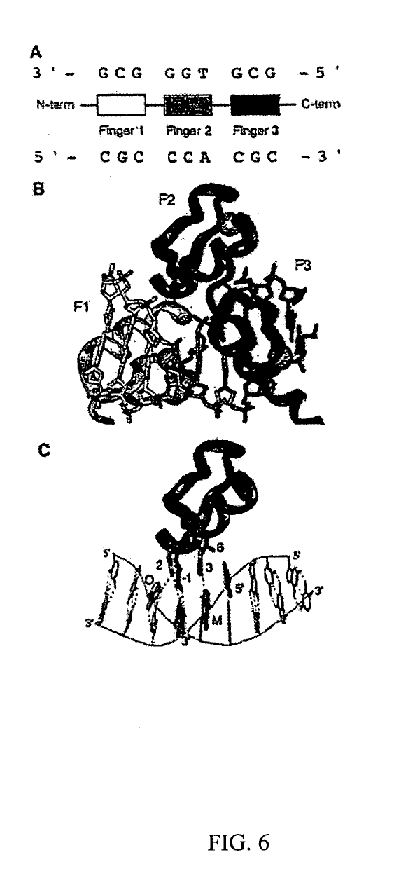

The technology that has made these advances possible is based on the DNA-recognition properties of one particular class of DNA-binding domains, the Cys₂-His₂ zinc finger (FIG. 6). FIG. 6 shows representations of zinc finger-DNA interactions, based on the structure of Zif268 (14). (A) Diagram showing the anti-parallel orientation of a 3-finger protein to its DNA target. The target sequence is shown as the top strand. (B) A structural representation of a 3-finger protein bound to nine bp of DNA. The protein and DNA are colored as in (A). Zinc ions are shown as spheres. (C) The DNA-contacting residues of finger 2 and the bases typically contacted in the major grove. The residues are numbered (−1, 2, 3, 6) with respect to the α-helix. The 5′ (“5′”), middle (“M”), and 3′ (“3′”) nucleotides that comprise the binding triplet for that domain are on one strand of the DNA. The nucleotide typically involved in target site overlap interactions (“O”) is on the opposite strand. This domain is the most common DNA-binding motif found in eukaryotes and is by far the most prevalent type of domain found in the human genome, with over 4,500 examples identified (12). Each 30-amino acid domain contains a single amphipathic α-helix stabilized by zinc ligation to two β-strands (FIG. 6B). Sequence-specific recognition is provided by contact of amino acids of the N-terminal portion of the α-helix with base edges of predominantly one strand in the major grove of the DNA (FIG. 6C). Among naturally occurring zinc finger domains, DNA-interactions can be grouped as canonical and non-canonical types (13). Two examples of proteins with canonical type DNA-recognition are the transcription factors Zif268 (14, 15) and Sp1 (16). In these proteins, each domain recognizes essentially a three nucleotide subsite. Amino acids in positions −1, 3, and 6 (numbered with respect to the start of the α-helix) contact the 3′, middle, and 5′ nucleotides, respectively. Positions 2, 1, and 5 are often involved in direct or water-mediated contacts to the phosphate backbone. Position 4 is typically a leucine residue that packs in the hydrophobic core of the domain. Position 2 has been shown to interact with other helix residues and with bases depending on the protein and DNA sequences.

In previous work, combinatorial mutagenesis and selection methods were used to modify the binding specificity of naturally occurring zinc finger domains (17-19). Starting with a canonical-type 3-finger protein, amino acids in positions −2 through 6 of the central domain were randomized. Proteins that could specifically recognize a new three-nucleotide subsite were selected by phage display, then optimized by site-directed mutagenesis. Domains that bind with high affinity and specificity to the 16 members of the 5′-GNN-3′ set of DNA triplets and 14 of the 16 5′-ANN-3′ sequences have been reported. The selection of domains recognizing 5′-CNN-3′ and 5′-TNN-3′ sequences is in progress. These accomplishments have brought the art within reach of the ability to specifically recognize any of the 64 possible three-nucleotide subsites. Zinc finger domains are useful for the construction of new DNA-binding proteins because they are organized in tandem arrays, allowing recognition of extended, non-palindromic DNA sequences. Consequently, optimized domains are assembled into 6-finger proteins, which have the theoretical capacity to recognize an 18-bp target site (4, 17, 20, 21). A site of this length has the potential to be unique in the human genome, as well as all other known genomes. The published 5′(G/A)NN-3′ domains (17-19) allow for the rapid construction of more than one billion unique proteins, potentially capable of targeting one unique site for every 32 base pairs of DNA. These domains can therefore be incorporated into zinc finger tags and used in fusion proteins according to the present invention.

The zinc finger domains used to construct polydactyl proteins were initially selected and optimized as the finger 2 domain (F2) of a 3-finger protein (17-19). The binding specificity of each domain was determined in this “F2 context” using a stringent multi-target ELISA assay. One goal of the current study was to determine if the domains maintain their exquisite specificity when repositioned at finger 1 or 3 positions, and when they are incorporated into polydactyl 6-finger proteins. The potential of three different frameworks (the non-DNA-contacting regions of zinc finger domains) for arranging the domains into multi-finger proteins was previously examined (20). The F2 domains were linked in tandem (F2-backbone) or just the DNA-contacting residues of the domain were transplanted to the framework of the 3-finger proteins Zif268 or Sp1C (a consensus framework based on the Sp1 protein (22)). Proteins with an Sp1C-backbone were generally found to have a higher affinity than those with the other two. In a published example, the affinity of the 6-finger protein E2C improved 50-fold by displaying the same DNA-contacting residues in an Sp1C-rather than a F2-backbone (20). However, increased affinity often correlates with decreased specificity. Therefore, another goal of the current study was to investigate if the use of a F2-, Zif- and Sp1C-backbone affected specificity.

Finally, others in the field have observed that some domains in fact recognize a four-nucleotide subsite, with the fourth nucleotide overlapping the first nucleotide of the next site (5, 6, 23-27). This concern, referred to as target site overlap, would limit the ability to assemble the domains in any desired order. To address this concern, other groups have developed randomization and selection strategies in which two or more domains are modified simultaneously (28, 29), or each domain is selected sequentially in the “context” of a previously-selected domain (30). Construction of new DNA-binding proteins by these procedures is laborious because new and/or multiple randomized libraries must be screened for each DNA target sequence. In contrast, this approach enables the rapid construction of multi-domain proteins, but requires that each domain be modular and independent. Therefore, there was interest in examining the extent to which target site overlap affects domain modularity and binding specificity of polydactyl proteins assembled using this methodology.

The studies of a large number of modularly assembled proteins demonstrates that the zinc finger domains generally maintain their specificity regardless of their new position. Effects due to target site overlap were evident but typically limited to predictable cases. In 3-finger proteins, specificity was found to be as good as or better than for proteins constructed by other methods. The recognition patterns of the 6-finger proteins were more complex. Potential explanations, such as framework restrictions and increased affinity, are discussed. Overall, these results validate the modular assembly strategy as a robust method for the generation of new high-affinity, site-specific DNA-binding proteins.

Methods and Materials

Assembly of 3- and 6-finger proteins. Proteins were assembled from oligonucleotides using domain sequences and methods previously described. Genes for polydactyl proteins were cloned into a modified pMAL-c2 bacterial expression vector (New England Biolabs). Expressed proteins contained a maltose-binding protein (MBP) purification tag at the N-terminus and an Hemophilus influenzae hemagglutinin CIA) epitope tag at the C-terminus.

Multi-target specificity assays. These assays were performed as described (19). Essentially, freeze/thaw extracts containing the overexpressed maltose-binding protein zinc-finger fusion proteins were prepared from IPTG-induced cultures using the Protein Fusion and Purification System (New England Biolabs) in Zinc Buffer A (ZBA; 10 mM Tris, pH7.5/90 mM KCl, 1 mM MgCl₂, 90 μM ZnCl₂). Streptavidin (0.2 μg) was applied to a 96-well ELISA plate, followed by the indicated DNA targets (0.025 μg). Biotinylated hairpin oligonucleotides containing the indicated target sequences were immobilized on streptavidin-coated 96-well ELISA plates. Target hairpin oligonucleotides had the sequence 5′-Biotin-GGAN¹′N¹′N¹′N²′N²′N²′N³′N³′N³′GGG TTTT CCC N³N³N³N²N²N²N¹N¹N¹TCC-3′ (SEQ ID NO: 710), where N¹N¹N¹ was the 3-nucleotide finger-1 target sequence and N¹′N¹′N¹′ its complement. The plates were blocked with ZBA/3% BSA. Eight 2-fold serial dilutions of the extracts were applied in 1× Binding Buffer (ZBA/1% BSA/5 mM DTT/0.12 μg/μl sheared herring sperm DNA), and bound protein was detected by mAb mouse anti-maltose binding protein (Sigma) and mAb goat-anti-mouse IgG conjugated to alkaline phosphatase (Sigma). Alkaline phosphatase substrate (Sigma) was applied, and the OD₄₀₅ was quantitated with SOFTmax 2.35 (Molecular Devices). All titration data were background subtracted from ELISA wells containing extract but no oligonucleotide.

CAST assays. Fusion proteins were purified over amylose resin to >90% homogeneity using the Protein Fusion and Purification System (New England Biolabs) according to the manufacturer's recommendations, except that ZBA/5 mM DTT was used as the column buffer. Proteins were eluted with 10 mM maltose, concentrated, and stored in ZBA containing 50% glycerol//5 mM DTT at −20° C. Protein purity and concentration were determined from Coomassie blue-stained SDS-PAGE gels by comparison to BSA standards.

Randomized libraries of double-stranded DNA were created by PCR amplification of 150 pmole of a library oligonucleotide, 5′-GAGCTCATGGAAGTACCATAG-(N)_{10, 12, or 21}-GAACGTCGATCACTCGAG-3′ (SEQ ID NO: 711, 712, and 713), with the primers 5′-GAGCTCATGGAAGTACCATAG-3′ (SEQ ID NO: 714) and 5′-CTCGAGTGATCGACGTTC-3′ (SEQ ID NO: 715) (10 cycles; 15 seconds @ 94°, 15 seconds @ 70° C., 60 seconds at 72° C.). Libraries were trace labeled by inclusion of 10 μCi [α-³²P]-dATP in the PCR reaction. Proteins were incubated with 1 pM DNA library in 1× Binding Buffer/10% glycerol for one hour at room temperature, then separated on a 5% polyacrylamide gel in 0.5×TBE buffer. Imaging of dried gels was performed using a PhosphorImager and ImageQuant software (Molecular Dynamics). The mobility of faint protein/DNA complexes was determined from positive controls in early rounds. Complexes were eluted from excised gel fragments in elution buffer (0.1% SDS/0.5M NH₃OAc/10 mM MgOAc) overnight at 37° C., then reamplified by 15 cycles of PCR as described above.

Protein concentration was approximately 1 or 0.1 μM (for 3- or 6-finger proteins, respectively) in the first round, then decreased in subsequent rounds as protein/DNA complexes became visible. CAST selections were repeated until 50% of the input library formed protein/DNA complexes (typically 5-12 rounds). For sequence determination, amplified DNA was cloned without restriction digest into pCR2.1-TOPO (Invitrogen) by topoisomerase-mediated ligation. Data for the 6-finger E2C(S) protein are a composite of two sets of oligonucleotides, one in which the first 9-bp (Half-Site 1, HS1) of the target site was fixed (12 bp randomized) and another in which HS2 was fixed (12 bp randomized). Data for the 6-finger Aart(S) protein are from one oligonucleotide pool with 21 bp randomized. Data for all 3-finger proteins were based on an oligonucleotide pool with 10 bp randomized.

Results and Discussion

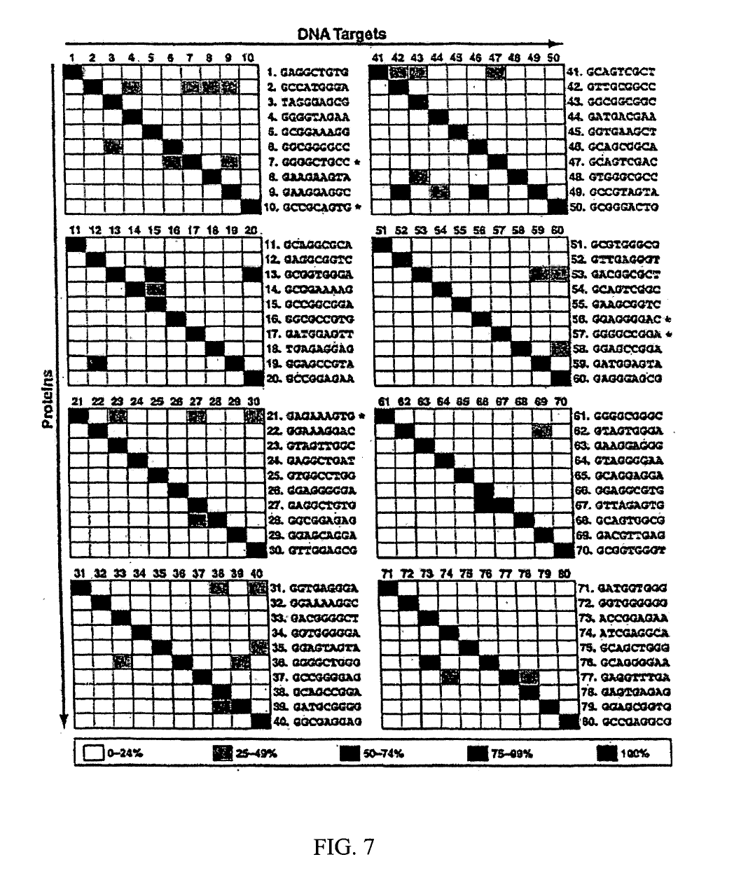

Multi-target ELISA specificity assays. To assess the validity of this modular approach, a cursory analysis on a large sample of proteins was first performed. Eighty 3-finger proteins were chosen randomly from the hundreds of multi-finger proteins previously assembled. The proteins contained domains recognizing not only 5′-GNN-3′ type sequences but also 5′-ANN-3′ and 5′-TNN-3′ sequences. As a reference, the protein Zif268 was also included (FIG. 7, #51). They were divided into eight sets of 10 proteins, and their relative affinity for the 10 DNA-target sites in their set was measured in a multi-target ELISA assay (FIG. 7). The intention was to determine the extent to which proteins generated by the modular approach could bind their cognate (intended) target, and to assess the specificity of that interaction.

FIG. 7 shows the specificity of 80 proteins based on the multi-target ELISA assay. Eight sets of ten 3-finger proteins were tested for binding to ten DNA targets. The numbered list to the right of each set corresponds to both the intended recognition sequence of the proteins and the sequences of the DNA targets. Proteins used for CAST analysis are indicated by an asterisk (*). The maximum binding signal for each protein was normalized to be 100%. Shading indicates the normalized signal intensity according to the scale at the bottom. Experiments were performed in duplicates. The standard deviation of the measurements was typically less than 25% (not shown).

The primary result was that all of the 80 proteins tested were able to bind their cognate target DNA. Most proteins also displayed excellent specificity for their cognate target, with little or no affinity for any of the other targets in the set. In only 5 cases (proteins 13, 19, 49, 67, and 76) did a protein bind a non-cognate target with an affinity at or above 75% of the maximum binding signal. Protein 13 actually preferred binding targets 15 and 20 over its cognate target. There is no obvious explanation for why the 5 proteins showed increased affinity for some of the non-cognate targets. An alignment of the bound cognate and non-cognate target sites (not shown) often revealed a match of 5-6 bp between the 9 bp sites. However, such matches also exist between other targets for which there was no cross-reaction. More to the point, none of the proteins corresponding to the bound, non-cognate targets cross-reacted with any other target in the set (that is, protein 76 bound target 73, but protein 73 did not bind target 76 nor any other non-cognate target). From this it can be concluded that the observed promiscuity is a property of these particular proteins and not related to general factors such as the number of matches (within limits) or the number of guanines in the target sequences.

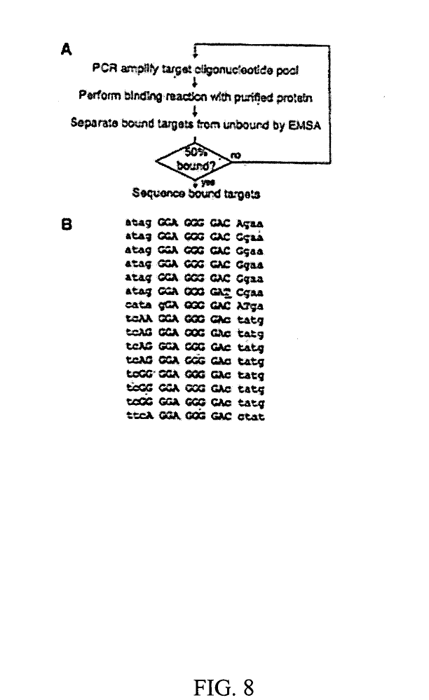

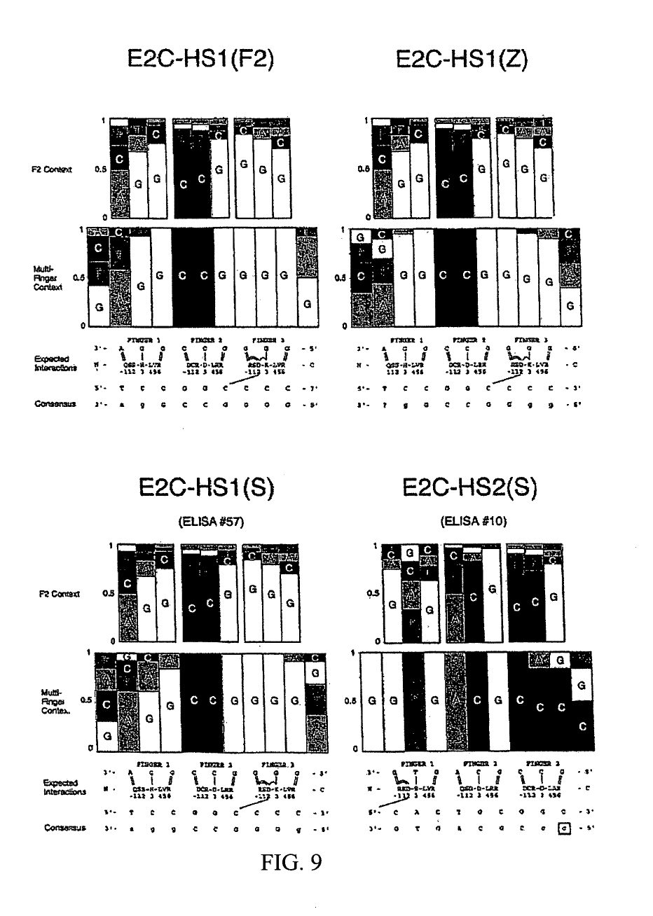

Target site selection experiments. The multi-target ELISA specificity study found only 5 of 80 proteins (6.25%) to have extraordinary promiscuity, and only one (1.25%) to have inappropriate specificity. Although these results suggest that more than 90% of proteins created by the modular approach bind their cognate target with very high specificity, it should be noted that the 10 DNA targets in each set represent only 0.003% of all possible 9-bp targets. To provide a more detailed analysis of binding specificity, a Cyclical Amplification and Selection of Targets (CAST) assay was performed (31). CAST is a common and accurate method for determining the preferred binding site(s) for DNA-binding proteins, and has been used to examine the specificity of naturally occurring zinc finger proteins such as Zif268 (32) and Sp1 (33-35), as well as several created by selection or design (36-40). In the current study, a cycle commenced with an in vitro binding reaction containing purified protein and a pool of randomized DNA targets (see Methods and Materials and FIG. 5A). The bound targets were separated from unbound by a gel electrophoresis mobility shift assay (EMSA). The DNA targets had been designed with primer sites flanking the randomized region, therefore allowing the bound targets to be amplified by PCR and used as input in subsequent cycles. CAST was performed for 5-12 cycles until 50% of the input DNA formed DNA/protein complexes, after which members of the pool were sequenced (as an example, FIG. 8B). In general, the quality of the data improved only slightly with more rounds (data not shown).

FIG. 8 shows an overview of the CAST assay. (A) A flow diagram describing the steps of the CAST assay, (B) Raw data from the CAST analysis of B3-HS2(S). Randomized regions are in capital letters, flanking regions are in lower case. Nucleotides not matching the expected target site are underlined.

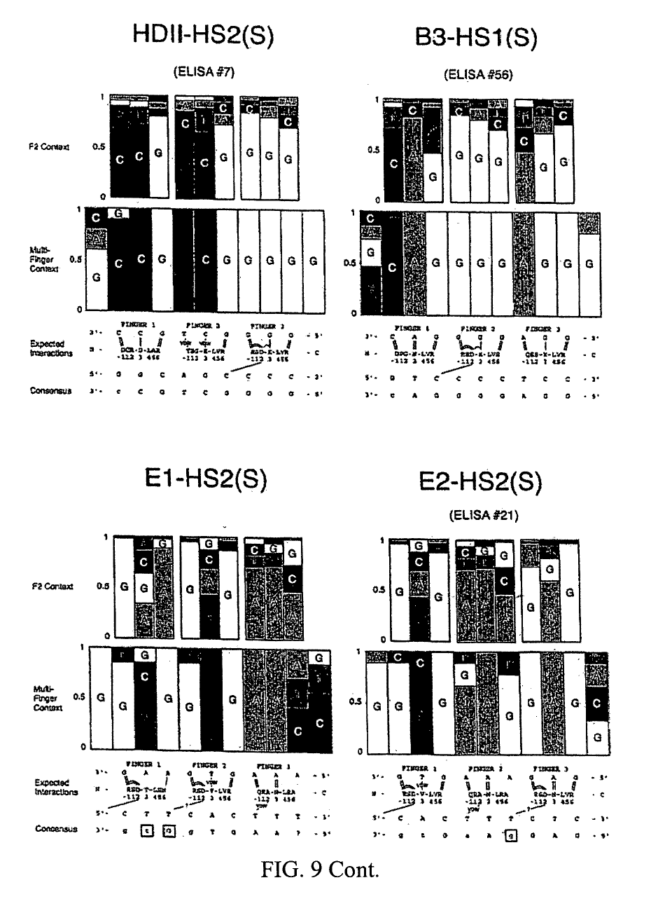

FIG. 9 shows results of the CAST assay. The name of the protein and a cross-reference (if available) to its position in the results of the multi-target ELISA specificity assay (FIG. 7) are shown above each graph. Below the titles are bar graphs showing recalculated specificity data previously determined (17-19) when the domains were initially developed as finger 2 in a 3-finger protein (F2 context). The bars are shaded by nucleotide; their height represents the frequency with which each nucleotide was selected. Below the F2-context graphs are the CAST data of the domains assembled in multi-finger proteins. Below this are the protein sequences, DNA target sequences, and expected interactions. Amino acids are numbered with respect to their position in the α-helix. The interactions are based on previous computer models and analysis (17, 18). Lines indicate expected hydrogen bonds. “VDW” indicates expected van der Waals interactions. “?” indicates an interaction that could potentially be destabilizing. The three asterisks next to nucleotides in the E2C(S) interactions indicate the positions that differ between the E2C and E3 binding sites (4). The consensus DNA-binding site is shown at the bottom. Capital letters indicate 100% conservation, lower case letters indicate 50-99% conservation, and a question mark indicates less than 50% conservation. Boxes denote disagreement between the expected and observed nucleotides.

CAST data were collected for 10 proteins eight 3-finger and two 6-finger proteins (FIG. 9). The 6-finger protein E2C was assayed, as were the two 3-finger proteins used to construct it, E2C-HS1 and E2C-HS2 (20). For E2C-HS1, F2-, Zif- and Sp1C-framework versions were analyzed (designated E2C-HS1 (F2), (Z) and (S), respectively, in FIG. 4). For all other proteins, only the Sp1C-backbone was used. The 6-finger Aart protein, composed of domains recognizing 5′-ANN-3′ and 5′-TNN-3′ type sequences (17), was also assayed. Although this protein had an affinity of 7.5 pM, its component 3-finger proteins had affinities below detection and were not analyzed. The remaining 3-finger proteins provide additional examples of domains that recognize 5′-GNN-3′ and 5′-ANN-3′ type sequences. Some domains appear in two or more proteins in different positions and contexts (i.e., different neighboring domains and DNA sequences).

General aspects of specificity. Overall, the CAST analysis demonstrates that the modular approach can create proteins that bind with excellent specificity (FIG. 9). This more detailed analysis fully supports conclusions of the broad-based multi-target ELISA study (FIG. 7). The specificity of the 3-finger proteins tested here is as good or better than that of proteins produced by other methods such as sequential selection (39), bipartite library selection (29), zinc finger recognition codes (36, 37, 41), or other combinations of rational design and selection approaches (40). Specificity degenerates most frequently at the ends of the protein, consistent with observations by others (42). This is likely due to “breathing” between the terminal DNA-contacting residues and the ends of the oligonucleotide target. In some cases, such as HDII-HS2(S) and B3-HS1(S), only a single, terminal nucleotide was incorrectly specified in just one of the 10 or 15 target sequences recovered from CAST.

Other proteins displayed varying degrees of specificity. Examples can be found of poor specificity, non-specificity, and even inappropriate specificity (denoted in the Consensus sequence as lowercase letters, question marks, and boxes, respectively). In most cases the observed specificity can be understood in terms of the expected interactions (or lack of interaction) combined with a dominating target site overlap effect. Several exceptions are discussed below.

Target site overlap. Structural and biochemical analysis of the protein Zif268 found that aspartate in position 2 (Asp²) of one α-helix can hydrogen bond to a nucleotide on the less-heavily contacted strand in the binding site of a neighboring domain (14, 23, 26). The hydrogen bond required an extracyclic amine group on the contacted nucleotide (either C or A), thereby influencing the 5′ nucleotide in the neighboring site to be G or T. This type of phenomenon, known as target site overlap, has led to the suggestion that zinc finger domains may more generally recognize a four bp site. Indeed, recent structural data demonstrate that some domains in canonical, Zif-backbone proteins can recognize a four or even five bp site (25). The implications suggest dire consequences for a modular approach based on a three bp site.

The CAST data generally support target site overlap by Asp². When Asp² occurs in the finger 1 position, as in E2C-HS2(S), E1-HS2(S) and E2-HS2(S), the neighboring nucleotide is specified as G. Interestingly, T was not specified. The overlap effect is less dramatic for the 6-finger proteins, but that may be due to increased “breathing” at the ends of the longer protein. Internally, the effects of Asp² can be seen in cases where the neighboring domain does a poor job of specifying its 5′ nucleotide. For example, Ala⁶ in finger 2 of E2-HS2(S) was not expected to contact its 5′ nucleotide (17). Asp² in finger 3 specifies the nucleotide to be G or T. This domain previously demonstrated cross-reactivity to 5′ G (17), and the additional contact in the current context further enforces the cross-reaction. Similarly, Asn⁶ in finger 1 of E1-HS2(S) was expected to contact N7 of either A or C (17). Asp² in finger 2 ensures specificity of G. The interactions in the 6-finger Aart(S) are less clear. Asp² in finger 6 seems to specify G or T in the finger 5 subsite, but the effect of Asp² in finger 5 is more ambiguous.

CAST data did not reveal strong evidence for target site overlap by an amino acid in position 2 other than Asp². Ser² (in finger 1 of the three E2C-HS1 proteins studied) and Gly² in finger 1 of B3-HS1(S)) do not specify any particular neighboring nucleotide G is partially specified as the neighboring nucleotide when Arg² appears in finger 1 of HDII-HS2(S); however, the neighboring nucleotide is mis-specified as A when Arg² appears in finger 3 of E2C(S). Similarly, A is strongly specified as the neighboring nucleotide when Ala² appears in finger 4 of Aart(S); however, the neighboring nucleotide is mis-specified as G when Ala² appears in finger 3 of Aart(S). Lys² in finger 2 of Aart(S) could potentially be responsible for the partial mis-specification of a neighboring C, but that would require further investigation.

These results are consistent with other CAST studies. Ser² in finger 1 of the protein Sp1 failed to specify a neighboring nucleotide (33, 34). Ser², which is present in 50% of all known zinc finger domains, has been shown to interact with all four nucleotides at the overlap position (43). A weak selection for C as the neighboring nucleotide was observed with His² in finger 1 of the sequentially-selected protein NRE_{ZF}, but this preference was diminished when His² appeared in finger 1 of p53_{ZF} (39). Thr² failed to specify a neighboring nucleotide in TATA_{ZF} (39). Unlike Asp² in E1-HS2(S) of this study, Ala² did not dominate the neighboring Gln⁻¹ recognition of 5′A in the code-derived protein Sint1 (36).

However, target site overlap is not only a consequence of the residue in position 2. Recent structural data suggest that the amino acid in position 1 can participate under some circumstances (25). In particular, Leu¹ in finger 3 of the sequentially-selected TATA_{ZF} was shown to interact with nucleotides on the opposite strand within the finger 2 triplet. Finger 2 contained an Ala⁶, which did not contact any base in the structure (as expected) and therefore could not contribute to specificity. However, CAST analysis of this protein showed strong selection for a 5′ A in the finger 2 triplet, suggesting that the Leu¹ interactions from finger 3 were indeed specifying the base. It is intriguing to note that a similar situation exists in the case of finger 3 of Aart(S). Ala⁶ of this domain is not expected to specify a 5′ nucleotide, and in fact none is specified when the domain appears as finger 3 of E2-HS2(S). However, 5′ A is strongly specified in the finger 3 triplet of Aart(S). Finger 4 of Aart(S) contains a Leu¹, which, by analogy to TATA_{ZF}, is likely to be responsible for the observed specificity. The caveat is that the two Leu¹-containing domains were created in different contexts. The entire recognition helix of finger 3 of TATA_{ZF} was selected in a finger 3 context with A as the neighboring nucleotide, while finger 4 of Aart(S) was originally selected in a finger 2 context with G as the neighboring nucleotide. It is not clear how a Leu selected in the latter context can so strongly specify A in the current context. Therefore, further studies will be required to determine if Leu or any other residue in position 1 is involved in a target site overlap interaction in the proteins described here.

As a whole, these results suggest that only target site overlap by Asp² presents an obstacle for modular construction. Asp² can not be simply replaced in these domains. Aside from its undesired participation in target site overlap, Asp² forms buttressing contacts with Arg⁻¹ that are thought to stabilize its orientation with respect to the DNA. Domains containing Arg⁻¹ without Asp² display severely impaired specificity (18). However, it should be emphasized that Asp² appears in only ¼ of all modular domains (those recognizing 5′-NNG-3′ sequences), and that complications are anticipated only when the neighboring nucleotide is A or C.

It should be noted that another recent study arrived at a contradictory conclusion, reporting biochemical evidence that Ser² is involved in target site overlap interactions (44). A potential explanation for this discrepancy may lie in the fact that the recognition helices examined here were displayed on the structurally regular Sp1C framework, while the other study investigated helices on finger 1 of the wild type Sp1 framework, which is known to interact with DNA differently than fingers 2 and 3. The structural differences underlying the two sets of observations would be insightful and deserve further study.

It is also interesting to note that in some instances a form of target site overlap appears to apply in the reverse direction. In particular, C was strongly specified 5′ to finger 3 of HDII-HS2(S) and finger 3 of B3-HS1(S). A similar interaction was described in the structure of the first three fingers of TFIIIA, in which a G 5′ to the finger 3-triplet is specified by an Arg in position 10 of the finger 3-helix (27). In these proteins, the residue at position 10 is always Thr, but at position 9 it is Arg. The C-terminal portion of the helix in finger 3 of TFIIIA is α-helical in nature, whereas this region in finger 3 of Zif268, Sp1, and these proteins is more likely to form a more compact 3₁₀ helix (45). It is therefore possible that Arg⁹ in the proteins could participate in a reverse target site overlap interaction to specify C. However, such a contact has not been reported in structural studies of Zif268 (13, 14) or Sp1 (46), and none of the other proteins in the current study exhibit this behavior. Another explanation is that Arg⁶, unsupported by an Asp²-type buttressing interaction, could be free to interact with nucleotides 5′ to the binding site. However, Arg⁶ also failed to specify a neighboring nucleotide in any other protein in the current study, and in E2C(S) there seems to be a weak preference for C. Two studies by other groups found that T was strongly selected as the 5′ neighbor to the finger 3 triplet of Zif268 (32, 39). Finger 3 of this protein contains Arg⁶ and Lys⁹. In the protein NRE_{ZF}, there is a preference for G or A as the 5′ neighbor to the finger 3 triplet (finger 3 contains Ala⁶ and Lys⁹), and in p53_{ZF} there is a weak preference for C (finger 3 contains Gln⁶ and Lys⁹) (39). CAST analysis of Sp1 has produced contradictory results on this issue (34, 35). It is also possible that the nucleotides are conserved due to structural features of the DNA rather than a reverse target site overlap interaction from the protein. The basis for the apparent specificity remains unclear.

These studies further highlight the need for both structural and biochemical studies. Explanations for observed biochemical effects are weak without structural data, but structural studies alone are equally insufficient. For example, many structural studies have shown base contacts by Ser², but biochemical studies such as this one demonstrate that these contacts are not determinants of specificity. Claims that zinc finger domains specify a four bp, overlapping subsite have been largely exaggerated, due primarily to over-interpretation of too little or only one type of data.

Specificity as modular units. In general, the domains studied here maintained their original high specificity when placed in different positions in a new protein. The specificity data determined when the domain was created as finger 2 of a 3-finger protein (“F2 context” bar graphs in FIG. 9) are excellent predictors of the specificity observed when that domain appears in a new polydactyl protein (“Multi-finger context” bar graphs). In several cases, the specificity in the new context was actually better, such as for the 5′-GTG-3′-recognition domains in finger 1 of E2C-HS2(S) and finger 2 of E1-HS2(S), the 5′-GGA-3′-recognition domain in finger 4 of E2C(S), and the 5′-ATG-3′-recognition domain in finger 6 of Aart(S). An interesting case where the specificity seems dependent on context is the 5′-GCC-3′-recognition domain. When this domain appears in finger 2 of E2C-HS1(S) it has perfect specificity, as it did in the original F2 context. In both cases a target site overlap interaction aids, perhaps, in the specification of a 5′ G. When the domain appears in finger 3 of E2C-HS2(S), the specificity changed to 5′-CCC-3′. There is no target site overlap to aid the specification of 5′ G. However, structurally it is not clear why this would be necessary. There is also no expected target site overlap when the same domain appears in finger 3 of E2C(S), yet the specificity for 5′ G has been restored. Finally, the domain which had perfect specificity as finger 2 of the 3-finger E2C-HS1(S) has rather poor specificity as finger 5 of the 6-finger E2C(S). The structural basis for these observations is unclear. Possible explanations include context-dependent reorientation of the α-helix or increased sensitivity to differences in local DNA structure.

Another recent study involving analysis of zinc finger domains derived from rational design and selection, similar in many cases to those described here, also reported exceptionally specific recognition based on CAST analysis (40). The similarity of the domains used suggests that CAST analysis may generally produce a “cleaner” specificity profile than non-iterative techniques such as the multi-target ELISA assay used in earlier work (17-19). This caveat should be considered when interpreting the results from all such studies More importantly, the other study demonstrated a clear positional dependence for many of the domains, a result in contrast to the findings reported here. However, the positional effects seemed to be restricted exclusively to finger 1 of their 3-finger constructs, which again may be a consequence of using a wild-type Sp1 framework. As noted above, finger 1 of Sp1 is known to interact with DNA differently than fingers 2 and 3. The resolution of this issue has important implications for the application of modular assembly and deserves further investigation.

5′-ANN-3′-recognition domains also maintained their original specificity well, but their performance was somewhat obscured by the fact that recognition of 5′ A is much less robust than for 5′ G. None of the various interactions that emerged from the previous study (17), small hydrophobics, Glu⁶, Gln⁶, or Arg⁶, were able to stringently specify 5′ A in the current study. Consequently, specificity of this nucleotide can often be dominated by target site overlap interactions. In the absence of such interactions, results were confusing. Arg⁶, which had been strongly selected to recognize 5′-ACN-3′ type sequences, reverted in finger 2 of Aart(S) to its more traditional role of specifying 5′ G. This came as somewhat of a surprise, since others had shown that the bases of a 5′-ACN-3′ triplet were correctly specified when Arg⁶ appeared in finger 2 of the sequentially-selected p53_{ZF} (39). Gln⁶, which had poor 5′ specificity originally, unexplainably specified 5° C. in finger 1 of Aart(S), while Ala⁶, which also had poor specificity originally, was non-specific in finger 3 of E1-HS2(S). However, more interesting than the failures are examples in fingers 3, 4, and 6 of Aart(S) where 5′ A was correctly specified. In all three cases the position 6 residue was a small hydrophobic amino acid, which by computer modeling and structural analysis should be too far away from the DNA to influence specificity (13, 17). Correct specification of 5′ A in the finger 3 triplet may be due to a target site overlap interaction as mentioned earlier. In the case of finger 4, 5′ A was partially specified in spite of a target site overlap interaction from finger 5 that was expected to specify either G or T. 5′ A was strongly specified in the finger 6 triplet in the absence of any potential target site overlap. It is therefore not at all clear what structural features are responsible for the observed specificity. Structural analysis is indicated.

Framework effects and higher order proteins. The specificity of protein E2C-HS1 changed very little as the backbone was changed from F2, to Zif to Sp1C. A much more dramatic change occurred when E2C-HS1(S) and E2C-HS2(S) were linked together as E2C(S). In particular, it is not clear why fingers 1 and 2, which displayed perfect specificity in E2C-HS2(S), displayed diminished specificity in E2C(S). E2C-HS2(S) and fingers 1-3 of E2C(S) are the same, thus ruling out influences from neighboring domains or differences in local DNA structure. One explanation is that the increased number of contacts in the 6-finger protein elevates the binding energy to a point where individual residue:base mismatches are insufficient to prevent binding. Alternatively, the fact that so many contacts are made to one strand of the DNA may “pull” the protein towards that strand and mis-orient some fingers.

A third explanation is that the DNA-contacting residues of the longer protein fail to align properly with the DNA bases. This phenomenon is supported by a growing consensus in the field and is attributed to the use of consensus TGEKP (SEQ ID NO: 674) linkers between the domains. One consequence of the awkward alignment is that the protein exhibits lower affinity because binding energy is consumed contorting the DNA or simply lost due to missing DNA contacts. This concern was originally discussed when the first studies of 6-finger proteins were reported (21). Several subsequent studies have found that using longer linkers in various arrangements can produce proteins of higher affinity (47-49). Another logical consequence of framework-imposed misalignment could be the observed loss in specificity in the E2C(S) protein. However, since this work constitutes the first CAST analysis of a designed 6-finger protein, more research will be required to establish the relationship between framework constraints and specificity.

An interesting question raised by these results is whether the 6-finger proteins in this study can bind to more or less sequences than a 3-finger protein. A site for a 3-finger protein such as E2C-HS2(S), with near perfect specificity for its 9 bp site, should occur every 2.6×10⁵ bp in a genome of random nucleotides ([4×{1=the frequency of consensus nucleotide}]⁹), or around 13,000 times in the human genome (3.5×10⁹ bp). In theory, an 18-bp site should occur once every 6.9×10¹⁰ bp ([4×{1}]¹⁸), meaning it would be unique in the human genome. However, the degenerate specificity of E2C(S) would lower this number to around one every 5.3×10⁷ bp (4¹⁸×{0.57×0.29×0.43×0.43×0.57×0.57×0.71×0.86×0.71×1×1×0.86×0.43×0.57×1×1×1×0.86}) or roughly 66 times in human. A consensus site for Aart(S) would occur around once per 1.2×10⁸ bp (4¹⁸×{0.29×0.36×0.71×0.64×0.86×0.86×0.64×1×0.93×0.93×0.93×0.50×1×0.43×1×0.64×0.70}) or 29 times in human. Therefore, the data support that these 6-finger proteins are still significantly more specific than an ideal 3-finger protein.

It should also be emphasized that the number of available binding sites in the genome will be somewhat lower than the theoretical total because many of the sites will be inaccessible due to structure chromatin. Furthermore, since less than 1% of the human genome is coding region (12), most binding sites will occur in regions that will not affect the regulation of any gene. Previous studies have shown that only proteins that bind their target with an affinity of 10 nM or better are productive regulators. Therefore, even if a protein binds a site in a regulatory region that is related but non-consensus, it may not have sufficient affinity to elicit a biological response.

In another study, it was shown that E2C(S) can functionally discriminate in vivo at the level of endogenous gene regulation between its 18-bp cognate site in erbB-2 and another site, E3 in erbB-3, containing only three bp mismatches (4). In vitro, these three mismatches resulted in a 15-fold loss in affinity. The position of the mismatches are marked with asterisks on the expected interactions line of the E2C(S) CAST data (FIG. 9). The discrimination can be rationalized in light of the CAST results; all mismatches correspond to nucleotides that are more than 50% conserved, one is 100% conserved. However, the CAST data also suggest that mismatches in other positions would affect specificity differently.

Zinc finger domains are the largest single class of domain fold found in the human genome (over 4,500 examples identified), comprise the most common type of DNA-binding motif found in eukaryotes, and represent the best characterized and simplest DNA-binding fold. Although there is considerable heterogeneity in the way naturally-occurring zinc finger domains interact with DNA, many domains have been shown to interact in a manner similar to those used in this study. Therefore, the detailed analysis of these modified proteins should also contribute to understanding of how this most important class of natural proteins recognizes DNA.

In conclusion, vast arrays of 3-finger proteins can be rapidly and reliably assembled from pre-determined domains originally constructed in a F2-context. The 3-finger proteins constructed using this methodology generally recapitulate the specificity observed for each constituent domain. The robust domain specificity observed within 3-finger proteins weakens somewhat when two 3-finger proteins are directly linked. Even with some losses in domain specificity, the genomic targeting potential of 6-finger proteins is greatly improved over 3-finger proteins. The relationship between the longer proteins and specificity deserves further investigation. Since the loss of specificity clearly does not correlate with the original F2-context specificity of individual domains nor with the specificity of constituent 3-finger proteins, a higher order phenomenon must be responsible. Until better insight is obtained, the ability to predict in detail the specificity and affinity of 6-domain zinc finger proteins is limited. There would be cause for optimism if this framework explanation were proven true, for that would imply that specificity could be improved through further protein engineering. The alternative would be to accept that affinity and specificity are often opposing forces, and that one comes often comes at the expense of the other.

### REFERENCES

The following references are for Example 2 only:
1. Beerli, R. R. & Barbas III, C. F. (2002). Nat. Biotech. 20, 135-141.
2. Segal, D. & Barbas III, C. F. (2001). Curr. Opin. Biotech. 12, 632-637.
3. Segal, D. J. & Barbas III, C. F. (2000). Curr. Opin. Chem. Biol. 4, 34-39.
4. Beerli, R. R., Dreier, B. & Barbas III, C. F. (2000). Proc. Natl. Acad. Sci. USA 97, 1495-1500.
5. Liu, P. Q., Rebar, E. J., Zhang, L., Liu, Q., Jamieson, A. C., Liang, Y., Qi, H., Li, P. X., Chen, B., Mendel, M. C., et al. (2001). J. Biol. Chem. 276, 11323-11334.
6. Zhang, L., Spratt, S. K., Liu, Q., Johnstone, B., Qi, H., Raschke, E. E., Jamieson, A. C., Rebar, E. J., Wolffe, A. P. & Case, C. C. (2000). J. Biol. Chem. 275, 33850-33860.
7. Guan, X., Stege, J., Kim, M., Dahmani, Z., Fan, N., Heifetz, P., Barbas, C. F., III, and Briggs, S. P. (2002) Proc. Natl. Acad. Sci. USA 99, 13296-13301.
8. Ordiz, M. I., Barbas, C. F., III, and Beachy, R. N. (2002) Proc. Natl. Acad. Sci. USA 99, 13290-13295.
9. Xu, L., Zerby, D., Huang, Y., Ji, H., Nyanguile, O. F., de los Angeles, J. E. & Kadan, M. J. (2001). Mol. Ther. 3, 262-273.
10. Bibikova, M., Golic, M., Golic, K. G., and Carroll, D. (2002) Genetics 161, 1169-75.
11. Holmes-Son, M. L., Appa, R. S. & Chow, S. A. (2001). Adv. Genet. 43, 33-69.
12. Venter, J. C., Adams, M. D., Myers, E. W., Li, P. W., Mural, R. J., Sutton, G. G., Smith, H. O., Yandell, M., Evans, C. A., Holt, R. A., et al. (2001). Science 291, 1304-1351.
13. Pabo, C. O. & Nekludova, L. (2000). J. Mol. Biol. 301, 597-624.
14. Elrod-Erickson, M., Rould, M. A., Nekludova, L. & Pabo, C. O. (1996). Structure 4, 1171-1180.
15. Pavletich, N. P. & Pabo, C. O. (1991). Science 252, 809-817.
16. Narayan, V. A., Kriwacki, R. W. & Caradonna, J. P. (1997). J. Biol. Chem. 272, 7801-7809.
17. Dreier, B., Beerli, R. R., Segal, D. J., Flippin, J. D. & Barbas III, C. F. (2001). J. Biol. Chem. 276, 29466-29478.
18. Dreier, B., Segal, D. J. & Barbas III, C. F. (2000). J. Mol. Biol. 303, 489-502.
19. Segal, D. J., Dreier, B., Beerli, R. R. & Barbas III, C. F. (1999). Proc. Natl. Acad. Sci. USA 96, 2758-2763.
20. Beerli, R. R., Segal, D. J., Dreier, B. & Barbas III, C. F. (1998). Proc. Natl. Acad. Sci. USA 95, 14628-14633.
21. Liu, Q., Segal, D. J., Ghiara, J. B. & Barbas III, C. F. (1997). Proc. Natl. Acad. Sci. USA 94, 5525-5530.
22. Desjarlais, J. R. & Berg, J. M. (1993) Proc. Natl. Acad. Sci. USA 90, 2256-2260.
23. Isalan, M., Choo, Y. & Klug, A. (1997). Proc. Natl. Acad. Sci. USA 94, 5617-5621.
24. Jamieson, A. C., Wang, H. & Kim, S.-H. (1996). Proc. Natl. Acad. Sci. USA 93, 12834-12839.
25. Wolfe, S. A., Grant, R. A., Elrod-Erickson, M. & Pabo, C. O. (2001). Structure 9, 717-723.
26. Pabo, C. O., Peisach, E. & Grant, R. A. (2001). Annu. Rev. Biochem. 70, 313-340.
27. Wuttke, D. S., Foster, M. P., Case, D. A., Gottesfeld, J. M. & Wright, P. E. (1997). J. Mol. Biol. 273, 183-206.
28. Jamieson, A. C., Kim, S.-H. & Wells, J. A. (1994). Biochemistry 33, 5689-5695.
29. Isalan, M., Klug, A. & Choo, Y. (2001). Nat. Biotechnol. 19, 656-660.
30. Greisman, H. A. & Pabo, C. O. (1997). Science 275, 657-661.
31. Wright, W. E., Binder, M. & Funk, W. (1991). Mol. Cell. Biol 11, 4104-4110.
32. Swimoff, A. H. & Milbrandt, J. (1995). Mol. Cell. Biol. 15, 2275-2287.
33. Thiesen, H. J. & Bach, C. (1990). Nucleic Acids Res. 18, 3203-3209.
34. Shi, Y. & Berg, J. M. (1995). Chem. Biol 2, 83-89.
35. Nagaoka, N., Shiraishi, Y. & Sugiura, Y. (2001) Nucleic Acids Res. 29, 4920-4929.
36. Corbi, N., Libri, V., Fanciulli, M. & Passananti, C. (1998). Biochem. Biophys. Res. Commun. 253, 686-692.
37. Corbi, N., Perez, M., Maione; R. & Passananti, C. (1997). FEBS Lett. 417, 71-74.
38. Desjarlais, J. R. & Berg, J. M. (1992). Proteins: Struct., Funct., Genet. 12, 101-104.
39. Wolfe, S. A., Greisman, H. A., Ramm, E. I. & Pabo, C. O. (1999). J. Mol. Biol. 285, 1917-1934.
40. Liu, Q., Xia, Z., Zhong, X. & Case, C. C. (2002) J. Biol. Chem. 277, 3850-3856.
41. Corbi, N., Libri, V., Fanciulli, M., Tinsley, J. M., Davies, K. E. & Passananti, C. (2000). Gene Ther. 7, 1076-1083.
42. Choo, Y. (1998). Nucleic Acids Res. 26, 554-557.
43. Kim, C. A. & Berg, J. M. (1995). J. Mol. Biol 252, 1-5.
44. Nagaoka, M., Shiraishi, Y., Uno, Y., Nomura, W. & Sugiura, Y. (2002) Biochemistry 41, 8819-8825.
45. Laity, J. H., Dyson, H. J. & Wright, P. E. (2000). J. Mol. Biol. 295, 719-727.
46. Kim, C. A. & Berg, J. M. (1996). Nat. Struct. Biol. 3, 940-945.
47. Kim, J. S. & Pabo, C. O. (1998). Proc. Natl. Acad. Sci. USA 95, 2812-2817.
48. Moore, M., Klug, A. & Choo, Y. (2001). Proc. Natl. Acad. Sci. USA 98, 1437-1441.
49. Nagaoka, M., Nomura, W., Shiraishi, Y. & Sugiura, Y. (2001). Biochem. Biophys. Res. Commun. 282, 1001-1007.

All sequences recited herein in the specification and/or the drawings are included in Table 3. These sequences are included in the Sequence Listing but are also included here for convenience.

TABLE 3
SEQUENCES INCLUDED IN SEQUENCE LISTING
Zinc Finger Modules

ANN-specific

| | |
|---|---|
| STNTKLHA | (SEQ ID NO: 1) |
| SSDRTLRR | (SEQ ID NO: 2) |
| STKERLKT | (SEQ ID NO: 3) |
| SQRANLRA | (SEQ ID NO: 4) |
| SSPADLTR | (SEQ ID NO: 5) |
| SSHSDLVR | (SEQ ID NO: 6) |
| SNGGELIR | (SEQ ID NO: 7) |
| SNQLILLK | (SEQ ID NO: 8) |
| SSRMDLKR | (SEQ ID NO: 9) |
| SRSDHLTN | (SEQ ID NO: 10) |
| SQLAHLRA | (SEQ ID NO: 11) |
| SQASSLKA | (SEQ ID NO: 12) |
| SQKSSLIA | (SEQ ID NO: 13) |
| SRKDNLKN | (SEQ ID NO: 14) |
| SDSGNLRV | (SEQ ID NO: 15) |
| SDRRNLRR | (SEQ ID NO: 16) |
| SDKKDLSR | (SEQ ID NO: 17) |
| SDASHLHT | (SEQ ID NO: 18) |
| STNSGLKN | (SEQ ID NO: 19) |
| STRMSLST | (SEQ ID NO: 20) |
| SNHDALRA | (SEQ ID NO: 21) |
| SRRSACRR | (SEQ ID NO: 22) |
| SRRSSCRK | (SEQ ID NO: 23) |
| SRSDTLSN | (SEQ ID NO: 24) |
| SRMGNLIR | (SEQ ID NO: 25) |
| SRSDTLRD | (SEQ ID NO:26) |
| SRAHDLVR | (SEQ ID NO: 27) |
| SRSDHLAE | (SEQ ID NO: 28) |
| SRRDALNV | (SEQ ID NO: 29) |
| STTGNLTV | (SEQ ID NO: 30) |
| STSGNLLV | (SEQ ID NO: 31) |
| STLTILKN | (SEQ ID NO: 32) |
| SRMSTLRH | (SEQ ID NO: 33) |
| STRSDLLR | (SEQ ID NO: 34) |
| STKTDLKR | (SEQ ID NO: 35) |
| STHIDLIR | (SEQ ID NO: 36) |
| SHRSTLLN | (SEQ ID NO: 37) |
| STSHGLTT | (SEQ ID NO: 38) |
| SHKNALQN | (SEQ ID NO: 39) |
| QRANLRA | (SEQ ID NO: 40) |
| DSGNLRV | (SEQ ID NO: 41) |
| RSDTLSN | (SEQ ID NO: 42) |
| TTGNLTV | (SEQ ID NO: 43) |
| SPADLTR | (SEQ ID NO: 44) |
| DKKDLTR | (SEQ ID NO: 45) |
| RTDTLRD | (SEQ ID NO: 46) |
| THLDLIR | (SEQ ID NO: 47) |
| QLAHLRA | (SEQ ID NO: 48) |
| RSDHLAE | (SEQ ID NO: 49) |
| HRTTLLN | (SEQ ID NO: 50) |
| QKSSLIA | (SEQ ID NO: 51) |
| RRDALNV | (SEQ ID NO: 52) |
| HKNALQN | (SEQ ID NO: 53) |
| RSDNLSN | (SEQ ID NO: 54) |
| RKDNLKN | (SEQ ID NO: 55) |
| TSGNLLV | (SEQ ID NO: 56) |
| RSDHLTN | (SEQ ID NO: 57) |
| HRTTLTN | (SEQ ID NO: 58) |
| SHSDLVR | (SEQ ID NO: 59) |
| NGGELIR | (SEQ ID NO: 60) |
| STKDLKR | (SEQ ID NO: 61) |
| RRDELNV | (SEQ ID NO: 62) |
| QASSLKA | (SEQ ID NO: 63) |
| TSHGLTT | (SEQ ID NO: 64) |
| QSSHLVR | (SEQ ID NO: 65) |
| QSSNLVR | (SEQ ID NO: 66) |
| DPGALRV | (SEQ ID NO: 67) |
| RSDNLVR | (SEQ ID NO: 68) |
| QSGDLRR | (SEQ ID NO: 69) |
| DCRDLAR | (SEQ ID NO: 70) |
| AGC-Specific | |
| DPGALIN | (SEQ ID NO: 71) |
| ERSHLRE | (SEQ ID NO: 72) |
| DPGHLTE | (SEQ ID NO: 73) |
| EPGALIN | (SEQ ID NO: 74) |
| DRSHLRE | (SEQ ID NO: 75) |
| EPGHLTE | (SEQ ID NO: 76) |
| ERSLLRE | (SEQ ID NO: 77) |
| DRSKLRE | (SEQ ID NO: 78) |
| DPGKLTE | (SEQ ID NO: 79) |
| EPGKLTE | (SEQ ID NO: 80) |
| DPGWLIN | (SEQ ID NO: 81) |
| DPGTLIN | (SEQ ID NO: 82) |
| DPGHLIN | (SEQ ID NO: 83) |
| ERSWLIN | (SEQ ID NO: 84) |
| ERSTLIN | (SEQ ID NO: 85) |
| DPGWLTE | (SEQ ID NO: 86) |
| DPGTLTE | (SEQ ID NO: 87) |
| EPGWLIN | (SEQ ID NO: 88) |
| EPGTLIN | (SEQ ID NO: 89) |
| EPGHLIN | (SEQ ID NO: 90) |
| DRSWLRE | (SEQ ID NO: 91) |
| DRSTLRE | (SEQ ID NO: 92) |
| EPGWLTE | (SEQ ID NO: 93) |
| EPGTLTE | (SEQ ID NO: 94) |
| ERSWLRE | (SEQ ID NO: 95) |
| ERSTLRE | (SEQ ID NO: 96) |
| DPGALRE | (SEQ ID NO: 97) |
| DPGALTE | (SEQ ID NO: 98) |
| ERSHLIN | (SEQ ID NO: 99) |
| ERSHLTE | (SEQ ID NO: 100) |
| DPGHLIN | (SEQ ID NO: 101) |
| DPGHLRE | (SEQ ID NO: 102) |
| EPGALRE | (SEQ ID NO: 103) |
| EPGALTE | (SEQ ID NO: 104) |
| DRSHLIN | (SEQ ID NO: 105) |
| DRSHLTE | (SEQ ID NO: 106) |
| EPGHLRE | (SEQ ID NO: 107) |
| ERSKLIN | (SEQ ID NO: 108) |
| ERSKLTE | (SEQ ID NO: 109) |
| DRSKLIN | (SEQ ID NO: 110) |
| DRSKLTE | (SEQ ID NO: 111) |
| DPGKLIN | (SEQ ID NO: 112) |
| DPGKLRE | (SEQ ID NO: 113) |
| EPGKLIN | (SEQ ID NO: 114) |
| EPGKLRE | (SEQ ID NO: 115) |
| DPGWLRE | (SEQ ID NO: 116) |
| DPGTLRE | (SEQ ID NO: 117) |
| DPGHLRE | (SEQ ID NO: 118) |
| DPGHLTE | (SEQ ID NO: 119) |
| ERSWLTE | (SEQ ID NO: 120) |
| ERSTLTE | (SEQ ID NO: 121) |
| EPGWLRE | (SEQ ID NO: 122) |
| EPGTLRE | (SEQ ID NO: 123) |
| DRSWLIN | (SEQ ID NO: 124) |
| DRSWLTE | (SEQ ID NO: 125) |
| DRSTLIN | (SEQ ID NO: 126) |
| DRSTLTE | (SEQ ID NO: 127) |
| CNN-Specific | |
| QRHNLTE | (SEQ ID NO: 128) |
| QSGNLTE | (SEQ ID NO: 129) |
| NLQHLGE | (SEQ ID No: 130) |
| RADNLTE | (SEQ ID NO: 131) |
| RADNLAI | (SEQ ID NO: 132) |
| NTTHLEH | (SEQ ID NO: 133) |
| SKKHLAE | (SEQ ID NO: 134) |
| RNDTLTE | (SEQ ID NO: 135) |
| RNDTLQA | (SEQ ID NO: 136) |
| QSGHLTE | (SEQ ID NO: 137) |
| QLAHLKE | (SEQ ID NO: 138) |
| QRAHLTE | (SEQ ID NO: 139) |
| HTGHLLE | (SEQ ID NO: 140) |
| RSDHLTE | (SEQ ID NO: 141) |
| RSDKLTE | (SEQ ID NO: 142) |
| RSDHLTD | (SEQ ID NO: 143) |
| RSDHLTN | (SEQ ID NO: 144) |
| SRRTCRA | (SEQ ID NO: 145) |
| QLRHLRE | (SEQ ID NO: 146) |
| QRHSLTE | (SEQ ID NO: 147) |
| QLAHLKR | (SEQ ID NO: 148) |
| NLQHLGE | (SEQ ID NO: 149) |
| RNDALTE | (SEQ ID NO: 150) |
| TKQTLTE | (SEQ ID NO: 151) |
| QSGDLTE | (SEQ ID NO: 152) |
| GNN-Specific | |
| QSSNLVR | (SEQ ID NO: 153) |
| DPGNLVR | (SEQ ID NO: 154) |
| RSDNLVR | (SEQ ID NO: 155) |
| TSGNLVR | (SEQ ID NO: 156) |
| QSGDLRR | (SEQ ID NO: 157) |
| DCRDLAR | (SEQ ID NO: 158) |
| RSDDLVK | (SEQ ID NO: 159) |
| TSGELVR | (SEQ ID NO: 160) |
| QRAHLER | (SEQ ID NO: 161) |
| DPGHLVR | (SEQ ID NO: 162) |
| RSDKLVR | (SEQ ID NO: 163) |
| TSGHLVR | (SEQ ID NO: 164) |
| QSSSLVR | (SEQ ID NO: 165) |
| DPGALVR | (SEQ ID NO: 166) |
| RSDELVR | (SEQ ID NO: 167) |
| TSGSLVR | (SEQ ID NO: 168) |
| QRSNLVR | (SEQ ID NO: 169) |
| QSGNLVR | (SEQ ID NO: 170) |
| QPGNLVR | (SEQ ID NO: 171) |
| DPGNLKR | (SEQ ID NO: 172) |
| RSDNLRR | (SEQ ID NO: 173) |
| KSANLVR | (SEQ ID NO: 174) |
| RSDNLVK | (SEQ ID NO: 175) |
| KSAQLVR | (SEQ ID NO: 176) |
| QSSTLVR | (SEQ ID NO: 177) |
| QSGTLRR | (SEQ ID NO: 178) |
| QPGDLVR | (SEQ ID NO: 179) |
| QGPDLVR | (SEQ ID NO: 180) |
| QAGTLMR | (SEQ ID NO: 181) |
| QPGTLVR | (SEQ ID NO: 182) |
| QGPELVR | (SEQ ID NO: 183) |
| GCRELSR | (SEQ ID NO: 184) |
| DPSTLKR | (SEQ ID NO: 185) |
| DPSDLKR | (SEQ ID NO: 186) |
| DSGDLVR | (SEQ ID NO: 187) |
| DSGELVR | (SEQ ID NO: 188) |
| DSGELKR | (SEQ ID NO: 189) |
| RLDTLGR | (SEQ ID NO: 190) |
| RPGDLVR | (SEQ ID NO: 191) |
| RSDTLVR | (SEQ ID NO: 192) |
| KSADLKR | (SEQ ID NO: 193) |
| RSDDLVR | (SEQ ID NO: 194) |
| RSDTLVK | (SEQ ID NO: 195) |
| KSAELKR | (SEQ ID NO: 196) |
| KSAELVR | (SEQ ID NO: 197) |
| RGPELVR | (SEQ ID NO: 198) |
| KPGELVR | (SEQ ID NO: 199) |
| SSQTLTR | (SEQ ID NO: 200) |
| TPGELVR | (SEQ ID NO: 201) |
| TSGDLVR | (SEQ ID NO: 202) |
| SSQTLVR | (SEQ ID NO: 203) |
| TSQTLTR | (SEQ ID NO: 204) |
| TSGELKR | (SEQ ID NO: 205) |
| QSSDLVR | (SEQ ID NO: 206) |
| SSGTLVR | (SEQ ID NO: 207) |
| TPGTLVR | (SEQ ID NO: 208) |
| TSQDLKR | (SEQ ID NO: 209) |
| TSGTLVR | (SEQ ID NO: 210) |
| QSSHLVR | (SEQ ID NO: 211) |
| QSGHLVR | (SEQ ID NO: 212) |
| QPGHLVR | (SEQ ID NO: 213) |
| ERSKLAR | (SEQ ID NO: 214) |
| DPGHLAR | (SEQ ID NO: 215) |
| QRAKLER | (SEQ ID NO: 216) |
| QSSKLVR | (SEQ ID NO: 217) |
| DRSKLAR | (SEQ ID NO: 218) |
| DPGKLAR | (SEQ ID NO: 219) |
| RSKDLTR | (SEQ ID NO: 220) |
| RSDHLTR | (SEQ ID NO: 221) |
| KSAKLER | (SEQ ID NO: 222) |
| TADHLSR | (SEQ ID NO: 223) |
| TADKLSR | (SEQ ID NO: 224) |
| TPGHLVR | (SEQ ID NO: 225) |
| TSSHLVR | (SEQ ID NO: 226) |
| TSGKLVR | (SEQ ID NO: 227) |
| QPGELVR | (SEQ ID NO: 228) |
| QSGELVR | (SEQ ID NO: 229) |
| QSGELRR | (SEQ ID NO: 230) |
| DPGSLVR | (SEQ ID NO: 231) |
| RKDSLVR | (SEQ ID NO: 232) |
| RSDVLVR | (SEQ ID NO: 233) |
| RHDSLLR | (SEQ ID NO: 234) |
| RSDALVR | (SEQ ID NO: 235) |
| RSSSLVR | (SEQ ID NO: 236) |
| RSSSHVR | (SEQ ID NO: 237) |
| RSDELVK | (SEQ ID NO: 238) |
| RSDALVK | (SEQ ID NO: 239) |
| RSDVLVK | (SEQ ID NO: 240) |
| RSSALVR | (SEQ ID NO: 241) |
| RKDSLVK | (SEQ ID NO: 242) |
| RSASLVR | (SEQ ID NO: 243) |
| RSDSLVR | (SEQ ID NO: 244) |
| RIHSLVR | (SEQ ID NO: 245) |
| RPGSLVR | (SEQ ID NO: 246) |
| RGPSLVR | (SEQ ID NO: 247) |
| RPGALVR | (SEQ ID NO: 248) |
| KSASKVR | (SEQ ID NO: 249) |
| KSAALVR | (SEQ ID NO: 250) |
| KSAVLVR | (SEQ ID NO: 251) |
| TSGSLTR | (SEQ ID NO: 252) |
| TSQSLVR | (SEQ ID NO: 253) |
| TSSSLVR | (SEQ ID NO: 254) |
| TPGSLVR | (SEQ ID NO: 255) |
| TSGALVR | (SEQ ID NO: 256) |
| TPGALVR | (SEQ ID NO: 257) |
| TGGSLVR | (SEQ ID NO: 258) |
| TSGELVR | (SEQ ID NO: 259) |
| TSGELTR | (SEQ ID NO: 260) |
| TSSALVK | (SEQ ID NO: 261) |
| TSSALVR | (SEQ ID NO: 262) |
| TNN-Specific | |
| QASNLIS | (SEQ ID NO: 263) |
| SRGNLKS | (SEQ ID NO: 264) |
| RLDNLQT | (SEQ ID NO: 265) |
| ARGNLRT | (SEQ ID NO: 266) |
| RKDALRG | (SEQ ID NO: 267) |
| REDNLHT | (SEQ ID NO: 268) |
| ARGNLKS | (SEQ ID NO: 269) |
| RSDNLTT | (SEQ ID NO: 270) |
| VRGNLKS | (SEQ ID NO: 271) |
| VRGNLRT | (SEQ ID NO: 272) |
| RLRALDR | (SEQ ID NO: 273) |
| DMGALEA | (SEQ ID NO: 274) |
| EKDALRG | (SEQ ID NO: 275) |
| RSDHLTT | (SEQ ID NO: 276) |
| AQQLLMW | (SEQ ID NO: 277) |
| RSDERKR | (SEQ ID NO: 278) |
| DYQSLRQ | (SEQ ID NO: 279) |
| CFSRLVR | (SEQ ID NO: 280) |
| GDGGLWE | (SEQ ID NO: 281) |
| LQRPLRG | (SEQ ID NO: 282) |
| QGLACAA | (SEQ ID NO: 283) |
| WVGWLGS | (SEQ ID NO: 284) |
| RLRDIQF | (SEQ ID NO: 285) |
| GRSQLSC | (SEQ ID NO: 286) |
| GWQRLLT | (SEQ ID NO: 287) |
| SGRPLAS | (SEQ ID NO: 288) |
| APRLLGP | (SEQ ID NO: 289) |
| APKALGW | (SEQ ID NO: 290) |
| SVHELQG | (SEQ ID NO: 291) |
| AQAALSW | (SEQ ID NO: 292) |
| GANALRR | (SEQ ID NO: 293) |
| QSLLLGA | (SEQ ID NO: 294) |
| HRGTLGG | (SEQ ID NO: 295) |
| QVGLLAR | (SEQ ID NO: 296) |
| GARGLRG | (SEQ ID NO: 297) |
| DKHMLDT | (SEQ ID NO: 298) |
| DLGGLRQ | (SEQ ID NO: 299) |
| QCYRLER | (SEQ ID NO: 300) |
| AEAELQR | (SEQ ID NO: 301) |
| QGGVLAA | (SEQ ID NO: 302) |
| QGRCLVT | (SEQ ID NO: 303) |
| HPEALDN | (SEQ ID NO: 304) |
| GRGALQA | (SEQ ID NO: 305) |
| LASRLQQ | (SEQ ID NO: 306) |
| REDNLIS | (SEQ ID NO: 307) |
| RGGWLQA | (SEQ ID NO: 308) |
| DASNLIS | (SEQ ID NO: 309) |
| EASNLIS | (SEQ ID NO: 310) |
| RASNLIS | (SEQ ID NO: 311) |
| TASNLIS | (SEQ ID NO: 312) |
| SASNLIS | (SEQ ID NO: 313) |
| QASTLIS | (SEQ ID NO: 314) |
| QASDLIS | (SEQ ID NO: 315) |
| QASELIS | (SEQ ID NO: 316) |
| QASHLIS | (SEQ ID NO: 317) |
| QASKLIS | (SEQ ID NO: 318) |
| QASSLIS | (SEQ ID NO: 319) |
| QASALIS | (SEQ ID NO: 320) |
| DASTLIS | (SEQ ID NO: 321) |
| DASDLIS | (SEQ ID NO: 322) |
| DASELIS | (SEQ ID NO: 323) |
| DASHLIS | (SEQ ID NO: 324) |
| DASKLIS | (SEQ ID NO: 325) |
| DASSLIS | (SEQ ID NO: 326) |
| DASALIS | (SEQ ID NO: 327) |
| EASTLIS | (SEQ ID NO: 328) |
| EASDLIS | (SEQ ID NO: 329) |
| EASELIS | (SEQ ID NO: 330) |
| EASHLIS | (SEQ ID NO: 331) |
| EASKLIS | (SEQ ID NO: 332) |
| EASSLIS | (SEQ ID NO: 333) |
| EASALIS | (SEQ ID NO: 334) |
| RASTLIS | (SEQ ID NO: 335) |
| RASDLIS | (SEQ ID NO: 336) |
| RASELIS | (SEQ ID NO: 337) |
| RASHLIS | (SEQ ID NO: 338) |
| RASKLIS | (SEQ ID NO: 339) |
| RASSLIS | (SEQ ID NO: 340) |
| RASALIS | (SEQ ID NO: 341) |
| TASTLIS | (SEQ ID NO: 342) |
| TASDLIS | (SEQ ID NO: 343) |
| TASELIS | (SEQ ID NO: 344) |
| TASHLIS | (SEQ ID NO: 345) |
| TASKLIS | (SEQ ID NO: 346) |
| TASSLIS | (SEQ ID NO: 347) |
| TASALIS | (SEQ ID NO: 348) |
| SASTLIS | (SEQ ID NO: 349) |
| SASDLIS | (SEQ ID NO: 350) |
| SASELIS | (SEQ ID NO: 351) |
| SASHLIS | (SEQ ID NO: 352) |
| SASKLIS | (SEQ ID NO: 353) |
| SASSLIS | (SEQ ID NO: 354) |
| SASALIS | (SEQ ID NO: 355) |
| QLDNLQT | (SEQ ID NO: 356) |
| DLDNLQT | (SEQ ID NO: 357) |
| ELDNLQT | (SEQ ID NO: 358) |
| TLDNLQT | (SEQ ID NO: 359) |
| SLDNLQT | (SEQ ID NO: 360) |
| RLDTLQT | (SEQ ID NO: 361) |
| RLDDLQT | (SEQ ID NO: 362) |
| RLDELQT | (SEQ ID NO: 363) |
| RLDHLQT | (SEQ ID NO: 364) |
| RLDKLQT | (SEQ ID NO: 365) |
| RLDSLQT | (SEQ ID NO: 366) |
| RLDALQT | (SEQ ID NO: 367) |
| QLDTLQT | (SEQ ID NO: 368) |
| QLDDLQT | (SEQ ID NO: 369) |
| QLDELQT | (SEQ ID NO: 370) |
| QLDHLQT | (SEQ ID NO: 371) |
| QLDKLQT | (SEQ ID NO: 372) |
| QLDSLQT | (SEQ ID NO: 373) |
| QLDALQT | (SEQ ID NO: 374) |
| DLDTLQT | (SEQ ID NO: 375) |
| DLDDLQT | (SEQ ID NO: 376) |
| DLDELQT | (SEQ ID NO: 377) |
| DLDHLQT | (SEQ ID NO: 378) |
| DLDKLQT | (SEQ ID NO: 379) |
| DLDSLQT | (SEQ ID NO: 380) |
| DLDALQT | (SEQ ID NO: 381) |
| ELDTLQT | (SEQ ID NO: 382) |
| ELDDLQT | (SEQ ID NO: 383) |
| ELDELQT | (SEQ ID NO: 384) |
| ELDHLQT | (SEQ ID NO: 385) |
| ELDKLQT | (SEQ ID NO: 386) |
| ELDSLQT | (SEQ ID NO: 387) |
| ELDALQT | (SEQ ID NO: 388) |
| TLDTLQT | (SEQ ID NO: 389) |
| TLDDLQT | (SEQ ID NO: 390) |
| TLDELQT | (SEQ ID NO: 391) |
| TLDHLQT | (SEQ ID NO: 392) |
| TLDKLQT | (SEQ ID NO: 393) |
| TLDSLQT | (SEQ ID NO: 394) |
| TLDALQT | (SEQ ID NO: 395) |
| SLDTLQT | (SEQ ID NO: 396) |
| SLDDLQT | (SEQ ID NO: 397) |
| SLDELQT | (SEQ ID NO: 398) |
| SLDHLQT | (SEQ ID NO: 399) |
| SLDKLQT | (SEQ ID NO: 400) |
| SLDSLQT | (SEQ ID NO: 401) |
| SLDALQT | (SEQ ID NO: 402) |
| ARGTLRT | (SEQ ID NO: 403) |
| ARGDLRT | (SEQ ID NO: 404) |
| ARGELRT | (SEQ ID NO: 405) |
| ARGHLRT | (SEQ ID NO: 406) |
| ARGKLRT | (SEQ ID NO: 407) |
| ARGSLRT | (SEQ ID NO: 408) |
| ARGALRT | (SEQ ID NO: 409) |
| SRGTLRT | (SEQ ID NO: 410) |
| SRGDLRT | (SEQ ID NO: 411) |
| SRGELRT | (SEQ ID NO: 412) |
| SRGHLRT | (SEQ ID NO: 413) |
| SRGKLRT | (SEQ ID NO: 414) |
| SRGSLRT | (SEQ ID NO: 415) |
| SRGALRT | (SEQ ID NO: 416) |
| QKDALRG | (SEQ ID NO: 417) |
| DKDALRG | (SEQ ID NO: 418) |
| EKDALRG | (SEQ ID NO: 419) |
| TKDALRG | (SEQ ID NO: 420) |
| SKDALRG | (SEQ ID NO: 421) |
| RKDNLRG | (SEQ ID NO: 422) |
| RKDTLRG | (SEQ ID NO: 423) |
| RKDDLRG | (SEQ ID NO: 424) |
| RKDELRG | (SEQ ID NO: 425) |
| RKDHLRG | (SEQ ID NO: 426) |
| RKDKLRG | (SEQ ID NO: 427) |
| RKDSLRG | (SEQ ID NO: 428) |
| QKDNLRG | (SEQ ID NO: 429) |
| QKDTLRG | (SEQ ID NO: 430) |
| QKDDLRG | (SEQ ID NO: 431) |
| QKDELRG | (SEQ ID NO: 432) |
| QKDHLRG | (SEQ ID NO: 433) |
| QKDKLRG | (SEQ ID NO: 434) |
| QKDSLRG | (SEQ ID NO: 435) |
| DRDNLRG | (SEQ ID NO: 436) |
| DKDTLRG | (SEQ ID NO: 437) |
| DKDDLRG | (SEQ ID NO: 438) |
| DKDELRG | (SEQ ID NO: 439) |
| DKDHLRG | (SEQ ID NO: 440) |
| DKDKLRG | (SEQ ID NO: 441) |
| DKDSLRG | (SEQ ID NO: 442) |
| EKDNLRG | (SEQ ID NO: 443) |
| EKDTLRG | (SEQ ID NO: 444) |
| EKDDLRG | (SEQ ID NO: 445) |
| EKDELRG | (SEQ ID NO: 446) |
| ERDHLRG | (SEQ ID NO: 447) |
| EKDKLRG | (SEQ ID NO: 448) |
| EKDSLRG | (SEQ ID NO: 449) |
| TKDNLRG | (SEQ ID NO: 450) |
| TKDTLRG | (SEQ ID NO: 451) |
| TKDDLRG | (SEQ ID NO: 452) |
| TKDELRG | (SEQ ID NO: 453) |
| TKDHLRG | (SEQ ID NO: 454) |
| TKDKLRG | (SEQ ID NO: 455) |
| TKDSLRG | (SEQ ID NO: 456) |
| SKDNLRG | (SEQ ID NO: 457) |
| SKDTLRG | (SEQ ID NO: 458) |
| SKDDLRG | (SEQ ID NO: 459) |
| SKDELRG | (SEQ ID NO: 460) |
| SKDHLRG | (SEQ ID NO: 461) |
| SKDKLRG | (SEQ ID NO: 462) |
| SKDSLRG | (SEQ ID NO: 463) |
| VRGTLRT | (SEQ ID NO: 464) |
| VRGDLRT | (SEQ ID NO: 465) |
| VRGELRT | (SEQ ID NO: 466) |
| VRGHLRT | (SEQ ID NO: 467) |
| VRGKLRT | (SEQ ID NO: 468) |
| VRGSLRT | (SEQ ID NO: 469) |
| VRGTLRT | (SEQ ID NO: 470) |
| QLRALDR | (SEQ ID NO: 471) |
| DLRALDR | (SEQ ID NO: 472) |
| ELRALDR | (SEQ ID NO: 473) |
| TLRALDR | (SEQ ID NO: 474) |
| SLRALDR | (SEQ ID NO: 475) |
| RSDNRKR | (SEQ ID NO: 476) |
| RSDTRKR | (SEQ ID NO: 477) |
| RSDDRKR | (SEQ ID NO: 478) |
| RSDHRKR | (SEQ ID NO: 479) |
| RSDKRKR | (SEQ ID NO: 480) |
| RSDSRKR | (SEQ ID NO: 481) |
| RSDARKR | (SEQ ID NO: 482) |
| QYQSLRQ | (SEQ ID NO: 483) |
| EYQSLRQ | (SEQ ID NO: 484) |
| RYQSLRQ | (SEQ ID NO: 485) |
| TYQSLRQ | (SEQ ID NO: 486) |
| SYQSLRQ | (SEQ ID NO: 487) |
| RLRNIQF | (SEQ ID NO: 488) |
| RLRTIQF | (SEQ ID NO: 489) |
| RLREIQF | (SEQ ID NO: 490) |
| RLRHIQF | (SEQ ID NO: 491) |
| RLRKIQF | (SEQ ID NO: 492) |
| RLRSIQF | (SEQ ID NO: 493) |
| RLRAIQF | (SEQ ID NO: 494) |
| DSLLLGA | (SEQ ID NO: 495) |
| ESLLLGA | (SEQ ID NO: 496) |
| RSLLLGA | (SEQ ID NO: 497) |
| TSLLLGA | (SEQ ID NO: 498) |
| SSLLLGA | (SEQ ID NO: 499) |
| HRGNLGG | (SEQ ID NO: 500) |
| HRGDLGG | (SEQ ID NO: 501) |
| HRGELGG | (SEQ ID NO: 502) |
| HRGHLGG | (SEQ ID NO: 503) |
| HRGKLGG | (SEQ ID NO: 504) |
| HRGSLGG | (SEQ ID NO: 505) |
| HRGALGG | (SEQ ID NO: 506) |
| QKHMLDT | (SEQ ID NO: 507) |
| EKHMLDT | (SEQ ID NO: 508) |
| RKHMLDT | (SEQ ID NO: 509) |
| TKHMLDT | (SEQ ID NO: 510) |
| SKHMLDT | (SEQ ID NO: 511) |
| QLGGLRQ | (SEQ ID NO: 512) |
| ELGGLRQ | (SEQ ID NO: 513) |
| RLGGLRQ | (SEQ ID NO: 514) |
| TLGGLRQ | (SEQ ID NO: 515) |
| SLGGLRQ | (SEQ ID NO: 516) |
| AEANLQR | (SEQ ID NO: 517) |
| AEATLQR | (SEQ ID NO: 518) |
| AEADLQR | (SEQ ID NO: 519) |
| AEAHLQR | (SEQ ID NO: 520) |
| AEAKLQR | (SEQ ID NO: 521) |
| AEASLQR | (SEQ ID NO: 522) |
| AEAALQR | (SEQ ID NO: 523) |
| DGRCLVT | (SEQ ID NO: 524) |
| EGRCLVT | (SEQ ID NO: 525) |
| RGRCLVT | (SEQ ID NO: 526) |
| TGRCLVT | (SEQ ID NO: 527) |
| SGRCLVT | (SEQ ID NO: 528) |
| QEDNLHT | (SEQ ID NO: 529) |
| DEDNLHT | (SEQ ID NO: 530) |
| EEDNLHT | (SEQ ID NO: 531) |
| SEDNLHT | (SEQ ID NO: 532) |
| REDTLHT | (SEQ ID NO: 533) |
| REDDLHT | (SEQ ID NO: 534) |
| REDELHT | (SEQ ID NO: 535) |
| REDHLHT | (SEQ ID NO: 536) |
| REDKLHT | (SEQ ID NO: 537) |
| REDSLHT | (SEQ ID NO: 538) |
| REDALHT | (SEQ ID NO: 539) |
| QEDTLHT | (SEQ ID NO: 540) |
| QEDDLHT | (SEQ ID NO: 541) |
| QEDELHT | (SEQ ID NO: 542) |
| QEDHLHT | (SEQ ID NO: 543) |
| QEDKLHT | (SEQ ID NO: 544) |
| QEDSLHT | (SEQ ID NO: 545) |
| QEDALHT | (SEQ ID NO: 546) |
| DEDTLHT | (SEQ ID NO: 547) |
| DEDDLHT | (SEQ ID NO: 548) |
| DEDELHT | (SEQ ID NO: 549) |
| DEDHLHT | (SEQ ID NO: 550) |
| DEDKLHT | (SEQ ID NO: 551) |
| DEDSLHT | (SEQ ID NO: 552) |
| DEDALHT | (SEQ ID NO: 553) |
| EEDTLHT | (SEQ ID NO: 554) |
| EEDDLHT | (SEQ ID NO: 555) |
| EEDELHT | (SEQ ID NO: 556) |
| EEDHLHT | (SEQ ID NO: 557) |
| EEDKLHT | (SEQ ID NO: 558) |
| EEDSLHT | (SEQ ID NO: 559) |
| EEDALHT | (SEQ ID NO: 560) |
| TEDTLHT | (SEQ ID NO: 561) |
| TEDDLHT | (SEQ ID NO: 562) |
| TEDELHT | (SEQ ID NO: 563) |
| TEDHLHT | (SEQ ID NO: 564) |
| TEDKLHT | (SEQ ID NO: 565) |
| TEDSLHT | (SEQ ID NO: 566) |
| TEDALHT | (SEQ ID NO: 567) |
| SEDTLHT | (SEQ ID NO: 568) |
| SEDDLHT | (SEQ ID NO: 569) |
| SEDELHT | (SEQ ID NO: 570) |
| SEDHLHT | (SEQ ID NO: 571) |
| SEDKLHT | (SEQ ID NO: 572) |
| SEDSLHT | (SEQ ID NO: 573) |
| SEDALHT | (SEQ ID NO: 574) |
| QEDNLIS | (SEQ ID NO: 575) |
| DEDNLIS | (SEQ ID NO: 576) |
| EEDNLIS | (SEQ ID NO: 577) |
| SEDNLIS | (SEQ ID NO: 578) |
| REDTLIS | (SEQ ID NO: 579) |
| REDDLIS | (SEQ ID NO: 580) |
| REDELIS | (SEQ ID NO: 581) |
| REDHLIS | (SEQ ID NO: 582) |
| REDKLIS | (SEQ ID NO: 583) |
| REDSLIS | (SEQ ID NO: 584) |
| REDALIS | (SEQ ID NO: 585) |
| QEDTLIS | (SEQ ID NO: 586) |
| QEDDLIS | (SEQ ID NO: 587) |
| QEDELIS | (SEQ ID NO: 588) |
| QEDHLIS | (SEQ ID NO: 589) |
| QEDKLIS | (SEQ ID NO: 590) |
| QEDSLIS | (SEQ ID NO: 591) |
| QEDALIS | (SEQ ID NO: 592) |
| DEDTLIS | (SEQ ID NO: 593) |
| DEDDLIS | (SEQ ID NO: 594) |
| DEDELIS | (SEQ ID NO: 595) |
| DEDHLIS | (SEQ ID NO: 596) |
| DEDKLIS | (SEQ ID NO: 597) |
| DEDSLIS | (SEQ ID NO: 598) |
| DEDALIS | (SEQ ID NO: 599) |
| EEDTLIS | (SEQ ID NO: 600) |
| EEDDLIS | (SEQ ID NO: 601) |
| EEDELIS | (SEQ ID NO: 602) |
| EEDHLIS | (SEQ ID NO: 603) |
| EEDKLIS | (SEQ ID NO: 604) |
| EEDSLIS | (SEQ ID NO: 605) |
| EEDALIS | (SEQ ID NO: 606) |
| TEDTLIS | (SEQ ID NO: 607) |
| TEDDLIS | (SEQ ID NO: 608) |
| TEDELIS | (SEQ ID NO: 609) |
| TEDHLIS | (SEQ ID NO: 610) |
| TEDKLIS | (SEQ ID NO: 611) |
| TEDSLIS | (SEQ ID NO: 612) |
| TEDALIS | (SEQ ID NO: 613) |
| SEDTLIS | (SEQ ID NO: 614) |
| SEDDLIS | (SEQ ID NO: 615) |
| SEDELIS | (SEQ ID NO: 616) |
| SEDHLIS | (SEQ ID NO: 617) |
| SEDKLIS | (SEQ ID NO: 618) |
| SEDSLIS | (SEQ ID NO: 619) |
| SEDALIS | (SEQ ID NO: 620) |
| TGGWLQA | (SEQ ID NO: 621) |
| SGGWLQA | (SEQ ID NO: 622) |
| DGGWLQA | (SEQ ID NO: 623) |
| EGGWLQA | (SEQ ID NO: 624) |
| QGGWLQA | (SEQ ID NO: 625) |
| RGGTLQA | (SEQ ID NO: 626) |
| RGQDLQA | (SEQ ID NO: 627) |
| RGGELQA | (SEQ ID NO: 628) |
| RGGNLQA | (SEQ ID NO: 629) |
| RGGHLQA | (SEQ ID NO: 630) |
| RGGKLQA | (SEQ ID NO: 631) |
| RGGSLQA | (SEQ ID NO: 632) |
| RGGALQA | (SEQ ID NO: 633) |
| TGGTLQA | (SEQ ID NO: 634) |
| TGGDLQA | (SEQ ID NO: 635) |
| TGGELQA | (SEQ ID NO: 636) |
| TGGNLQA | (SEQ ID NO: 637) |
| TGGHLQA | (SEQ ID NO: 638) |
| TGGKLQA | (SEQ ID NO: 639) |
| TGGSLQA | (SEQ ID NO: 640) |
| TGGALQA | (SEQ ID NO: 641) |
| SGGTLQA | (SEQ ID NO: 642) |
| SGGDLQA | (SEQ ID NO: 643) |
| SGGFLQA | (SEQ ID NO: 644) |
| SGGNLQA | (SEQ ID NO: 645) |
| SGGHLQA | (SEQ ID NO: 646) |
| SGGKLQA | (SEQ ID NO: 647) |
| SGGSLQA | (SEQ ID NO: 648) |
| SGGALQA | (SEQ ID NO: 649) |
| DGGTLQA | (SEQ ID NO: 650) |
| DGGDLQA | (SEQ ID NO: 651) |
| DGGELQA | (SEQ ID NO: 652) |
| DGGNLQA | (SEQ ID NO: 653) |
| DGGHLQA | (SEQ ID NO: 654) |
| DGGKLQA | (SEQ ID NO: 655) |
| DGGSLQA | (SEQ ID NO: 656) |
| DGGALQA | (SEQ ID NO: 657) |
| EGGTLQA | (SEQ ID NO: 658) |
| EGGDLQA | (SEQ ID NO: 659) |
| EGGELQA | (SEQ ID NO: 660) |
| EGGNLQA | (SEQ ID NO: 661) |
| EGGHLQA | (SEQ ID NO: 662) |
| EGGKLQA | (SEQ ID NO: 663) |
| EGGSLQA | (SEQ ID NO: 664) |
| EGGALQA | (SEQ ID NO: 665) |
| QGGTLQA | (SEQ ID NO: 666) |
| QGGDLQA | (SEQ ID NO: 667) |
| QGGELQA | (SEQ ID NO: 668) |
| QGGNLQA | (SEQ ID NO: 669) |
| QGGHLQA | (SEQ ID NO: 670) |
| QGGRLQA | (SEQ ID NO: 671) |
| QGGSLQA | (SEQ ID NO: 672) |
| QGGALQA | (SEQ ID NO: 673) |
| Linkers | |
| TGEKP | (SEQ ID NO: 674) |
| TGGGGSGGGGTGEKP | (SEQ ID NO: 675) |
| LRQKDGGGSERP | (SEQ ID NO: 676) |
| LRQKDGERP | (SEQ ID NO: 677) |
| GGRGRGRGRQ | (SEQ ID NO: 678) |
| QNKKGGSGDGKKKKQHI | (SEQ ID NO: 679) |
| TGGERP | (SEQ ID NO: 680) |
| ATGEKP | (SEQ ID NO: 681) |
| GGGSGGGGEGP | (SEQ ID NO: 682) |
| Other DNA and Protein Sequences | |
| RSDXLVR | (SEQ ID NO: 683) |
| GCGTGGGCG | (SEQ ID NO: 684) |
| GCGNNNGCG | (SEQ ID NO: 685) |
| RSDELKR | (SEQ ID NO: 686) |
| GATCNNGCG | (SEQ ID NO: 687) |
| SPADLTN | (SEQ ID NO: 688) |
| HISNFCR | (SEQ ID NO: 689) |
| GCGTGGGCG | (SEQ ID NO: 690) |
| GATANNGCG | (SEQ ID NO: 691) |
| ERSKLRA | (SEQ ID NO: 692) |
| DPGHLRV | (SEQ ID NO: 693) |
| DPGSLRV | (SEQ ID NO: 694) |
| RSDNLKN | (SEQ ID NO: 695) |
| SRDALNV | (SEQ ID NO: 696) |
| VKDYLTK | (SEQ ID NO: 697) |
| KNWKLQA | (SEQ ID NO: 698) |
| AQYMLVV | (SEQ ID NO: 699) |
| QSTNLKS | (SEQ ID NO: 700) |
| LDFNLRT | (SEQ ID NO: 701) |
| RKDNMTA | (SEQ ID NO: 702) |
| QSSNLIT | (SEQ ID NO: 703) |
| QRSALTV | (SEQ ID NO: 704) |
| QSGSLTR | (SEQ ID NO: 705) |
| AGGAGGU | (SEQ ID NO: 706) |
| TCAGAACTCACCTGTTAGAC | (SEQ ID NO: 707) |
| TATATAGCGNNNGCGTATATATCAAGTCAATCGG | (SEQ ID NO: 708) |
| TCC | |
| GGACCGATTGACTTGA | (SEQ ID NO: 709) |
| GGAN¹′N¹′N¹′N²′N²′N²′N³′N³′N³′GGG | (SEQ ID NO: 710) |
| TTTT CCC N³N³N³N²N²N²N¹N¹N¹TCC | |
| GAGCTCATGGAAGTACCATAG(N)₁₀GAACGTCG | (SEQ ID NO: 711) |
| ATCACTCGAG-3′ | |
| GAGCTCATGGAAGTACCATAG(N)₁₂GAACGTCG | (SEQ ID NO: 712) |
| ATCACTCGAG-3′ | |
| GAGCTCATGGAAGTACCATAG(N)₂₁GAACGTCG | (SEQ ID NO: 713) |
| ATCACTCGAG-3′ | |
| GAGCTCATGGAAGTACCATAG | (SEQ ID NO: 714) |
| CTCGAGTGATCGACGTTC | (SEQ ID NO: 715) |

### ADVANTAGES OF THE INVENTION

The present invention provides a widely useful and flexible method of labeling peptides, polypeptides, and proteins with zinc finger tags and for using the labeled peptides, polypeptides, or proteins for many functions, including monitoring their location in cells, the labeling of cells by incorporating labeled cell-surface proteins, the assembly of a protein array that can be used to study the activity of the proteins bound to the array, or the analysis of double-stranded DNA for binding to zinc finger tags. The present invention also provides fusion proteins useful in carrying out these methods.

The present invention provides the ability to monitor the intracellular location and activity of proteins with less perturbation of their structure or function than currently available methods. The present invention also provides for the rapid construction of protein arrays without the need for independent protein expression and purification.

The fusion proteins, arrays, and methods of the present invention possess industrial applicability for the detection of components of the proteome and the analysis of activity of components of the proteome, including monitoring locations of these in cells and the assembly of protein arrays. These fusion proteins, arrays, and methods also possess industrial applicability for the preparation of medicaments to treat diseases and conditions that can be treated by the appropriate administration of such fusion proteins.

With respect to ranges of values, the invention encompasses each intervening value between the upper and lower limits of the range to at least a tenth of the lower limit's unit, unless the context clearly indicates otherwise. Moreover, the invention encompasses any other stated intervening values and ranges including either or both of the upper and lower limits of the range, unless specifically excluded from the stated range.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test this invention.

The publications and patents discussed herein are provided solely for their disclosure prior to the filing date of the present application. Nothing herein is to be construed as an admission that the present invention is not entitled to antedate such publication by virtue of prior invention. Further the dates of publication provided may be different from the actual publication dates which may need to be independently confirmed.

All the publications cited are incorporated herein by reference in their entireties, including all published patents, patent applications, literature references, as well as those publications that have been incorporated in those published documents. However, to the extent that any publication incorporated herein by reference refers to information to be published, applicants do not admit that any such information published after the filing date of this application to be prior art.

As used in this specification and in the appended claims, the singular forms include the plural forms. For example the terms “a,” “an,” and “the” include plural references unless the content clearly dictates otherwise. Additionally, the term “at least” preceding a series of elements is to be understood as referring to every element in the series. The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein. In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. Those skilled in the art will recognize, or will be able to ascertain using no more than routine experimentation, many equivalents to the specific embodiments of the invention described. Such equivalents are intended to be encompassed by the following claims.

I claim:

1. An array comprising:
(a) a solid support;
(b) a plurality of nucleotide sequences attached to the solid support; and
(c) a plurality of fusion proteins specifically and noncovalently bound to the plurality of nucleotide sequences, each fusion protein comprising: (1) a protein, peptide, or polypeptide of interest; and (2) a zinc finger protein tag, wherein each zinc finger protein tag has specific binding affinity for only one of the nucleotide sequences attached to the solid support.

2. The array of claim 1 wherein the plurality of nucleotide sequences are DNA sequences.

3. The array of claim 2 wherein each of the plurality of nucleotide sequences is of a length selected from the group consisting of 3 base pairs, 6 base pairs, 9 base pairs, 12 base pairs, 15 base pairs, and 18 base pairs.

4. The array of claim 3 wherein each of the plurality of nucleotide sequences is 18 base pairs.

5. The array of claim 1 wherein the solid support is glass.

6. The array of claim 5 wherein the glass is activated by reaction with 1,4-diphenylene-diisothiocyanate.

7. The array of claim 1 wherein each fusion protein includes the same peptide, polypeptide, or protein of interest.

8. The array of claim 7 wherein the peptide, polypeptide, or protein of interest is an antibody molecule.

9. The array of claim 1 in which all of the nucleotide sequences and zinc finger tags are identical.

10. The array of claim 1 in which a plurality of different nucleotide sequences are attached to the solid support in defined locations, and a plurality of different zinc finger tags is used, each zinc finger tag used specifically binding a particular nucleotide sequence.

11. The array of claim 1 wherein at least one of the zinc finger protein tags of the fusion proteins has at least one zinc finger DNA binding domain therein specifically binding a DNA subsite of the structure 5′-ANN-3′.

12. The array of claim 11 wherein the at least one zinc finger DNA binding domain specifically binding a DNA subsite of the structure 5′-ANN-3′ is selected from the group consisting of SEQ ID NO: 1 through SEQ ID NO: 70.

13. The array of claim 1 wherein at least one of the zinc finger protein tags of the fusion proteins has at least one zinc finger DNA binding domain therein specifically binding a DNA subsite of the structure 5′-AGC-3′.

14. The array of claim 13 wherein the at least one zinc finger DNA binding domain specifically binding a DNA subsite of the structure 5′-AGC-3′ is selected from the group consisting of SEQ ID NO: 70 through SEQ ID NO: 127.

15. The array of claim 1 wherein at least one of the zinc finger protein tags of the fusion proteins has at least one zinc finger DNA binding domain therein specifically binding a DNA subsite of the structure 5′-CNN-3′.

16. The array of claim 15 wherein the at least one zinc finger DNA binding domain specifically binding a DNA sub site of the structure 5′-CNN-3′ is selected from the group consisting of SEQ ID NO: 128 through SEQ ID NO: 152.

17. The array of claim 1 wherein at least one of the zinc finger protein tags of the fusion proteins has at least one zinc finger DNA binding domain therein specifically binding a DNA subsite of the structure 5′-GNN-3′.

18. The array of claim 17 wherein the at least one zinc finger DNA binding domain specifically binding a DNA subsite of the structure 5′-GNN-3′ is selected from the group consisting of SEQ ID NO: 153 through SEQ ID NO: 262.

19. The array of claim 1 wherein at least one of the zinc finger protein tags of the fusion proteins has at least one zinc finger DNA binding domain therein specifically binding a DNA subsite of the structure 5′-TNN-3′.

20. The array of claim 19 wherein the at least one zinc finger DNA binding domain specifically binding a DNA subsite of the structure 5′-TNN-3′ is selected from the group consisting of SEQ ID NO: 263 through SEQ ID NO: 673.

21. The array of claim 1 wherein at least one of the zinc finger protein tags of the fusion proteins has at least one zinc finger DNA binding domain therein specifically binding at least one DNA subsite of the structure 5′-ANN-3′ and at least one zinc finger DNA binding domain therein specifically binding at least one DNA subsite of a structure selected from the group consisting of 5′-CNN-3′, 5′-GNN-3′, and 5′-TNN-3′.

22. The array of claim 1 wherein at least one of the zinc finger protein tags of the fusion proteins has at least one zinc finger DNA binding domain therein specifically binding at least one DNA subsite of the structure 5′-CNN-3′ and at least one zinc finger DNA binding domain therein specifically binding at least one DNA subsite of a structure selected from the group consisting of 5′-ANN-3′, 5′-GNN-3′, and 5′-TNN-3′.

23. The array of claim 1 wherein at least one of the zinc finger protein tags of the fusion proteins has at least one zinc finger DNA binding domain therein specifically binding at least one DNA subsite of the structure 5′-GNN-3′ and at least one zinc finger DNA binding domain therein specifically binding at least one DNA subsite of a structure selected from the group consisting of 5′-ANN-3′, 5′-CNN-3′, and 5′-TNN-3′.

24. The array of claim 1 wherein at least one of the zinc finger protein tags of the fusion proteins has at least one zinc finger DNA binding domain therein specifically binding at least one DNA subsite of the structure 5′-TNN-3′ and at least one zinc finger DNA binding domain therein specifically binding at least one DNA subsite of a structure selected from the group consisting of 5′-ANN-3′, 5′-CNN-3′, and 5′-GNN-3′.

25. The array of claim 1 wherein at least one of the zinc finger tags of the fusion proteins has a C₂H₂ framework subdomain.

26. The array of claim 1 wherein at least one of the zinc finger tags of the fusion proteins has a framework subdomain selected from the group consisting of C₃H, C₄, H₄, CH₃, and C₆.

27. The array of claim 1 wherein at least one of the zinc finger tags of the fusion proteins has a framework subdomain that is based on aPP.

28. The array of claim 1 wherein at least one of the fusion proteins includes a linker therein.

29. A method for assaying activity of a peptide, polypeptide, or protein of interest comprising the steps of:
(a) providing the array of claim 1;
(b) contacting the array with a reagent that reacts with a peptide, polypeptide, or protein of interest that may or not be present in the array to produce a detectable product; and
(c) determining the location of a peptide, polypeptide, or protein in the array by determining the location of the detectable product in order to identify the location of a peptide, polypeptide, or protein that has a defined activity associated with the production of the detectable product.

30. The method of claim 29 wherein the defined activity is selected from the group consisting of enzymatic activity, binding activity, and regulatory activity.

31. A fusion protein comprising:
(a) a protein, polypeptide, or peptide of interest; and
(b) at least one zinc finger tag in a single polypeptide;
such that the protein, polypeptide, or protein of interest substantially maintains its three-dimensional conformation and activity, and the zinc finger tag substantially maintains its sequence-specific nucleotide sequence binding activity.

32. The fusion protein of claim 31 wherein the zinc finger tag specifically binds a nucleotide sequence that is 3, 6, 9, 12, 15, or 18 bases long.

33. The fusion protein of claim 32 wherein the zinc finger tag specifically binds a nucleotide sequence that is 18 bases long.

34. The fusion protein of claim 31 wherein the peptide, polypeptide or protein of interest and the zinc finger tag are joined end-to-end in a single reading frame.

35. The fusion protein of claim 31 wherein the peptide, polypeptide or protein of interest and the zinc finger tag are joined through a linker.

36. The fusion protein of claim 35 wherein the fusion protein further includes a purification tag.

37. The fusion protein of claim 31 wherein the fusion protein further includes a detectable protein moiety.

38. The fusion protein of claim 31 wherein the fusion protein includes a protein of interest that is selected from the group consisting of an antibody, an enzyme, a reporter protein, a receptor protein, a ligand for a receptor protein, a regulatory protein, and a membrane protein.

39. The fusion protein of claim 38 wherein the protein of interest is an antibody.

40. The fusion protein of claim 31 wherein the protein of interest is a peptide.

41. The fusion protein of claim 40 wherein the peptide is selected from the group consisting of a neurotransmitter and a hormone.

42. The fusion protein of claim 31 wherein the zinc finger protein tag of the fusion protein has at least one zinc finger DNA binding domain therein specifically binding a DNA subsite of the structure 5′-ANN-3′.

43. The fusion protein of claim 42 wherein the at least one zinc finger DNA binding domain specifically binding a DNA subsite of the structure 5′-ANN-3′ is selected from the group consisting of SEQ ID NO: 1 through SEQ ID NO: 70.

44. The fusion protein of claim 31 wherein the zinc finger protein tag of the fusion protein has at least one zinc finger DNA binding domain therein specifically binding a DNA subsite of the structure 5′-AGC-3′.

45. The fusion protein of claim 44 wherein the at least one zinc finger DNA binding domain specifically binding a DNA subsite of the structure 5′-AGC-3′ is selected from the group consisting of SEQ ID NO: 70 through SEQ ID NO: 127.

46. The fusion protein of claim 31 wherein the zinc finger protein tag of the fusion protein has at least one zinc finger DNA binding domain therein specifically binding a DNA subsite of the structure 5′-CNN-3′.

47. The fusion protein of claim 46 wherein the at least one zinc finger DNA binding domain specifically binding a DNA subsite of the structure 5′-CNN-3′ is selected from the group consisting of SEQ ID NO: 128 through SEQ ID NO: 152.

48. The fusion protein of claim 31 wherein the zinc finger protein tag of the fusion protein has at least one zinc finger DNA binding domain therein specifically binding a DNA subsite of the structure 5′-GNN-3′.

49. The fusion protein of claim 48 wherein the at least one zinc finger DNA binding domain specifically binding a DNA subsite of the structure 5′-GNN-3′ is selected from the group consisting of SEQ ID NO: 153 through SEQ ID NO: 262.

50. The fusion protein of claim 31 wherein the zinc finger protein tag of the fusion protein has at least one zinc finger DNA binding domain therein specifically binding a DNA subsite of the structure 5′-TNN-3′.

51. The fusion protein of claim 31 wherein the at least one zinc finger DNA binding domain specifically binding a DNA subsite of the structure 5′-TNN-3′ is selected from the group consisting of SEQ ID NO: 263 through SEQ ID NO: 673.

52. The fusion protein of claim 31 wherein the zinc finger protein tag of the fusion protein has at least one zinc finger DNA binding domain therein specifically binding at least one DNA subsite of the structure 5′-ANN-3′ and at least one zinc finger DNA binding domain therein specifically binding at least one DNA subsite of a structure selected from the group consisting of 5′-CNN-3′, 5′-GNN-3′, and 5′-TNN-3′.

53. The fusion protein of claim 31 wherein the zinc finger protein tag of the fusion protein has at least one zinc finger DNA binding domain therein specifically binding at least one DNA subsite of the structure 5′-CNN-3′ and at least one zinc finger DNA binding domain therein specifically binding at least one DNA subsite of a structure selected from the group consisting of 5′-ANN-3′, 5′-GNN-3′, and 5′-TNN-3′.

54. The fusion protein of claim 31 wherein the zinc finger protein tag of the fusion protein has at least one zinc finger DNA binding domain therein specifically binding at least one DNA subsite of the structure 5′-GNN-3′ and at least one zinc finger DNA binding domain therein specifically binding at least one DNA subsite of a structure selected from the group consisting of 5′-ANN-3′, 5′-CNN-3′, and 5′-TNN-3′.

55. The fusion protein of claim 31 wherein the zinc finger protein tag of the fusion protein has at least one zinc finger DNA binding domain therein specifically binding at least one DNA subsite of the structure 5′-TNN-3′ and at least one zinc finger DNA binding domain therein specifically binding at least one DNA subsite of a structure selected from the group consisting of 5′-ANN-3′, 5′-CNN-3′, and 5′-GNN-3′.

56. The fusion protein of claim 31 wherein the zinc finger tag of the fusion protein has a C₂H₂ framework subdomain.

57. The fusion protein of claim 31 wherein the zinc finger tag of the fusion protein has a framework subdomain selected from the group consisting of C₃H, C₄, H₄, CH₃, and C₆.

58. The fusion protein of claim 31 wherein the zinc finger tag of the fusion protein has a framework subdomain that is based on aPP.

59. The fusion protein of claim 31 wherein the fusion protein includes a linker therein.

60. A polynucleotide encoding the fusion protein of claim 31.

61. The polynucleotide of claim 60 that is DNA.

62. A vector including the DNA of claim 61.

63. The vector of claim 62 wherein the vector further includes a reporter gene.

64. The vector of claim 62 wherein the vector further includes a positive selection marker.

65. The vector of claim 62 wherein the vector is a recombinant DNA (rDNA) molecule containing a nucleotide sequence that codes for and is capable of expressing a fusion polypeptide containing, in the direction of amino- to carboxy-terminus, (1) a prokaryotic secretion signal domain, (2) a heterologous polypeptide, and (3) a filamentous phage membrane anchor domain.

66. A host cell transformed or transfected with the vector of claim 62.

67. A method of expressing a fusion protein comprising the steps of:
(a) introducing the vector of claim 62 into a compatible host cell; and
(b) causing the fusion protein to be expressed in the host cell; and
(c) isolating the expressed fusion protein.

68. A method for in vivo localization of a target protein in a cell comprising the steps of;
(a) expressing the fusion protein of claim 31 in a cell, the target protein being incorporated in the fusion protein;
(b) introducing a DNA molecule into the cell that is specifically bound by the zinc finger tag of the fusion protein, wherein the DNA molecule is covalently labeled with a fluorescent indicator molecule;
(c) incubating the cell so that the DNA molecule binds to the fusion protein; and
(d) localizing the target protein in the cell by locating the fluorescent indicator molecule.

69. The method of claim 68 wherein the DNA molecule is in a hairpin conformation with a stem and loop in which the stem is double-stranded and the loop has unpaired bases.

70. The method of claim 68 wherein the fluorescent indicator molecule is covalently bound to the DNA molecule.

71. The method of claim 68 wherein the target protein is localized in a cellular organelle selected from the group consisting of the nucleus, the nucleolus, the endoplasmic reticulum, the nuclear membrane, the cell membrane, the Golgi apparatus, the mitochondria, the chloroplast, the peroxisome.

72. The method of claim 68 wherein the target protein is selected from the group consisting of an antibody, an enzyme, a reporter protein, a receptor protein, a ligand for a receptor protein, a regulatory protein, and a membrane protein.

73. A method for labeling the cell membrane of a cell comprising the steps of:
(a) transforming or transfecting a host cell with a nucleic acid sequence that encodes a fusion protein that is a fusion of a membrane protein with a zinc finger tag such that the cell expresses the fusion protein;
(b) culturing the transformed or transfected cell under conditions such that the fusion protein is expressed and is incorporated in the cell membrane of the cell;
(c) contacting the cell expressing the fusion protein incorporated in the membrane with a labeled DNA molecule that binds the zinc finger tag of the fusion protein in a sequence-specific manner; and
(d) detecting the label of the labeled DNA molecule on the cell surface.

74. The method of claim 73 wherein the membrane protein is a transmembrane protein that includes an extracellular domain, a transmembrane domain, and an intracellular domain.

75. A cell including therein the fusion protein of claim 46 wherein the fusion protein includes therein a membrane protein, such that the fusion protein is incorporated into the cell membrane.

76. A method of cross-linking cells comprising the steps of:
(a) providing cells of claim 75;
(b) labeling the cells with DNA;
(c) arraying the cells on DNA surfaces; and
(d) cross-linking the cells on the DNA surfaces.

77. The method of claim 76 further comprising the step of contacting the cross-linked cells with a probe to study cell surface interactions.

78. The method of claim 77 wherein the probe is selected from the group consisting of a labeled antibody and a labeled receptor ligand.

79. A method of analyzing double-stranded DNA comprising the steps of:
(a) providing a plurality of fusion proteins of claim 31;
(b) binding the fusion proteins to a solid support, each fusion protein being attached at a defined nonoverlapping location on the solid support, to produce a fusion protein microarray;
(c) exposing the fusion protein to a sample containing one or more double-stranded DNA molecules so that any double-stranded DNA molecules possessing a defined nucleotide sequence bound by a zinc finger tag incorporated in a fusion protein is bound; and
(d) analyzing the binding of DNA molecules to the fusion proteins in order to determine whether DNA molecules possessing any of the defined nucleotide sequences are present in the sample.

80. The method of claim 79 wherein the fusion proteins are bound covalently to the solid support.

81. The method of claim 79 wherein the fusion proteins are bound noncovalently to the solid support.

82. An array comprising:
(a) a solid support;
(b) a plurality of fusion proteins of claim 31 attached to the solid support.
